SEQUENCE LISTING (10 sequences)

SEQ ID NO: 1 - DNA, 1136 bp, Homo Sapiens
gtagaagggattacaaagtggaaatagacaccaaatcttactggaaggcacttggcatctccccattccatgagcatgcagaggtggtattcacagccaacgactccggcccccgccgctacaccattgccgccctgctgagcccctactcctattccaccacggctgtcgtcaccaatcccaaggaatgagggacttctcctccagtggacctgaaggacgagggatgggatttcatgtaaccaagagtattccatttttactaaagcagtgttttcacctcatatgctatgttagaagtccaggcagagacaataaaacattcctgtgaaaggcaaaaaaaaaaaaaaaaaactcgaggtcgacggtatcgataagcttgatatcgaattcggcacgagcggcccttcaggatgaaagctgcggtgctgaccttggccgtgctcttcctgacggggagccaggctcggcatttctggcagcaagatgaacccccccagagcccctgggatcgagtgaaggacctggccactgtgtacgtggatgtgctcaaagacagcggcagagactatgtgtcccagtttgaaggctccgccttgggaaaacagctaaacctaaagctccttgacaactgggacagcgtgacctccaccttcagcaagctgcgcgaacagctcggccctgtgacccaggagttctgggataacctggaggccaaggtgcagccctacctggacgacttccagaagaagtggcaggaggagatggagctctaccgccagaaggtggagccgctgcgcgcagagctccaagagggcgcgcgccagaagctgcacgagctgcaagagaagctgagcccactgggcgaggagatgcgcgaacgcgcgcgcgcccatgtggacgcgctgcgcacgcatctggcccccctacagcgacgagctgcgccagcgcttggccgcgcgcccttgaggctctcaaggagaacggcggcgccagactggccgagtaccacgccaaggccaccgagcatctgagcacgctcagcgagaaggccaagcccgcgctcgaggacctcccgccaggcctgctgcccgtgctggagagcttcaggtcagctcctgagactctccaagata

SEQ ID NO: 2 - protein, 216 aa, Homo Sapiens
YRIRHERPFRMKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEAKVQPYLDDFQKKWQEEMELYRQKVEPLRAELQEGARQKLHELQEKLSPLGEEMRERARAHVDALRTHLAPLQRRAAPALGRAPLRLSRRTAAPDWPSTTPRPPSI

SEQ ID NO: 3 - DNA, 1868 bp, Homo Sapiens
ctcccagcaaggccggatgccagtttagggcatgatgttggcgacgctcctccacagggcgggaaggttgatgaaaggcgtgttgaccgaggccccagaggctaacggcgtggttgtgcccctccttgatggctggaagatccgctttgatggttgccattcagcagctggttggcctccttgctggcttggttgaccccattatgagcattctgcagctccttcccggcctggccagcagcatggtgggcaccttggccaagcttctccacttcctttccagcctggtcagcagcatggttgaccccttggccaagtttctctgcttccttcccagcctggtggaccccagtgtggaacccttggaccgctttgtctgcttccttcccggcctgttcaagggtatggtgaactccctggccaaactgccctgcctccttcccggcctcgtggaccccaggttggacaccatgaactgctatgtctccctctttcccagcctgccctgctgtgtggtgaatgtcgtggccaaactgccccccctcccttccagcctgactaacgccatgatgcgaggccttgggacgcactgctgtgcgtgcctgccttccgccatccgtgcgagtgcagcatggtggacatccctggccaaacttctctgcttccttccaggcctcattaaccccagggtggaccccagggccgagcttctctgtttccttcccaaattgactggcagcatggtggaccccctggccaaacttctctgtctccttccagccctcactgagaccatgggggaccccgctgggcccaacctccaagcctcatttccggcctgccccgcagcagtggtgggccccctggccaaatctcccttgcctcatttccgggcctgccccgcagcatggtgggccccctggccaaacttctctgcctccttcccaacctgaccagcagtatggtggaccccctggccaaacttctctgtctccttccagccctcactgagaccatggtggaccccctggccaaatctccctgcctcatttccggcctgccccgcagcatggtgggccccctggccaaacttctctgcctccttcccaacctgaccggcagcatggtggatcccctggccaaacttctctgtctccttccagccctcactgagaccatggtgggccccctggccaaatctcccagcctcatttccggcctgccctgcagcatggtggactccctggccaaacctcccagcctcatttccggcctgccctgcagcatggtggactccctggccaaacctcccagcctcatttccagcctgccctgcagcattgtcgactccctggccaaacttccctgcctcacttcccgcctggttggccccgtgatggaccccatgatggatcagtttgtctgcctccttcccaacctgtccagcagcgttgttgaccccatggccaagcttctctgcttcctttcctgcttgtccaataccatggttgatctcatgggcaaccttgtccatgccgtggttgagcccccggacgcctttgtccaactccttgccggtgtggctccccatgttgctaagtccgttgaaaaccttctccacttcccttccggcatgcgtgattccactgttgatgccatccagggccttgcccacctctctctctgcattgctcagccctcggttgatcccttcaatgaccttctcaatggggtcatcgctggccgcccatccagacagggcccccagtagcagaaggagggagcaggagccgaccagacgtgcaagatgcatattgctgggaaggtcgggaaggatgcagagaggagccagggaagccacgctgcta

SEQ ID NO: 4 - protein, 327 aa, Homo Sapiens
SSVASLAPLCILPDLPSNMHLARLVGSCSLLLLLGALSGWAASDDPIEKVIEGINRGLSNAEREVGKALDGINSGITHAGREVEKVFNGLSNMGSHTGKELDKGVRGLNHGMDKVAHEINHGIGQAGKEAEKLGHGVNNAAGQVGKEADKLIHHGVHHGANQAGSEAGKFGQGVDNAAGQAGNEAGRFGQGVHHAAGQAGNEAGRFGQGVHHAAGQAGNEAGRFGQGAHHGLSEGWKETEKFGQGIHHAAGQVGKEAEKFGQGAHHAAGQAGNEAGRFGQGVHHGLSEGWKETEKFGQGVHHTAGQVGKEAEKFGQGAHHAAGQARK

SEQ ID NO: 5 - DNA, 468 bp, Homo Sapiens
  misc_feature (1)...(468): n = A,T,C or G
acgcgtccgggaagcaacactggtggtgcctcagccatggcctggaccgttctcctcctcggcctcctctctcactgcacaggctctgtgacgtccaatgtgctgactcagccaccctcggtgtcagtggccccaggaaggacggccaggatcacctgtgagagagacaacattgaaagtaaaagtgttcactggtaccagcagaagccaggccaggcccctgtgttggtcgtctacgatgatcgtgagcggccctcagggatccctgaccggttctctggctccaactctgggaacacggccatcctcaccatcagcagggtcgaagccggggatgaggccgactatttttgtcacgtgtgggatggtgacagagatcaagcgcatgtggttttcggcngggggaccaagctgaccgtcctagtcagcccaaggctgccccctcggtcactctgttcccgccctcct

SEQ ID NO: 6 - protein, 156 aa, Homo Sapiens
  VARIANT (1)...(156): Xaa = Any Amino Acid
TRPGSNTGGASAMAWTVLLLGLLSHCTGSVTSNVLTQPPSVSVAPGRTARITCERDNIESKSVHWYQQKPGQAPVLVVYDDRERPSGIPDRFSGSNSGNTAILTISRVEAGDEADYFCHVWDGDRDQAHVVFGXGTKLTVLVSPRLPPRSLCSRPP

SEQ ID NO: 7 - DNA, 564 bp, Homo Sapiens
cgcgatctagaactgacaagtccaactcttaccggcatcgtgaagtctctttcttggcattggatgaacagaaagtttgctccgctcaggatgttgccagggattactccaatcccaaatgggatgaaacctcacttggcttcctcgaaaagcaaagtgatcttgaagaggtgaaaggacaagaaacagttgctcccaggctcagcaggggaccgctgagagtggacaagcatgaaatcccccaggagtcactggatggatgttgcttgactccttccatccttcctgacctgactccctcctaccacccttattggagcactttgtactcttttgaagacaagcaagtcagcttggctcttgtagacaaaattaaaaaggatcaagaggagatagaagaccaaagcccaccatgccccaggctcagccaggagctgccagaggtgaaggagcaggaagtcccagaggactctgtgaatgaagtttacttgactccctcagttcaccatgacgtgtctgactgccaccagccttatagcagcaccttgtcctcattggaggatc

SEQ ID NO: 8 - protein, 188 aa, Homo Sapiens
  VARIANT (1)...(188): Xaa = Any Amino Acid
RSRTDKSNSYRHREVSFLALDEQKVCSAQDVARDYSNPKWDETSLGFLEKQSDLEEVKGQETVAPRLSRGPLRVDKHEIPQESLDGCCLTPSILPDLTPSYHPYWSTLYSFEDKQVSLALVDKIKKDQEEIEDQSPPCPRLSQELPEVKEQEVPEDSVNEVYLTPSVHHDVSDCHQPYSSTLSSLEDX

SEQ ID NO: 9 - DNA, 870 bp, Homo Sapiens
  misc_feature (1)...(870): n = A,T,C or G
nngtatcccggggagtaagaacctagggtacagtgtcccccatgggaaagcagtgcttaccctcttggatgtagcttgggaccacagtaacagaagtatttacagtttgatttttgtaacaggagaaatcatattttcctctaagttgagacacattcttccatcttcctaccttacaccctcccccattcctggcagctctgaaacacttgctcaagccttgcttaatatagcaaaagcagctaatctgttatcatctgaggtcagaactaacattcagaaagttctcctaagggtttgggtcttcattccaagtcagtcatcctacccaggagaccacacctcacccaccttcatgcactccacactccaatcccacacaccttctccccactccccccccacacaaaccctaaaccctaagcagaatgacattaagataataatttgtaattgcaatacacacagcaggcattcaaaaatggaagaagaaaggaaaagctgcagaaaaatctttaagtactccacaaactggcacaagccacagacggattggaattaatcaacgctggactttcctagggaaaatagcattggctatttaaaacaggctggagtcatccaaatattaggcagttataaaccaaactttctacatcatccagtctagtgggtctggcaaacataccctatggaccaggtccaacctggctggcctggtttttggtaaatttaactgggtcatcagggcataacttcacacccatttggtttacctggctaatctggtaaacagaagtttcagcggaatagttccctcttttaaaagcagagttgaatagttctggagagtaggctctgcagggatgn

SEQ ID NO: 10 - protein, 186 aa, Homo Sapiens
  VARIANT (1)...(186): Xaa = Any Amino Acid
XIPGSKNLGYSVPHGKAVLTLLDVAWDHSNRSIYSLIFVTGEIIFSSKLRHILPSSYLTPSPIPGSSETLAQALLNIAKAANLLSSEVRTNIQKVLLRVWVFIPSQSSYPGDHTSPTFMHSTLQSHTPSPHSPPTQTLNPKQNDIKIIICNCNTHSRHSKMEEERKSCRKIFKYSTNWHKPQTDWN

(19) United States
(12) Patent Application Publication
(10) Pub. No.: US 2003/0224386 A1
(43) Pub. Date: Dec. 4, 2003

(54) Compositions, kits, and methods for identification, assessment, prevention, and therapy of rheumatoid arthritis

(75) Inventors: Braydon C. Guild, Concord, MA (US); Hua Liao, Newton, MA (US); Michael D. Jones, Arlington, MA (US); Johannes W. Zolg, Weilheim (DE); Jiang Wu, Waltham, MA (US)

Correspondence Address: Theodore R. Allen, Millennium Pharmaceuticals, Inc., 75 Sidney Street, Cambridge, MA, 02139, US

(73) Assignee: Millennium Pharmaceuticals, Inc.

(21) Appl. No.: 10/320,352

(22) Filed: Dec. 16, 2002

Related U.S. Application Data
(60) Provisional application No. 60/341,942, filed on Dec. 19, 2001.

(51) Int. Cl. C12Q 1/68

(52) U.S. Cl. 435/6

(57) ABSTRACT

The invention relates to compositions, kits, and methods for detecting, characterizing, preventing, and treating human Rheumatoid Arthritis (RA). A variety of newly-identified markers are provided, wherein changes in the levels of expression of one or more of the markers is correlated with RA.

### RELATED APPLICATIONS

The present application claims priority from U.S. provisional patent application serial No. 60/341,942, filed on Dec. 19, 2001. The above application is expressly incorporated by reference.

### FIELD OF THE INVENTION

The field of the invention is rheumatoid arthritis, including diagnosis, characterization, management, and therapy of rheumatoid arthritis.

### BACKGROUND OF THE INVENTION

Rheumatoid arthritis (“RA”) is a chronic, inflammatory, systemic disease that produces its most prominent manifestations in the diarthrodial joints. Persistent and progressive synovitis develops in peripheral joints. RA encompasses a wide spectrum of features, from self-limiting disease to progressively chronic disease with varying degrees of joint destruction to clinically evident extra-articular manifestations. Genetic and environmental factors control the progression, extent, and pattern of the inflammatory response and are thereby responsible for the heterogeneous clinical features.

RA has a worldwide distribution and involves all ethnic groups. Although the disease can occur at any age, the prevalence increases with age and the peak incidence is between the fourth and sixth decade, although data from population-based prevalence and incidence studies have to be interpreted cautiously because there is no laboratory test, histologic finding, or radiographic-feature to confirm a diagnosis of RA.

The most widely used system to classify RA is the American College of Rheumatology 1987 revised criteria for the classification of RA. Arnett F C, et al., 1988, The American Rheumatism Association 1987 revised criteria for the classifaction of rheumatoid arthritis. Arthritis Rheum 31:315-324. According to the criteria, a patient is said to have RA if the patient satisfies at least four of the following seven criteria and criteria 1-4 must be present for at least six weeks: 1) morning stiffness, 2) arthritis of three or more joint areas, 3) arthritis of hand joints, 4) symmetrical arthritis, 5) rheumatoid nodules, 6) serum rheumatoid factor (“RF”), and 7) radiographic changes. These criteria have a sensitivity and specificity of approximately 90%. Depending on the stringency of the criteria, prevalence estimates vary from 0.3% to 1.5% in the North American population. The prevalence is about 2.5 times higher in females than in males.

The histologic changes in RA are not disease-specific but largely depend on the organ involved. The primary inflammatory joint lesion involves the synovium. The earliest changes are injury to the synovial microvasculature with occlusion of the lumen, swelling of endothelial cells, and gaps between endothelial cells, as documented by electron microscopy. This stage is usually associated with mild proliferation of the superficial lining cell layer. Two cell types constitute the synovial lining: bone marrow-derived type A synoviocyte, which has macrophage features, and mesenchymal type B synoviocyte. Both cell types contribute to the synovial hyperplasia, suggesting a paracrine interaction between these two cell types. This stage of inflammation is associated with congestion, edema, and fibrin exudation. Cellular infiltration occurs in early disease and initially consists mainly of T lymphocytes. As a consequence of inflammation, the synovium becomes hypertrophic from the proliferation of blood vessels and synovial fibroblasts and from multiplication and enlargement of the synovial lining layers. Granulation tissue extends to the cartilage and is known as pannus. The tissue actively invades and destroys the periarticular bone and cartilage at the margin between synovium and bone, known as erosive RA.

The articular manifestations of RA can be placed in two categories: reversible signs and symptoms related to inflammatory synovitis and irreversible structural damage caused by synovitis. This concept is useful not only for staging disease and determining prognosis but also for selecting medical or surgical treatment. Structural damage in the typical patient usually begins sometime between the first and second year of the disease. Van der Heijde, D M, et al., 1982, Arthritis Rheum 25:361-365. Although synovitis tends to follow a fluctuating pattern, structural damage progresses as a linear function of the amount of prior synovitis.

The etiology of the early events in RA remains elusive. The possibility of a bacterial or viral infection has been vigorously pursued. All efforts to associate an infectious agent with RA by isolation, electron microscopy, or molecular biology have failed. It is possible that there is no single primary cause of RA and that different mechanisms may lead to the initial tissue injury and precipitate synovial inflammation.

Clinical signs of synovitis may be subtle and are often subjective. Warm, swollen, obviously inflamed joints are usually seen only in the most active phases of inflammatory synovitis. Cartilage loss and erosion of periarticular bone are the characteristic features of structural damage. The clinical features related to structural damage are marked by progressive deterioration functionally and anatomically. Structural damage to the joint is irreversible and additive.

Data from longitudinal clinical and epidemiologic studies provide guidelines for treatment. These studies emphasize 1) the need for early diagnosis, 2) identification of prognostic factors, and 3) early aggressive treatment. Earlier diagnosis and treatment, preferably within the first several months after onset of symptoms, may help prevent irreversible joint damage. The present invention provides such methods and reagents for the diagnosis, characterization, prognosis, monitoring and treatment of RA.

### SUMMARY OF THE INVENTION

The present invention is directed to the methods of determining or diagnosing whether patients are afflicted with inflammatory disorders, e.g., joint disorders, i.e., rheumatoid arthritis. The present invention also provides methods for determining or diagnosing whether patients are afflicted with erosive RA. Erosive RA is characterized by erosions or pits in the surface of the bone adjacent to the articular surface. In particular, in erosive RA, the granulation tissue actively invades and destroys the periarticular bone and cartilage at the margin between the synovium and the bone. These methods typically include the step of obtaining a sample of the patient's bodily fluids, determining the level of expression of one or more markers in these fluids, and identifying whether the patient's fluids include a pattern or profile of expression of a marker set (a pattern or profile of expression is also referred to herein as the “expression or marker profile” of the marker set) which correlates with inflammatory disorders, e.g. rheumatoid arthritis.

The present invention therefore provides methods, reagents and kits for diagnosing, characterizing, prognosing, monitoring, and treating RA, including identifying erosive and non-erosive RA.

In one aspect, the invention relates to various diagnostic, monitoring, test and other methods related to RA detection and therapy. In one embodiment, the invention provides a diagnostic method of assessing whether a patient has RA or has higher than normal risk for developing RA, comprising the steps of comparing the level of expression of a marker of the invention in a patient sample and the normal level of expression of the marker in a control, e.g., a sample from a patient without RA or the expression level of the marker in a population-average. A significantly higher level of expression of the marker in the patient sample as compared to the normal level is an indication that the patient is afflicted with RA or has higher than normal risk for developing RA. It will be appreciated that the “level of expression” includes a quantitative measurement, i.e., the sample may be analyzed quantitatively, wherein the abundance of one or more of the markers in a sample is determined and compared to the normal abundance of the one or more markers.

According to the invention, the marker(s) are selected such that the positive predictive value of the methods of the invention is at least about 10%, preferably about 25%, more preferably about 50% and most preferably about 90%. Also preferred are embodiments of the method wherein the marker is over- or under-expressed by at least two-fold in at least about 20% of fast-progressing RA.

In the methods of the present invention, the samples or patient samples may comprise RA-associated body fluids. Such fluids include, for example, blood fluids, (e.g., whole blood, blood serum, plasma, blood having platelets removed therefrom, etc.), urine, saliva, tears, and synovial fluid. The patient samples may also comprise cells, e.g., cells obtained from the patient. The cells may be endothelial cells, white blood cells and synovium cells, osteoclasts, osteoblasts, chondrocytes as well other cells found in joints. In a further embodiment, the patient sample is in vivo.

The methods of the present invention are particularly useful for patients with identified inflammatory synovitis or other symptoms associated with RA. The methods of the present invention can also be of particular use with patients having an enhanced risk of developing RA (e.g., patients having a familial history of RA, patients identified as having a RF, patients at least about 40-60 years of age and female patients at least about 40-60 years of age). The methods of the present invention may further be of particular use in monitoring the efficacy of treatment of a RA patient (e.g. the efficacy of nonsteroidal anti-inflammatory drugs (NSAIDs), corticosteroids, and disease-modifying antirheumatic drugs (DMARDs)).

In accordance with the methods of the present invention, the level of expression of the marker in a sample can be assessed, for example, by detecting the presence in the sample of:

a marker protein (e.g., a protein having a sequence selected from the group consisting of SEQ ID NOs:2, 4, 6, 8, and 10), or a fragment of the protein (e.g. using a reagent, such as an antibody, an antibody derivative, or an antibody fragment, which binds specifically with the marker protein or a fragment of the protein)

a metabolite which is produced directly (i.e., catalyzed) or indirectly by a marker protein

a transcribed polynucleotide (e.g. an mRNA or a cDNA, including a polynucleotide selected from the group consisting of SEQ ID NOs:1, 3, 5, 7, and 9), or fragment thereof, having at least a portion with which the marker nucleic acid is substantially homologous (e.g. by contacting a mixture of transcribed polynucleotides obtained from the sample with a substrate having one or more of the marker nucleic acids fixed thereto at selected positions)

a transcribed polynucleotide or fragment thereof, wherein the polynucleotide anneals with the marker nucleic acid under stringent hybridization conditions.

According to the invention, any of the aforementioned methods may be performed using a plurality (e.g. 2, 3, 5, or 10 or more) of RA markers, including RA markers known in the art. In such methods, the level of expression in the sample of each of a plurality of markers, at least one of which is a marker of the invention, is compared with the normal level of expression of each of the plurality of markers in samples of the same type obtained from control humans not afflicted with RA. A significantly altered (i.e., increased or decreased as specified in the above-described methods using a single marker) level of expression in the sample of one or more markers of the invention, or some combination thereof, relative to that marker's corresponding normal or control level, is an indication that the patient is afflicted with RA. For all of the aforementioned methods, the marker(s) are preferably selected such that the positive predictive value of the method is at least about 10%.

In a further aspect, the invention provides an antibody, an antibody derivative, or an antibody fragment, which binds specifically with a marker protein or a fragment of the protein. The invention also provides methods for making such antibody, antibody derivative, and antibody fragment. Such methods may comprise immunizing a mammal with a protein or peptide comprising the entirety, or a segment of 10 or more amino acids, of a marker protein, wherein the protein or peptide may be obtained from a cell or by chemical synthesis. The methods of the invention also encompass producing monoclonal and single-chain antibodies, which would further comprise isolating splenocytes from the immunized mammal, fusing the isolated splenocytes with an immortalized cell line to form hybridomas, and screening individual hybridomas for those that produce an antibody that binds specifically with a marker protein or a fragment of the protein.

In another aspect, the invention relates to various diagnostic and test kits. In one embodiment, the invention provides a kit for assessing whether a patient is afflicted with RA. The kit comprises a reagent for assessing expression of a marker of the invention. In another embodiment, the invention provides a kit for assessing the suitability of a chemical or biologic agent for inhibiting RA in a patient. Such a kit comprises a reagent for assessing expression of a marker of the invention, and may also comprise one or more of such agents. Such kits may comprise an antibody, an antibody derivative, or an antibody fragment, which binds specifically with a marker protein, or a fragment of the protein. Such kits may also comprise a plurality of antibodies, antibody derivatives, or antibody fragments wherein the plurality of such antibody agents binds specifically with a marker protein, or a fragment of the protein. In an additional embodiment, the kit comprises a nucleic acid probe that binds specifically with a marker nucleic acid or a fragment of the nucleic acid. The kit may also comprise a plurality of probes, wherein each of the probes binds specifically with a marker nucleic acid, or a fragment of the nucleic acid.

In a further aspect, the invention relates to methods for treating a patient afflicted with or at risk of developing RA. Such methods may comprise reducing the expression and/or interfering with the biological function of a marker of the invention. In one embodiment, the method comprises providing to the patient an antisense oligonucleotide or polynucleotide complementary to a marker nucleic acid, or a segment thereof. For example, an antisense polynucleotide may be provided to the patient through the delivery of a vector that expresses an anti-sense polynucleotide of a marker nucleic acid or a fragment thereof. In another embodiment, the method comprises providing to the patient an antibody, an antibody derivative, or antibody fragment, which binds specifically with a marker protein or a fragment of the protein.

It will be appreciated that the methods and kits of the present invention may also include known RA markers, i.e., the markers of the present invention may be used alone, in combination, and in combination with known RA markers.

### DETAILED DESCRIPTION OF THE INVENTION

The invention relates to newly discovered markers associated with RA. It has been discovered that a higher than normal level of expression of individual markers and combinations of markers described herein correlates with RA. Methods are provided for detecting the presence of RA, the absence of RA, the type of RA (e.g., erosive versus non-erosive), and other characteristics of RA that are relevant to prevention, diagnosis, characterization, and therapy of RA.

Definitions

As used herein, each of the following terms has the meaning associated with it in this section.

The “a” and “an” are use herein to refer to one or to more than one (i.e. to at least one) of the grammatical object of the article. By way of example, “an element” means one element or more than one element.

A “marker” is a naturally-occurring polymer corresponding to at least one of the proteins listed in Tables 1-5. Markers further include, without limitation, sense and anti-sense strands of genomic DNA (i.e. including any introns occurring therein), RNA generated by transcription of genomic DNA (i.e. prior to splicing), RNA generated by splicing of RNA transcribed from genomic DNA, and proteins generated by translation of spliced RNA (e.g. including proteins both before and after cleavage of normally cleaved regions such as transmembrane signal sequences). As used herein, “marker” may also include a cDNA made by reverse transcription of an RNA generated by transcription of genomic DNA (including spliced RNA).

A “marker set” is a group of more than one marker.

“Proteins of the invention” encompass marker proteins and their fragments; variant marker proteins and their fragments; peptides and polypeptides comprising an at least 15 amino acid segment of a marker or variant marker protein; and fusion proteins comprising a marker or variant marker protein, or an at least 15 amino acid segment of a marker or variant marker protein.

Unless otherwise specified herewithin, the terms “antibody” and “antibodies” broadly encompass naturally-occurring forms of antibodies (e.g., IgG, IgA, IgM, IgE) and recombinant antibodies such as single-chain antibodies, chimeric and humanized antibodies and multi-specific antibodies, as well as fragments and derivatives of all of the foregoing, which fragments and derivatives have at least an antigenic binding site. Antibody derivatives may comprise a protein or chemical moiety conjugated to an antibody.

As used herein a “polynucleotide corresponds to” another (a first) polynucleotide if it is related to the first polynucleotide by any of the following relationships: 1) The second polynucleotide comprises the first polynucleotide and the second polynucleotide encodes a gene product. 2) The second polynucleotide is 5′ or 3′ to the first polynucleotide in cDNA, RNA, genomic DNA, or fragment of any of these polynucleotides. For example, a second polynucleotide may be fragment of a gene that includes the first and second polynucleotides. The first and second polynucleotides are related in that they are components of the gene coding for a gene product, such as a protein or antibody. However, it is not necessary that the second polynucleotide comprises or overlaps with the first polynucleotide to be encompassed within the definition of “corresponding to” as used herein. For example, the first polynucleotide may be a fragment of a 3′ untranslated region of the second polynucleotide. The first and second polynucleotide may be fragments of a gene coding for a gene product. The second polynucleotide may be an exon of the gene while the first polynucleotide may be an intron of the gene. 3) The second polynucleotide is the complement of the first polynucleotide.

The term “probe” refers to any molecule which is capable of selectively binding to a specifically intended target molecule, for example a marker of the invention. Probes can be either synthesized by one skilled in the art, or derived from appropriate biological preparations. For purposes of detection of the target molecule, probes may be specifically designed to be labeled, as described herein. Examples of molecules that can be utilized as probes include, but are not limited to, RNA, DNA, proteins, antibodies, and organic monomers.

An “RA-associated” body fluid or “patient sample” includes, without limitation, blood fluids (e.g. whole blood, blood serum, plasma, blood having platelets removed therefrom, etc.), synovial fluid, urine, saliva and tears.

“Expression” refers to the presence or abundance of a marker protein or a fragment of the protein in a sample as well as the presence of a marker nucleic acid, i.e., a transcribed polynucleotide (e.g., an mRNA or a cDNA), or a fragment thereof, in a sample.

“Over-expression” and “under-expression” of a marker refers to expression of the marker in a sample, at a greater or lesser level, respectively, than the normal level of expression of the marker (e.g. at least two-fold greater or lesser level). The marker is said to be over-expressed or under-expressed if either the marker protein or marker nucleic acid is present at a greater or lesser level, respectively, than the normal level in a patient sample.

“Erosive RA” is RA characterized by erosions or pits in the surface of the bone adjacent to the articular surface. In particular, in erosive RA, the granulation tissue actively invades and destroys the periarticular bone and cartilage at the margin between the synorium and the bone.

“Non-erosive RA” is RA that does not exhibit erosive RA characteristics.

As used herein, the term “promoter/regulatory sequence” means a nucleic acid sequence which is required for expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue-specific manner.

A “constitutive” promoter is a nucleotide sequence which, when operably linked with a polynucleotide which encodes or specifies a gene product, causes the gene product to be produced in a living human cell under most or all physiological conditions of the cell.

An “inducible” promoter is a nucleotide sequence which, when operably linked with a polynucleotide which encodes or specifies a gene product, causes the gene product to be produced in a living human cell substantially only when an inducer, which corresponds to the promoter, is present in the cell.

A “tissue-specific” promoter is a nucleotide sequence which, when operably linked with a polynucleotide which encodes or specifies a gene product, causes the gene product to be produced in a living human cell substantially only if the cell is a cell of the tissue type corresponding to the promoter.

A “transcribed polynucleotide” is a polynucleotide (e.g. an RNA, a cDNA, or an analog of one of an RNA or cDNA) which is complementary to or homologous with all or a portion of a mature RNA made by transcription of a genomic DNA corresponding to a marker of the invention and normal post-transcriptional processing (e.g. splicing), if any, of the transcript.

“Complementary” refers to the broad concept of sequence complementarity between regions of two nucleic acid strands or between two regions of the same nucleic acid strand. It is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. Preferably, the first region comprises a first portion and the second region comprises a second portion, whereby, when the first and second portions are arranged in an antiparallel fashion, at least about 50%, and preferably at least about 75%, at least about 90%, or at least about 95% of the nucleotide residues of the first portion are capable of base pairing with nucleotide residues in the second portion. More preferably, all nucleotide residues of the first portion are capable of base pairing with nucleotide residues in the second portion.

“Homologous” as used herein, refers to nucleotide sequence similarity between two regions of the same nucleic acid strand or between regions of two different nucleic acid strands. When a nucleotide residue position in both regions is occupied by the same nucleotide residue, then the regions are homologous at that position. A first region is homologous to a second region if at least one nucleotide residue position of each region is occupied by the same residue. Homology between two regions is expressed in terms of the proportion of nucleotide residue positions of the two regions that are occupied by the same nucleotide residue. By way of example, a region having the nucleotide sequence 5′-ATTGCC-3′ and a region having the nucleotide sequence 5′-TATGGC-3′ share 50% homology. Preferably, the first region comprises a first portion and the second region comprises a second portion, whereby, at least about 50%, and preferably at least about 75%, at least about 90%, or at least about 95% of the nucleotide residue positions of each of the portions are occupied by the same nucleotide residue. More preferably, all nucleotide residue positions of each of the portions are occupied by the same nucleotide residue.

A marker is “fixed” to a substrate if it is covalently or non-covalently associated with the substrate such the substrate can be rinsed with a fluid (e.g. standard saline citrate, pH 7.4) without a substantial fraction of the marker dissociating from the substrate.

As used herein, a “naturally-occurring” nucleic acid molecule refers to an RNA or DNA molecule having a nucleotide sequence that occurs in nature (e.g. encodes a natural protein).

The term “isoform” as used herein refers to variants of a polypeptide that are encoded by the same gene, but that differ in their pI or MW, or both. Such isoforms can differ in their amino acid composition (e.g., as a result of alternative mRNA or premRNA processing, e.g. alternative splicing or limited proteolysis) and in addition, or in the alternative, may arise from differential post-translational modification (e.g., glycosylation, acylation, phosphorylation).

Expression of a marker in a patient is “significantly” higher or lower than the normal level of expression of a marker if the level of expression of the marker is greater or less, respectively, than the normal level by an amount greater than the standard error of the assay employed to assess expression, and preferably at least twice, and more preferably three, four, five or ten times that amount. Alternately, expression of the marker in the patient can be considered “significantly” higher or lower than the normal level of expression if the level of expression is at least about two, and preferably at least about three, four, or five times, higher or lower, respectively, than the normal level of expression of the marker.

RA is “inhibited” if at least one symptom of the RA is alleviated, terminated, slowed, or prevented. As used herein, RA is “inhibited” if recurrence of RA is reduced, slowed, delayed, or prevented or RA remission is induced or maintained.

A kit is any manufacture (e.g. a package or container) comprising at least one reagent, e.g. a probe, for specifically detecting a marker of the invention. The manufacture may be promoted, distributed, or sold as a unit for performing the methods of the present invention.

Description

The present invention is based, in part, on newly identified markers which are differently expressed in RA patients as compared to normal individuals (i.e., individuals not afflicted by RA). The markers of the invention correspond to polypeptide and nucleic acid molecules which can be detected in one or both of normal samples and diseased patient samples. The presence, absence, or level of expression of one or more of these markers in patient samples is herein correlated with the rheumatoid arthritic state of the patient.

The present invention also provides markers which are differently expressed in patients with erosive RA. Erosive RA is characterized by erosions or pits in the surface of the bone adjacent to the articular surface. In particular, in erosive RA, the granulation tissue actively invades and destroys the periarticular bone and cartilage at the margin between the synovium and the bone.

The compositions, kits, and methods of the invention have the following uses, among others:

1) assessing whether a patient is afflicted with RA;

2) assessing the stage of RA in a patient;

3) assessing the progressive nature of RA in a patient;

4) assessing whether a patient has erosive RA;

5) assessing whether a patient has non-erosive RA;

6) making an isolated hybridoma which produces an antibody useful for assessing whether a patient is afflicted with RA;

7) assessing the efficacy of one or more test compounds for inhibiting RA in a patient;

8) assessing the efficacy of a therapy for inhibiting RA in a patient;

9) assessing the efficacy of a therapy for inhibiting erosive RA in a patient;

10) assessing the efficacy of a therapy for inhibiting non-erosive RA in a patient;

11) monitoring the progression of RA in a patient;

12) selecting a composition or therapy for inhibiting RA in a patient;

13) selecting a composition or therapy for inhibiting erosive RA in a patient;

14) selecting a composition or therapy for inhibiting non-erosive RA in a patient;

15) developing agents effective in treating synovitis;

16) developing agents effective in treating erosive RA;

17) developing agents effective in treating non-erosive RA;

18) treating a patient afflicted with RA;

19) inhibiting RA in a patient;

20) assessing the rhematoid arthritic progressive potential of a test compound; and

21) inhibiting RA in a patient at risk for developing RA.

The methods of the present invention comprise the step of comparing the level of expression of a marker in a patient sample, with the normal level of expression of the marker. A significant difference between the level of expression of the marker in the patient sample and the normal level is an indication that the patient is afflicted with RA. A “normal” level of expression refers to the expression level of the marker in the control, such as in a sample from an individual without RA. Subjects that are not afflicted with RA can include normal subjects with no known disease or condition, or subjects with joint diseases or conditions other than RA, including gout, osteoarthritis, or synovitis (e.g., traumatic synovitis). Alternatively, and particularly as further information becomes available as a result of routine performance of the methods described herein, population-average values for expression of the markers of the invention may be used as the “normal” level of expression. For example, a laboratory may establish reference ranges for the level of the marker for subjects with and without RA, as well as for subjects with erosive and non-erosive forms of RA, as is conventional in the diagnostic art.

As used herein the term “expression” refers to the presence or abundance of a marker protein or a fragment of the protein in a sample as well as the presence of a marker nucleic acid, i.e., a transcribed polynucleotide (e.g., an mRNA or a cDNA), or a fragment thereof, in a sample. In a method of determining the abundance of a marker in a sample compared to a normal or control, i.e., to identify markers that are differentially present, the relative abundance may be determined by normalizing the signal obtained upon detecting the marker in a sample by reference to a suitable background parameter, e.g., to the total protein in the sample being analyzed to an invariant marker, i.e., a marker whose abundance is known to be similar in the sample being compared, or to the total signal detected from all proteins in the sample.

Table 1 lists all of the markers of the invention (and comprises markers listed in Tables 2-5), which are over-expressed in patients with RA compared to normal individuals (i.e., individuals who are not afflicted with RA). Table 2 lists markers that are newly-associated with RA and are over-expressed in patients diagnosed with erosive or non-erosive RA. Table 3 lists preferred markers of the present invention. Table 3A lists markers which are over-expressed in serum samples of patients with RA compared to normal individuals (i.e., individuals who are not afflicted with RA). Table 4 lists markers which are especially useful for new detection (“screening”) and detection of recurrence of RA. Table 5 lists newly-identified markers (SEQ ID NOs: 1-10) that are over-expressed in patients with RA. All of these tables include a marker identification number (“Marker”), the gene corresponding to the marker (“Gene Name”), the data generated for each synovial fluid sample (E=Erosive and N=Non-Erosive), the corresponding molecular weight (“MW (Da)”), the corresponding GenBank GI Number (“GI number”), and where indicated, the sequence listing identifier of the cDNA sequence of a nucleotide transcript encoded by or corresponding to the marker (“SEQ ID NO (nts)”) and the sequence listing identifier of the amino acid sequence of a protein encoded by or corresponding to the marker (“SEQ ID NO (AA)”). Table 6 lists protein concentration of Calgranulin A in a pool of Size Exclusion Chromatography (SEC) fractions 7, 8 and 9 of human serum. Table 7 lists protein concentration of Calgranulin B in a pool of SEC fractions 7, 8 and 9 of human serum. Tables 8A and 8B list protein concentration of Calgranulin C in a pool of SEC fractions 7, 8 and 9 of human serum. Table 9 lists the average protein concentration values in human serum and the significance test results of Serum Amyloid A (SAA) protein.

In a preferred diagnostic method of assessing whether a patient is afflicted with RA (e.g., new detection (“screening”) and detection of recurrence), the method comprises comparing:

a) the level of expression of a marker of the invention in a patient sample, and

b) the normal level of expression of the marker in a control.

A significantly higher level of expression of the marker in the patient sample as compared to the normal level is an indication that the patient is afflicted with RA. In one embodiment, the marker is listed in Table 2. In an alternative embodiment, the marker is listed in Table 4.

In further preferred diagnostic method of assessing whether a patient is afflicted with erosive RA, the method comprises comparing:

a) the level of expression of a marker of the invention in a patient sample, and

b) the normal level of expression of the marker in a control.

A significantly higher level of expression of the marker in the patient sample as compared to the normal level is an indication that the patient is afflicted with erosive RA. In one embodiment, the marker is listed in Table 2. In an alternative embodiment, the marker is listed in Table 3A.

The invention also provides diagnostic methods for assessing the efficacy of a therapy for inhibiting RA in a patient. Such methods comprise comparing:

a) expression of a marker of the invention in a first sample obtained from the patient prior to providing at least a portion of the therapy to the patient, and

b) expression of the marker in a second sample obtained from the patient following provision of the portion of the therapy.

A significantly lower level of expression of the marker in the second sample relative to that in the first sample is an indication that the therapy is efficacious for inhibiting RA in the patient. It will be appreciated that in these methods the “therapy” may be any therapy for treating RA including, but not limited to, anti-inflammatory drugs, disease-modifying drugs and gene therapy. Thus, the methods of the invention may be used to evaluate a patient before, during and after therapy, for example, to evaluate the efficacy of treatment.

In a preferred embodiment, the methods are directed to therapy using a chemical or biologic agent. These methods comprise comparing:

a) expression of a marker of the invention in a first sample obtained from the patient and maintained in the presence of the chemical or biologic agent, and

b) expression of the marker in a second sample obtained from the patient and maintained in the absence of the agent.

A significantly lower level of expression of the marker in the first sample relative to that in the second sample is an indication that the agent is efficacious for inhibiting RA in the patient. In one embodiment, the first and second samples can be portions of a single sample obtained from the patient or portions of pooled samples obtained from the patient.

The invention additionally provides a monitoring method for assessing the progression of RA in a patient, the method comprising:

a) detecting in a patient sample at a first time point, the expression of a marker of the invention;

b) repeating step a) at a subsequent point in time; and

c) comparing the level of expression detected in steps a) and b), and therefrom monitoring the progression of RA in the patient.

A significantly higher level of expression of the marker in the sample at the subsequent time point from that of the sample at the first time point is an indication that the RA has progressed, whereas a significantly lower level of expression is an indication that the RA has regressed.

The invention moreover provides a test method for selecting a composition for inhibiting RA in a patient. This method comprises the steps of:

a) obtaining a sample from the patient;

b) separately maintaining aliquots of the sample in the presence of a plurality of test compositions;

c) comparing expression of a marker of the invention in each of the aliquots; and

d) selecting one of the test compositions which significantly reduces the level of expression of the marker in the aliquot containing that test composition, relative to the levels of expression of the marker in the presence of the other test compositions.

In addition, the invention further provides a method of inhibiting RA in a patient. This method comprises the steps of:

a) obtaining a sample from the patient;

b) separately maintaining aliquots of the sample in the presence of a plurality of compositions;

c) comparing expression of a marker of the invention in each of the aliquots; and

d) administering to the patient at least one of the compositions which significantly lowers the level of expression of the marker in the aliquot containing that composition, relative to the level of expression of the marker in the presence of the other compositions.

Any marker or combination of markers listed in the tables, as well as any known markers in combination with the markers listed in the tables, may be used in the compositions, kits, and methods of the present invention. In general, it is preferable to use markers for which the difference between the level of expression of the marker in RA patient samples and the level of expression of the same marker in normal samples is as great as possible. Although this difference can be as small as the limit of detection of the method for assessing expression of the marker, it is preferred that the difference be at least greater than the standard error of the assessment method, and preferably a difference of at least 2-, 3-, 4-, 5-, 6-, 7-, 8-, 9-, 10-, 15-, 20-, 25-, 100-, 500-, 1000-fold or greater.

It will be appreciated that patient samples containing bodily fluids (e.g., blood fluid, whole blood, serum, blood having platelets removed therefrom etc., and synovial fluid) may be used in the methods of the present invention. In these embodiments, the level of expression of the marker can be assessed by assessing the amount or abundance (e.g. absolute amount or concentration) of a marker product (e.g., protein and RNA transcript encoding said protein, fragments of the protein, isoforms of the protein, and RNA transcript) in a sample. The sample can, of course, be subjected to a variety of well-known post-collection preparative and storage techniques (e.g. fixation, storage, freezing, lysis, homogenization, DNA or RNA extraction, ultrafiltration, concentration, evaporation, centrifugation, etc.) prior to assessing the amount of the marker in the sample.

Preferred in vivo techniques for detection of a marker protein of the invention include introducing into a subject an antibody that specifically hinds the protein, isoform of the protein, or protein fragment. In certain embodiments, the antibody can be labeled with a radioactive molecule whose presence and location in a subject can be detected by standard imaging techniques.

Expression of a marker of the invention may be assessed by any of a wide variety of well known methods for detecting expression of a protein or transcribed molecule. Non-limiting examples of such methods include immunological methods for detection of secreted, cell-surface, cytoplasmic, or nuclear proteins, protein purification methods, protein function or activity assays, nucleic acid hybridization methods, nucleic acid reverse transcription methods, and nucleic acid amplification methods. Such methods may also include physical methods such as liquid and gas chromatography, mass spectroscopy, nuclear magnetic resonance and other imaging technologies.

In a preferred embodiment, expression of a marker protein is assessed using an antibody (e.g. a radio-labeled, chromophore-labeled, fluorophore-labeled, or enzyme-labeled antibody), an antibody derivative (e.g. an antibody conjugated with a substrate or with the protein or ligand of a protein-ligand pair {e.g. biotin-streptavidin}), or an antibody fragment (e.g. a single-chain antibody, an isolated antibody hypervariable domain, etc.) which binds specifically with a marker protein, isoform of the marker protein, or a fragment of the protein, wherein the protein may have undergone none, all or a portion of its normal post-translational modification and/or proteolysis during the course of its secretion or release from cells.

In another preferred embodiment, expression of a marker is assessed by preparing mRNA/cDNA (i.e. a transcribed polynucleotide) from cells in a patient sample, and by hybridizing the mRNA/cDNA with a reference polynucleotide which comprises the marker nucleic acid sequence or its complement, or a fragment of said sequence or complement. cDNA can, optionally, be amplified using any of a variety of polymerase chain reaction methods prior to hybridization with the reference polynucleotide. Expression of one or more marker nucleic acid can likewise be detected using quantitative PCR to assess the level of RNA transcripts encoded by the marker(s).

In a related embodiment, a mixture of transcribed polynucleotides obtained from the sample is contacted with a substrate having fixed thereto a polynucleotide complementary to or homologous with at least a portion (e.g. at least 7, 10, 15, 20, 25, 30, 40, 50, 100, 500, or more nucleotide residues) of a RNA transcript encoded by a marker of the invention. If polynucleotides complementary to or homologous with a RNA transcript encoded by the marker of the invention are differentially detectable on the substrate (e.g. detectable using radioactivity, different chromophores or fluorophores), are fixed to different selected positions, then the levels of expression of a plurality of markers can be assessed simultaneously using a single substrate (e.g. a “gene chip” microarray of polynucleotides fixed at selected positions). When a method of assessing marker expression is used which involves hybridizaton of one nucleic acid with another, it is preferred that the hybridization be performed under stringent hybridization conditions.

Because the compositions, kits, and methods of the invention rely on detection of a difference in expression levels of one or more markers of the invention, it is preferable that the level of expression of the marker is significantly greater than the minimum detection limit of the method used to assess expression in a normal or control sample.

It is understood that by routine screening of additional patient samples for the expression levels of one or more of the markers of the invention, it will be realized that certain of the markers are expressed at varying levels based on the progressiveness of disease. Thus the markers and methods of the present invention may be used to identify a non-progressive to progressive gradient. Such gradient would be especially useful in characterizing, managing and treating RA.

It is recognized that certain markers correspond to proteins which are secreted from patient samples (i.e. synovial fluid, endothelial cells, synovium cells, serum, plasma) to the extracellular space surrounding the cells. These markers are preferably used in certain embodiments of the compositions, kits, and methods of the invention, owing to the fact that the protein corresponding to each of these markers can be detected in an RA-associated body fluid sample, which may be easily collected from a human patient. It will be appreciated, however, that intracellular markers are also included within the markers of the present invention and are also useful in the methods of the present invention.

It is a simple matter for the skilled artisan to determine whether any particular marker corresponds to a secreted protein. In order to make this determination, the protein corresponding to a marker is expressed in a test cell, extracellular fluid is collected, and the presence or absence of the protein in the extracellular fluid is assessed (e.g. using a labeled antibody which binds specifically with the protein).

The compositions, kits, and methods of the invention can also be used to detect expression of markers corresponding to proteins having at least one portion which is displayed on the surface of cells which express it. It is a simple matter for the skilled artisan to determine whether the protein corresponding to any particular marker comprises a cell-surface protein. For example, immunological methods may be used to detect such proteins on whole cells, or well known computer-based sequence analysis methods (e.g. the SIGNALP program; Nielsen et al., 1997, Protein Engineering 10:1-6) may be used to predict the presence of at least one extracellular domain (i.e. including both secreted proteins and proteins having at least one cell-surface domain). Expression of a marker corresponding to a protein having at least one portion which is displayed on the surface of a cell which expresses it may be detected without necessarily lysing the cell (e.g. using a labeled antibody which binds specifically with a cell-surface domain of the protein).

When a plurality of markers of the invention are used in the compositions, kits, and methods of the invention, the level of expression of each marker in a patient sample can be compared with the normal level of expression of each of the plurality of markers in RA samples of the same type, either in a single reaction mixture (i.e. using reagents, such as different fluorescent probes, for each marker) or in individual reaction mixtures corresponding to one or more of the markers. In one embodiment, a significantly enhanced level of expression of more than one of the plurality of markers in the sample, relative to the corresponding normal levels, is an indication that the patient is afflicted with RA. When a plurality of markers is used, it is preferred that 2, 3, 4, 5, 8, 10, 12, 15, 20, 30, or 50 or more individual markers be used, wherein fewer markers are preferred.

Prior to the present invention, only a limited number of markers were known to be associated with RA (e.g., RF, complement factor B, and C-reactive protein). These markers may be used together with one or more markers of the invention in a panel of markers. For example, a sample may be assayed to determine the presence and/or expression levels of known markers in combination with the markers of the present invention. The presence, over- and/or under-expression of markers, such as RF in combination with the presence, over- and/or underexpression of the markers of the present invention, may be used to further characterize RA.

It is recognized that the compositions, kits, and methods of the invention will be of particular utility to patients having an enhanced risk of developing RA and their medical advisors. Patients recognized as having an enhanced risk of developing RA include, for example, patients having a familial history of RA, patients identified as having a RF, patients of advancing age and women of advancing age (i.e. between 40 and 60 years).

The level of expression of a marker in normal (i.e. an individual who is not afflicted with RA) individuals or a control can be assessed in a variety of ways. As further information becomes available as a result of routine performance of the methods described herein, population-average values for expression of the markers of the invention may be used. In other embodiments, the ‘normal’ level of expression of a marker may be determined by assessing expression of the marker in a patient sample obtained from a non-RA-afflicted patient, from a patient sample obtained from a patient before the suspected onset of RA in the patient, from archived patient samples, and the like.

The invention includes compositions, kits, and methods for assessing the presence of RA in a sample (e.g. an archived tissue sample or a sample obtained from a patient). These compositions, kits, and methods are substantially the same as those described above, except that, where necessary, the compositions, kits, and methods are adapted for use with samples other than patient samples. For example, when the sample to be used is a parafinized, archived human tissue sample, it can be necessary to adjust the ratio of compounds in the compositions of the invention, in the kits of the invention, or the methods used to assess levels of marker expression in the sample. Such methods are well known in the art and within the skill of the ordinary artisan.

The invention includes a kit for assessing the presence of RA (e.g. in a sample such as a patient sample). The kit comprises a plurality of reagents, each of which is capable of binding specifically with a nucleic acid or polypeptide corresponding to a marker of the invention. Suitable reagents for binding with a polypeptide corresponding to a marker of the invention include antibodies, antibody derivatives, antibody fragments, and the like. Suitable reagents for binding with a nucleic acid (e.g. a genomic DNA, an mRNA, a spliced mRNA, a cDNA, or the like) include complementary nucleic acids. For example, the nucleic acid reagents may include oligonucleotides (labeled or non-labeled) fixed to a substrate, labeled oligonucleotides not bound with a substrate, pairs of PCR primers, molecular beacon probes, and the like.

The kit of the invention may optionally comprise additional components useful for performing the methods of the invention. By way of example, the kit may comprise fluids (e.g. SSC buffer) suitable for annealing complementary nucleic acids or for binding an antibody with a protein with which it specifically binds, one or more sample compartments, an instructional material which describes performance of a method of the invention, a sample from a normal individual, a sample from a RA patient, and the like.

The invention also includes a method of making an isolated hybridoma which produces an antibody useful for assessing whether patient is afflicted with RA. In this method, a marker protein of the invention is isolated (e.g. by purification from a cell in which it is expressed or by transcription and translation of a nucleic acid encoding the protein in vivo or in vitro using known methods). A vertebrate, preferably a mammal such as a mouse, rat, rabbit, or sheep, is immunized using the isolated protein or protein fragment. The vertebrate may optionally (and preferably) be immunized at least one additional time with the isolated protein or protein fragment, so that the vertebrate exhibits a robust immune response to the protein or protein fragment. Splenocytes are isolated from the immunized vertebrate and fused with an immortalized cell line to form hybridomas, using any of a variety of methods well known in the art. Hybridomas formed in this manner are then screened using standard methods to identify one or more hybridomas which produce an antibody which specifically binds with the protein or protein fragment. The invention also includes hybridomas made by this method and antibodies made using such hybridomas.

The invention also includes a method of assessing the efficacy of a test compound for inhibiting RA. As described above, differences in the level of expression of the markers of the invention correlate with the rheumatoid arthritic state of the patient. Although it is recognized that changes in the levels of expression of certain of the markers of the invention likely result from the rheumatoid arthritic state of patient, it is likewise recognized that changes in the levels of expression of other of the markers of the invention induce, maintain, and promote the rheumatoid arthritic state of those patients. Thus, compounds which inhibit RA in a patient will cause the level of expression of one or more of the markers of the invention to change to a level nearer the normal level of expression for that marker (i.e. the level of expression for the marker in RA patients).

This method thus comprises comparing expression of a marker in a first patient sample and maintained in the presence of the test compound and expression of the marker in a second patient sample and maintained in the absence of the test compound. A significant decrease in the level of expression of a marker may be an indication that the test compound inhibits RA. The patient samples may, for example, be aliquots of a single sample obtained from a patient, pooled normal samples obtained an individual, cells of a normal individual, aliquots of a single sample obtained from a RA patient, pooled samples from a RA patient, cells of a RA patient, or the like. In one embodiment, the samples from a RA patient and a plurality of compounds known to be effective for inhibiting RA are tested in order to identify the compound which is likely to best inhibit the RA in the patient.

This method may likewise be used to assess the efficacy of a therapy for inhibiting RA in a patient. In this method, the level of expression of one or more markers of the invention in a pair of samples (one subjected to the therapy, the other not subjected to the therapy) is assessed. As with the method of assessing the efficacy of test compounds, if the therapy induces a significant decrease in the level of expression of a marker, or blocks induction of a marker, then the therapy may be efficacious for inhibiting RA. As above, if samples from a selected patient are used in this method, then alternative therapies can be assessed in vitro in order to select a therapy most likely to be efficacious for inhibiting RA in the patient.

The present invention further provides methods for identifying the presence of erosive and non-erosive RA by detecting expression of a marker listed in Tables 3-5, wherein over-expression of one or a plurality of the markers is correlated with erosive RA. By identifying whether a patient sample is afflicted with erosive or non-erosive RA, therapy may be customized to better treat the specific type of RA.

Expression of a marker can be inhibited in a number of ways generally known in the art. For example, an antisense oligonucleotide can be provided to the patient samples in order to inhibit transcription, translation, or both, of the marker(s). Alternately, a polynucleotide encoding an antibody, an antibody derivative, or an antibody fragment, and operably linked with an appropriate promoter/regulator region, can be provided to the patient sample in order to generate intracellular antibodies which will inhibit the function or activity of the protein. Using the methods described herein, a variety of molecules, particularly including molecules sufficiently small that they are able to cross the cell membrane, can be screened in order to identify molecules which inhibit expression of the marker(s). The compound so identified can be provided to the patient in order to inhibit expression of the marker(s) in the patient.

Expression of a marker can be enhanced in a number of ways generally known in the art. For example, a polynucleotide encoding the marker and operably linked with an appropriate promoter/regulator region can be provided to patient samples in order to induce enhanced expression of the protein (and mRNA) corresponding to the marker therein. Alternatively, if the protein is capable of crossing the cell membrane, inserting itself in the cell membrane, or is normally a secreted protein, then expression of the protein can be enhanced by providing the protein (e.g. directly or by way of the bloodstream) to the patient sample.

As described above, the rheumatoid arthritic state of the patient is correlated with changes in the levels of expression of the markers of the invention. The invention thus includes a method for assessing the RA promoting or progression potential of a test compound. This method comprises maintaining separate aliquots of patient samples in the presence and absence of the test compound. Expression of a marker of the invention in each of the aliquots is compared. A significant increase in the level of expression of a marker in the aliquot maintained in the presence of the test compound (relative to the aliquot maintained in the absence of the test compound) may be an indication that the test compound possesses RA promoting or progression potential. The relative RA promoting or progression potentials of various test compounds can be assessed by comparing the degree of enhancement or inhibition of the level of expression of the relevant markers, by comparing the number of markers for which the level of expression is enhanced or inhibited, or by comparing both.

Various aspects of the invention are described in further detail in the following subsections.

I. Isolated Proteins and Antibodies

One aspect of the invention pertains to marker proteins which are isolated proteins biologically active portions thereof, isoforms, as well as polypeptide fragments suitable for use as immunogens to raise antibodies directed against a polypeptide of the invention. In one embodiment, the native polypeptide corresponding to a marker can be isolated from cells or tissue sources by an appropriate purification scheme using standard protein purification techniques. In another embodiment, polypeptides corresponding to a marker of the invention are produced by recombinant DNA techniques. Alternative to recombinant expression, a polypeptide corresponding to a marker of the invention can be synthesized chemically using standard peptide synthesis techniques.

An “isolated” or “purified” protein or biologically active portion thereof is substantially free of cellular material or other contaminating proteins from the cell or tissue source from which the protein is derived, or substantially free of chemical precursors or other chemicals when chemically synthesized. The language “substantially free of cellular material” includes preparations of protein in which the protein is separated from cellular components of the cells from which it is isolated or recombinantly produced. Thus, protein that is substantially free of cellular material includes preparations of protein having less than about 30%, 20%, 10%, or 5% (by dry weight) of heterologous protein (also referred to herein as a “contaminating protein”). When the protein or biologically active portion thereof is recombinantly produced, it is also preferably substantially free of culture medium, i.e., culture medium represents less than about 20%, 10%, or 5% of the volume of the protein preparation. When the protein is produced by chemical synthesis, it is preferably substantially free of chemical precursors or other chemicals, i.e., it is separated from chemical precursors or other chemicals which are involved in the synthesis of the protein. Accordingly such preparations of the protein have less than about 30%, 20%, 10%, 5% (by dry weight) of chemical precursors or compounds other than the polypeptide of interest.

Biologically active portions of a polypeptide corresponding to a marker of the invention include polypeptides comprising amino acid sequences sufficiently identical to or derived from the amino acid sequence of the protein corresponding to the marker, which include fewer amino acids than the full length protein, and exhibit at least one activity of the corresponding full-length protein. Typically, biologically active portions comprise a domain or motif with at least one activity of the corresponding protein. A biologically active portion of a protein of the invention can be a polypeptide which is, for example, 10, 25, 50, 100 or more amino acids in length. Moreover, other biologically active portions, in which other regions of the protein are deleted, can be prepared by recombinant techniques and evaluated for one or more of the functional activities of the native form of a polypeptide of the invention.

Preferred polypeptides have amino acid sequences encoded by the nucleic acid sequences described herein. Other useful proteins are substantially identical (e.g., at least about 40%, preferably 50%, 60%, 70%, 80%, 90%, 95%, or 99%) to one of these sequences and retain the functional activity of the protein of the corresponding naturally-occurring protein yet differ in amino acid sequence due to natural allelic variation or mutagenesis.

To determine the percent identity of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=# of identical positions/total # of positions (e.g., overlapping positions)×100). In one embodiment the two sequences are the same length.

The determination of percent identity between two sequences can be accomplished using a mathematical algorithm. A preferred, non-limiting example of a mathematical algorithm utilized for the comparison of two sequences is the algorithm of Karlin and Altschul (1990) Proc. Natl. Acad. Sci. USA 87:2264-2268, modified as in Karlin and Altschul (1993) Proc. Natl. Acad. Sci. USA 90:5873-5877. Such an algorithm is incorporated into the NBLAST and XBLAST programs of Altschul, et al. (1990) J. Mol. Biol. 215:403-410. BLAST nucleotide searches can be performed with the NBLAST program, score=100, wordlength=12 to obtain nucleotide sequences homologous to a nucleic acid molecules of the invention. BLAST protein searches can be performed with the XBLAST program, score=50, wordlength=3 to obtain amino acid sequences homologous to a protein molecules of the invention. To obtain gapped alignments for comparison purposes, Gapped BLAST can be utilized as described in Altschul et al. (1997) Nucleic Acids Res. 25:3389-3402. Alternatively, PSI-Blast can be used to perform an iterated search which detects distant relationships between molecules. When utilizing BLAST, Gapped BLAST, and PSI-Blast programs, the default parameters of the respective programs (e.g., XBLAST and NBLAST) can be used. Another preferred, non-limiting example of a mathematical algorithm utilized for the comparison of sequences is the algorithm of Myers and Miller, (1988) CABIOS 4:11-17. Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. Yet another useful algorithm for identifying regions of local sequence similarity and alignment is the FASTA algorithm as described in Pearson and Lipman (1988) Proc. Natl. Acad. Sci. USA 85:2444-2448. When using the FASTA algorithm for comparing nucleotide or amino acid sequences, a PAM120 weight residue table can, for example, be used with a k-tuple value of 2.

The percent identity between two sequences can be determined using techniques similar to those described above, with or without allowing gaps. In calculating percent identity, only exact matches are counted.

The invention also provides chimeric or fusion proteins corresponding to a marker of the invention. As used herein, a “chimeric protein” or “fusion protein” comprises all or part (preferably a biologically active part) of a polypeptide corresponding to a marker of the invention operably linked to a heterologous polypeptide (i.e., a polypeptide other than the polypeptide corresponding to the marker). Within the fusion protein, the term “operably linked” is intended to indicate that the polypeptide of the invention and the heterologous polypeptide are fused in-frame to each other. The heterologous polypeptide can be fused to the amino-terminus or the carboxyl-terminus of the polypeptide of the invention.

One useful fusion protein is a GST fusion protein in which a polypeptide corresponding to a marker of the invention is fused to the carboxyl terminus of GST sequences. Such fusion proteins can facilitate the purification of a recombinant polypeptide of the invention.

In another embodiment, the fusion protein contains a heterologous signal sequence at its amino terminus. For example, the native signal sequence of a polypeptide corresponding to a marker of the invention can be removed and replaced with a signal sequence from another protein. For example, the gp67 secretory sequence of the baculovirus envelope protein can be used as a heterologous signal sequence (Ausubel et al., ed., Current Protocols in Molecular Biology, John Wiley & Sons, NY, 1992). Other examples of eukaryotic heterologous signal sequences include the secretory sequences of melittin and human placental alkaline phosphatase (Stratagene; La Jolla, Calif.). In yet another example, useful prokaryotic heterologous signal sequences include the phoA secretory signal (Sambrook et al., supra) and the protein A secretory signal (Pharmacia Biotech; Piscataway, N.J.).

In yet another embodiment, the fusion protein is an immunoglobulin fusion protein in which all or part of a polypeptide corresponding to a marker of the invention is fused to sequences derived from a member of the immunoglobulin protein family. The immunoglobulin fusion proteins of the invention can be incorporated into pharmaceutical compositions and administered to a subject to inhibit an interaction between a ligand (soluble or membrane-bound) and a protein on the surface of a cell (receptor), to thereby suppress signal transduction in vivo. The immunoglobulin fusion protein can be used to affect the bioavailability of a cognate ligand of a polypeptide of the invention. Inhibition of ligand/receptor interaction can be useful therapeutically, both for treating proliferative and differentiative disorders and for modulating (e.g. promoting or inhibiting) cell survival. Moreover, the immunoglobulin fusion proteins of the invention can be used as immunogens to produce antibodies directed against a polypeptide of the invention in a subject, to purify ligands and in screening assays to identify molecules which inhibit the interaction of receptors with ligands.

Chimeric and fusion proteins of the invention can be produced by standard recombinant DNA techniques. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and re-amplified to generate a chimeric gene sequence (see, e.g., Ausubel et al., supra). Moreover, many expression vectors are commercially available that already encode a fusion moiety (e.g., a GST polypeptide). A nucleic acid encoding a polypeptide of the invention can be cloned into such an expression vector such that the fusion moiety is linked in-frame to the polypeptide of the invention.

A signal sequence can be used to facilitate secretion and isolation of the secreted protein or other proteins of interest. Signal sequences are typically characterized by a core of hydrophobic amino acids which are generally cleaved from the mature protein during secretion in one or more cleavage events. Such signal peptides contain processing sites that allow cleavage of the signal sequence from the mature proteins as they pass through the secretory pathway. Thus, the invention pertains to the described polypeptides having a signal sequence, as well as to polypeptides from which the signal sequence has been proteolytically cleaved (i.e., the cleavage products). In one embodiment, a nucleic acid sequence encoding a signal sequence can be operably linked in an expression vector to a protein of interest, such as a protein which is ordinarily not secreted or is otherwise difficult to isolate. The signal sequence directs secretion of the protein, such as from a eukaryotic host into which the expression vector is transformed, and the signal sequence is subsequently or concurrently cleaved. The protein can then be readily purified from the extracellular medium by art recognized methods. Alternatively, the signal sequence can be linked to the protein of interest using a sequence which facilitates purification, such as with a GST domain.

It will be appreciated that as an alternative to recombinant expression, the marker proteins of the present invention may be chemically synthesized using standard peptide synthesis techniques.

The present invention also pertains to variants of the polypeptides corresponding to individual markers of the invention. Such variants have an altered amino acid sequence, e.g., amino acid substitutions or insertions can be made using naturally occurring or non-naturally occurring amino acids, including L- and D-amino acids. Such variants can function as either agonists (mimetics) or as antagonists. Variants can be generated by mutagenesis, e.g., discrete point mutation or truncation. An agonist can retain substantially the same, or a subset, of the biological activities of the naturally occurring form of the protein. An antagonist of a protein can inhibit one or more of the activities of the naturally occurring form of the protein by, for example, competitively binding to a downstream or upstream member of a cellular signaling cascade which includes the protein of interest. Thus, specific biological effects can be elicited by treatment with a variant of limited function. Treatment of a subject with a variant having a subset of the biological activities of the naturally occurring form of the protein can have fewer side effects in a subject relative to treatment with the naturally occurring form of the protein.

Variants of a protein of the invention which function as either agonists (mimetics) or as antagonists can be identified by screening combinatorial libraries of mutants, e.g., truncation mutants, of the protein of the invention for agonist or antagonist activity. In one embodiment, a variegated library of variants is generated by combinatorial mutagenesis at the nucleic acid level and is encoded by a variegated gene library. A variegated library of variants can be produced by, for example, enzymatically ligating a mixture of synthetic oligonucleotides into gene sequences such that a degenerate set of potential protein sequences is expressible as individual polypeptides, or alternatively, as a set of larger fusion proteins (e.g., for phage display). There are a variety of methods which can be used to produce libraries of potential variants of the polypeptides of the invention from a degenerate oligonucleotide sequence. Methods for synthesizing degenerate oligonucleotides are known in the art (see, e.g., Narang, 1983, Tetrahedron 39:3; Itakura et al., 1984, Annu. Rev. Biochem. 53:323; Itakura et al., 1984, Science 198:1056; Ike et al., 1983 Nucleic Acid Res. 11:477).

In addition, libraries of fragments of the coding sequence of a polypeptide corresponding to a marker of the invention can be used to generate a variegated population of polypeptides for screening and subsequent selection of variants. For example, a library of coding sequence fragments can be generated by treating a double stranded PCR fragment of the coding sequence of interest with a nuclease under conditions wherein nicking occurs only about once per molecule, denaturing the double stranded DNA, renaturing the DNA to form double stranded DNA which can include sense/antisense pairs from different nicked products, removing single stranded portions from reformed duplexes by treatment with S1 nuclease, and ligating the resulting fragment library into an expression vector. By this method, an expression library can be derived which encodes amino terminal and internal fragments of various sizes of the protein of interest.

Several techniques are known in the art for screening gene products of combinatorial libraries made by point mutations or truncation, and for screening cDNA libraries for gene products having a selected property. The most widely used techniques, which are amenable to high through-put analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a technique which enhances the frequency of functional mutants in the libraries, can be used in combination with the screening assays to identify variants of a protein of the invention (Arkin and Yourvan, 1992, Proc. Natl. Acad. Sci. USA 89:7811-7815; Delgrave et al., 1993, Protein Engineering 6(3):327-331).

The present invention also pertains to human orthologs for any non-human nucleic acid or amino acid sequences. The identification of such human orthologs may be determined through conventional Molecular Biology techniques known to someone of ordinary skill in the art, such as blast analysis or library screening, as discussed throughout.

An isolated polypeptide corresponding to a marker of the invention, or a fragment thereof, can be used as an immunogen to generate antibodies using standard techniques for polyclonal and monoclonal antibody preparation. The full-length polypeptide or protein can be used or, alternatively, the invention provides antigenic peptide fragments for use as immunogens. The antigenic peptide of a protein of the invention comprises at least 8 (preferably 10, 15, 20, or 30 or more) amino acid residues of the amino acid sequence of one of the polypeptides of the invention, and encompasses an epitope of the protein such that an antibody raised against the peptide forms a specific immune complex with a marker of the invention to which the protein corresponds. Preferred epitopes encompassed by the antigenic peptide are regions that are located on the surface of the protein, e.g., hydrophilic regions. Hydrophobicity sequence analysis, hydrophilicity sequence analysis, or similar analyses can be used to identify hydrophilic regions.

An immunogen typically is used to prepare antibodies by immunizing a suitable (i.e. immunocompetent) subject such as a rabbit, goat, mouse, or other mammal or vertebrate. An appropriate immunogenic preparation can contain, for example, recombinantly-expressed or chemically-synthesized polypeptide. The preparation can further include an adjuvant, such as Freund's complete or incomplete adjuvant, or a similar immunostimulatory agent.

Accordingly, another aspect of the invention pertains to antibodies directed against a polypeptide of the invention. The terms “antibody” and “antibody substance” as used interchangeably herein refer to immunoglobulin molecules and immunologically active portions of immunoglobulin molecules, i.e., molecules that contain an antigen binding site which specifically binds an antigen, such as a polypeptide of the invention, e.g., an epitope of a polypeptide of the invention. A molecule which specifically binds to a given polypeptide of the invention is a molecule which binds the polypeptide, but does not substantially bind other molecules in a sample, e.g., a biological sample, which naturally contains the polypeptide. Examples of immunologically active portions of immunoglobulin molecules include F(ab) and F(ab′)₂ fragments which can be generated by treating the antibody with an enzyme such as pepsin. The invention provides polyclonal and monoclonal antibodies. The term “monoclonal antibody” or “monoclonal antibody composition”, as used herein, refers to a population of antibody molecules that contain only one species of an antigen binding site capable of immunoreacting with a particular epitope.

Polyclonal antibodies can be prepared as described above by immunizing a suitable subject with a polypeptide of the invention as an immunogen. Preferred polyclonal antibody compositions are ones that have been selected for antibodies directed against a polypeptide or polypeptides of the invention. Particularly preferred polyclonal antibody preparations are ones that contain only antibodies directed against a polypeptide or polypeptides of the invention. Particularly preferred immunogen compositions are those that contain no other human proteins such as, for example, immunogen compositions made using a non-human host cell for recombinant expression of a polypeptide of the invention. In such a manner, the only human epitope or epitopes recognized by the resulting antibody compositions raised against this immunogen will be present as part of a polypeptide or polypeptides of the invention.

The antibody titer in the immunized subject can be monitored over time by standard techniques, such as with an enzyme linked immunosorbent assay (ELISA) using immobilized polypeptide. If desired, the antibody molecules can be harvested or isolated from the subject (e.g., from the blood, plasma, or serum of the subject) and further purified by well-known techniques, such as protein A chromatography to obtain the IgG fraction. Alternatively, antibodies specific for a protein or polypeptide of the invention can be selected or (e.g., partially purified) or purified by, e.g., affinity chromatography. For example, a recombinantly expressed and purified (or partially purified) protein of the invention is produced as described herein, and covalently or non-covalently coupled to a solid support such as, for example, a chromatography column. The column can then be used to affinity purify antibodies specific for the proteins of the invention from a sample containing antibodies directed against a large number of different epitopes, thereby generating a substantially purified antibody composition, i.e., one that is substantially free of contaminating antibodies. By a substantially purified antibody composition is meant, in this context, that the antibody sample contains at most only 30% (by dry weight) of contaminating antibodies directed against epitopes other than those of the desired protein or polypeptide of the invention, and preferably at most 20%, yet more preferably at most 10%, and most preferably at most 5% (by dry weight) of the sample is contaminating antibodies. A purified antibody composition means that at least 99% of the antibodies in the composition are directed against the desired protein or polypeptide of the invention.

At an appropriate time after immunization, e.g., when the specific antibody titers are highest, antibody-producing cells can be obtained from the subject and used to prepare monoclonal antibodies by standard techniques, such as the hybridoma technique originally described by Kohler and Milstein (1975) Nature 256:495-497, the human B cell hybridoma technique (see Kozbor et al., 1983, Immunol. Today 4:72), the EBV-hybridoma technique (see Cole et al., pp. 77-96 In Monoclonal Antibodies and Cancer Therapy, Alan R. Liss, Inc. 1985) or trioma techniques. The technology for producing hybridomas is well known (see generally Current Protocols in Irnmunology, Coligan et al. ed., John Wiley & Sons, New York, 1994). Hybridoma cells producing a monoclonal antibody of the invention are detected by screening the hybridoma culture supernatants for antibodies that bind the polypeptide of interest, e.g., using a standard ELISA assay.

Alternative to preparing monoclonal antibody-secreting hybridomas, a monoclonal antibody directed against a polypeptide of the invention can be identified and isolated by screening a recombinant combinatorial immunoglobulin library (e.g., an antibody phage display library) with the polypeptide of interest. Kits for generating and screening phage display libraries are commercially available (e.g., the Pharmacia Recombinant Phage Antibody System, Catalog No. 27-9400-01; and the Stratagene SurfZAP Phage Display Kit, Catalog No. 240612). Additionally, examples of methods and reagents particularly amenable for use in generating and screening antibody display library can be found in, for example, U.S. Pat. No. 5,223,409; PCT Publication No. WO 92/18619; PCT Publication No. WO 91/17271; PCT Publication No. WO 92/20791; PCT Publication No. WO 92/15679; PCT Publication No. WO 93/01288; PCT Publication No. WO 92/01047; PCT Publication No. WO 92/09690; PCT Publication No. WO 90/02809; Fuchs et al. (1991) Bio/Technology 9:1370-1372; Hay et al. (1992) Hum. Antibod. Hybridomas 3:81-85; Huse et al. (1989) Science 246:1275-1281; Griffiths et al. (1993) EMBO J. 12:725-734.

Additionally, recombinant antibodies, such as chimeric and humanized monoclonal antibodies, comprising both human and non-human portions, which can be made using standard recombinant DNA techniques, are within the scope of the invention. A chimeric antibody is a molecule in which different portions are derived from different animal species, such as those having a vanrable region derived from a murine mAb and a human immunoglobulin constant region. (See, e.g., Cabilly et al., U.S. Pat. No. 4,816,567; and Boss et al., U.S. Pat. No. 4,816,397, which are incorporated herein by reference in their entirety.) Humanized antibodies are antibody molecules from non-human species having one or more complementarily determining regions (CDRs) from the non-human species and a framework region from a human immunoglobulin molecule. (See, e.g., Queen, U.S. Pat. No. 5,585,089, which is incorporated herein by reference in its entirety.) Such chimeric and humanized monoclonal antibodies can be produced by recombinant DNA techniques known in the art, for example using methods described in PCT Publication No. WO 87/02671; European Patent Application 184,187; European Patent Application 171,496; European Patent Application 173,494; PCT Publication No. WO 86/01533; U.S. Pat. No. 4,816,567; European Patent Application 125,023; Better et al. (1988) Science 240:1041-1043; Liu et al. (1987) Proc. Natl. Acad. Sci. USA 84:3439-3443; Liu et al. (1987) J. Immunol. 139:3521-3526; Sun et al. (1987) Proc. Natl. Acad. Sci. USA 84:214-218; Nishimura et al. (1987) Cancer Res. 47:999-1005; Wood et al. (1985) Nature 314:446-449; and Shaw et al. (1988) J. Natl. Cancer Inst. 80:1553-1559); Morrison (1985) Science 229:1202-1207; Oi et al. (1986) Bio/Techniques 4:214; U.S. Pat. No. 5,225,539; Jones et al. (1986) Nature 321:552-525; Verhoeyan et al. (1988) Science 239:1534; and Beidler et al. (1988) J. Immunol. 141:4053-4060.

Antibodies of the invention may be used as therapeutic agents in treating RA. In a preferred embodiment, completely human antibodies of the invention are used for therapeutic treatment of human RA patients, particularly those having erosive and non-erosive RA. Such antibodies can be produced, for example, using transgenic mice which are incapable of expressing endogenous immunoglobulin heavy and light chains genes, but which can express human heavy and light chain genes. The transgenic mice are immunized in the normal fashion with a selected antigen, e.g., all or a portion of a polypeptide corresponding to a marker of the invention. Monoclonal antibodies directed against the antigen can be obtained using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA and IgE antibodies. For an overview of this technology for producing human antibodies, see Lonberg and Huszar (1995) Int. Rev. Immunol. 13:65-93). For a detailed discussion of this technology for producing human antibodies and human monoclonal antibodies and protocols for producing such antibodies, see, e.g., U.S. Pat. Nos. 5,625,126; 5,633,425; 5,569,825; 5,661,016; and 5,545,806. In addition, companies such as Abgenix, Inc. (Freemont, Calif.), can be engaged to provide human antibodies directed against a selected antigen using technology similar to that described above.

Completely human antibodies which recognize a selected epitope can be generated using a technique referred to as “guided selection.” In this approach a selected non-human monoclonal antibody, e.g., a murine antibody, is used to guide the selection of a completely human antibody recognizing the same epitope (Jespers et al., 1994, Bio/technology 12:899-903).

An antibody directed against a polypeptide corresponding to a marker of the invention (e.g., a monoclonal antibody) can be used to isolate the polypeptide by standard techniques, such as affinity chromatography or immunoprecipitation. Moreover, such an antibody can be used to detect the marker (e.g., in a cellular lysate or cell supernatant) in order to evaluate the level and pattern of expression of the marker. The antibodies can also be used diagnostically to monitor protein levels in tissues or body fluids (e.g. in an ovary-associated body fluid) as part of a clinical testing procedure, e.g., to, for example, determine the efficacy of a given treatment regimen. Detection can be facilitated by coupling the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

Further, an antibody (or fragment thereof) can be conjugated to a therapeutic moiety such as a cytotoxin, a therapeutic agent or a radioactive metal ion. A cytotoxin or cytotoxic agent includes any agent that is detrimental to cells. Examples include, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

The conjugates of the invention can be used for modifying a given biological response, the drug moiety is not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin, or diphtheria toxin; a protein such as tumor necrosis factor, .alpha.-interferon, .beta.-interferon, nerve growth factor, platelet derived growth factor, tissue plasminogen activator; or, biological response modifiers such as, for example, lymphokines, interleukin-1 (“IL-1”), interleukin-2 (“IL-2”), interleukin-6 (“IL-6”), granulocyte macrophase colony stimulating factor (“GM-CSF”), granulocyte colony stimulating factor (“G-CSF”), or other growth factors.

Techniques for conjugating such therapeutic moiety to antibodies are well known, see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., “The Preparation And Cytotoxic Properties Of Antibody-Toxin Conjugates”, Immunol. Rev., 62:119-58 (1982).

Alternatively, an antibody can be conjugated to a second antibody to form an antibody heteroconjugate as described by Segal in U.S. Pat. No. 4,676,980. Accordingly, in one aspect, the invention provides substantially purified antibodies or fragments thereof, and non-human antibodies or fragments thereof, which antibodies or fragments specifically bind to a polypeptide comprising an amino acid sequence selected from the group consisting of the amino acid sequences of the present invention, an amino acid sequence encoded by the cDNA of the present invention, a fragment of at least 15 amino acid residues of an amino acid sequence of the present invention, an amino acid sequence which is at least 95% identical to the amino acid sequence of the present invention (wherein the percent identity is determined using the ALIGN program of the GCG software package with a PAM120 weight residue table, a gap length penalty of 12, and a gap penalty of 4) and an amino acid sequence which is encoded by a nucleic acid molecule which hybridizes to a nucleic acid molecule consisting of the nucleic acid molecules of the present invention, or a complement thereof, under conditions of hybridization of 6×SSC at 45° C. and washing in 0.2×SSC, 0.1% SDS at 65° C. In various embodiments, the substantially purified antibodies of the invention, or fragments thereof, can be human, non-human, chimeric and/or humanized antibodies.

In another aspect, the invention provides non-human antibodies or fragments thereof, which antibodies or fragments specifically bind to a polypeptide comprising an amino acid sequence selected from the group consisting of: the amino acid sequence of the present invention, an amino acid sequence encoded by the cDNA of the present invention, a fragment of at least 15 amino acid residues of the amino acid sequence of the present invention, an amino acid sequence which is at least 95% identical to the amino acid sequence of the present invention (wherein the percent identity is determined using the ALIGN program of the GCG software package with a PAM120 weight residue table, a gap length penalty of 12, and a gap penalty of 4) and an amino acid sequence which is encoded by a nucleic acid molecule which hybridizes to a nucleic acid molecule consisting of the nucleic acid molecules of the present invention, or a complement thereof, under conditions of hybridization of 6×SSC at 45° C. and washing in 0.2×SSC, 0.1% SDS at 65° C. Such non-human antibodies can be goat, mouse, sheep, horse, chicken, rabbit, or rat antibodies. Alternatively, the non-human antibodies of the invention can be chimeric and/or humanized antibodies. In addition, the non-human antibodies of the invention can be polyclonal antibodies or monoclonal antibodies.

In still a further aspect, the invention provides monoclonal antibodies or fragments thereof, which antibodies or fragments specifically bind to a polypeptide comprising an amino acid sequence selected from the group consisting of the amino acid sequences of the present invention, an amino acid sequence encoded by the cDNA of the present invention, a fragment of at least 15 amino acid residues of an amino acid sequence of the present invention, an amino acid sequence which is at least 95% identical to an amino acid sequence of the present invention (wherein the percent identity is determined using the ALIGN program of the GCG software package with a PAM120 weight residue table, a gap length penalty of 12, and a gap penalty of 4) and an amino acid sequence which is encoded by a nucleic acid molecule which hybridizes to a nucleic acid molecule consisting of the nucleic acid molecules of the present invention, or a complement thereof, under conditions of hybridization of 6×SSC at 45° C. and washing in 0.2×SSC, 0.1% SDS at 65° C. The monoclonal antibodies can be human, humanized, chimeric and/or non-human antibodies.

The substantially purified antibodies or fragments thereof may specifically bind to a signal peptide, a secreted sequence, an extracellular domain, a transmembrane or a cytoplasmic domain or cytoplasmic membrane of a polypeptide of the invention. In a particularly preferred embodiment, the substantially purified antibodies or fragments thereof, the non-human antibodies or fragments thereof, and/or the monoclonal antibodies or fragments thereof, of the invention specifically bind to a secreted sequence or an extracellular domain of the amino acid sequences of the present invention. Any of the antibodies of the invention can be conjugated to a therapeutic moiety or to a detectable substance. Non-limiting examples of detectable substances that can be conjugated to the antibodies of the invention are an enzyme, a prosthetic group, a fluorescent material, a luminescent material, a bioluminescent material, and a radioactive material.

The invention also provides a kit containing an antibody of the invention conjugated to a detectable substance, and instructions for use. Still another aspect of the invention is a pharmaceutical composition comprising an antibody of the invention and a pharmaceutically acceptable carrier. In preferred embodiments, the pharmaceutical composition contains an antibody of the invention, a therapeutic moiety, and a pharmaceutically acceptable carrier.

Still another aspect of the invention is a method of making an antibody that specifically recognizes a polypeptide of the present invention, the method comprising immunizing a mammal with a polypeptide. The polypeptide used as an immunogen comprises an amino acid sequence selected from the group consisting of the amino acid sequence of the present invention, an amino acid sequence encoded by the cDNA of the nucleic acid molecules of the present invention, a fragment of at least 15 amino acid residues of the amino acid sequence of the present invention, an amino acid sequence which is at least 95% identical to the amino acid sequence of the present invention (wherein the percent identity is determined using the ALIGN program of the GCG software package with a PAM120 weight residue table, a gap length penalty of 12, and a gap penalty of 4) and an amino acid sequence which is encoded by a nucleic acid molecule which hybridizes to a nucleic acid molecule consisting of the nucleic acid molecules of the present invention, or a complement thereof, under conditions of hybridization of 6×SSC at 45° C. and washing in 0.2×SSC, 0.1% SDS at 65° C. After immunization, a sample is collected from the mammal that contains an antibody that specifically recognizes the polypeptide. Preferably, the polypeptide is recombinantly produced using a non-human host cell. Optionally, the antibodies can be further purified from the sample using techniques well known to those of skill in the art. The method can further comprise producing a monoclonal antibody-producing cell from the cells of the mammal. Optionally, antibodies are collected from the antibody-producing cell.

II. Isolated Nucleic Acid Molecules

Another aspect of the invention pertains to isolated nucleic acid molecules that correspond to a marker of the invention, including nucleic acids which encode a marker protein of the invention or a portion of such a polypeptide. Isolated nucleic acids of the invention also include nucleic acid molecules sufficient for use as hybridization probes to identify nucleic acid molecules that correspond to a marker of the invention, including nucleic acids which encode a polypeptide corresponding to a marker of the invention, and fragments of such nucleic acid molecules, e.g., those suitable for use as PCR primers for the amplification or mutation of nucleic acid molecules. As used herein, the term “nucleic acid molecule” is intended to include DNA molecules (e.g., cDNA or genomic DNA) and RNA molecules (e.g., mRNA) and analogs of the DNA or RNA generated using nucleotide analogs. The nucleic acid molecule can be single-stranded or double-stranded, but preferably is double-stranded DNA.

An “isolated” nucleic acid molecule is one which is separated from other nucleic acid molecules which are present in the natural source of the nucleic acid molecule. Preferably, an “isolated” nucleic acid molecule is free of sequences (preferably protein-encoding sequences) which naturally flank the nucleic acid (i.e., sequences located at the 5′ and 3′ ends of the nucleic acid) in the genomic DNA of the organism from which the nucleic acid is derived. For example, in various embodiments, the isolated nucleic acid molecule can contain less than about 5 kB, 4 kB, 3 kB, 2 kB, I kB, 0.5 kB or 0.1 kB of nucleotide sequences which naturally flank the nucleic acid molecule in genomic DNA of the cell from which the nucleic acid is derived. Moreover, an “isolated” nucleic acid molecule, such as a cDNA molecule, can be substantially free of other cellular material, or culture medium when produced by recombinant techniques, or substantially free of chemical precursors or other chemicals when chemically synthesized.

A nucleic acid molecule of the present invention, e.g., a nucleic acid encoding a marker protein can be isolated using standard molecular biology techniques and the sequence information in the database records described herein. Using all or a portion of such nucleic acid sequences, nucleic acid molecules of the invention can be isolated using standard hybridization and cloning techniques (e.g., as described in Sambrook et al., ed., Molecular Cloning: A Laboratory Manual, 2nd ed., Cold Spring Harbor Laboratory Press, Cold Spring Harbor, N.Y., 1989).

A process for identifying the full-length coding sequence of a marker of the present invention is thus also provided. Any conventional recombinant DNA techniques applicable for isolating polynucleotides may also be employed. One such method involves the 5′-RACE-PCR technique, in which the poly-A mRNA that contains the coding sequence of particular interest is first reverse transcribed with a 3′-primer comprising a sequence disclosed herein. The newly synthesized cDNA strand is then tagged with an anchor primer with a known sequence, which preferably contains a convenient cloning restriction site attached at the 5′end. The tagged cDNA is then internal sequences of the coding region) and the 5′-anchor primer. The amplification may be conducted under conditions of various levels of stringency to optimize the amplification specificity. 5′-RACE-PCR can be readily performed using commercial kits (available from, e.g., BRL Life Technologies Inc., Clontech) according to the manufacturer's instructions.

Isolating the complete coding sequence of a gene can also be carried out in a hybridization assay using a suitable probe. The probe preferably comprises at least 10 nucleotides, and more preferably exhibits sequence homology to the polynucleotides of the markers of the present invention. Other high throughput screens for cDNAs, such as those involving gene chip technology, can also be employed in obtaining the complete cDNA sequence.

In addition, databases exist that reduce the complexity of ESTs by assembling contiguous EST sequences into tentative genes. For example, TIGR has assembled human ESTs into a datable called THC for tentative human consensus sequences. The THC database allows for a more definitive assignment compared to ESTs alone. Software programs exist (TIGR assembler and TIGEM EST assembly machine and contig assembly program (see Huang, X., 1996, Genomes 33:21-23)) that allow for assembling ESTs into contiguous sequences from any organism.

Alternatively, mRNA from a sample preparation is used to construct cDNA library in the ZAP Express vector following the procedure described in Velculescu et al., 1997, Science 270:484. The ZAP Express cDNA synthesis kit (Stratagene) is used accordingly to the manufacturer's protocol. Plates containing 250 to 2000 plaques are hybridized as described in Rupert et al., 1988, Mol. Cell. Bio. 8:3104 to oligonucleotide probes with the same conditions previously described for standard probes except that the hybridization temperature is reduced to a room temperature. Washes are performed in 6×standard-saline-citrate 0.1% SDS for 30 minutes at room temperature. The probes are labeled with ³²P-ATP trough use of T4 polynucleotide kinase.

A partial cDNA (3′ fragment) can be isolated by 3′ directed PCR reaction. This procedure is a modification of the protocol described in Polyak et al., 1997, Nature 389:300. Briefly, the procedure uses SAGE tags in PCR reaction such that the resultant PCR product contains the SAGE tag of interest as well as additional cDNA, the length of which is defined by the position of the tag with respect to the 3′ end of the cDNA. The cDNA product derived from such a transcript driven PCR reaction can be used for many applications.

RNA from a source to express the cDNA corresponding to a given tag is first converted to double-stranded cDNA using any standard cDNA protocol. Similar conditions used to generate cDNA for SAGE library construction can be employed except that a modified oligo-dT primer is used to derive the first strand synthesis. For example, the oligonucleotide of composition 5′-B-TCC GGC GCG CCG TTT TCC CAG TCA CGA(30)-3′, contains a poly-T stretch at the 3′end for hybridization and priming from poly-A tails, an M13 priming site for use in subsequent PCR steps, a 5′ Biotin label (B) for capture to strepavidin-coated magnetic beads, and an AscI restriction endonuclease site for releasing the cDNA from the strepavidin-coated magnetic beads. Theoretically, any sufficiently-sized DNA region capable of hybridizing to a PCR primer can be used as well as any other 8 base pair recognizing endonuclease. cDNA constructed utilizing this or similar modified oligo-dT primer is then processed exactly as described in U.S. Pat. No. 5,695,937 up until adapter ligation where only one adapter is ligated to the cDNA pool. After Adapter ligation, the cDNA is released from the streptavidin-coated magnetic beads and is then used as a template for cDNA amplification.

Various PCR protocols can be employed using PCR priming sites within the 3′ modified oligo-dT primer and the SAGE tag. The SAGE tag-derived PCR/primer employed can be of varying length dictated by 5′ extension of the tag into the adaptor sequence. cDNA products are now available for a variety of applications.

This technique can be further modified by: (1) altering the length and/or content of the modified oligo-dT primer; (2) ligating adaptors other than that previously employed within the SAGE protocol; (3) performing PCR from template retained on the streptavidin-coated magnetic beads; and (4) priming first strand cDNA synthesis with non-oligo-dT based primers.

Gene trapper technology can also be used. The reagents and manufacturer's instructions for this technology are commercially available from Life Technologies, Inc., Gaithsburg, Maryland. Briefly, a complex population of single-stranded phagemid DNA containing directional cDNA inserts is enriched for the target sequence by hybridization in solution to a biotinylated oligonucleotide probe complementary to the target sequence. The hybrids are captured on streptavidin-coated paramagnetic beads. A magnet retrieves the paramagnetic beads from the solution, leaving nonhybridized single-stranded DNAs behind. Subsequently, the captured single-stranded DNA target is released from the biotinylated oligonucleotide. After release, the cDNA clone is further enriched by using a nonbiotinylated target oligonucleotide to specifically prime conversion of the single-stranded DNA. Following transformation and plating, typically 20% to 100% of the colonies represent the cDNA clone of interest. To identify the desired cDNA clone, the colonies may be screened by colony hybridization using the ³²P-labeled oligonucleotide as described above for solution hybridization, or alternatively by DNA sequencing and alignment of all sequences obtained from numerous clones to determine a consensus sequence.

A nucleic acid molecule of the invention can be amplified using cDNA, mRNA, or genomic DNA as a template and appropriate oligonucleotide primers according to standard PCR amplification techniques. The nucleic acid so amplified can be cloned into an appropriate vector and characterized by DNA sequence analysis. Furthermore, oligonucleotides corresponding to all or a portion of a nucleic acid molecule of the invention can be prepared by standard synthetic techniques, e.g., using an automated DNA synthesizer.

In another preferred embodiment, an isolated nucleic acid molecule of the invention comprises a nucleic acid molecule which has a nucleotide sequence complementary to the nucleotide sequence of a nucleic acid corresponding to a marker of the invention or to the nucleotide sequence of a nucleic acid encoding a protein which corresponds to a marker of the invention. A nucleic acid molecule which is complementary to a given nucleotide sequence is one which is sufficiently complementary to the given nucleotide sequence that it can hybridize to the given nucleotide sequence thereby forming a stable duplex.

Moreover, a nucleic acid molecule of the invention can comprise only a portion of a nucleic acid sequence, wherein the full length nucleic acid sequence comprises a marker of the invention or which encodes a polypeptide corresponding to a marker of the invention. Such nucleic acids can be used, for example, as a probe or primer. The probe/primer typically is used as one or more substantially purified oligonucleotides. The oligonucleotide typically comprises a region of nucleotide sequence that hybridizes under stringent conditions to at least about 7, preferably about 15, more preferably about 25, 50, 75, 100, 125, 150, 175, 200, 250, 300, 350, or 400 or more consecutive nucleotides of a nucleic acid of the invention.

Probes based on the sequence of a nucleic acid molecule of the invention can be used to detect transcripts or genomic sequences corresponding to one or more markers of the invention. The probe comprises a label group attached thereto, e.g., a radioisotope, a fluorescent compound, an enzyme, or an enzyme co-factor. Such probes can be used as part of a diagnostic test kit for identifying cells or tissues which mis-express the protein, such as by measuring levels of a nucleic acid molecule encoding the protein in a sample of cells from a subject, e.g., detecting mRNA levels or determining whether a gene encoding the protein has been mutated or deleted.

The invention further encompasses nucleic acid molecules that differ, due to degeneracy of the genetic code, from the nucleotide sequence of nucleic acids encoding a protein which corresponds to a marker of the invention, and thus encode the same protein.

In addition to the nucleotide sequences described herein, it will be appreciated by those skilled in the art that DNA sequence polymorphisms that lead to changes in the amino acid sequence can exist within a population (e.g., the human population). Such genetic polymorphisms can exist among individuals within a population due to natural allelic variation. An allele is one of a group of genes which occur alternatively at a given genetic locus. In addition, it will be appreciated that DNA polymorphisms that affect RNA expression levels can also exist that may affect the overall expression level of that gene (e.g., by affecting regulation or degradation).

As used herein, the phrase “allelic variant” refers to a nucleotide sequence which occurs at a given locus or to a polypeptide encoded by the nucleotide sequence.

As used herein, the terms “gene” and “recombinant gene” refer to nucleic acid molecules comprising an open reading frame encoding a polypeptide corresponding to a marker of the invention. Such natural allelic variations can typically result in 1-5% variance in the nucleotide sequence of a given gene. Alternative alleles can be identified by sequencing the gene of interest in a number of different individuals. This can be readily carried out by using hybridization probes to identify the same genetic locus in a variety of individuals. Any and all such nucleotide variations and resulting amino acid polymorphisms or variations that are the result of natural allelic variation and that do not alter the functional activity are intended to be within the scope of the invention.

In another embodiment, an isolated nucleic acid molecule of the invention is at least 7, 15, 20, 25, 30, 40, 60, 80, 100, 150, 200, 250, 300, 350, 400, 450, 550, 650, 700, 800, 900, 1000, 1200, 1400, 1600, 1800, 2000, 2200, 2400, 2600, 2800, 3000, 3500, 4000, 4500, or more nucleotides in length and hybridizes under stringent conditions to a nucleic acid corresponding to a marker of the invention or to a nucleic acid encoding a protein corresponding to a marker of the invention. As used herein, the term “hybridizes under stringent conditions” is intended to describe conditions for hybridization and washing under which nucleotide sequences at least 75% (80%, 85%, preferably 90%) identical to each other typically remain hybridized to each other. Such stringent conditions are known to those skilled in the art and can be found in sections 6.3.1-6.3.6 of Current Protocols in Molecular Biology, John Wiley & Sons, N.Y. (1989). A preferred, non-limiting example of stringent hybridization conditions for annealing two single-stranded DNA each of which is at least about 100 bases in length and/or for annealing a single-stranded DNA and a single-stranded RNA each of which is at least about 100 bases in length, are hybridization in 6×sodium chloride/sodium citrate (SSC) at about 45° C., followed by one or more washes in 0.2×SSC, 0.1% SDS at 50-65° C. Further preferred hybridization conditions are taught in Lockhart, et al., Nature Biotechnology, Volume 14, 1996 August:1675-1680; Breslauer, et al., Proc. Natl. Acad. Sci. USA, Volume 83, 1986 June: 3746-3750; Van Ness, et al., Nucleic Acids Research, Volume 19, No. 19, 1991 September: 5143-5151; McGraw, et al., BioTechniques, Volume 8, No. 6 1990: 674-678; and Milner, et al., Nature Biotechnology, Volume 15, 1997 June: 537-541, all expressly incorporated by reference.

In addition to naturally-occurring allelic variants of a nucleic acid molecule of the invention that can exist in the population, the skilled artisan will further appreciate that sequence changes can be introduced by mutation thereby leading to changes in the amino acid sequence of the encoded protein, without altering the biological activity of the protein encoded thereby. For example, one can make nucleotide substitutions leading to amino acid substitutions at “non-essential” amino acid residues. A “non-essential” amino acid residue is a residue that can be altered from the wild-type sequence without altering the biological activity, whereas an “essential” amino acid residue is required for biological activity. For example, amino acid residues that are not conserved or only semi-conserved among homologs of various species may be non-essential for activity and thus would be likely targets for alteration. Alternatively, amino acid residues that are conserved among the homologs of various species (e.g., murine and human) may be essential for activity and thus would not be likely targets for alteration.

Accordingly, another aspect of the invention pertains to nucleic acid molecules encoding a polypeptide of the invention that contain changes in amino acid residues that are not essential for activity. Such polypeptides differ in amino acid sequence from the naturally-occurring proteins which correspond to the markers of the invention, yet retain biological activity. In one embodiment, such a protein has an amino acid sequence that is at least about 40% identical, 50%, 60%, 70%, 80%, 90%, 95%, or 98% identical to the amino acid sequence of one of the proteins which correspond to the markers of the invention.

An isolated nucleic acid molecule encoding a variant protein can be created by introducing one or more nucleotide substitutions, additions or deletions into the nucleotide sequence of nucleic acids of the invention, such that one or more amino acid residue substitutions, additions, or deletions are introduced into the encoded protein. Mutations can be introduced by standard techniques, such as site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), non-polar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Alternatively, mutations can be introduced randomly along all or part of the coding sequence, such as by saturation mutagenesis, and the resultant mutants can be screened for biological activity to identify mutants that retain activity. Following mutagenesis, the encoded protein can be expressed recombinantly and the activity of the protein can be determined.

The present invention encompasses antisense nucleic acid molecules, i.e., molecules which are complementary to a sense nucleic acid of the invention, e.g., complementary to the coding strand of a double-stranded cDNA molecule corresponding to a marker of the invention or complementary to an mRNA sequence corresponding to a marker of the invention. Accordingly, an antisense nucleic acid of the invention can hydrogen bond to (i.e. anneal with) a sense nucleic acid of the invention. The antisense nucleic acid can be complementary to an entire coding strand, or to only a portion thereof, e.g., all or part of the protein coding region (or open reading frame). An antisense nucleic acid molecule can also be antisense to all or part of a non-coding region of the coding strand of a nucleotide sequence encoding a polypeptide of the invention. The non-coding regions (“5′ and 3′ untranslated regions”) are the 5′ and 3′ sequences which flank the coding region and are not translated into amino acids.

An antisense oligonucleotide can be, for example, about 5, 10, 15, 20, 25, 30, 35, 40, 45, or 50 or more nucleotides in length. An antisense nucleic acid of the invention can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xanthine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been sub-cloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/or genomic DNA encoding a polypeptide corresponding to a selected marker of the invention to thereby inhibit expression of the marker, e.g., by inhibiting transcription and/or translation. The hybridization can be by conventional nucleotide complementarity to form a stable duplex, or, for example, in the case of an antisense nucleic acid molecule which binds to DNA duplexes, through specific interactions in the major groove of the double helix. Examples of a route of administration of antisense nucleic acid molecules of the invention includes direct injection at a tissue site or infusion of the antisense nucleic acid into an RA-associated body fluid. Alternatively, antisense nucleic acid molecules can be modified to target selected cells and then administered systemically. For example, for systemic administration, antisense molecules can be modified such that they specifically bind to receptors or antigens expressed on a selected cell surface, e.g., by linking the antisense nucleic acid molecules to peptides or antibodies which bind to cell surface receptors or antigens. The antisense nucleic acid molecules can also be delivered to cells using the vectors described herein. To achieve sufficient intracellular concentrations of the antisense molecules, vector constructs in which the antisense nucleic acid molecule is placed under the control of a strong pol II or pol III promoter are preferred.

An antisense nucleic acid molecule of the invention can be an 0-anomeric nucleic acid molecule. An α-anomeric nucleic acid molecule forms specific double-strands stranded hybrids with complementary RNA in which, contrary to the usual α-units, the run parallel to each other (Gaultier et al., 1987, Nucleic Acids Res. 15:6625-6641). The antisense nucleic acid molecule can also comprise a 2′-o-methylribonucleotide (Inoue et al., 1987, Nucleic Acids Res. 15:6131-6148) or a chimeric RNA-DNA analogue (Inoue et al., 1987, FEBS Lett. 215:327-330).

The invention also encompasses ribozymes. Ribozymes are catalytic RNA molecules with ribonuclease activity which are capable of cleaving a single-stranded nucleic acid, such as an mRNA, to which they have a complementary region. Thus, ribozymes (e.g., hammerhead ribozymes as described in Haselhoff and Gerlach, 1988, Nature 334:585-591) can be used to catalytically cleave mRNA transcripts to thereby inhibit translation of the protein encoded by the mRNA. A ribozyme having specificity for a nucleic acid molecule encoding a polypeptide corresponding to a marker of the invention can be designed based upon the nucleotide sequence of a cDNA corresponding to the marker. For example, a derivative of a Tetrahymena L-19 IVS RNA can be constructed in which the nucleotide sequence of the active site is complementary to the nucleotide sequence to be cleaved (see Cech et al. U.S. Pat. No. 4,987,071; and Cech et al. U.S. Pat. No. 5,116,742). Alternatively, an mRNA encoding a polypeptide of the invention can be used to select a catalytic RNA having a specific ribonuclease activity from a pool of RNA molecules (see, e.g., Bartel and Szostak, 1993, Science 261:1411-1418).

The invention also encompasses nucleic acid molecules which form triple helical structures. For example, expression of a polypeptide of the invention can be inhibited by targeting nucleotide sequences complementary to the regulatory region of the gene encoding the polypeptide (e.g., the promoter and/or enhancer) to form triple helical structures that prevent transcription of the gene in target cells. See generally Helene (1991) Anticancer Drug Des. 6(6):569-84; Helene (1992) Ann. N. Y. Acad. Sci. 660:27-36; and Maher (1992) Bioassays 14(12):807-15.

In various embodiments, the nucleic acid molecules of the invention can be modified at the base moiety, sugar moiety or phosphate backbone to improve, e.g., the stability, hybridization, or solubility of the molecule. For example, the deoxyribose phosphate backbone of the nucleic acids can be modified to generate peptide nucleic acids (see Hyrup et al., 1996, Bioorganic & Medicinal Chemistry 4(1): 5-23). As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup et al. (1996), supra; Perry-O'Keefe et al. (1996) Proc. Natl. Acad. Sci. USA 93:14670-675.

PNAs can be used in therapeutic and diagnostic applications. For example, PNAs can be used as antisense or antigene agents for sequence-specific modulation of gene expression by, e.g., inducing transcription or translation arrest or inhibiting replication. PNAs can also be used, e.g., in the analysis of single base pair mutations in a gene by, e.g., PNA directed PCR clamping; as artificial restriction enzymes when used in combination with other enzymes, e.g., S1 nucleases (Hyrup (1996), supra; or as probes or primers for DNA sequence and hybridization (Hyrup, 1996, supra; Perry-O'Keefe et al., 1996, Proc. Natl. Acad. Sci. USA 93:14670-675).

In another embodiment, PNAs can be modified, e.g., to enhance their stability or cellular uptake, by attaching lipophilic or other helper groups to PNA, by the formation of PNA-DNA chimeras, or by the use of liposomes or other techniques of drug delivery known in the art. For example, PNA-DNA chimeras can be generated which can combine the advantageous properties of PNA and DNA. Such chimeras allow DNA recognition enzymes, e.g., RNASE H and DNA polymerases, to interact with the DNA portion while the PNA portion would provide high binding affinity and specificity. PNA-DNA chimeras can be linked using linkers of appropriate lengths selected in terms of base stacking, number of bonds between the nucleobases, and orientation (Hyrup, 1996, supra). The synthesis of PNA-DNA chimeras can be performed as described in Hyrup (1996), supra, and Finn et al. (1996) Nucleic Acids Res. 24(17):3357-63. For example, a DNA chain can be synthesized on a solid support using standard phosphoramidite coupling chemistry and modified nucleoside analogs. Compounds such as 5′-(4-methoxytrityl)amino-5′-deoxy-thymidine phosphoramidite can be used as a link between the PNA and the 5′ end of DNA (Mag et al., 1989, Nucleic Acids Res. 17:5973-88). PNA monomers are then coupled in a step-wise manner to produce a chimeric molecule with a 5′ PNA segment and a 3′ DNA segment (Finn et al., 1996, Nucleic Acids Res. 24(17):3357-63). Alternatively, chimeric molecules can be synthesized with a 5′DNA segment and a 3′PNA segment (Peterser et al., 1975, Bioorganic Med. Chem. Lett. 5:1119-11124).

In other embodiments, the oligonucleotide can include other appended groups such as peptides (e.g., for targeting host cell receptors in vivo), or agents facilitating transport across the cell membrane (see, e.g., Letsinger et al., 1989, Proc. Natl. Acad. Sci. USA 86:6553-6556; Lemaitre et al., 1987, Proc. Natl. Acad. Sci. USA 84:648-652; PCT Publication No. WO 88/09810) or the blood-brain barrier (see, e.g., PCT Publication No. WO 89/10134). In addition, oligonucleotides can be modified with hybridization-triggered cleavage agents (see, e.g., Krol et al., 1988, Bio/Techniques 6:958-976) or intercalating agents (see, e.g., Zon, 1988, Pharm. Res. 5:539-549). To this end, the oligonucleotide can be conjugated to another molecule, e.g., a peptide, hybridization triggered cross-linking agent, transport agent, hybridization-triggered cleavage agent, etc.

The invention also includes molecular beacon nucleic acids having at least one region which is complementary to a nucleic acid of the invention, such that the molecular beacon is useful for quantitating the presence of the nucleic acid of the invention in a sample. A “molecular beacon” nucleic acid is a nucleic acid comprising a pair of complementary regions and having a fluorophore and a fluorescent quencher associated therewith. The fluorophore and quencher are associated with different portions of the nucleic acid in such an orientation that when the complementary regions are annealed with one another, fluorescence of the fluorophore is quenched by the quencher. When the complementary regions of the nucleic acid are not annealed with one another, fluorescence of the fluorophore is quenched to a lesser degree. Molecular beacon nucleic acids are described, for example, in U.S. Pat. No. 5,876,930.

III. Recombinant Expression Vectors and Host Cells

Another aspect of the invention pertains to vectors, preferably expression vectors, containing a nucleic acid encoding a polypeptide corresponding to a marker of the invention (or a portion of such a polypeptide). As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors, namely expression vectors, are capable of directing the expression of genes to which they are operably linked. In general, expression vectors of utility in recombinant DNA techniques are often in the form of plasmids (vectors). However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell. This means that the recombinant expression vectors include one or more regulatory sequences, selected on the basis of the host cells to be used for expression, which is operably linked to the nucleic acid sequence to be expressed. Within a recombinant expression vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to include promoters, enhancers and other expression control elements (e.g., polyadenylation signals). Such regulatory sequences are described, for example, in Goeddel, Methods in Enzymology: Gene Expression Technology vol.185, Academic Press, San Diego, Calif. (1991). Regulatory sequences include those which direct constitutive expression of a nucleotide sequence in many types of host cell and those which direct expression of the nucleotide sequence only in certain host cells (e.g., tissue-specific regulatory sequences). It will be appreciated by those skilled in the art that the design,of the expression vector can depend on such factors as the choice of the host cell to be transformed, the level of expression of protein desired, and the like. The expression vectors of the invention can be introduced into host cells to thereby produce proteins or peptides, including fusion proteins or peptides, encoded by nucleic acids as described herein.

The recombinant expression vectors of the invention can be designed for expression of a polypeptide corresponding to a marker of the invention in prokaryotic (e.g., E. coli) or eukaryotic cells (e.g., insect cells {using baculovirus expression vectors}, yeast cells or mammalian cells). Suitable host cells are discussed further in Goeddel, supra. Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith and Johnson, 1988, Gene 67:31-40), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein.

Examples of suitable inducible non-fusion E. coli expression vectors include pTrc (Amann et al., 1988, Gene 69:301-315) and pET 11d (Studier et al., p. 60-89, In Gene Expression Technology: Methods in Enzymology vol.185, Academic Press, San Diego, Calif., 1991). Target gene expression from the pTrc vector relies on host RNA polymerase transcription from a hybrid trp-lac fusion promoter. Target gene expression from the pET 11d vector relies on transcription from a T7 gn10-lac fusion promoter mediated by a co-expressed viral RNA polymerase (T7 gnl). This viral polymerase is supplied by host strains BL21(DE3) or HMS174(DE3) from a resident prophage harboring a T7 gnl gene under the transcriptional control of the lacUV 5 promoter.

One strategy to maximize recombinant protein expression in E. coli is to express the protein in a host bacteria with an impaired capacity to proteolytically cleave the recombinant protein (Gottesman, p. 119-128, In Gene Expression Technology: Methods in Enzymology vol. 185, Academic Press, San Diego, Calif., 1990. Another strategy is to alter the nucleic acid sequence of the nucleic acid to be inserted into an expression vector so that the individual codons for each amino acid are those preferentially utilized in E. coli (Wada et al., 1992, Nucleic Acids Res. 20:2111-2118). Such alteration of nucleic acid sequences of the invention can be carried out by standard DNA synthesis techniques.

In another embodiment, the expression vector is a yeast expression vector. Examples of vectors for expression in yeast S. cerevisiae include pYepSecl (Baldari et al., 1987, EMBO J. 6:229-234), pMFa (Kurjan and Herskowitz, 1982, Cell 30:933-943), pJRY88 (Schultz et al., 1987, Gene 54:113-123), pYES2 (Invitrogen Corporation, San Diego, Calif.), and pPicZ (Invitrogen Corp, San Diego, Calif.).

In another embodiment, the methods of the present invention include the generation of markers of the invention by direct chemical synthesis, rather than by production from DNA, using the protein synthetic machinery of living organisms or cell extracts containing such machinery.

Alternatively, the expression vector is a baculovirus expression vector. Baculovirus vectors available for expression of proteins in cultured insect cells (e.g., Sf 9 cells) include the pAc series. (Smith et al., 1983, Mol. Cell Biol. 3:2156-2165) and the pVL series (Lucklow and Summers, 1989, Virology 170:31-39).

In yet another embodiment, a nucleic acid of the invention is expressed in mammalian cells using a mammalian expression vector. Examples of mammalian expression vectors include pCDM8 (Seed, 1987, Nature 329:840) and pMT2PC (Kaufman et al., 1987, EMBO J. 6:187-195). When used in mammalian cells, the expression vector's control functions are often provided by viral regulatory elements. cytomegalovirus and Simian Virus 40. For other suitable expression systems for both prokaryotic and eukaryotic cells see chapters 16 and 17 of Sambrook et al., supra.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert et al., 1987, Genes Dev. 1:268-277), lymphoid-specific promoters (Calame and Eaton, 1988, Adv. Immunol. 43:235-275), in particular promoters of T cell receptors (Winoto and Baltimore, 1989, EMBO J. 8:729-733) and immunoglobulins (Banerji et al., 1983, Cell 33:729-740; Queen and Baltimore, 1983, Cell 33:741-748), neuron-specific promoters (e.g., the neurofilament promoter; Byrne and Ruddle, 1989, Proc. Natl. Acad. Sci. USA 86:5473-5477), pancreas-specific promoters (Edlund et al., 1985, Science 230:912-916), and mammary gland-specific promoters (e.g., milk whey promoter; U.S. Pat. No. 4,873,316 and European Application Publication No. 264,166). Developmentally-regulated promoters are also encompassed, for example the murine hox promoters (Kessel and Gruss, 1990, Science 249:374-379) and the α-fetoprotein promoter (Camper and Tilghman, 1989, Genes Dev. 3:537-546).

The invention further provides a recombinant expression vector comprising a DNA molecule of the invention cloned into the expression vector in an antisense orientation. That is, the DNA molecule is operably linked to a regulatory sequence in a manner which allows for expression (by transcription of the DNA molecule) of an RNA molecule which is antisense to the mRNA encoding a polypeptide of the invention. Regulatory sequences operably linked to a nucleic acid cloned in the antisense orientation can be chosen which direct the continuous expression of the antisense RNA molecule in a variety of cell types, for instance viral promoters and/or enhancers, or regulatory sequences can be chosen which direct constitutive, tissue-specific or cell type specific expression of antisense RNA. The antisense expression vector can be in the form of a recombinant plasmid, phagemid, or attenuated virus in which antisense nucleic acids are produced under the control of a high efficiency regulatory region, the activity of which can be determined by the cell type into which the vector is introduced. For a discussion of the regulation of gene expression using antisense genes see Weintraub et al., 1986, Trends in Genetics, Vol. 1(1).

Another aspect of the invention pertains to host cells into which a recombinant expression vector of the invention has been introduced. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic (e.g., E. coli) or eukaryotic cell (e.g., insect cells, yeast or mammalian cells).

Vector.DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid into a host cell, including calcium phosphate or calcium chloride co-precipitation, DEAE-dextran-mediated transfection, lipofection, or electroporation. Suitable methods for transforming or transfecting host cells can be found in Sambrook, et al. (supra), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., for resistance to antibiotics) is generally introduced into the host cells along with the gene of interest. Preferred selectable markers include those which confer resistance to drugs, such as G418, hygromycin and methotrexate. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce a polypeptide corresponding to a marker of the invention. Accordingly, the invention further provides methods for producing a polypeptide corresponding to a marker of the invention using the host cells of the invention. In one embodiment, the method comprises culturing the host cell of invention (into which a recombinant expression vector encoding a polypeptide of the invention has been introduced) in a suitable medium such that the marker is produced. In another embodiment, the method further comprises isolating the marker polypeptide from the medium or the host cell.

The host cells of the invention can also be used to produce nonhuman transgenic animals. For example, in one embodiment, a host cell of the invention is a fertilized oocyte or an embryonic stem cell into which a sequences encoding a polypeptide corresponding to a marker of the invention have been introduced. Such host cells can then be used to create non-human transgenic animals in which exogenous sequences encoding a marker of the invention have been introduced into their genome or homologous recombinant animals in which endogenous gene(s) encoding a polypeptide corresponding to a marker of the invention sequences have been altered. Such animals are useful for studying the function and/or activity of the polypeptide corresponding to the marker and for identifying and/or evaluating modulators of polypeptide activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, etc. A transgene is exogenous DNA which is integrated into the genome of a cell from which a transgenic animal develops and which remains in the genome of the mature animal, thereby directing the expression of an encoded gene product in one or more cell types or tissues of the transgenic animal. As used herein, an “homologous recombinant animal” is a non-human animal, preferably a mammal, more preferably a mouse, in which an endogenous gene has been altered by homologous recombination between the endogenous gene and an exogenous DNA molecule introduced into a cell of the animal, e.g., an embryonic cell of the animal, prior to development of the animal.

A transgenic animal of the invention can be created by introducing a nucleic acid encoding a polypeptide corresponding to a marker of the invention into the male pronuclei of a fertilized oocyte, e.g., by microinjection, retroviral infection, and allowing the oocyte to develop in a pseudopregnant female foster animal. Intronic sequences and polyadenylation signals can also be included in the transgene to increase the efficiency of expression of the transgene. A tissue-specific regulatory sequence(s) can be operably linked to the transgene to direct expression of the polypeptide of the invention to particular cells. Methods for generating transgenic animals via embryo manipulation and microinjection, particularly animals such as mice, have become conventional in the art and are described, for example, in U.S. Pat. Nos. 4,736,866 and 4,870,009, U.S. Pat. No. 4,873,191 and in Hogan, Manipulating the Mouse Embryo, Cold Spring Harbor Laboratory Press, Cold Spring Harbor, N.Y., 1986. Similar methods are used for production of other transgenic animals. A transgenic founder animal can be identified based upon the presence of the transgene in its genome and/or expression of mRNA encoding the transgene in tissues or cells of the animals. A transgenic founder animal can then be used to breed additional animals carrying the transgene. Moreover, transgenic animals carrying the transgene can further be bred to other transgenic animals carrying other transgenes.

To create an homologous recombinant animal, a vector is prepared which contains at least a portion of a gene encoding a polypeptide corresponding to a marker of the invention into which a deletion, addition or substitution has been introduced to thereby alter, e.g., functionally disrupt, the gene. In a preferred embodiment, the vector is designed such that, upon homologous recombination, the endogenous gene is functionally disrupted (i.e., no longer encodes a functional protein; also referred to as a “knock out” vector). Alternatively, the vector can be designed such that, upon homologous recombination, the endogenous gene is mutated or otherwise altered but still encodes functional protein (e.g., the upstream regulatory region can be altered to thereby alter the expression of the endogenous protein). In the homologous recombination vector, the altered portion of the gene is flanked at its 5′ and 3′ ends by additional nucleic acid of the gene to allow for homologous recombination to occur between the exogenous gene carried by the vector and an endogenous gene in an embryonic stem cell. The additional flanking nucleic acid sequences are of sufficient length for successful homologous recombination with the endogenous gene. Typically, several kilobases of flanking DNA (both at the 5′ and 3′ ends) are included in the vector (see, e.g., Thomas and Capecchi, 1987, Cell 51:503 for a description of homologous recombination vectors). The vector is introduced into an embryonic stem cell line (e.g., by electroporation) and cells in which the introduced gene has homologously recombined with the endogenous gene are selected (see, e.g., Li et al., 1992, Cell 69:915). The selected cells are then injected into a blastocyst of an animal (e.g., a mouse) to form aggregation chimeras (see, e.g., Bradley, Teratocarcinomas and Embryonic Stem Cells: A Practical Approach, Robertson, Ed., IRL, Oxford, 1987, pp. 113-152). A chimeric embryo can then be implanted into a suitable pseudopregnant female foster animal and the embryo brought to term. Progeny harboring the homologously recombined DNA in their germ cells can be used to breed animals in which all cells of the animal contain the homologously recombined DNA by germline transmission of the transgene. Methods for constructing homologous recombination vectors and homologous recombinant animals are described further in Bradley (1991) Current Opinion in Bio/Technology 2:823-829 and in PCT Publication NOS. WO 90/11354, WO 91/01140, WO 92/0968, and WO 93/04169.

In another embodiment, transgenic non-human animals can be produced which contain selected systems which allow for regulated expression of the transgene. One example of such a system is the cre/loxP recombinase system of bacteriophage P1. For a description of the cre/loxP recombinase system, see, e.g., Lakso et al. (1992) Proc. Natl. Acad. Sci. USA 89:6232-6236. Another example of a recombinase system is the FLP recombinase system of Saccharomyces cerevisiae (O'Gorman et al., 1991, Science 251:1351-1355). If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut et al. (1997) Nature 385:810-813 and PCT Publication NOS. WO 97/07668 and WO 97/07669.

IV. Pharmaceutical Compositions

The nucleic acid molecules, polypeptides, and antibodies (also referred to herein as “active compounds”) corresponding to a marker of the invention can be incorporated into pharmaceutical compositions suitable for administration. Such compositions typically comprise the nucleic acid molecule, protein, or antibody and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

The invention includes methods for preparing pharmaceutical compositions for modulating the expression or activity of a polypeptide or nucleic acid corresponding to a marker of the invention. Such methods comprise formulating a pharmaceutically acceptable carrier with an agent which modulates expression or activity of a polypeptide or nucleic acid corresponding to a marker of the invention. Such compositions can further include additional active agents. Thus, the invention further includes methods for preparing a pharmaceutical composition by formulating a pharmaceutically acceptable carrier with an agent which modulates expression or activity of a polypeptide or nucleic acid corresponding to a marker of the invention and one or more additional active compounds.

The invention also provides methods (also referred to herein as “screening assays”) for identifying modulators, i.e., candidate or test compounds or agents (e.g., peptides, peptidomimetics, peptoids, small molecules or other drugs) which (a) bind to the marker, or (b) have a modulatory (e.g., stimulatory or inhibitory) effect on the activity of the marker or, more specifically, (c) have a modulatory effect on the interactions of the marker with one or more of its natural substrates (e.g., peptide, protein, hormone, co-factor, or nucleic acid), or (d) have a modulatory effect on the expression of the marker. Such assays typically comprise a reaction between the marker and one or more assay components. The other components may be either the test compound itself, or a combination of test compound and a natural binding partner of the marker.

The test compounds of the present invention may be obtained from any available source, including systematic libraries of natural and/or synthetic compounds. Test compounds may also be obtained by any of the numerous approaches in combinatorial library methods known in the art, including: biological libraries; peptoid libraries (libraries of molecules having the functionalities of peptides, but with a novel, non-peptide backbone which are resistant to enzymatic degradation but which nevertheless remain bioactive; see, e.g., Zuckermann et al., 1994, J. Med. Chem. 37:2678-85); spatially addressable parallel solid phase or solution phase libraries; synthetic library methods requiring deconvolution; the ‘one-bead one-compound’ library method; and synthetic library methods using affinity chromatography selection. The biological library and peptoid library approaches are limited to peptide libraries, while the other four approaches are applicable to peptide, non-peptide oligomer or small molecule libraries of compounds (Lam, 1997, Anticancer Drug Des. 12:145).

Examples of methods for the synthesis of molecular libraries can be found in the art, for example in: DeWitt et al. (1993) Proc. Natl. Acad. Sci. U.S.A. 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and in Gallop et al. (1994) J. Med. Chem. 37:1233.

Libraries of compounds may be presented in solution (e.g., Houghten, 1992, Biotechniques 13:412-421), or on beads (Lam, 1991, Nature 354:82-84), chips (Fodor, 1993, Nature 364:555-556), bacteria and/or spores, (Ladner, U.S. Pat. Ser. No. 5,223,409), plasmids (Cull et al, 1992, Proc Natl Acad Sci USA 89:1865-1869) or on phage (Scott and Smith, 1990, Science 249:386-390; Devlin, 1990, Science 249:404-406; Cwirla et al, 1990, Proc. Natl. Acad. Sci. 87:6378-6382; Felici, 1991, J. Mol. Biol. 222:301-310; Ladner, supra.).

In one embodiment, the invention provides assays for screening candidate or test compounds which are substrates of a marker or biologically active portion thereof. In another embodiment, the invention provides assays for screening candidate or test compounds which bind to a marker or biologically active portion thereof. Determining the ability of the test compound to directly bind to a marker can be accomplished, for example, by coupling the compound with a radioisotope or enzymatic label such that binding of the compound to the marker can be determined by detecting the labeled marker compound in a complex. For example, compounds (e.g., marker substrates) can be labeled with ¹²⁵I, ³⁵S, ¹⁴C, or ³H, either directly or indirectly, and the radioisotope detected by direct counting of radioemission or by scintillation counting. Alternatively, assay components can be enzymatically labeled with, for example, horseradish peroxidase, alkaline phosphatase, or luciferase, and the enzymatic label detected by determination of conversion of an appropriate substrate to product.

In another embodiment, the invention provides assays for screening candidate or test compounds which modulate the activity of a marker or a biologically active portion thereof. In all likelihood, the marker can, in vivo, interact with one or more molecules, such as but not limited to, peptides, proteins, hormones, cofactors and nucleic acids. For the purposes of this discussion, such cellular and extracellular molecules are referred to herein as “binding partners” or marker “substrate”.

One necessary embodiment of the invention in order to facilitate such screening is the use of the marker to identify its natural in vivo binding partners. There are many ways to accomplish this which are known to one skilled in the art. One example is the use of the marker as “bait protein” in a two-hybrid assay or three-hybrid assay (see, e.g., U.S. Pat. No. 5,283,317; Zervos et al, 1993, Cell 72:223-232; Madura et al, 1993, J. Biol. Chem. 268:12046-12054; Bartel et al ,1993, Biotechniques 14:920-924; Iwabuchi et al, 1993 Oncogene 8:1693-1696; Brent WO94/10300) in order to identify other proteins which bind to or interact with the marker (binding partners) and, therefore, are possibly involved in the natural function of the marker. Such marker binding partners are also likely to be involved in the propagation of signals by the marker or downstream elements of a marker-mediated signaling pathway. Alternatively, such marker binding partners may also be found to be inhibitors of the marker.

The two-hybrid system is based on the modular nature of most transcription factors, which consist of separable DNA-binding and activation domains. Briefly, the assay utilizes two different DNA constructs. In one construct, the gene that encodes a marker fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming a marker-dependent complex, the DNA-binding and activation domains of the transcription factor are brought into close proximity. This proximity allows transcription of a reporter gene (e.g., LacZ) which is operably linked to a transcriptional regulatory site responsive to the transcription factor. Expression of the reporter gene can be readily detected and cell colonies containing the functional transcription factor can be isolated and used to obtain the cloned gene which encodes the protein which interacts with the marker.

In a further embodiment, assays may be devised through the use of the invention for the purpose of identifying compounds which modulate (e.g., affect either positively or negatively) interactions between a marker and its substrates and/or binding partners. Such compounds can include, but are not limited to, molecules such as antibodies, peptides, hormones, oligonucleotides, nucleic acids, and analogs thereof. Such compounds may also be obtained from any available source, including systematic libraries of natural and/or synthetic compounds. The preferred assay components for use in this embodiment is an RA marker identified herein, the known binding partner and/or substrate of same, and the test compound. Test compounds can be supplied from any source.

The basic principle of the assay systems used to identify compounds that interfere with the interaction between the marker and its binding partner involves preparing a reaction mixture containing the marker and its binding partner under conditions and for a time sufficient to allow the two products to interact and bind, thus forming a complex. In order to test an agent for inhibitory activity, the reaction mixture is prepared in the presence and absence of the test compound. The test compound can be initially included in the reaction mixture, or can be added at a time subsequent to the addition of the marker and its binding partner. Control reaction mixtures are incubated without the test compound or with a placebo. The formation of any complexes between the marker and its binding partner is then detected. The formation of a complex in the control reaction, but less or no such formation in the reaction mixture containing the test compound, indicates that the compound interferes with the interaction of the marker and its binding partner. Conversely, the formation of more complex in the presence of compound than in the control reaction indicates that the compound may enhance interaction of the marker and its binding partner.

The assay for compounds that interfere with the interaction of the marker with its binding partner may be conducted in a heterogenous or homogeneous format. Heterogeneous assays involve anchoring either the marker or its binding partner onto a solid phase and detecting complexes anchored to the solid phase at the end of the reaction. In homogeneous assays, the entire reaction is carried out in a liquid phase. In either approach, the order of addition of reactants can be varied to obtain different information about the compounds being tested. For example, test compounds that interfere with the interaction between the markers and the binding partners (e.g., by competition) can be identified by conducting the reaction in the presence of the test substance, i.e., by adding the test substance to the reaction mixture prior to or simultaneously with the marker and its interactive binding partner. Alternatively, test compounds that disrupt preformed complexes, e.g., compounds with higher binding constants that displace one of the components from the complex, can be tested by adding the test compound to the reaction mixture after complexes have been formed. The various formats are briefly described below.

In a heterogeneous assay system, either the marker or its binding partner is anchored onto a solid surface or matrix, while the other corresponding non-anchored component may be labeled, either directly or indirectly. In practice, microtitre plates are often utilized for this approach. The anchored species can be immobilized by a number of methods, either non-covalent or covalent, that are typically well known to one who practices the art. Non-covalent attachment can often be accomplished simply by coating the solid surface with a solution of the marker or its binding partner and drying. Alternatively, an immobilized antibody specific for the assay component to be anchored can be used for this purpose. Such surfaces can often be prepared in advance and stored.

In related embodiments, a fusion protein can be provided which adds a domain that allows one or both of the assay components to be anchored to a matrix. For example, glutathione-S-transferase/marker fusion proteins or glutathione-S-transferase/binding partner can be adsorbed onto glutathione sepharose beads (Sigma Chemical, St. Louis, Mo.) or glutathione derivatized microtiter plates, which are then combined with the test compound or the test compound and either the non-adsorbed marker or its binding partner, and the mixture incubated under conditions conducive to complex formation (e.g., physiological conditions). Following incubation, the beads or microtiter plate wells are washed to remove any unbound assay components, the immobilized complex assessed either directly or indirectly, for example, as described above. Alternatively, the complexes can be dissociated from the matrix, and the level of marker binding or activity determined using standard techniques.

Other techniques for immobilizing proteins on matrices can also be used in the screening assays of the invention. For example, either a marker or a marker binding partner can be immobilized utilizing conjugation of biotin and streptavidin. Biotinylated marker or target molecules can be prepared from biotin-NHS (N-hydroxy-succinimide) using techniques known in the art (e.g., biotinylation kit, Pierce Chemicals, Rockford, Ill.), and immobilized in the wells of streptavidin-coated 96 well plates (Pierce Chemical). In certain embodiments, the protein-immobilized surfaces can be prepared in advance and stored.

In order to conduct the assay, the corresponding partner of the immobilized assay component is exposed to the coated surface with or without the test compound. After the reaction is complete, unreacted assay components are removed (e.g., by washing) and any complexes formed will remain immobilized on the solid surface. The detection of complexes anchored on the solid surface can be accomplished in a number of ways. Where the non-immobilized component is pre-labeled, the detection of label immobilized on the surface indicates that complexes were formed. Where the non-immobilized component is not pre-labeled, an indirect label can be used to detect complexes anchored on the surface; e.g., using a labeled antibody specific for the initially non-immobilized species (the antibody, in turn, can be directly labeled or indirectly labeled with, e.g., a labeled anti-Ig antibody). Depending upon the order of addition of reaction components, test compounds which modulate (inhibit or enhance) complex formation or which disrupt preformed complexes can be detected.

In an alternate embodiment of the invention, a homogeneous assay may be used. This is typically a reaction, analogous to those mentioned above, which is conducted in a liquid phase in the presence or absence of the test compound. The formed complexes are then separated from unreacted components, and the amount of complex formed is determined. As mentioned for heterogeneous assay systems, the order of addition of reactants to the liquid phase can yield information about which test compounds modulate (inhibit or enhance) complex formation and which disrupt preformed complexes.

In such a homogeneous assay, the reaction producis m iay be separUated fom unreacted assay components by any of a number of standard techniques, including but not limited to: differential centrifugation, chromatography, electrophoresis and immunoprecipitation. In differential centrifugation, complexes of molecules may be separated from uncomplexed molecules through a series of centrifugal steps, due to the different sedimentation equilibria of complexes based on their different sizes and densities (see, for example, Rivas, G., and Minton, A. P., Trends Biochem Sci 1993 Aug;18(8):284-7). Standard chromatographic techniques may also be utilized to separate complexed molecules from uncomplexed ones. For example, gel filtration chromatography separates molecules based on size, and through the utilization of an appropriate gel filtration resin in a column format, for example, the relatively larger complex may be separated from the relatively smaller uncomplexed components. Similarly, the relatively different charge properties of the complex as compared to the uncomplexed molecules may be exploited to differentially separate the complex from the remaining individual reactants, for example through the use of ion-exchange chromatography resins. Such resins and chromatographic techniques are well known to one skilled in the art (see, e.g., Heegaard, 1998, J Mol. Recognit. 11: 141-148; Hage and Tweed, 1997, J. Chromatogr. B. Biomed. Sci. Appl., 699:499-525). Gel electrophoresis may also be employed to separate complexed molecules from unbound species (see, e.g., Ausubel et al (eds.), In: Current Protocols in Molecular Biology, J. Wiley & Sons, New York. 1999). In this technique, protein or nucleic acid complexes are separated based on size or charge, for example. In order to maintain the binding interaction during the electrophoretic process, non-denaturing gels in the absence of reducing agent are typically preferred, but conditions appropriate to the particular interactants will be well known to one skilled in the art. Immunoprecipitation is another common technique utilized for the isolation of a protein-protein complex from solution (see, e.g., Ausubel et al (eds.), In: Current Protocols in Molecular Biology, J. Wiley & Sons, New York. 1999). In this technique, all proteins binding to an antibody specific to one of the binding molecules are precipitated from solution by conjugating the antibody to a polymer bead that may be readily collected by centrifugation. The bound assay components are released from the beads (through a specific proteolysis event or other technique well known in the art which will not disturb the protein-protein interaction in the complex), and a second immunoprecipitation step is performed, this time utilizing antibodies specific for the correspondingly different interacting assay component. In this manner, only formed complexes should remain attached to the beads. Variations in complex formation in both the presence and the absence of a test compound can be compared, thus offering information about the ability of the compound to modulate interactions between the marker and its binding partner.

Also within the scope of the present invention are methods for direct detection of interactions between the marker and its natural binding partner and/or a test compound in a homogeneous or heterogeneous assay system without further sample manipulation. For example, the technique of fluorescence energy transfer may be utilized (see, e.g., Lakowicz et al, U.S. Pat. No. 5,631,169; Stavrianopoulos et al, U.S. Pat. No. 4,868,103). Generally, this technique involves the addition of a fluorophore label on a first ‘donor’ molecule (e.g., marker or test compound) such that its emitted fluorescent energy will be absorbed by a fluorescent label on a second, ‘acceptor’ molecule (e.g., marker or test compound), which in turn is able to fluoresce due to the absorbed energy. Alternately, the ‘donor’ protein molecule may simply utilize the natural fluorescent energy of tryptophan residues. Labels are chosen that emit different wavelengths of light, such that the ‘acceptor’ molecule label may be differentiated from that of the ‘donor’. Since the efficiency of energy transfer between the labels is related to the distance separating the molecules, spatial relationships between the molecules can be assessed. In a situation in which binding occurs between the molecules, the fluorescent emission of the ‘acceptor’ molecule label in the assay should be maximal. An FET binding event can be conveniently measured through standard fluorometric detection means well known in the art (e.g., using a fluorimeter). A test substance which either enhances or hinders participation of one of the species in the preformed complex will result in the generation of a signal variant to that of background. In this way, test substances that modulate interactions between a marker and its binding partner can be identified in controlled assays.

In another embodiment, modulators of marker expression are identified in a method wherein a cell is contacted with a candidate compound and the expression of mRNA or protein, corresponding to a marker in the cell, is determined. The level of expression of mRNA or protein in the presence of the candidate compound is compared to the level of expression of mRNA or protein in the absence of the candidate compound. The candidate compound can then be identified as a modulator of marker expression based on this comparison. For example, when expression of marker mRNA or protein is greater (statistically significantly greater) in the presence of the candidate compound than in its absence, the candidate compound is identified as a stimulator of marker mRNA or protein expression. Conversely, when expression of marker mRNA or protein is less (statistically significantly less) in the presence of the candidate compound than in its absence, the candidate compound is identified as an inhibitor of marker mRNA or protein expression. The level of marker mRNA or protein expression in the cells can be determined by methods described herein for detecting marker mRNA or protein.

In another aspect, the invention pertains to a combination of two or more of the assays described herein. For example, a modulating agent can be identified using a cell-based or a cell free assay, and the ability of the agent to modulate the activity of a marker can be further confirmed in vivo, e.g., in a whole animal model for cellular transformation.

This invention further pertains to novel agents identified by the above-described screening assays. Accordingly, it is within the scope of this invention to further use an agent identified as described herein in an appropriate animal model. For example, an agent identified as described herein (e.g., an marker modulating agent, an antisense marker nucleic acid molecule, an marker-specific antibody, or an marker-binding partner) can be used in an animal model to determine the efficacy, toxicity, or side effects of treatment with such an agent. Alternatively, an agent identified as described herein can be used in an animal model to determine the mechanism of action of such an agent. Furthermore, this invention pertains to uses of novel agents identified by the above-described screening assays for treatments as described herein.

It is understood that appropriate doses of small molecule agents and protein or polypeptide agents depends upon a number of factors within the knowledge of the ordinarily skilled physician, veterinarian, or researcher. The dose(s) of these agents will vary, for example, depending upon the identity, size, and condition of the subject or sample being treated, further depending upon the route by which the composition is to be administered, if applicable, and the effect which the practitioner desires the agent to have upon the nucleic acid of polypeptide of the invention. Exemplary doses of a small molecule include milligram or microgram amounts per kilogram of subject or sample weight (e.g. about 1 microgram per kilogram to about 500 milligrams per kilogram, about 100 micrograms per kilogram to about 5 milligrams per kilogram, or about 1 microgram per kilogram to about 50 micrograms per kilogram). Exemplary doses of a protein or polypeptide include gram, milligram or microgram amounts per kilogram of subject or sample weight (e.g. about 1 microgram per kilogram to about 5 grams per kilogram, about 100 micrograms per kilogram to about 500 milligrams per kilogram, or about 1 milligram per kilogram to about 50 milligrams per kilogram). It is furthermore understood that appropriate doses of one of these agents depend upon the potency of the agent with respect to the expression or activity to be modulated. Such appropriate doses can be determined using the assays described herein. When one or more of these agents is to be administered to an animal (e.g. a human) in order to modulate expression or activity of a polypeptide or nucleic acid of the invention, a physician, veterinarian, or researcher can, for example, prescribe a relatively low dose at first, subsequently increasing the dose until an appropriate response is obtained. In addition, it is understood that the specific dose level for any particular animal subject will depend upon a variety of factors including the activity of the specific agent employed, the age, body weight, general health, gender, and diet of the subject, the time of administration, the route of administration, the rate of excretion, any drug combination, and the degree of expression or activity to be modulated.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediamine-tetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersions. For intravenous administration, suitable carriers include physiological saline, bacteriostatic water, Cremophor EL (BASF; Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, or sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., a polypeptide or antibody) in the required amount in an appropriate solvent with one or a combination of ingredients enumerated above, as required, followed by filtered sterilization. Generally, dispersions are prepared by incorporating the active compound into a sterile vehicle which contains a basic dispersion medium, and then incorporating the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously steriie-fiitered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed.

Pharmaceutically compatible binding agents, and/or adjuvant materials can be included as part of the composition. The tablets, pills, capsules, troches, and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermnint, methyl salicylate, or orange flavoring.

For administration by inhalation, the compounds are delivered in the form of an aerosol spray from a pressurized container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes having monoclonal antibodies incorporated therein or thereon) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

For antibodies, the preferred dosage is 0.1 mg/kg to 100 mg/kg of body weight (generally 10 mg/kg to 20 mg/kg). If the antibody is to act in the brain, a dosage of 50 mg/kg to 100 mg/kg is usually appropriate. Generally, partially human antibodies and fully human antibodies have a longer half-life within the human body than other antibodies. Accordingly, lower dosages and less frequent administration is often possible. Modifications such as lipidation can be used to stabilize antibodies and to enhance uptake and tissue penetration. A method for lipidation of antibodies is described by Cruikshank et al. (1997) J. Acquired Immune Deficiency Syndromes and Human Retrovirology 14:193.

The nucleic acid molecules corresponding to a marker of the invention can be inserted into vectors and used as gene therapy vectors. Gene therapy vectors can be delivered to a subject by, for example, intravenous injection, local administration (U.S. Pat. No. 5,328,470), or by stereotactic injection (see, e.g., Chen et al., 1994, Proc. Natl. Acad. Sci. USA 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g. retroviral vectors, the pharmaceutical preparation can include one or more cells which produce the gene delivery system.

The pharmaceutical compositions can be included in a container, pack, or dispenser together with instructions for administration.

V. Monitoring the Effectiveness of an Anti-RA Agent

As discussed above, the markers of the present invention can be used to assess whether RA has become refractory to an ongoing treatment (e.g., a therapeutic treatment). This embodiment of the present invention relies on comparing two or more samples obtained from a patient undergoing anti-RA treatment. In general, it is preferable to obtain a first sample from the patient prior to beginning therapy and one or more samples during treatment. In such a use, a baseline of expression prior to therapy is determined and then changes in the baseline state of expression is monitored during the course of therapy. Alternatively, two or more successive samples obtained during treatment can be used without the need of a pre-treatment baseline sample. In such a use, the first sample obtained from the subject is used as a baseline for determining whether the expression of a particular gene is increasing or decreasing.

In general, when monitoring the effectiveness of a therapeutic treatment, two or more samples from the patient are examined. Preferably, three or more successively obtained samples are used, including at least one pretreatment sample.

VI. Electronic Apparatus Readable Media and Arrays

Electronic apparatus readable media comprising a marker of the present invention is also provided. As uFsed herein, “electronic apparatus readable media” refers to any suitable medium for storing, holding or containing data or information that can be read and accessed directly by an electronic apparatus. Such media can include, but are not limited to: magnetic storage media, such as floppy discs, hard disc storage medium, and magnetic tape; optical storage media such as compact disc; electronic storage media such as RAM, ROM, EPROM, EEPROM and the like; general hard disks and hybrids of these categories such as magnetic/optical storage media. The medium is adapted or configured for having recorded thereon a marker of the present invention.

As used herein, the term “electronic apparatus” is intended to include any suitable computing or processing apparatus or other device configured or adapted for storing data or information. Examples of electronic apparatus suitable for use with the present invention include stand-alone computing apparatus; networks, including a local area network (LAN), a wide area network (WAN) Internet, Intranet, and Extranet; electronic appliances such as a personal digital assistants (PDAs), cellular phone, pager and the like; and local and distributed processing systems.

As used herein, “recorded” refers to a process for storing or encoding information on the electronic apparatus readable medium. Those skilled in the art can readily adopt any of the presently known methods for recording information on known media to generate manufactures comprising the markers of the present invention.

A variety of software programs and formats can be used to store the marker information of the present invention on the electronic apparatus readable medium. For example, the nucleic acid sequence corresponding to the markers can be represented in a word processing text file, formatted in commercially-available software such as WordPerfect and MicroSoft Word, or represented in the form of an ASCII file, stored in a database application, such as DB2, Sybase, Oracle, or the like, as well as in other forms. Any number of dataprocessor structuring formats (e.g., text file or database) may be employed in order to obtain or create a medium having recorded thereon the markers of the present invention.

By providing the markers of the invention in readable form, one can routinely access the marker sequence information for a variety of purposes. For example, one skilled in the art can use the nucleotide or amino acid sequences of the present invention in readable form to compare a target sequence or target structural motif with the sequence information stored within the data storage means. Search means are used to identify fragments or regions of the sequences of the invention which match a particular target sequence or target motif.

The present invention therefore provides a medium for holding instructions for performing a method for determining whether a subject has RA or a pre-disposition to RA, wherein the method comprises the steps of determining the presence or absence of a RA marker and based on the presence or absence of the RA marker, determining whether the subject has RA or a pre-disposition to RA and/or recommending a particular treatment for the RA or pre-RA condition.

The present invention further provides in an electronic system and/or in a network, a method for determining whether a subject has RA or a pre-disposition to RA associated with a RA marker wherein the method comprises the steps of determining the presence or absence of the RA marker, and based on the presence or absence of the RA marker, determining whether the subject has RA or a pre-disposition to RA, and/or recommending a particular treatment for the RA or pre-RA condition. The method may further comprise the step of receiving phenotypic information associated with the subject and/or acquiring from a network phenotypic information associated with the subject.

The present invention also provides in a network, a method for determining whether a subject has RA or a pre-disposition to RA associated with a RA marker, said method comprising the steps of receiving information associated with the RA marker receiving phenotypic information associated with the subject, acquiring information from the network corresponding to the RA marker and/or RA, and based on one or more of the phenotypic information, the RA marker, and the acquired information, determining whether the subject has RA or a pre-disposition to RA. The method may further comprise the step of recommending a particular treatment for the RA or pre-RA condition

The present invention also provides a business method for determining whether a subject has RA or a pre-disposition to RA, said method comprising the steps of receiving information associated with the RA marker, receiving phenotypic information associated with the subject, acquiring information from the network corresponding to the RA marker and/or RA, and based on one or more of the phenotypic information, the RA marker, and the acquired information, determining whether the subject has RA or a pre-disposition to RA. The method may further comprise the step of recommending a particular treatment for the RA or pre-RA condition.

The invention also includes gene and protein arrays comprising a RA marker of the present invention. The arrays can be used to assay expression of one or more genes or to assay expression of one or more proteins in the arrays. In one embodiment, the gene arrays can be used to assay gene expression in a tissue to ascertain tissue specificity of genes in the array. In another embodiment, the protein arrays can be used to assay protein expression in a tissue to ascertain tissue specificity of proteins in the array. In this manner, several thousands of genes or proteins can be simultaneously assayed for expression. This allows a profile to be developed showing a battery of genes or proteins specifically expressed in one or more tissues.

In addition to such qualitative determination, the invention allows the quantitation of gene or protein expression. Thus, not only tissue specificity, but also the level of expression of a battery of genes or proteins in the tissue is ascertainable. Thus, genes or proteins can be grouped on the basis of their tissue expression per se and level of expression in that tissue. This is useful, for example, in ascertaining the relationship of gene or protein expression between or among tissues. Thus, one tissue can be perturbed and the effect on gene or protein expression in a second tissue can be determined. In this context, the effect of one cell type on another cell type in response to a biological stimulus can be determined. Such a determination is useful, for example, to know the effect of cell-cell interaction at the level of gene or protein expression. If an agent is administered therapeutically to treat one cell type but has an undesirable effect on another cell type, the invention provides an assay to determine the molecular basis of the undesirable effect and thus provides the opportunity to co-administer a counteracting agent or otherwise treat the undesired effect. Similarly, even within a single cell type, undesirable biological effects can be determined at the molecular level. Thus, the effects of an agent on expression of other than the target gene can be ascertained and counteracted.

In another embodiment, the arrays can be used to monitor the time course of expression of one or more genes or proteins in the array. This can occur in various biological contexts, as disclosed herein, for example development of RA, progression of RA, and processes, such a cellular transformation associated with RA.

The arrays are also useful for ascertaining the effect of the expression of a gene or protein on the expression of other genes or proteins in the same cell or in different cells. This provides, for example, for a selection of alternate molecular targets for therapeutic intervention if the ultimate or downstream target cannot be regulated.

The arrays are also useful for ascertaining differential expression patterns of one or more genes or proteins in normal and abnormal cells. This provides a battery of genes or proteins that could serve as a molecular target for diagnosis or therapeutic intervention.

VII. Predictive Medicine

The present invention pertains to the field of predictive medicine in which diagnostic assays, prognostic assays, pharmacogenomics, and monitoring clinical trails are used for prognostic (predictive) purposes to thereby treat an individual prophylactically. Accordingly, one aspect of the present invention relates to diagnostic assays for determining the level of expression of polypeptides or nucleic acids corresponding to one or more markers of the invention, in order to determine whether an individual is at risk of developing RA. Such assays can be used for prognostic or predictive purposes to thereby prophylactically treat an individual prior to the onset of the RA.

Yet another aspect of the invention pertains to monitoring the influence of agents (e.g., drugs or other compounds administered either to inhibit RA or to treat or prevent any other disorder {i.e. in order to understand any RA progressive effects that such treatment may have}) on the expression or activity of a marker of the invention in clinical trials. These and other agents are described in further detail in the following sections.

A. Diagnostic Assays

An exemplary method for detecting the presence or absence of a polypeptide or nucleic acid corresponding to a marker of the invention in a biological sample involves obtaining a biological sample (e.g. a RA-associated body fluid) from a test subject and contacting the biological sample with a compound or an agent capable of detecting the polypeptide or nucleic acid (e.g., mRNA, genomic DNA, or cDNA). The detection methods of the invention can thus be used to detect mRNA, protein, cDNA, or genomic DNA, for example, in a biological sample in vitro as well as in vivo. For example, in vitro techniques for detection of mRNA include Northern hybridizations and in situ hybridizations. In vitro techniques for detection of a polypeptide corresponding to a marker of the invention include enzyme linked immunosorbent assays (ELISAs), Western blots, immunoprecipitations and immunofluorescence, liquid and gas chromatography, mass spectroscopy, and nuclear magnetic resonance, as well as other imaging technologies. In vitro techniques for detection of genomic DNA include Southern hybridizations. Furthermore, in vivo techniques for detection of a polypeptide corresponding to a marker of the invention include introducing into a subject a labeled antibody directed against the polypeptide. For example, the antibody can be labeled with a radioactive marker whose presence and location in a subject can be detected by standard imaging techniques.

A general principle of such diagnostic and prognostic assays involves preparing a sample or reaction mixture that may contain a marker, and a probe, under appropriate conditions and for a time sufficient to allow the marker and probe to interact and bind, thus forming a complex that can be removed and/or detected in the reaction mixture. These assays can be conducted in a variety of ways.

For example, one method to conduct such an assay would involve anchoring the marker or probe onto a solid phase support, also referred to as a substrate, and detecting target marker/probe complexes anchored on the solid phase at the end of the reaction. In one embodiment of such a method, a sample from a subject, which is to be assayed for presence and/or concentration of marker, can be anchored onto a carrier or solid phase support. In another embodiment, the reverse situation is possible, in which the probe can be anchored to a solid phase and a sample from a subject can be allowed to react as an unanchored component of the assay.

There are many established methods for anchoring assay components to a solid phase. These include, without limitation, marker or probe molecules which are immobilized through conjugation of biotin and streptavidin. Such biotinylated assay components can be prepared from biotin-NHS (N-hydroxy-succinimide) using techniques known in the art (e.g., biotinylation kit, Pierce Chemicals, Rockford, Ill.), and immobilized in the wells of streptavidin-coated 96 well plates (Pierce Chemical). In certain embodiments, the surfaces with immobilized assay components can be prepared in advance and stored.

Other suitable carriers or solid phase supports for such assays include any material capable of binding the class of molecule to which the marker or probe belongs. Well-known supports or carriers include, but are not limited to, glass, polystyrene, nylon, polypropylene, nylon, polyethylene, dextran, amylases, natural and modified celluloses, polyacrylamides, gabbros, and magnetite.

In order to conduct assays with the above mentioned approaches, the non-immobilized component is added to the solid phase upon which the second component is anchored. After the reaction is complete, uncomplexed components may be removed (e.g., by washing) under conditions such that any complexes formed will remain immobilized upon the solid phase. The detection of marker/probe complexes anchored to the solid phase can be accomplished in a number of methods outlined herein.

In a preferred embodiment, the probe, when it is the unanchored assay component, can be labeled for the purpose of detection and readout of the assay, either directly or indirectly, with detectable labels discussed herein and which are well-known to one skilled in the art.

It is also possible to directly detect marker/probe complex formation without further manipulation or labeling of either component (marker or probe), for example by utilizing the technique of fluorescence energy transfer (see, for example, Lakowicz et al., U.S. Pat. No. 5,631,169; Stavrianopoulos, et al., U.S. Pat. No. 4,868,103). A fluorophore label on the first, ‘donor’ molecule is selected such that, upon excitation with incident light of appropriate wavelength, its emitted fluorescent energy will be absorbed by a fluorescent label on a second ‘acceptor’ molecule, which in turn is able to fluoresce due to the absorbed energy. Alternately, the ‘donor’ protein molecule may simply utilize the natural fluorescent energy of tryptophan residues. Labels are chosen that emit different wavelengths of light, such that the ‘acceptor’ molecule label may be differentiated from that of the ‘donor’. Since the efficiency of energy transfer between the labels is related to the distance separating the molecules, spatial relationships between the molecules can be assessed. In a situation in which binding occurs between the molecules, the fluorescent emission of the ‘acceptor’ molecule label in the assay should be maximal. An FET binding event can be conveniently measured through standard fluorometric detection means well known in the art (e.g., using a fluorimeter).

In another embodiment, determination of the ability of a probe to recognize a marker can be accomplished without labeling either assay component (probe or marker) by utilizing a technology such as real-time Biomolecular Interaction Analysis (BIA) (see, e.g., Sjolander, S. and Urbaniczky, C., 1991, Anal. Chem. 63:2338-2345 and Szabo et al., 1995, Curr. Opin. Struct. Biol. 5:699-705). As used herein, “BIA” or “surface plasmon resonance” is a technology for studying biospecific interactions in real time, without labeling any of the interactants (e.g., BIAcore). Changes in the mass at the binding surface (indicative of a binding event) result in alterations of the refractive index of light near the surface (the optical phenomenon of surface plasmon resonance (SPR)), resulting in a detectable signal which can be used as an indication of real-time reactions between biological molecules.

Alternatively, in another embodiment, analogous diagnostic and prognostic assays can be conducted with marker and probe as solutes in a liquid phase. In such an assay, the complexed marker and probe are separated from uncomplexed components by any of a number of standard techniques, including but not limited to: differential centrifugation, chromatography, electrophoresis and immunoprecipitation. In differential centrifugation, marker/probe complexes may be separated from uncomplexed assay components through a series of centrifugal steps, due to the different sedimentation equilibria of complexes based on their different sizes and densities (see, for example, Rivas, G., and Minton, A.P., 1993, Trends Biochem Sci. 18(8):284-7). Standard chromatographic techniques may also be utilized to separate complexed molecules from uncomplexed ones. For example, gel filtration chromatography separates molecules based on size, and through the utilization of an appropriate gel filtration resin in a column format, for example, the relatively larger complex may be separated from the relatively smaller uncomplexed components. Similarly, the relatively different charge properties of the marker/probe complex as compared to the uncomplexed components may be exploited to differentiate the complex from uncomplexed components, for example through the utilization of ion-exchange chromatography resins. Such resins and chromatographic techniques are well known to one skilled in the art (see, e.g., Heegaard, N. H., 1998, J. Mol. Recognit. Winter 11(1-6): 141-8; Hage, D. S., and Tweed, S. A. J Chromatogr B Biomed Sci Appl (1997 Oct. 10);699(1-2):499-525). Gel electrophoresis may also be employed to separate complexed assay components from unbound components (see, e.g., Ausubel et al., ed., Current Protocols in Molecular Biology, John Wiley & Sons, New York, 1987-1999). In this technique, protein or nucleic acid complexes are separated based on size or charge, for example. In order to maintain the binding interaction during the electrophoretic process, non-denaturing gel matrix materials and conditions in the absence of reducing agent are typically preferred. Appropriate conditions to the particular assay and components thereof will be well known to one skilled in the art.

In a particular embodiment, the level of mRNA corresponding to the marker can be determined both by in situ and by in vitro formats in a biological sample using methods known in the art. The term “biological sample” is intended to include tissues, cells, biological fluids and isolates thereof, isolated from a subject, as well as tissues, cells and fluids present within a subject. Many expression detection methods use isolated RNA. For in vitro methods, any RNA isolation technique that does not select against the isolation of mRNA can be utilized for the purification of RNA from RA-associated body fluids (see, e.g., Ausubel et al., ed., Current Protocols in Molecular Biology, John Wiley & Sons, New York 1987-1999). Additionally, large numbers of tissue samples can readily be processed using techniques well known to those of skill in the art, such as, for example, the single-step RNA isolation process of Chomczynski (1989, U.S. Pat. No. 4,843,155).

The isolated mRNA can be used in hybridization or amplification assays that include, but are not limited to, Southern or Northern analyses, polymerase chain reaction analyses and probe arrays. One preferred diagnostic method for the detection of mRNA levels involves contacting the isolated mRNA with a nucleic acid molecule (probe) that can hybridize to the mRNA encoded by the gene being detected. The nucleic acid probe can be, for example, a full-length cDNA, or a portion thereof, such as an oligonucleotide of at least 7, 15, 30, 50, 100, 250 or 500 nucleotides in length and sufficient to specifically hybridize under stringent conditions to a mRNA or genomic DNA encoding a marker of the present invention. Other suitable probes for use in the diagnostic assays of the invention are described herein. Hybridization of an mRNA with the probe indicates that the marker in question is being expressed.

In one format, the mRNA is immobilized on a solid surface and contacted with a probe, for example by running the isolated mRNA on an agarose gel and transferring the mRNA from the gel to a membrane, such as nitrocellulose. In an alternative format, the probe(s) are immobilized on a solid surface and the mRNA is contacted with the probe(s), for example, in an Affymetrix gene chip array. A skilled artisan can readily adapt known mRNA detection methods for use in detecting the level of mRNA encoded by the markers of the present invention.

An alternative method for determining the level of mRNA corresponding to a marker of the present invention in a sample involves the process of nucleic acid amplification, e.g., by rtPCR (the experimental embodiment set forth in Mullis, 1987, U.S. Pat. No. 4,683,202), ligase chain reaction (Barany, 1991, Proc. Natl. Acad. Sci. USA, 88:189-193), self sustained sequence replication (Guatelli et al., 1990, Proc. Natl. Acad. Sci. USA 87:1874-1878), transcriptional amplification system (Kwoh et al., 1989, Proc. Natl. Acad. Sci. USA 86:1173-1177), Q-Beta Replicase (Lizardi et al., 1988, Bio/Technology 6:1197), rolling circle replication (Lizardi et al., U.S. Pat. No. 5,854,033) or any other nucleic acid amplification method, followed by the detection of the amplified molecules using techniques well known to those of skill in the art. These detection schemes are especially useful for the detection of nucleic acid molecules if such molecules are present in very low numbers. As used herein, amplification primers are defined as being a pair of nucleic acid molecules that can anneal to 5′ or 3′ regions of a gene (plus and minus strands, respectively, or vice-versa) and contain a short region in between. In general, amplification primers are from about 10 to 30 nucleotides in length and flank a region from about 50 to 200 nucleotides in length. Under appropriate conditions and with appropriate reagents, such primers penmit the amplification of a nucleic acid molecule comprising the nucleotide sequence flanked by the primers.

For in situ methods, mRNA does not need to be isolated from the patient sample prior to detection. In such methods, a cell or tissue sample is prepared/processed using known histological methods. The sample is then immobilized on a support, typically a glass slide, and then contacted with a probe that can hybridize to mRNA that encodes the marker.

As an alternative to making determinations based on the absolute expression level of the marker, determinations may be based on the normalized expression level of the marker. Expression levels are normalized by correcting the absolute expression level of a marker by comparing its expression to the expression of a gene that is not a marker, e.g., a housekeeping gene that is constitutively expressed. Suitable genes for normalization include housekeeping genes such as the actin gene, or epithelial cell-specific genes. This normalization allows the comparison of the expression level in one sample, e.g., a patient sample, to another sample, e.g., a non-RA sample, or between samples from different sources.

In a method of determining the abundance of a marker in a sample compared to the normal or control, i.e., to identify markers that are differentially present, the relative abundance may be determined by normalizing the signal obtained upon detecting the marker in a sample by reference to a suitable background parameter, e.g., to the total protein in the sample being analyzed to an invariant marker, i.e., a marker whose abundance is known to be similar in the sample being compared, or to the total signal detected from all proteins in the sample.

Alternatively, the expression level can be provided as a relative expression level. To determine a relative expression level of a marker, the level of expression of the marker is determined for 10 or more samples of normal versus RA patient sample isolates, preferably 50 or more samples, prior to the determination of the expression level for the sample in question. The mean expression level of each of the genes assayed in the larger number of samples is determined and this is used as a baseline expression level for the marker. The expression level of the marker determined for the test sample (absolute level of expression) is then divided by the mean expression value obtained for that marker. This provides a relative expression level.

Preferably, the samples used in the baseline determination will be from RA or from non-RA patient samples. The choice of the cell source is dependent on the use of the relative expression level. Using expression found in normal tissues as a mean expression score aids in validating whether the marker assayed is RA specific (versus normal cells). In addition, as more data is accumulated, the mean expression value can be revised, providing improved relative expression values based on accumulated data. Expression data from RA patient samples provides a means for grading the severity of the RA state.

In another embodiment of the present invention, a polypeptide corresponding to a marker is detected. A preferred agent for detecting a polypeptide of the invention is an antibody capable of binding to a polypeptide corresponding to a marker of the invention, preferably an antibody with a detectable label. Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin.

Proteins from patient samples can be isolated using techniques that are well known to those of skill in the art. The protein isolation methods employed can, for example, be such as those described in Harlow and Lane (Harlow and Lane, 1988, Antibodies: A Laboratory Manual, Cold Spring Harbor Laboratory Press, Cold Spring Harbor, New York).

A variety of formats can be employed to determine whether a sample contains a protein that binds to a given antibody. Examples of such formats include, but are not limited to, enzyme immunoassay (EIA), radioimmunoassay (RIA), Western blot analysis, protein arrays, antibody arrays, enzyme linked immunoabsorbant assay (ELISA), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays and protein A immunoassays. A skilled artisan can readily adapt known protein/antibody detection methods for use in determining whether a patient sample expresses a marker of the present invention.

In one format, antibodies, or antibody fragments, can be used in methods such as Western blots, antibody arrays or immunofluorescence techniques to detect the expressed proteins. In such uses, it is generally preferable to immobilize either the antibody or proteins on a solid support. Suitable solid phase supports or carriers include any support capable of binding an antigen or an antibody. Well-known supports or carriers include glass, polystyrene, polypropylene, polyethylene, dextran, nylon, amylases, natural and modified celluloses, polyacrylamides, gabbros, and magnetite. For protein and antibody arrays see, e.g. U.S. Pat. Nos. 6,365,418, 6,329,209, 6,406,921, 6,475,808 and 6,475,809.

One skilled in the art will know many other suitable carriers for binding antibody or antigen, and will be able to adapt such support for use with the present invention. For example, protein isolated from a patient sample can be run on a polyacrylamide gel electrophoresis and immobilized onto a solid phase support such as nitrocellulose. The support can then be washed with suitable buffers followed by treatment with the detectably labeled antibody. The solid phase support can then be washed with the buffer a second time to remove unbound antibody. The amount of bound label on the solid support can then be detected by conventional means.

The invention also encompasses kits for detecting the presence of a polypeptide or nucleic acid corresponding to a marker of the invention in a biological sample (e.g. an RA-associated body fluid). Such kits can be used to determine if a subject is suffering from or is at increased risk of developing RA. For example, the kit can comprise a labeled compound or agent capable of detecting a polypeptide or an mRNA encoding a polypeptide corresponding to a marker of the invention in a biological sample and means for determining the amount of the polypeptide or mRNA in the sample (e.g., an antibody which binds the polypeptide or an oligonucleotide probe which binds to DNA or mRNA encoding the polypeptide). Kits can also include instructions for interpreting the results obtained using the kit.

For antibody-based kits, the kit can comprise, for example: (1) a first antibody (e.g., attached to a solid support) which binds to a polypeptide corresponding to a marker of the invention; and, optionally, (2) a second, different antibody which binds to either the polypeptide or the first antibody and is conjugated to a detectable label.

For oligonucleotide-based kits, the kit can comprise, for example: (1) an oligonucleotide, e.g., a detectably labeled oligonucleotide, which hybridizes to a nucleic acid sequence encoding a polypeptide corresponding to a marker of the invention or (2) a pair of primers useful for amplifying a nucleic acid molecule corresponding to a marker of the invention. The kit can also comprise, e.g., a buffering agent, a preservative, or a protein stabilizing agent. The kit can further comprise components necessary for detecting the detectable label (e.g., an enzyme or a substrate). The kit can also contain a control sample or a series of control samples which can be assayed and compared to the test sample. Each component of the kit can be enclosed within an individual container and all of the various containers can be within a single package, along with instructions for interpreting the results of the assays performed using the kit.

B. Pharmacogenomics

Agents or modulators which have a stimulatory or inhibitory effect on expression of a marker of the invention can be administered to individuals to treat (prophylactically or therapeutically) RA in the patient. In conjunction with such treatment, the pharmacogenomics (i.e., the study of the relationship between an individual's genotype and that individual's response to a foreign compound or drug) of the individual may be considered. Differences in metabolism of therapeutics can lead to severe toxicity or therapeutic failure by altering the relation between dose and blood concentration of the pharmacologically active drug. Thus, the pharmacogenomics of the individual permits the selection of effective agents (e.g., drugs) for prophylactic or therapeutic treatments based on a consideration of the individual's genotype. Such pharmacogenomics can further be used to determine appropriate dosages and therapeutic regimens. Accordingly, the level of expression of a marker of the invention in an individual can be determined to thereby select appropriate agent(s) for therapeutic or prophylactic treatment of the individual.

Pharmacogenomics deals with clinically significant variations in the response to drugs due to altered drug disposition and abnormal action in affected persons. See, e.g., Linder (1997) Clin. Chem. 43(2):254-266. In general, two types of pharmacogenetic conditions can be differentiated. Genetic conditions transmitted as a single factor altering the way drugs act on the body are referred to as “altered drug action.” Genetic conditions transmitted as single factors altering the way the body acts on drugs are referred to as “altered drug metabolism”. These pharmacogenetic conditions can occur either as rare defects or as polymorphisms. For example, glucose-6-phosphate dehydrogenase (G6PD) deficiency is a common inherited enzymopathy in which the main clinical complication is hemolysis after ingestion of oxidant drugs (anti-malarials, sulfonamides, analgesics, nitrofurans) and consumption of fava beans.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome P450 enzymes CYP2D6 and CYP2C19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C19 quite frequently experience exaggerated drug response and side effects when they receive standard doses. If a metabolite is the active therapeutic moiety, a PM will show no therapeutic response, as demonstrated for the analgesic effect of codeine mediated by its CYP2D6-formed metabolite morphine. The other extreme are the so called ultra-rapid metabolizers who do not respond to standard doses. Recently, the molecular basis of ultra-rapid metabolism has been identified to be due to CYP2D6 gene amplification.

Thus, the level of expression of a marker of the invention in an individual can be determined to thereby select appropriate agent(s) for therapeutic or prophylactic treatment of the individual. In addition, pharmacogenetic studies can be used to apply genotyping of polymorphic alleles encoding drug-metabolizing enzymes to the identification of an individual's drug responsiveness phenotype. This knowledge, when applied to dosing or drug selection, can avoid adverse reactions or therapeutic failure and thus enhance therapeutic or prophylactic efficiency when treating a subject with a modulator of expression of a marker of the invention.

This invention also provides a process for preparing a database comprising at least one of the markers. For example, the polynucleotide sequences are stored in a digital storage medium such that a data processing system for standardized representation of the genes that identify a RA cell is compiled. The data processing system is useful to analyze gene expression between two cells by first selecting a cell suspected of being of a neoplastic phenotype or genotype and then isolating polynucleotides from the cell. The isolated polynucleotides are sequenced. The sequences from the sample are compared with the sequence(s) present in the database using homology search techniques. Greater than 90%, more preferably greater than 95% and more preferably, greater than or equal to 97% sequence identity between the test sequence and the polynucleotides of the present invention is a positive indication that the polynucleotide has been isolated from a RA cell as defined above.

In an alternative embodiment, the polynucleotides of this invention are sequenced and the information regarding sequence and in some embodiments, relative expression, is stored in any functionally relevant program, e.g., in Compare Report using the SAGE software (available though Dr. Ken Kinzler at John Hopkins University). The Compare Report provides a tabulation of the polynucleotide sequences and their abundance for the samples normalized to a defined number of polynucleotides per library (say 25,000). This is then imported into MS-ACCESS either directly or via copying the data into an Excel spreadsheet first and then from there into MS-ACCESS for additional manipulations. Other programs such as SYBASE or Oracle that permit the comparison of polynucleotide numbers could be used as alternatives to MS-ACCESS. Enhancements to the software can be designed to incorporate these additional functions. These functions consist in standard Boolean, algebraic, and text search operations, applied in various combinations to reduce a large input set of polynucleotides to a manageable subset of a polynucleotide of specifically defined interest.

C. Monitoring Clinical Trials

Monitoring the influence of agents (e.g., drug compounds) on the level of expression of a marker of the invention can be applied not only in basic drug screening, but also in clinical trials. For example, the effectiveness of an agent to affect marker expression can be monitored in clinical trials of subjects receiving treatment for RA. In a preferred embodiment, the present invention provides a method for monitoring the effectiveness of treatment of a subject with an agent (e.g., an agonist, antagonist, peptidomimetic, protein, peptide, nucleic acid, small molecule, or other drug candidate) comprising the steps of (i) obtaining a pre-administration sample from a subject prior to administration of the agent; (ii) detecting the level of expression of one or more selected markers of the invention in the pre-administration sample; (iii) obtaining one or more post-administration samples from the subject; (iv) detecting the level of expression of the marker(s) in the post-administration samples; (v) comparing the level of expression of the marker(s) in the pre-administration sample with the level of expression of the marker(s) in the post-administration sample or samples; and (vi) altering the administration of the agent to the subject accordingly. For example, increased administration of the agent can be desirable to increase expression of the marker(s) to higher levels than detected, i.e., to increase the effectiveness of the agent. Alternatively, decreased administration of the agent can be desirable to decrease expression of the marker(s) to lower levels than detected, i.e., to decrease the effectiveness of the agent.

D. Surrogate Markers

The markers of the invention may serve as surrogate markers for one or more disorders or disease states or for conditions leading up to disease states, and in particular, RA. As used herein, a “surrogate marker” is an objective biochemical marker which correlates with the absence or presence of a disease or disorder, or with the progression of a disease or disorder (e.g., with the presence or absence of RA symptoms). While the presence or quantity of such markers is independent of the disease, changes in the absence or presence or quantity of the marker serve as a reflection of the disease or its treatment. Therefore, these markers may serve to indicate whether a particular course of treatment is effective in lessening a disease state or disorder. Surrogate markers are of particular use when the presence or extent of a disease state or disorder is difficult to assess through standard methodologies (e.g., early stage RA), or when an assessment of disease progression is desired before a potentially dangerous clinical endpoint is reached (e.g., an assessment of cardiovascular disease may be made using cholesterol levels as a surrogate marker, and an analysis of HIV infection may be made using HIV RNA levels as a surrogate marker, well in advance of the undesirable clinical outcomes of myocardial infarction or fully-developed AIDS). Examples of the use of surrogate markers in the art include: Koomen et al. (2000) J. Mass. Spectrom. 35: 258-264; and James (1994) AIDS Treatment News Archive 209.

The markers of the invention are also useful as pharmacodynamic markers. As used herein, a “pharmacodynamic marker” is an objective biochemical marker which correlates specifically with drug effects. The presence or quantity of a pharmacodynamic marker is not related to the disease state or disorder for which the drug is being administered; therefore, the presence or quantity of the marker is indicative of the presence or activity of the drug in a subject. For example, a pharmacodynamic marker may be indicative of the concentration of the drug in a biological tissue, in that the marker is either expressed or transcribed or not expressed or transcribed in that tissue in relationship to the level of the drug. In this fashion, the distribution or uptake of the drug may be monitored by the pharmacodynamic marker. Similarly, the presence or quantity of the pharmacodynamic marker may be related to the presence or quantity of the metabolic product of a drug, such that the presence or quantity of the marker is indicative of the relative breakdown rate of the drug in vivo. Pharmacodynamic markers are of particular use in increasing the sensitivity of detection of drug effects, particularly when the drug is administered in low doses. Since even a small amount of a drug may be sufficient to activate multiple rounds of marker transcription or expression, the amplified marker may be in a quantity which is more readily detectable than the drug itself. Also, the marker may be more easily detected due to the nature of the marker itself; for example, using the methods described herein, antibodies may be employed in an immune-based detection system for a protein marker, or marker-specific radiolabeled probes may be used to detect a mRNA marker. Furthermore, the use of a pharmacodynamic marker may offer mechanism-based prediction of risk due to drug treatment beyond the range of possible direct observations. Examples of the use of pharmacodynamic markers in the art include: Matsuda et al. U.S. Pat. No. 6,033,862; Hattis et al. (1991) Env. Health Perspect. 90: 229-238; Schentag (1999) Am. J. Health-Syst. Pharm. 56 Suppl. 3: S21-S24; and Nicolau (1999) Am, J. Health-Syst. Pharm. 56 Suppl. 3: S16-S20.

The markers of the invention are also useful as pharmacogenomic markers. As used herein, a “pharmacogenomic marker” is an objective biochemical marker which correlates with a specific clinical drug response or susceptibility in a subject (see, e.g., McLeod et al. (1999) Eur. J. Cancer 35(12): 1650-1652). The presence or quantity of the pharmacogenomic marker is related to the predicted response of the subject to a specific drug or class of drugs prior to administration of the drug. By assessing the presence or quantity of one or more pharmacogenomic markers in a subject, a drug therapy which is most appropriate for the subject, or which is predicted to have a greater degree of success, may be selected. For example, based on the presence or quantity of RNA or protein for specific RA markers in a subject, a drug or course of treatment may be selected that is optimized for the treatment of the specific RA likely to be present in the subject. Similarly, the presence or absence of a specific sequence mutation in marker DNA may correlate with drug response. The use of pharmacogenomic markers therefore permits the application of the most appropriate treatment for each subject without having to administer the therapy.

### EXPERIMENTAL PROTOCOL

In normal human joints, a thin film of synovial fluid covers the surfaces of synovium and cartilage within the joint space. Only in disease or injury does the volume of this fluid increase to produce a clinically apparent effusion that may be aspirated easily for study. For this reason, most knowledge of human synovial fluid comes not from normal subjects, but from patients with joint disease or injury.

The intrasynovial concentration of any protein represents the net contributions of plasma concentration, synovial blood flow, microvascular permeability, and lymphatic removal. In addition, specific proteins may be produced or consumed within the joint space. For example, lubricin is normally synthesized within synovial cells and released into synovial fluid where it facitilates boundary layer lubrication of the cartilage-on-cartilage bearing. In disease, additional proteins may be synthesized (e.g., IgG rheumatoid factor in RA) or released from inflammatory cells or articular tissues. Simkin, P A, et al., 1995, Curr Opin Rheumatol. 7:346-351. In contrast, intra-articular proteins may be depleted by local consumption, as are complement components in rheumatoid disease.

The markers of the present invention were thus initially identified in the synovial fluid of human patients who have been diagnosed with either erosive or non-erosive RA. The markers were identified by mass spectrometry after synovial fluid samples were subjected to digestion of hyaluronic acid followed by a series of protein depletion and fractionation steps to enrich subsets of proteins from the original synovial fluid samples. Some of these identified markers were then validated in serum of patients who have been diagnosed with either erosive or non-erosive RA. The following materials and methods describe the fundamental technologies/methodologies that were used in the marker discovery process.

Patients

The synovial fluid and serum samples of patients were sorted into erosive and non-erosive samples by the following inclusion criteria: 1) diagnosis of RA via the accepted American College of Rheumatology criteria, and 2) the age of onset of symptoms between 25-50. The exclusion criteria consisted of 1) a history or evidence (X-ray) of osteo arthritis, 2) systemic lupus erythematosus (SLE), 3) psoriasis or psoriatic arthritis, and 4) JRA, except in those cases with elevated rheumatoid factor.

The study design involved synovial fluid and serum samples from subjects with erosive RA that had “late” disease, wherein the onset of the symptoms was five years or more. Likewise, the study design also included subjects with non-erosive RA with “new” disease, wherein the onset of the symptoms was less than five years, and subjects with non-erosive RA with “late” disease, wherein the onset of the symptoms was five years or more.

I. Synovial Fluid Studies

Methods

Erosive and non-erosive synovial fluid patient samples were digested with a fungal hyaluronidase enzyme, which allowed for nearly complete digestion of hyaluronic acid present in the synovial fluid samples. Then, in order to reduce the complexity of the protein mixture, predominant protein species, or highly abundant proteins, such as albumin and immunoglobulins, were removed from the samples. This depletion step involved running the synovial fluid samples over protein G and Cibacron blue resins, respectively. To further increase the number of total detectable proteins by mass spectrometry, the depleted samples were then also treated with Protein L to remove kappa light chain containing antibodies that were present in the protein mixture. The depleted synovial fluid samples were introduced into 8M urea, then reduced and alkylated to reduce endogenous protein aggregates. The samples were then subsequently subjected to sizing exchange chromatography (SEC) to fractionate the samples by size. Fractionated protein samples were then subjected to trypsin digestion and prepared for online reverse phase liquid chromatography prior to introduction into the ion trap mass spectrometer by nanospray electrospray ionization. For the more complex protein fractions, 20-centimeter columns and 3-hour gradients were run in conjunction with two-dimensional chromatography with salt step elutions prior to reverse phase separation introduction of the eluted sample peptides into the mass spectrometer. For the less complex fractions, 10-centimeter columns and a 2-hour gradient with single-dimension chromatography was used to introduce sample into the mass spectrometer.

Protein Identirication

The raw output of mass spectra was processed using software proprietary to Millennium Pharmaceuticals Inc., called SpectrumMill. The output obtained from SpectrumMill provides an analysis of proteins present in individual SEC fractions of the original SF samples. Spectra were searched against a non-redundant NCBI mammals database. Validation of peptides was performed by either using SpectrumMill's “Automatic Validation of MS-Tag Results”, by validating spectra manually or by running ID SDS PAGE gels on depleted synovial fluid samples. In all, a total of 490 proteins (Table 1) were identified in all 10 synovial fluid samples, namely five erosive synovial fluid samples and 5 non-erosive samples.

Discovery of newly-identified markers (Table 5) from the Millennium EST contig database. Novel Millennium EST's and public EST's from the dbEST data base were clustered and assembled into contigs using tools supplied by DoubleTwist, Inc. Each contig was translated into all six reading frames. Protein sequences were removed if they did not represent open reading frames (ORF's) of sufficient length. Spectra that had not produced valid tags to known proteins in the non-redundant mammals protein database were searched against the translated contig database using the SpectrumMill software. Entries were selected if they showed differential expression between the erosive and non-erosive sample sets. Entries were removed if 1) they showed 98% identity to known proteins in the non-redundant mammals protein database by BLAST analysis and 2) if the identified peptides were represented in Table 1. If the entry was not highly identical to a known protein in the non-redundant mammals protein database but was identified only by peptides that were represented in proteins that were listed in Table 1, the entry was also removed. The annotations of the remaining entries were created from dbEST and GenPept entries or inferred from BLAST results.

II. Serum Studies (S100Proteins)

Materials

The triple quadrupole mass spectrometer (API-3000 equipped with Analyst software version 1.1, Applied Biosystems, Foster City, Calif.) has nearly a 100% duty cycle and greater sensitivity than ion trap mass spectrometers when used for high throughput peptide analysis in multiple reaction monitoring (MRM) experiments. In order to use the triple quadrupole mass spectrometer to detect the endogenous/native tryptic peptides that represent peptide fragments of the calgranulin proteins of interest, it is necessary to create a set of “tune files” in the triple quadrupole mass spectrometer. These files serve to identify the mass/charge ratios and other physical parameters, including e.g. optimized voltages that allow an operator to identify the best fragment/transition ions that are unique to the tryptic peptides one seeks to identify in serum. In order to create tune files to a specific tryptic peptide, one prepares a synthetic version of that peptide and uses that synthetic peptide to select a set of optimized parameters that are unique to the molecule. These optimized parameters are then programmed into the triple quad mass spectrometer where they serve as reference standards for the identification of matching endogenous tryptic peptides in samples of processed serum. It is this set of physical parameters including the mass to charge ration of the parent ion or starting tryptic peptide and the fragment ions that are produced by collision associated dissociation that are measured by the triple quad mass spectrometer.

In this application, the mass spectrometer was tuned using synthetic peptides based on the selected theoretical tryptic cleavage sites of candidate proteins. The following tryptic peptide sequences were used for Calgranulin A (S100 A8/M44), Calgranulin B (S100 A9/M31) and Calgranulin C (S100A12/M60):

Calgranulin A=S100 A8: Tryptic fragment LLETECPQYIR

Calgranulin B=S100 A9; Tryptic fragment LGHPDTLNQGEFK

Calgranulin C=S100 A12; Tryptic fragments ELANTIK and GHFDTLSK

For peptides containing leucine(s) within their amino acid sequences, a corresponding synthetic peptide with a uniformly labeled [¹³C (U)]-leucine was synthesized. This stable heavy isotope-labeled peptide was used as the internal standard for quantification as shown below. The instrument equipped with a nanospray source (James A. Hill Instrument Services, Arlington, Mass.) was tuned to each synthetic peptide for a set of selected transition ions. This was accomplished by infusing a mixture of pure synthetic peptides that were diluted to a final concentration of 500 fmol/μL in acetonitrile: 0.1% formic acid (3:7) with a syringe pump (Harvard Apparatus, Holliston, Mass.) set to 0.2 μL/min. The mass to charge ratios (m/z) of these transition ions observed from ¹²C synthetic and ¹³C isotope-labeled peptides and their optimized voltages were transcribed respectively into a 110 minute liquid chromatography synchronized method (LCsync) in the Analyst 1.1 software. A reverse phase liquid chromatography (RPLC) method was programmed into the coupled Agilent 1100 IAPLC system (Agilent Technologies, Waldbronn, Germany). This system consisted of a binary pump and micro-well plate sampler. Verification of the system, selection of the best-ionizing peptides, and the limit of detection (LOD) was determined by injecting various levels (0 to 500 fmol/μL) of the heavy and light synthetic peptides onto a 150 mm×0.075 mm PicoFrit column (New Objective, Inc., Woburn, Mass.) packed with either Magic C₁₈ media, 5 μm particles (200 Å pore size) (Michrom Bioresources, Inc., Auburn, Calif.) or Vydac C₁₈ media, 10 μm particles (300 Å pore size) (Vydac, Hesperia, Calif.). Peptides were eluted from the column using an acetonitrile gradient (5% to 50% in 0.1% formic acid) run over 50 minutes at a final flow rate, post capillary splitter, of 200 nl/min.

Methods

Pooled serum samples (5 individuals/pool, 0.25 mL/individual) from healthy individuals and patients diagnosed with non-erosive or erosive rheumatoid arthritis (RA), were depleted of three abundant serum proteins using three types of affinity chromatography columns: a hemoglobin column for haptoglobin; HiTrap protein G columns for IgG removal; and Hitrap cibacron blue columns for removal of human serum albumin. After depletion of abundant proteins, samples were fractionated using size-exclusion chromatography (SEC).

Preparation of Hemoglobin Column

40 mg of hemoglobin (Sigma, cat #H0267) was dissolved in 1.5 mL of coupling buffer (0.2 M NaHCO₃, 0.5 M NaCl, pH 8.3). The solution was then desalted using a HiTrap Desalting column (Amersham Biosciences, cat# 17-1408-01) with the coupling buffer as the running buffer. The volume was adjusted to a concentration of 20 mg/mL of hemoglobin. A 1 mL HiTrap NHS-activated HP column (Amersham Biosciences, cat#17-0716-01) was washed with 5 mL of ice-cold 1 mM HCl, then immediately injected with 0.5 mL of the hemoglobin solution and incubated at room temperature (RT) for a minimum of 30 minutes. The column was washed with 5 mL of deactivation buffer (0.5 M ethanolamine, 0.5 M NaCl, pH 8.3) and incubated at RT for another 30 minutes. Finally the column was washed with 10 mL of depletion buffer (200 mM NH₄HCO₃, pH 7.8).

Depletion of Serum Samples

The three affinity columns were assembled in tandem in the following order: a hemoglobin column (1 ml) prepared as above; three protein G columns (1 mL each) (Amersham Biosciences, cat #17-0404-01); and one 5 mL Cibacron Blue column (Amersham Biosciences, cat #17-0413-01). The columns were washed with 90 mL of depletion buffer (200 mM NH₄HCO₃, pH 7.8).

1.25 mL of serum was diluted with the depletion buffer to a final volume of 3 mL, which was then loaded onto the assembled columns and washed with the depletion buffer (200 mM NH₄HCO₃, pH 7.8) at the flow rate of 1 mL/min. The flow-through was collected until A₂₈₀ returned to the baseline. The flow-through was freeze-dried for 48 hours, and the dry powder was stored at −20 ° C. for the next step.

SEC Fractionation of the Depleted Serum Samples

The lyophilized samples were dissolved in 2.0 mL of 8M urea, 200 mM ammonium bicarbonate. Six mgs of DTT was added (20 mM final concentration) and the mixture was incubated for 60 minutes at 60° C. After cooling to RT, 18.5 mg of iodoacetamide (final concentration 50 mM) was added to alkylate peptides. After 30 minutes of incubation in the dark at RT, the alkylated sample was immediately loaded onto the SEC column.

The column (Superdex 200 16/60, Amersham Biosciences, cat #17-1069-01) was pre-equilibrated with 240 mL of the running buffer (200 mM NH₄HCO₃, 8 M urea). With a flowrate of 0.5 mL/min, 5 mL fractions were collected 76 minutes after injection. Proteins with molecular weights below 40 kDa were collected in fractions #5 to #12.

The fractions were concentrated and diluted with water to final volumes of approximately 100 μL, with final buffer composition of 50 mM NH₄HCO₃ and 2 M urea. Centriplus YM-3 and CentriconYM-3 (Millipore, cat #4420 and 4203) were used for concentrating the fraction.

Preparation of Samples for Multiple Reaction Monitoring

Tubes containing fractionated proteins that spanned the native molecular weight range of candidate proteins of interest (fractions 7, 8 & 9) were pooled and subjected to trypsin digestion. After digestion by trypsin, a ¹³C-signature synthetic peptide representing selected known tryptic cleavage fragments of the candidate markers was added to the mixture at a final concentration of 500 fmol/μl. The addition of this “standard” and its detection by MRM can be used to establish a semi-quantitative measure of the levels of tryptic peptides derived from endogenous candidate proteins in patient serum samples.

The proteins S100 A8, -A9, -A12 have molecular weights within the 10,400 to 13,200 dalton range. Based upon the SEC chromatography of protein standards, pooling of SEC fractions 7, 8, and 9 facilitated the collection of these S100 proteins into one analyzed pool. A one microliter aliquot of each SEC pool was injected sequentially in triplicate with blank and standard samples onto the same microcapillary C₁₈ column. The extracted ion chromatogram (XIC) and total ion chromatogram (TIC) were analyzed for the tryptic peptides native to the serum samples (designated ¹²C) and ¹³C-labeled internal standard peptide of each target protein. The ratio of the TIC for the native peptide and the ¹³C labeled peptide, the protein molecular weight, and fraction volumes were used according to the following equation to calculate the target protein concentration per milliliter of serum: ((¹²C TIC/¹³C TIC)(500)(fraction volume (μL))(protein MW))/1,000,000=ng/mL.

Results

As described above, pooled SEC fractions (7, 8 & 9) taken from healthy individuals, patients with erosive rheumatoid arthritis or non-erosive arthritis, were analyzed in the triple quadrupole mass spectrometer for the presence of tryptic peptides representing fragments of the endogenous proteins calgranulin A, -B and -C. Three separate measurements were taken for each candidate peptide and the average ng/ml values were calculated.

Calgranulin A=S100 A8 (M44)

It was determined that the concentration of S 100 A8 was 3 to 4-fold higher in pooled serum samples taken from erosive RA patients as compared to pooled serum from healthy individuals or patients diagnosed with non-erosive RA (refer to Table 6).

Calgranulin B=S100 A9 (M31)

The concentration of S100 A9 was on average 14-fold higher in pooled serum samples taken from patients with erosive RA as compared to pooled serum samples taken from healthy individuals. S100 A9 concentration was also higher in samples of patients with erosive RA as compared to pooled serum samples taken from patients with non-erosive RA (refer to Table 7).

Calgranulin C=S100 A12 (M60)

Two different peptides were measured to determine the concentration of S100 A12. The concentrations of S100A12 were determined to be 15-fold or higher in pooled serum samples taken from patients with erosive RA versus pooled serum samples taken from healthy individuals (refer to Table 8A). S100A12 was determined to be 8 to 9-fold higher in pooled serum samples taken from patients with erosive RA versus pooled serum samples taken from patients with non-erosive RA (refer to Tables 8A and 8B).

Thus, three members of the S100 protein family, S100 A8, S100 A9, and S100 A12, are significantly elevated in the pooled serum of rheumatoid arthritis patients as compared to the pooled serum of healthy patients. Also, each of these proteins appears to be present in higher concentrations in patients with erosive RA versus non-erosive RA.

III. Serum Studies (SAA protein)

Serum amyloid A is an acute phase protein and it is known to be elevated in different diseases including rheumatoid arthritis. During the discovery phase of research, levels of this protein were identified as being increased in the synovial fluid of patients with erosive disease. For the determination of serum amyloid A (SAA) the N-Latex SAA assay from Dade Behring (Id.No. OQMP G11) was used and measured on the Behring Nephelometer II (Dade Behring) according to the package insert.

Briefly, this is a homogeneous immunoassay using polystyrene particles coated with antibodies raised against human SAA. Serum or synovial fluid samples are automatically diluted 1:400 with N Diluent (Dade Behring) by the instrument and the specific reagents are added automatically. After mixing of the samples with the polystyrene particles, agglutination takes place and the intensity of the scattered light is measured. The scattered light intensity is dependent on the concentration of the analyte in the sample and consequently its concentration can be determined by comparison with dilutions of a standard of known concentration. The sensitivity of the assay is 3 mg/l SAA.

Results

The results of this immunoassay analysis demonstrated that SAA is significantly increased in the serum of patients with rheumatoid arthritis versus healthy individuals and a small set of disease controls (osteoarthritis (6), chondrocalcinosis (3 patients), or psoriatic arthritis (1 patient). Tables 9A, 9B, and 9C list the average protein concentration values in human serum and the significance test results of the serum amyloid A (SAA) protein. Table 9A lists the average SAA concentration (mg/L) and the number of patient samples analyzed. The one-tail t-test values in table 9B are provided to demonstrate that the SAA concentration between erosive and non-erosive serum samples and the disease and healthy controls are significantly different, accounting for assay variability. Table 9C shows that the significance test comparison of SAA concentration values between erosive and non-erosive patient serum samples are also significantly from different populations when assay variability is considered.

Summary of the Data

Tables 1-5 list the markers obtained using the foregoing protocol. These Tables list the markers designated with a name (“Marker”), the name the gene is commonly known by, if applicable (“Gene Name”), the data generated for each synovial fluid sample (E=Erosive and N=Non-Erosive), the corresponding molecular weight (“MW (Da)”), the corresponding GenBank GI Number (“GI number”), the sequence listing identifier of the cDNA sequence of a nucleotide transcript encoded by or corresponding to the marker (“SEQ ID NO (nts)”) and the sequence listing identifier of the amino acid sequence of a protein encoded by or corresponding to the marker (“SEQ ID NO (AA)”). Tables 6-9 list the results obtained in serum samples for four of the markers initially identified in synovial fluids. Tables 6, 7, 8A and 8B list the “Sample Type”, the protein concentration after the “I^{st} Injection”, “2^{nd} Injection” and “3^{rd} Injection”, the “Average” of the three injections, as well as the Standard Deviation “% RSD”. Table 9A, 9B and 9C list the average concentration of SAA protein in samples of healthy, disease control, non-erosive and erosive human serum, one tail t-tests comparison of the SAA concentrations of erosive and non-erosive samples to healthy and disease controls, and the one tail t-test comparison of SAA concentrations in erosive and non-erosive serum samples.

Table 1 lists all of the markers of the invention (and comprises markers listed in Tables 2-5), which are over-expressed in patients with RA compared to normal individuals (i.e., individuals who are not afflicted with RA). Table 2 lists markers that are newly-associated with RA and are over-expressed in patients diagnosed with erosive or non-erosive RA. Table 3 lists preferred markers of the present invention. Table 3A lists markers which are over-expressed in serum samples of patients with RA compared to normal individuals (i.e., individuals who are not afflicted with RA). Table 4 lists markers which are especially useful for new detection (“screening”) and detection of recurrence of RA. Table 5 lists newly-identified markers that are over-expressed in patients with RA. Table 6 lists protein concentration of Calgranulin A in a pool of SEC fractions 7, 8 and 9 of human serum. Table 7 lists protein concentration of Calgranulin B in a pool of SEC fractions 7, 8 and 9 of human serum. Tables 8A and 8B list protein concentration of Calgranulin C in a pool of SEC fractions 7, 8 and 9 of human serum. Table 9 lists the average protein concentration values in human serum and the significance test results of the serum amyloid A (SAA) protein.

Other Embodiments

Those skilled in the art will recognize, or be able to ascertain using no more than routine experimentation, many equivalents to the specific embodiments of the invention described herein. Such equivalents are intended to be encompassed by the following claims.

The contents of all references, patents, published patent applications, and database records cited throughout this application are hereby incorporated by reference.

TABLE 1

| | | E_51 | E_59 | E_63 | E_67 | E_70 | N_52 | N_54 | N_58 | N_65 |
|---|---|---|---|---|---|---|---|---|---|---|
| Marker | Gene Name | Br38 | Br26 | Br35 | Br42 | Br43 | Br29 | Br28 | Br34 | Br36 |
| M1 | HAPTOGLOBIN-2 | +++ | +++ | +++ | +++ | +++ | +++ | +++ | +++ | +++ |
| | PRECURSOR | | | | | | | | | |
| M2 | SEROTRANSFERRIN | +++ | +++ | +++ | +++ | +++ | +++ | +++ | +++ | +++ |
| | PRECURSOR | | | | | | | | | |
| | (SIDEROPHILIN) (BETA-1- | | | | | | | | | |
| | METAL BINDING | | | | | | | | | |
| | GLOBULIN) | | | | | | | | | |
| M3 | SERUM ALBUMIN | +++ | +++ | +++ | +++ | +++ | +++ | +++ | +++ | +++ |
| | PRECURSOR | | | | | | | | | |
| M4 | COMPLEMENT C3 | ++ | +++ | +++ | +++ | +++ | +++ | +++ | +++ | +++ |
| | PRECURSOR [CONTAINS: | | | | | | | | | |
| | C3A ANAPHYLATOXIN] | | | | | | | | | |
| M5 | proapolipoprotein | +++ | +++ | +++ | +++ | +++ | +++ | +++ | +++ | +++ |
| M6 | ALPHA-1-ANTITRYPSIN | +++ | +++ | +++ | +++ | +++ | +++ | +++ | +++ | +++ |
| | PRECURSOR (ALPHA-1 | | | | | | | | | |
| | PROTEASE INHIBITOR) | | | | | | | | | |
| | (ALPHA-1- | | | | | | | | | |
| | ANTIPROTEINASE) | | | | | | | | | |
| M7 | FIBRINOGEN | +++ | +++ | +++ | +++ | +++ | +++ | +++ | +++ | ++ |
| | ALPHA/ALPHA-E CHAIN | | | | | | | | | |
| | PRECURSOR | | | | | | | | | |
| M8 | FIBRINOGEN BETA | +++ | ++ | +++ | +++ | +++ | +++ | +++ | +++ | ++ |
| | CHAIN PRECURSOR | | | | | | | | | |
| M9 | vitamin D-binding protein | +++ | +++ | +++ | +++ | +++ | +++ | ++ | +++ | ++ |
| M10 | TRANSTHYRETIN | +++ | +++ | +++ | +++ | +++ | +++ | +++ | +++ | +++ |
| | PRECURSOR | | | | | | | | | |
| | (PREALBUMIN) (TBPA) | | | | | | | | | |
| | (TTR) (ATTR) | | | | | | | | | |
| M11 | CERULOPLASMIN | ++ | +++ | +++ | +++ | +++ | +++ | ++ | +++ | ++ |
| | PRECURSOR | | | | | | | | | |
| | (FERROXIDASE) | | | | | | | | | |
| M12 | apolipoprotein A-IV | ++ | ++ | +++ | +++ | +++ | +++ | +++ | +++ | ++ |
| | precursor | | | | | | | | | |
| M13 | alpha 1-acid glycoprotein | +++ | +++ | +++ | +++ | +++ | +++ | +++ | +++ | +++ |
| M14 | hemopexin | +++ | +++ | +++ | +++ | +++ | +++ | ++ | +++ | ++ |
| M15 | macroglobulin alpha2 | ++ | +++ | ++ | +++ | ++ | ++ | ++ | +++ | ++ |
| M16 | IgG rearranged gamma chain | +++ | +++ | +++ | +++ | +++ | +++ | +++ | +++ | +++ |
| M17 | alpha-2-glycoprotein 1, zinc | +++ | +++ | +++ | +++ | +++ | +++ | +++ | +++ | +++ |
| M18 | Ig lambda chain | +++ | +++ | +++ | +++ | +++ | +++ | +++ | +++ | +++ |
| M19 | immunoglobulin alpha-1 | +++ | +++ | +++ | +++ | +++ | +++ | +++ | +++ | +++ |
| | heavy chain constant region | | | | | | | | | |
| M20 | HEMOGLOBIN BETA | +++ | +++ | +++ | +++ | ++ | ++ | ++ | +++ | − |
| | CHAIN | | | | | | | | | |
| M21 | ANTITHROMBIN-III | ++ | ++ | +++ | +++ | ++ | ++ | + | +++ | ++ |
| | PRECURSOR (ATIII) | | | | | | | | | |
| M22 | AMBP PROTEIN | ++ | +++ | +++ | +++ | +++ | +++ | ++ | +++ | +++ |
| | PRECURSOR [CONTAINS: | | | | | | | | | |
| | ALPHA-1- | | | | | | | | | |
| | MICROGLOBULIN | | | | | | | | | |
| | (PROTEIN HC) | | | | | | | | | |
| | (COMPLEX-FORMING | | | | | | | | | |
| | GLYCOPROTEIN | | | | | | | | | |
| | HETEROGENEOUS IN | | | | | | | | | |
| | CHARGE) | | | | | | | | | |
| M23 | PROTHROMBIN | ++ | +++ | +++ | +++ | +++ | ++ | ++ | +++ | ++ |
| | PRECURSOR | | | | | | | | | |
| | (COAGULATION FACTOR | | | | | | | | | |
| | II) | | | | | | | | | |
| M24 | ALPHA-2-HS- | +++ | +++ | +++ | +++ | +++ | +++ | +++ | +++ | +++ |
| | GLYCOPROTEIN | | | | | | | | | |
| | PRECURSOR (FETUIN-A) | | | | | | | | | |
| | (ALPHA-2-Z-GLOBULIN) | | | | | | | | | |
| | (BA-ALPHA-2- | | | | | | | | | |
| | GLYCOPROTEIN) | | | | | | | | | |
| M25 | actin beta | ++ | +++ | +++ | ++ | +++ | ++ | ++ | − | ++ |
| M26 | CLUSTERIN PRECURSOR | +++ | ++ | ++ | ++ | ++ | +++ | +++ | +++ | +++ |
| | (COMPLEMENT- | | | | | | | | | |
| | ASSOCIATED PROTEIN | | | | | | | | | |
| | SP-40, 40) (COMPLEMENT | | | | | | | | | |
| | CYTOLYSIS INHIBITOR) | | | | | | | | | |
| | (CLI) (NA1 AND NA2) | | | | | | | | | |
| | (APOLIPOPROTEIN J) | | | | | | | | | |
| | (APO-J) (TRPM-2) | | | | | | | | | |
| M27 | bA120D12.1 (CD5 antigen- | ++ | ++ | ++ | +++ | ++ | +++ | ++ | +++ | ++ |
| | like (scavenger receptor | | | | | | | | | |
| | cysteine rich family)) | | | | | | | | | |
| M28 | fibrinogen gamma-B chain | +++ | ++ | +++ | +++ | ++ | +++ | +++ | +++ | ++ |
| | precursor | | | | | | | | | |
| M29 | polyubiquitin 4 | ++ | +++ | ++ | ++ | + | ++ | ++ | − | − |
| M30 | KININOGEN PRECURSOR | ++ | − | − | − | − | ++ | ++ | + | ++ |
| | (ALPHA-2-THIOL | | | | | | | | | |
| | PROTEINASE INHIBITOR) | | | | | | | | | |
| | [CONTAINS: | | | | | | | | | |
| | BRADYKININ] | | | | | | | | | |
| M31 | CALGRANULIN B | +++ | +++ | +++ | +++ | +++ | +++ | +++ | ++ | ++ |
| | (MIGRATION | | | | | | | | | |
| | INHIBITORY FACTOR- | | | | | | | | | |
| | RELATED PROTEIN 14) | | | | | | | | | |
| | (MRP-14) (P14) | | | | | | | | | |
| | (LEUKOCYTE LI | | | | | | | | | |
| | COMPLEX HEAVY | | | | | | | | | |
| | CHAIN) (S100 CALCIUM- | | | | | | | | | |
| | BINDING PROTEIN A9) | | | | | | | | | |
| M32 | PLASMA RETINOL- | +++ | ++ | ++ | +++ | ++ | +++ | +++ | +++ | +++ |
| | BINDING PROTEIN | | | | | | | | | |
| | PRECURSOR (PRBP) (RBP) | | | | | | | | | |
| M33 | Ig mu chain precursor, | ++ | +++ | ++ | +++ | ++ | +++ | − | ++ | ++ |
| | membrane-bound (clone 201) | | | | | | | | | |
| M34 | APOLIPOPROTEIN A-II | ++ | +++ | +++ | +++ | +++ | +++ | +++ | +++ | +++ |
| | PRECURSOR (APO-AII) | | | | | | | | | |
| M35 | LEUCINE-RICH ALPHA-2- | ++ | +++ | ++ | +++ | +++ | +++ | ++ | +++ | ++ |
| | GLYCOPROTEIN (LRG) | | | | | | | | | |
| M36 | dJ34F7.4 (complement | ++ | − | ++ | +++ | ++ | + | ++ | − | ++ |
| | component 4A) | | | | | | | | | |
| M37 | similar to phosphoglycerate | ++ | ++ | ++ | ++ | ++ | ++ | ++ | − | − |
| | mutase 1 (brain) | | | | | | | | | |
| M38 | alpha-1-antichymotrypsin | − | ++ | ++ | ++ | ++ | ++ | − | ++ | + |
| | precursor | | | | | | | | | |
| M39 | ALPHA-1B- | ++ | ++ | +++ | +++ | ++ | +++ | ++ | +++ | ++ |
| | GLYCOPROTEIN | | | | | | | | | |
| M40 | HEMOGLOBIN ALPHA | ++ | +++ | +++ | +++ | ++ | + | ++ | +++ | − |
| | CHAIN | | | | | | | | | |
| M41 | lipoprotein CIII | ++ | ++ | ++ | ++ | +++ | +++ | +++ | +++ | +++ |
| M42 | TETRANECTIN | ++ | ++ | +++ | +++ | ++ | +++ | ++ | +++ | +++ |
| | PRECURSOR (TN) | | | | | | | | | |
| | (PLASMINOGEN- | | | | | | | | | |
| | KRINGLE 4 BINDING | | | | | | | | | |
| | PROTEIN) | | | | | | | | | |
| M43 | amyloid related serum | ++ | ++ | +++ | +++ | +++ | +++ | ++ | − | ++ |
| | protein SAA | | | | | | | | | |
| M44 | CALGRANULIN A | +++ | ++ | +++ | +++ | +++ | ++ | +++ | ++ | + |
| | (MIGRATION | | | | | | | | | |
| | INHIBITORY FACTOR- | | | | | | | | | |
| | RELATED PROTEIN 8) | | | | | | | | | |
| | (MRP-8) (CYSTIC | | | | | | | | | |
| | FIBROSIS ANTIGEN) | | | | | | | | | |
| | (CFAG) (P8) (LEUKOCYTE | | | | | | | | | |
| | LI COMPLEX LIGHT | | | | | | | | | |
| | CHAIN) (S100 CALCIUM- | | | | | | | | | |
| | BINDING PROTEIN A8) | | | | | | | | | |
| M45 | C-REACTIVE PROTEIN | − | +++ | ++ | +++ | +++ | ++ | + | − | ++ |
| | PRECURSOR | | | | | | | | | |
| M46 | RHO GDP-DISSOCIATION | ++ | ++ | +++ | ++ | ++ | ++ | ++ | − | + |
| | INHIBITOR 2 (RHO GDI 2) | | | | | | | | | |
| | (RHO-GDI BETA) (LY- | | | | | | | | | |
| | GDI) | | | | | | | | | |
| M47 | PROFILIN I | +++ | +++ | +++ | +++ | ++ | ++ | ++ | ++ | ++ |
| M48 | IMMUNOGLOBULIN J | +++ | ++ | ++ | +++ | +++ | +++ | ++ | +++ | +++ |
| | CHAIN | | | | | | | | | |
| M49 | APOLIPOPROTEIN D | ++ | − | ++ | − | ++ | +++ | ++ | +++ | +++ |
| | PRECURSOR | | | | | | | | | |
| M50 | inter-alpha-trypsin inhibitor | ++ | ++ | ++ | ++ | ++ | +++ | + | ++ | ++ |
| | family heavy chain-related | | | | | | | | | |
| | protein | | | | | | | | | |
| M51 | 14-3-3 PROTEIN | ++ | ++ | ++ | ++ | ++ | − | + | − | + |
| | BETA/ALPHA (PROTEIN | | | | | | | | | |
| | KINASE C INHIBITOR | | | | | | | | | |
| | PROTEIN-1) (KCIP-1) | | | | | | | | | |
| | (PROTEIN 1054) | | | | | | | | | |
| M52 | Ig G1 H Nie | − | ++ | ++ | ++ | ++ | − | − | − | − |
| M53 | OSTEOPONTIN | + | ++ | ++ | − | − | − | − | − | − |
| | PRECURSOR (BONE | | | | | | | | | |
| | SIALOPROTEIN 1) | | | | | | | | | |
| | (URINARY STONE | | | | | | | | | |
| | PROTEIN) (SECRETED | | | | | | | | | |
| | PHOSPHOPROTEIN 1) | | | | | | | | | |
| | (SPP-1) (NEPHROPONTIN) | | | | | | | | | |
| | (UROPONTIN) | | | | | | | | | |
| M54 | AFAMIN PRECURSOR | ++ | − | ++ | − | − | ++ | − | ++ | + |
| | (ALPHA-ALBUMIN) | | | | | | | | | |
| | (ALPHA-ALB) | | | | | | | | | |
| M55 | TRIOSEPHOSPHATE | ++ | ++ | ++ | − | ++ | − | − | − | − |
| | ISOMERASE (TIM) | | | | | | | | | |
| M56 | pre-serum amyloid P | ++ | − | − | − | ++ | +++ | − | − | − |
| | component | | | | | | | | | |
| M57 | COMP_HUMAN | ++ | ++ | ++ | − | + | ++ | ++ | ++ | − |
| M58 | prosaposin (variant Gaucher | − | ++ | ++ | ++ | − | ++ | − | − | − |
| | disease and variant | | | | | | | | | |
| | metachromatic | | | | | | | | | |
| | leukodystrophy) | | | | | | | | | |
| M59 | VITRONECTIN | ++ | ++ | ++ | ++ | ++ | ++ | ++ | +++ | ++ |
| | PRECURSOR (SERUM | | | | | | | | | |
| | SPREADING FACTOR) (S- | | | | | | | | | |
| | PROTEIN) [CONTAINS: | | | | | | | | | |
| | SOMATOMEDIN B] | | | | | | | | | |
| M60 | CALGRANULIN C (S100 | ++ | +++ | ++ | ++ | ++ | ++ | ++ | − | − |
| | A12 protein) | | | | | | | | | |
| M61 | cofilin 1 (non-muscle) | ++ | ++ | ++ | ++ | ++ | ++ | − | ++ | ++ |
| M62 | cathepsin B | ++ | ++ | ++ | ++ | ++ | − | − | − | − |
| M63 | plasmin (EC 3.4.21.7) | + | + | − | ++ | + | ++ | − | ++ | + |
| | precursor [validated] | | | | | | | | | |
| M64 | PLASMA GLUTATHIONE | ++ | ++ | ++ | +++ | ++ | +++ | ++ | +++ | ++ |
| | PEROXIDASE | | | | | | | | | |
| | PRECURSOR (GSHPX-P) | | | | | | | | | |
| M65 | lumican | − | ++ | ++ | ++ | ++ | − | ++ | +++ | ++ |
| M66 | apolipoprotein C-II | ++ | + | − | − | − | ++ | +++ | ++ | ++ |
| M67 | calmodulin 2 (phosphorylase | ++ | ++ | ++ | − | ++ | ++ | ++ | − | + |
| | kinase, delta) | | | | | | | | | |
| M68 | THYMOSIN BETA-4 | ++ | + | ++ | ++ | ++ | − | − | − | − |
| M69 | uracil DNA glycosylase | ++ | ++ | ++ | ++ | − | − | − | − | − |
| M70 | defensin alpha-3 precursor, | +++ | +++ | +++ | +++ | ++ | − | ++ | − | ++ |
| | neutrophil-specific | | | | | | | | | |
| | [validated] | | | | | | | | | |
| M71 | CD14 antigen | − | − | ++ | ++ | ++ | − | ++ | − | − |
| M72 | peptidylprolyl isomerase | ++ | ++ | ++ | ++ | ++ | − | − | − | ++ |
| | (EC 5.2.1.8) A | | | | | | | | | |
| M73 | similar to transgelin 2 | ++ | ++ | ++ | − | ++ | + | − | − | − |
| M74 | BETA-2-GLYCOPROTEIN | ++ | − | − | ++ | +++ | − | ++ | ++ | − |
| | I PRECURSOR | | | | | | | | | |
| | (APOLIPOPROTEIN H) | | | | | | | | | |
| | (APO-H) (B2GPI) | | | | | | | | | |
| | (BETA(2)GPI) | | | | | | | | | |
| | (ACTIVATED PROTEIN C- | | | | | | | | | |
| | BINDING PROTEIN) (APC | | | | | | | | | |
| | INHIBITOR) | | | | | | | | | |
| M75 | complement 9 | − | − | − | − | − | ++ | + | − | − |
| M76 | alpha2 plasmin inhibitor | ++ | ++ | ++ | ++ | ++ | + | ++ | − | − |
| M77 | FICOLIN 3 PRECURSOR | − | − | ++ | − | − | ++ | ++ | − | ++ |
| | (COLLAGEN/FIBRINOGEN | | | | | | | | | |
| | DOMAIN-CONTAINING | | | | | | | | | |
| | PROTEIN 3) | | | | | | | | | |
| | (COLLAGEN/FIBRINOGEN | | | | | | | | | |
| | DOMAIN-CONTAINING | | | | | | | | | |
| | LECTIN 3 P35) (HAKATA | | | | | | | | | |
| | ANTIGEN) | | | | | | | | | |
| M78 | glutathione transferase | ++ | ++ | ++ | − | − | ++ | − | − | − |
| M79 | Ig kappa chain | ++ | ++ | ++ | +++ | +++ | +++ | ++ | ++ | ++ |
| M80 | TRYPSIN PRECURSOR | ++ | ++ | ++ | ++ | ++ | ++ | ++ | ++ | ++ |
| M81 | complement factor B | ++ | ++ | ++ | ++ | ++ | +++ | ++ | − | − |
| M82 | CALGIZZARIN (S100C | ++ | ++ | ++ | ++ | ++ | − | − | − | + |
| | PROTEIN) (MLN 70) | | | | | | | | | |
| M83 | BETA-2- | ++ | +++ | +++ | +++ | +++ | ++ | ++ | +++ | ++ |
| | MICROGLOBULIN | | | | | | | | | |
| | PRECURSOR | | | | | | | | | |
| M84 | CYSTEINE-RICH | + | − | ++ | ++ | − | ++ | − | ++ | ++ |
| | SECRETORY PROTEIN-3 | | | | | | | | | |
| | PRECURSOR (CRISP-3) | | | | | | | | | |
| | (SGP28 PROTEIN) | | | | | | | | | |
| M85 | rearranged Ig kappa-chain | + | ++ | ++ | ++ | ++ | +++ | − | − | ++ |
| M86 | megakaryocyte stimulating | ++ | ++ | ++ | ++ | − | ++ | − | ++ | ++ |
| | factor | | | | | | | | | |
| M87 | inter-alpha-trypsin inhibitor | + | − | − | ++ | ++ | + | + | ++ | − |
| | heavy chain H1 precursor | | | | | | | | | |
| M88 | keratin 9, cytoskeletal | − | − | + | − | − | + | − | − | + |
| M89 | FERRITIN HEAVY CHAIN | − | ++ | ++ | − | − | + | + | − | − |
| | (FERRITIN H SUBUNIT) | | | | | | | | | |
| M90 | HISTONE H1.1 | + | ++ | ++ | − | + | − | ++ | − | − |
| M91 | keratin 1 | − | − | − | − | ++ | ++ | − | − | + |
| M92 | ferritin light subunit | − | ++ | ++ | ++ | − | + | + | − | − |
| M93 | PROTEASOME SUBUNIT | + | ++ | ++ | − | − | − | − | − | − |
| | ALPHA TYPE 2 | | | | | | | | | |
| | (PROTEASOME | | | | | | | | | |
| | COMPONENT C3) | | | | | | | | | |
| | (MACROPAIN SUBUNIT | | | | | | | | | |
| | C3) (MULTICATALYTIC | | | | | | | | | |
| | ENDOPEPTIDASE | | | | | | | | | |
| | COMPLEX SUBUNIT C3) | | | | | | | | | |
| M94 | CARBONIC ANHYDRASE | − | ++ | ++ | − | − | − | − | − | − |
| | I (CARBONATE | | | | | | | | | |
| | DEHYDRATASE I) (CA-I) | | | | | | | | | |
| M95 | LYMPHOCYTE-SPECIFIC | − | − | ++ | − | − | − | − | − | − |
| | PROTEIN LSP1 (PP52 | | | | | | | | | |
| | PROTEIN) (52 KDA | | | | | | | | | |
| | PHOSPHOPROTEIN) | | | | | | | | | |
| | (LYMPHOCYTE-SPECIFIC | | | | | | | | | |
| | ANTIGEN WP34) | | | | | | | | | |
| M96 | apolipoprotein F | ++ | − | ++ | − | − | ++ | ++ | − | ++ |
| M97 | immunoglobulin lambda | − | ++ | ++ | − | − | − | − | ++ | − |
| | chain variable region | | | | | | | | | |
| M98 | OSTEOINDUCTIVE | − | − | ++ | − | − | − | − | ++ | − |
| | FACTOR PRECURSOR | | | | | | | | | |
| | (OIF) (OSTEOGLYCIN) | | | | | | | | | |
| | (MIMECAN) | | | | | | | | | |
| M99 | APOLIPOPROTEIN E | − | ++ | − | + | − | ++ | ++ | ++ | − |
| | PRECURSOR (APO-E) | | | | | | | | | |
| M100 | PHOSPHATIDYLETHANO | ++ | + | ++ | − | − | − | − | − | − |
| | LAMINE-BINDING | | | | | | | | | |
| | PROTEIN (PEBP) | | | | | | | | | |
| | (NEUROPOLYPEPTIDE | | | | | | | | | |
| | H3) (HIPPOCAMPAL | | | | | | | | | |
| | CHOLINERGIC | | | | | | | | | |
| | NEUROSTIMULATING | | | | | | | | | |
| | PEPTIDE) (HCNP) (RAF | | | | | | | | | |
| | KINASE INHIBITOR | | | | | | | | | |
| | PROTEIN) (RKIP) | | | | | | | | | |
| M101 | SUPEROXIDE | ++ | − | + | − | + | ++ | − | − | − |
| | DISMUTASE [CU-ZN] | | | | | | | | | |
| M102 | HISTONE H1A (H1.1) | + | ++ | ++ | − | + | − | + | − | − |
| M103 | neutrophil lipocalin | ++ | − | ++ | − | − | − | − | − | − |
| M104 | serum | − | − | − | ++ | ++ | − | − | − | ++ |
| | paraoxonasearylesterase 1 | | | | | | | | | |
| M105 | CYTIDINE DEAMINASE | ++ | − | ++ | − | − | ++ | − | − | − |
| | (CYTIDINE | | | | | | | | | |
| | AMINOHYDROLASE) | | | | | | | | | |
| M106 | CYSTATIN B (LIVER | ++ | ++ | ++ | ++ | ++ | − | − | + | − |
| | THIOL PROTEINASE | | | | | | | | | |
| | INHIBITOR) (CPI-B) | | | | | | | | | |
| | (STEFIN B) | | | | | | | | | |
| M107 | immunoglobulin kappa light | − | − | − | ++ | ++ | + | − | − | − |
| | chain variable region | | | | | | | | | |
| M108 | angiotensinogen (serine (or | − | − | − | ++ | − | − | − | ++ | − |
| | cysteine) proteinase inhibitor, | | | | | | | | | |
| | clade A (alpha-1 | | | | | | | | | |
| | antiproteinase, antitrypsin), | | | | | | | | | |
| | member 8) | | | | | | | | | |
| M109 | COLLAGEN ALPHA 3(VI) | − | − | ++ | ++ | − | − | − | ++ | − |
| | CHAIN PRECURSOR | | | | | | | | | |
| M110 | beta galactoside binding | − | ++ | − | ++ | − | − | − | ++ | − |
| | lectin | | | | | | | | | |
| M111 | SH3BGRL3-like protein | ++ | ++ | ++ | ++ | − | − | − | ++ | − |
| M112 | proteoglycan link protein 2 | − | − | − | ++ | − | − | − | ++ | − |
| M113 | LYSOZYME C | − | ++ | − | ++ | − | − | − | − | − |
| | PRECURSOR (1,4-BETA-N- | | | | | | | | | |
| | ACETYLMURAMIDASE | | | | | | | | | |
| | C) | | | | | | | | | |
| M114 | cysteine-rich secreted A12- | − | + | ++ | ++ | − | − | − | − | − |
| | alpha-like protein 2 | | | | | | | | | |
| M115 | LEUKOCYTE ELASTASE | − | ++ | + | − | − | − | − | − | − |
| | INHIBITOR (LEI) | | | | | | | | | |
| | (MONOCYTE/NEUTROPHIL | | | | | | | | | |
| | ELASTASE INHIBITOR) | | | | | | | | | |
| | (M/NEI) (EI) | | | | | | | | | |
| M116 | INSULIN-LIKE GROWTH | + | + | − | ++ | ++ | − | − | ++ | − |
| | FACTOR BINDING | | | | | | | | | |
| | PROTEIN 4 PRECURSOR | | | | | | | | | |
| | (IGFBP-4) (IBP-4) (IGF- | | | | | | | | | |
| | BINDING PROTEIN 4) | | | | | | | | | |
| M117 | LOW AFFINITY | ++ | ++ | ++ | ++ | ++ | − | ++ | − | − |
| | IMMUNOGLOBULIN | | | | | | | | | |
| | GAMMA FC REGION | | | | | | | | | |
| | RECEPTOR III-A | | | | | | | | | |
| | PRECURSOR (IGG FC | | | | | | | | | |
| | RECEPTOR III-2) (FC- | | | | | | | | | |
| | GAMMA RIII-ALPHA) (FC- | | | | | | | | | |
| | GAMMA RIIIA) (FCRIIIA) | | | | | | | | | |
| | (FC-GAMMA RIII) (FCRIII) | | | | | | | | | |
| | (CD16-A) (FCR-10) | | | | | | | | | |
| M118 | sex hormone-binding | − | − | + | ++ | − | − | + | ++ | − |
| | globulin | | | | | | | | | |
| M119 | Annexin I (Lipocortin I) | − | + | ++ | ++ | − | − | ++ | − | − |
| | (Calpactin II) (Chromobindin | | | | | | | | | |
| | 9) (P35) (Phospholipase A2 | | | | | | | | | |
| | Inhibitory Protein) | | | | | | | | | |
| M120 | MnSOD precursor | − | − | ++ | − | + | + | − | − | − |
| M121 | EPIDIDYMAL | ++ | ++ | ++ | ++ | − | − | − | ++ | − |
| | SECRETORY PROTEIN E1 | | | | | | | | | |
| | PRECURSOR (NIEMANN- | | | | | | | | | |
| | PICK DISEASE TYPE C2 | | | | | | | | | |
| | PROTEIN) (EPI-1) (HE1) | | | | | | | | | |
| | (EPIDIDYMAL | | | | | | | | | |
| | SECRETORY PROTEIN | | | | | | | | | |
| | 14.6) (ESP14.6) | | | | | | | | | |
| M122 | monoclonal antibody HW1 | − | − | − | ++ | ++ | − | − | − | − |
| | immunoglobulin light chain | | | | | | | | | |
| | variable region | | | | | | | | | |
| M123 | METASTATIN | ++ | ++ | ++ | − | ++ | − | − | ++ | − |
| M124 | inter-alpha (globulin) | − | − | − | − | − | ++ | + | − | − |
| | inhibitor, H2 polypeptide | | | | | | | | | |
| M125 | alpha-1 type 1 collagen | − | ++ | + | ++ | − | ++ | − | ++ | ++ |
| M126 | GELSOLIN PRECURSOR, | − | ++ | − | − | − | − | − | ++ | − |
| | PLASMA (ACTIN- | | | | | | | | | |
| | DEPOLYMERIZING | | | | | | | | | |
| | FACTOR) (ADF) (BREVIN) | | | | | | | | | |
| | (AGEL) | | | | | | | | | |
| M127 | PROTEASOME SUBUNIT | − | ++ | ++ | − | − | − | − | − | − |
| | ALPHA TYPE 4 | | | | | | | | | |
| | (PROTEASOME | | | | | | | | | |
| | COMPONENT C9) | | | | | | | | | |
| | (MACROPAIN SUBUNIT | | | | | | | | | |
| | C9) (MULTICATALYTIC | | | | | | | | | |
| | ENDOPEPTIDASE | | | | | | | | | |
| | COMPLEX SUBUNIT C9) | | | | | | | | | |
| | (PROTEASOME SUBUNIT | | | | | | | | | |
| | L) | | | | | | | | | |
| M128 | SIMIALR TO COACTOSIN- | − | ++ | ++ | ++ | − | + | − | − | − |
| | LIKE PROTEIN | | | | | | | | | |
| M129 | THIOREDOXIN (ATL- | ++ | ++ | ++ | − | − | − | + | − | − |
| | DERIVED FACTOR) (ADF) | | | | | | | | | |
| | (SURFACE ASSOCIATED | | | | | | | | | |
| | SULPHYDRYL PROTEIN) | | | | | | | | | |
| | (SASP) | | | | | | | | | |
| M130 | K12 PROTEIN | − | ++ | − | ++ | − | − | − | ++ | − |
| | PRECURSOR | | | | | | | | | |
| M131 | VITAMIN-K DEPENDENT | − | − | − | − | − | ++ | + | − | − |
| | PROTEIN C PRECURSOR | | | | | | | | | |
| | (AUTOPROTHROMBIN | | | | | | | | | |
| | IIA) (ANTICOAGULANT | | | | | | | | | |
| | PROTEIN C) (BLOOD | | | | | | | | | |
| | COAGULATION FACTOR | | | | | | | | | |
| | XIV) | | | | | | | | | |
| M132 | titin, cardiac muscle | − | − | − | − | − | − | − | − | ++ |
| | [validated] | | | | | | | | | |
| M133 | MBL-associated protein | − | − | − | ++ | − | ++ | − | ++ | ++ |
| | MAp19 | | | | | | | | | |
| M134 | EOSINOPHIL CATIONIC | − | ++ | ++ | ++ | + | − | ++ | − | − |
| | PROTEIN PRECURSOR | | | | | | | | | |
| | (ECP) (RIBONUCLEASE 3) | | | | | | | | | |
| | (RNASE 3) | | | | | | | | | |
| M135 | This CDS feature is included | − | − | ++ | − | ++ | − | − | − | − |
| | to show the translation of the | | | | | | | | | |
| | corresponding V_region. | | | | | | | | | |
| | Presently translation | | | | | | | | | |
| | qualifiers on V_region | | | | | | | | | |
| | features are illegal | | | | | | | | | |
| M136 | enhancer protein | − | ++ | ++ | − | − | − | − | − | − |
| M137 | cysteine-rich protein 1 | − | ++ | − | ++ | − | + | − | ++ | − |
| M138 | anti-Gd cold agglutinin | − | ++ | − | ++ | ++ | ++ | ++ | ++ | − |
| | monoclonal IgMK light chain | | | | | | | | | |
| | variable region | | | | | | | | | |
| M139 | CYSTATIN A (STEFIN A) | + | − | − | ++ | ++ | − | − | − | − |
| | (CYSTATIN AS) | | | | | | | | | |
| M140 | ANNEXIN II | − | ++ | ++ | − | − | − | − | − | − |
| | (LIPOCORTIN II) | | | | | | | | | |
| | (CALPACTIN I HEAVY | | | | | | | | | |
| | CHAIN) (CHROMOBINDIN | | | | | | | | | |
| | 8) (P36) (PROTEIN I) | | | | | | | | | |
| | (PLACENTAL | | | | | | | | | |
| | ANTICOAGULANT | | | | | | | | | |
| | PROTEIN IV) (PAP-IV) | | | | | | | | | |
| M141 | diazepam binding inhibitor | − | ++ | ++ | + | − | − | − | − | − |
| M142 | bA139H14.1 (lymphocyte | − | − | ++ | − | − | − | − | − | + |
| | cytosolic protein 1 (L- | | | | | | | | | |
| | plastin)) | | | | | | | | | |
| M143 | heparan sulfate proteoglycan | − | − | − | ++ | − | − | − | ++ | − |
| | 2 (perlecan) | | | | | | | | | |
| M144 | cathepsin C | − | − | + | + | − | − | − | − | − |
| M145 | MYELOBLASTIN | + | − | − | − | ++ | + | − | − | − |
| | PRECURSOR | | | | | | | | | |
| | (LEUKOCYTE | | | | | | | | | |
| | PROTEINASE 3) (PR-3) | | | | | | | | | |
| | (PR3) (AGP7) | | | | | | | | | |
| | (WEGENER'S | | | | | | | | | |
| | AUTOANTIGEN) (P29) (C- | | | | | | | | | |
| | ANCA ANTIGEN) | | | | | | | | | |
| M146 | glutaredoxin | − | + | ++ | − | ++ | − | − | − | − |
| M147 | TRANSLATIONALLY | − | ++ | ++ | − | − | + | − | − | − |
| | CONTROLLED TUMOR | | | | | | | | | |
| | PROTEIN (TCTP) | | | | | | | | | |
| M148 | PROTEASOME | − | − | − | − | − | ++ | − | − | − |
| | COMPONENT C13 | | | | | | | | | |
| | PRECURSOR | | | | | | | | | |
| | (MACROPAIN SUBUNIT | | | | | | | | | |
| | C13) (MULTICATALYTIC | | | | | | | | | |
| | ENDOPEPTIDASE | | | | | | | | | |
| | COMPLEX SUBUNIT C13) | | | | | | | | | |
| M149 | ENDOTHELIAL PROTEIN | − | ++ | ++ | − | − | − | − | − | − |
| | C RECEPTOR | | | | | | | | | |
| | PRECURSOR | | | | | | | | | |
| | (ENDOTHELIAL CELL | | | | | | | | | |
| | PROTEIN C RECEPTOR) | | | | | | | | | |
| | (ACTIVATED PROTEIN C | | | | | | | | | |
| | RECEPTOR) (APC | | | | | | | | | |
| | RECEPTOR) | | | | | | | | | |
| M150 | Ig superfamily protein | − | − | ++ | ++ | − | − | − | ++ | − |
| M151 | D-DOPACHROME | ++ | + | ++ | − | + | − | − | − | − |
| | TAUTOMERASE | | | | | | | | | |
| | (PHENYLPYRUVATE | | | | | | | | | |
| | TAUTOMERASE II) | | | | | | | | | |
| M152 | p80 protein | − | ++ | − | − | − | − | − | − | − |
| M153 | COLLAGEN ALPHA 1(V) | − | − | ++ | − | − | − | − | ++ | − |
| | CHAIN PRECURSOR | | | | | | | | | |
| M154 | immunoglobulin light chain | − | − | ++ | − | ++ | ++ | − | − | − |
| | variable region | | | | | | | | | |
| M155 | rho GDP dissociation | − | − | ++ | − | − | − | − | − | − |
| | inhibitor (GDI) | | | | | | | | | |
| M156 | FIBRONECTIN | − | − | ++ | − | − | + | − | − | − |
| | PRECURSOR (FN) (COLD- | | | | | | | | | |
| | INSOLUBLE GLOBULIN) | | | | | | | | | |
| | (CIG) | | | | | | | | | |
| M157 | FK506-BIND[NG | ++ | ++ | − | − | − | − | − | − | − |
| | PROTEIN (FKBP-12) | | | | | | | | | |
| | (PEPTIDYL-PROLYL CIS- | | | | | | | | | |
| | TRANS ISOMERASE) | | | | | | | | | |
| | (PPIASE) (ROTAMASE) | | | | | | | | | |
| | (IMMUNOPHILIN FKBP12) | | | | | | | | | |
| M158 | IG KAPPA CHAIN V | ++ | − | − | ++ | ++ | ++ | − | − | ++ |
| | REGION GOM | | | | | | | | | |
| M159 | similar to osteoclast | ++ | − | − | − | − | − | − | − | − |
| | stimulating factor 1 (H. | | | | | | | | | |
| | sapiens) | | | | | | | | | |
| M160 | Ig heavy chain V-III region | + | − | ++ | ++ | − | − | ++ | ++ | ++ |
| | (TD-Vr) | | | | | | | | | |
| M161 | PEROXIREDOXIN 2 | ++ | − | + | − | − | − | − | − | − |
| | (THIOREDOXIN | | | | | | | | | |
| | PEROXIDASE 1) | | | | | | | | | |
| | (THIOREDOXIN- | | | | | | | | | |
| | DEPENDENT PEROXIDE | | | | | | | | | |
| | REDUCTASE 1) (THIOL- | | | | | | | | | |
| | SPECIFIC ANTIOXIDANT | | | | | | | | | |
| | PROTEIN) (TSA) (PRP) | | | | | | | | | |
| | (NATURAL KILLER CELL | | | | | | | | | |
| | ENHANCING FACTOR B) | | | | | | | | | |
| | (NKEF-B) | | | | | | | | | |
| M162 | myosin catalytic light chain | ++ | ++ | − | − | − | − | − | − | − |
| | LC17b | | | | | | | | | |
| M163 | protein SPY75 | + | + | − | − | − | − | − | − | − |
| M164 | 2-phosphopyruvate- | − | ++ | − | − | ++ | − | − | − | − |
| | hydratase alpha-enolase | | | | | | | | | |
| M165 | IG LAMBDA CHAIN V-V | − | ++ | ++ | − | − | − | − | − | − |
| | REGION DEL | | | | | | | | | |
| M166 | pancreatic secretory trypsin | − | + | − | ++ | + | − | − | − | − |
| | inhibitor | | | | | | | | | |
| M167 | GLIA MATURATION | ++ | − | + | ++ | − | − | − | − | − |
| | FACTOR GAMMA (GMF- | | | | | | | | | |
| | GAMMA) | | | | | | | | | |
| M168 | CALCYCLIN (LUNG 10 | − | + | ++ | − | − | + | − | − | − |
| | KDA PROTEIN) | | | | | | | | | |
| M169 | SH3 DOMAIN-BINDING | − | ++ | + | − | − | − | − | − | − |
| | GLUTAMIC ACID-RICH- | | | | | | | | | |
| | LIKE PROTEIN | | | | | | | | | |
| M170 | CHYMOTRYPSINOGEN A | − | − | − | − | − | ++ | − | − | − |
| M171 | DJ-1 protein | ++ | − | ++ | − | ++ | + | − | − | − |
| M172 | phosphoprotein enriched in | − | − | + | − | − | − | − | − | − |
| | astrocytes 15 | | | | | | | | | |
| M173 | EGF-CONTAINING | − | − | − | ++ | − | + | − | − | − |
| | FIBULIN-LIKE | | | | | | | | | |
| | EXTRACELLULAR | | | | | | | | | |
| | MATRIX PROTEIN 1 | | | | | | | | | |
| | PRECURSOR (FIBULIN-3) | | | | | | | | | |
| | (FIBL-3) (T16 PROTEIN) | | | | | | | | | |
| M174 | superoxide dismutase 3, | + | − | − | − | − | ++ | − | − | − |
| | extracellular | | | | | | | | | |
| M175 | CLARA CELL | − | + | + | ++ | − | − | − | ++ | − |
| | PHOSPHOLIPID-BINDING | | | | | | | | | |
| | PROTEIN PRECURSOR | | | | | | | | | |
| | (CCPBP) (CLARA CELLS | | | | | | | | | |
| | 10 KDA SECRETORY | | | | | | | | | |
| | PROTEIN) (CC10) | | | | | | | | | |
| | (UTEROGLOBIN) (URINE | | | | | | | | | |
| | PROTEIN 1) (UP1) | | | | | | | | | |
| M176 | Similar to LIM and SH3 | + | − | ++ | − | − | − | − | − | − |
| | protein 1 | | | | | | | | | |
| M177 | PROSTAGLANDIN-H2 D- | + | − | ++ | ++ | − | − | ++ | ++ | − |
| | ISOMERASE PRECURSOR | | | | | | | | | |
| | (PROSTAGLANDIN-D | | | | | | | | | |
| | SYNTHASE) | | | | | | | | | |
| | (GLUTATHIONE- | | | | | | | | | |
| | INDEPENDENT PGD | | | | | | | | | |
| | SYNTHETASE) | | | | | | | | | |
| | (PROSTAGLANDIN D2 | | | | | | | | | |
| | SYNTHASE) (PGD2 | | | | | | | | | |
| | SYNTHASE) (PGDS2) | | | | | | | | | |
| | (PGDS) (BETA-TRACE | | | | | | | | | |
| | PROTEIN) | | | | | | | | | |
| M178 | mannose 6- | − | ++ | − | − | − | − | − | − | − |
| | phosphate/insulin-like growth | | | | | | | | | |
| | factor II receptor | | | | | | | | | |
| M179 | OXYGEN-REGULATED | − | ++ | − | − | − | − | − | − | − |
| | PROTEIN 1 (RETINITIS | | | | | | | | | |
| | PIGMENTOSA RP1 | | | | | | | | | |
| | PROTEIN) (RETINITIS | | | | | | | | | |
| | PIGMENTOSA 1 PROTEIN) | | | | | | | | | |
| M180 | SERUM AMYLOID A | − | − | ++ | + | + | − | − | − | − |
| | PROTEIN (SAA) | | | | | | | | | |
| | [CONTAINS: AMYLOID | | | | | | | | | |
| | PROTEIN A (AMYLOID | | | | | | | | | |
| | FIBRIL PROTEIN AA)[ | | | | | | | | | |
| M181 | CARGO SELECTION | − | − | ++ | − | − | − | − | − | − |
| | PROTEIN TIP47 (47 KDA | | | | | | | | | |
| | MANNOSE 6-PHOSPHATE | | | | | | | | | |
| | RECEPTOR-BINDING | | | | | | | | | |
| | PROTEIN) (47 KDA MPR- | | | | | | | | | |
| | BINDING PROTEIN) | | | | | | | | | |
| | (PLACENTAL PROTEIN | | | | | | | | | |
| | 17) | | | | | | | | | |
| M182 | adenylyl cyclase-associated | − | − | ++ | − | − | ++ | − | − | − |
| | protein | | | | | | | | | |
| M183 | aldolase C | − | ++ | − | − | − | − | − | − | − |
| M184 | similar to PROTEASOME | − | − | ++ | − | − | − | − | − | − |
| | SUBUNIT ALPHA TYPE 6 | | | | | | | | | |
| | (PROTEASOME IOTA | | | | | | | | | |
| | CHAIN) (MACROPAIN | | | | | | | | | |
| | IOTA CHAIN) | | | | | | | | | |
| | (MULTICATALYTIC | | | | | | | | | |
| | ENDOPEPTIDASE | | | | | | | | | |
| | COMPLEX IOTA CHAIN) | | | | | | | | | |
| | (27 KDA PROSOMAL | | | | | | | | | |
| | PROTEIN) (PROS-27) | | | | | | | | | |
| | (P27K) (H. sapiens) | | | | | | | | | |
| M185 | immunoglobulin light chain | − | − | − | − | − | ++ | − | − | − |
| | variable region | | | | | | | | | |
| M186 | FOLLISTATIN-RELATED | − | − | + | ++ | ++ | ++ | − | − | − |
| | PROTEIN 1 PRECURSOR | | | | | | | | | |
| M187 | GAMMA-INTERFERON | − | ++ | ++ | − | − | ++ | − | − | − |
| | INDUCIBLE LYSOSOMAL | | | | | | | | | |
| | THIOL REDUCTASE | | | | | | | | | |
| | PRECURSOR (GAMMA- | | | | | | | | | |
| | INTERFERON-INDUCIBLE | | | | | | | | | |
| | PROTEIN IP-30) | | | | | | | | | |
| M188 | F-ACTIN CAPPING | ++ | ++ | − | − | − | − | ++ | − | − |
| | PROTEIN BETA SUBUNIT | | | | | | | | | |
| | (CAPZ BETA) | | | | | | | | | |
| M189 | coagulation factor X | ++ | − | − | − | − | − | − | − | + |
| M190 | histone H3 | − | − | − | − | − | − | ++ | − | − |
| M191 | adiponectin | − | − | − | − | ++ | − | − | − | − |
| M192 | 16G2 | − | + | + | − | − | − | − | ++ | − |
| M193 | Ig kappa chain precursor V | ++ | − | − | − | − | − | − | − | − |
| | region (A10) | | | | | | | | | |
| M194 | immunoglobulin lambda | − | − | + | − | − | ++ | − | − | − |
| | light chain variable region | | | | | | | | | |
| M195 | S-100P PROTEIN | ++ | + | − | − | + | − | − | − | − |
| M196 | INTERCELLULAR | − | + | − | ++ | − | − | − | ++ | − |
| | ADHESION MOLECULE-2 | | | | | | | | | |
| | PRECURSOR (ICAM-2) | | | | | | | | | |
| | (CD102) | | | | | | | | | |
| M197 | MANNOSYL- | − | − | − | ++ | − | − | − | − | − |
| | OLIGOSACCHARIDE | | | | | | | | | |
| | ALPHA-1,2- | | | | | | | | | |
| | MANNOSIDASE (MAN(9)- | | | | | | | | | |
| | ALPHA-MANNOSIDASE) | | | | | | | | | |
| M198 | NAD+-isocitrate | − | − | ++ | ++ | − | ++ | − | ++ | ++ |
| | dehydrogenase, alpha subunit | | | | | | | | | |
| M199 | CONNECTIVE TISSUE | − | − | − | − | − | − | − | ++ | − |
| | GROWTH FACTOR | | | | | | | | | |
| | PRECURSOR | | | | | | | | | |
| | (HYPERTROPHIC | | | | | | | | | |
| | CHONDROCYTE- | | | | | | | | | |
| | SPECIFIC PROTEIN 24) | | | | | | | | | |
| M200 | complement factor H-related | ++ | − | − | − | − | − | − | − | − |
| | protein FHR-2 | | | | | | | | | |
| M201 | RIBONUCLEASE, | − | − | − | − | − | ++ | − | − | − |
| | SEMINAL PRECURSOR | | | | | | | | | |
| | (SEMINAL RNASE) (S- | | | | | | | | | |
| | RNASE) (RIBONUCLEASE | | | | | | | | | |
| | BS-1) | | | | | | | | | |
| M202 | immunoglobulin kappa chain | − | − | − | − | ++ | + | − | − | − |
| M203 | putative | + | − | − | − | − | − | − | − | − |
| M204 | SERUM AMYLOID A-4 | − | − | − | − | − | ++ | − | − | − |
| | PROTEIN PRECURSOR | | | | | | | | | |
| | (CONSTITUTIVELY | | | | | | | | | |
| | EXPRESSED SERUM | | | | | | | | | |
| | AMYLOID A PROTEIN) | | | | | | | | | |
| | (C-SAA) | | | | | | | | | |
| M205 | HP1-BP74 | − | − | − | − | − | − | + | − | − |
| M206 | myristoylated alanine-rich | − | − | − | − | − | + | − | − | − |
| | C-kinase substrate | | | | | | | | | |
| M207 | CORTICOSTEROID- | − | ++ | − | − | − | + | − | ++ | − |
| | BINDING GLOBULIN | | | | | | | | | |
| | PRECURSOR (CBG) | | | | | | | | | |
| | (TRANSCORTIN) | | | | | | | | | |
| M208 | mannose-binding lectin | ++ | − | + | − | − | − | − | − | − |
| M209 | APOLIPOPROTEIN L | − | − | − | − | − | − | − | − | ++ |
| | PRECURSOR (APO-L) | | | | | | | | | |
| M210 | GLUTATHIONE | ++ | ++ | − | − | − | − | − | − | − |
| | TRANSFERASE OMEGA 1 | | | | | | | | | |
| | (GSTO 1-1) | | | | | | | | | |
| M211 | Somatomedin A | − | − | + | − | − | − | − | − | − |
| M212 | ganglioside M2 activator | − | ++ | − | − | − | − | − | − | + |
| | protein | | | | | | | | | |
| M213 | Ig heavy chain V region | + | − | − | ++ | − | ++ | − | ++ | + |
| | (174.2E10) | | | | | | | | | |
| M214 | FATTY ACID-BINDING | − | + | ++ | − | − | − | − | − | − |
| | PROTEIN, EPIDERMAL (E- | | | | | | | | | |
| | FABP) (PSORIASIS- | | | | | | | | | |
| | ASSOCIATED FATTY | | | | | | | | | |
| | ACID-BINDING PROTEIN | | | | | | | | | |
| | HOMOLOG) (PA-FABP) | | | | | | | | | |
| M215 | MYELOID CELL | − | − | + | − | − | − | − | − | − |
| | NUCLEAR | | | | | | | | | |
| | DIFFERENTIATION | | | | | | | | | |
| | ANTIGEN | | | | | | | | | |
| M216 | protein-L-isoaspartate(D- | ++ | − | + | − | − | − | − | − | − |
| | aspartate) O- | | | | | | | | | |
| | methyltransferase (EC | | | | | | | | | |
| | 2.1.1.77) splice form II | | | | | | | | | |
| M217 | pancreatic ribonuclease (EC | − | − | + | − | − | − | − | − | − |
| | 3.1.27.5) precursor | | | | | | | | | |
| M218 | IG KAPPA CHAIN V-I | − | − | − | ++ | − | − | − | − | − |
| | REGION MEV | | | | | | | | | |
| M219 | anti-MSP1 MAD20 block2 | − | − | − | ++ | − | − | − | − | − |
| | ScFv Ig light chain variable | | | | | | | | | |
| | region | | | | | | | | | |
| M220 | vimentin | + | − | ++ | − | + | − | − | − | + |
| M221 | HLA CLASS II | − | − | + | − | − | − | − | − | − |
| | HISTOCOMPATIBILITY | | | | | | | | | |
| | ANTIGEN, GAMMA | | | | | | | | | |
| | CHAIN (HLA-DR | | | | | | | | | |
| | ANTIGENS ASSOCIATED | | | | | | | | | |
| | INVARIANT CHAIN) (P33) | | | | | | | | | |
| | (CD74 ANTIGEN) | | | | | | | | | |
| M222 | IMP dehydrogenase | + | ++ | + | − | − | − | − | − | − |
| M223 | granulin | − | + | − | − | − | ++ | − | − | − |
| M224 | p97 | − | − | − | − | − | + | − | − | − |
| M225 | antigen HLA SB beta, MHC | − | − | + | − | − | − | − | − | − |
| | II | | | | | | | | | |
| M226 | immunoglobulin rearranged | ++ | − | ++ | − | − | − | − | − | − |
| | light chain | | | | | | | | | |
| M227 | kinesin like protein 9 | − | ++ | − | − | − | − | − | − | − |
| M228 | PROTEASOME | − | − | ++ | − | − | − | − | − | − |
| | COMPONENT MECL-1 | | | | | | | | | |
| | PRECURSOR | | | | | | | | | |
| | (MACROPAIN SUBUNIT | | | | | | | | | |
| | MECL-1) | | | | | | | | | |
| | (MULTICATALYTIC | | | | | | | | | |
| | ENDOPEPTIDASE | | | | | | | | | |
| | COMPLEX SUBUNIT | | | | | | | | | |
| | MECL-1) | | | | | | | | | |
| M229 | immunoglobulin kappa light | − | ++ | − | ++ | − | + | − | − | − |
| | chain variable region | | | | | | | | | |
| M230 | immunogloblin light chain | − | − | ++ | − | − | ++ | − | − | − |
| M231 | IG Lambda Chain V-IV | ++ | − | − | ++ | − | − | − | − | ++ |
| | Region Mol | | | | | | | | | |
| M232 | immunoglobulin variable | − | − | ++ | − | ++ | ++ | − | − | − |
| | region used by the ITC48 | | | | | | | | | |
| | kappa light chain (subgroup | | | | | | | | | |
| | V kappa IV) (anti- | | | | | | | | | |
| | cytomeglovirus glycoprotein | | | | | | | | | |
| | B(A | | | | | | | | | |
| M233 | hypothetical protein | − | − | − | ++ | − | − | − | − | − |
| | DKFZp761B15121.1 | | | | | | | | | |
| M234 | immunoglobulin kappa light | − | ++ | ++ | − | − | − | − | − | − |
| | chain variable region B3 | | | | | | | | | |
| M235 | VON EBNER'S GLAND | − | − | − | − | + | − | − | − | − |
| | PROTEIN PRECURSOR | | | | | | | | | |
| | (VEG PROTEIN) (TEAR | | | | | | | | | |
| | PREALBUMIN) (TP) | | | | | | | | | |
| | (TEAR LIPOCALIN) | | | | | | | | | |
| | (LIPOCALIN-1) | | | | | | | | | |
| M236 | Ribonuclease Pancreatic | − | − | − | − | − | + | − | − | − |
| | Precursor (RNASE 1) | | | | | | | | | |
| | (RNASE A) | | | | | | | | | |
| M237 | immunoglobulin kappa chain | − | − | − | ++ | − | ++ | − | − | − |
| | variable region | | | | | | | | | |
| M238 | immunoglobulin kappa light | − | ++ | − | ++ | − | − | − | − | − |
| | chain variable region | | | | | | | | | |
| M239 | immunoglobulin kappa chain | − | ++ | ++ | − | ++ | + | − | ++ | − |
| | V region | | | | | | | | | |
| M240 | Carbonic Anhydrase II | − | − | + | − | − | − | − | − | − |
| | (Carbonate Dehydratase II) | | | | | | | | | |
| | (CA-II) | | | | | | | | | |
| M241 | HRV Fab 027-VL | − | − | − | ++ | ++ | − | − | − | − |
| M242 | immunoglobulin variable | − | − | − | ++ | − | − | − | − | − |
| | region, kappa light chain | | | | | | | | | |
| M243 | IG Kappa Chain V-II Region | − | − | − | − | ++ | ++ | − | − | − |
| | MIL | | | | | | | | | |
| M244 | 364K Golgi complex- | − | − | + | − | − | − | − | − | − |
| | associated protein | | | | | | | | | |
| M245 | histone H2A.5 | + | − | − | − | − | − | + | − | − |
| M246 | antibody, light chain variable | − | − | − | ++ | − | − | − | − | − |
| | regin to HIV1 p25 | | | | | | | | | |
| M247 | immunoglobulin heavy- | + | − | + | ++ | − | − | ++ | ++ | + |
| | chain subgroup VIII V-D-J | | | | | | | | | |
| | region | | | | | | | | | |
| M248 | TRYPSINOGEN, | − | − | − | − | − | − | − | − | + |
| | CATIONIC PRECURSOR | | | | | | | | | |
| | (BETA-TRYPSIN) | | | | | | | | | |
| M249 | putative | − | − | ++ | − | − | − | − | ++ | − |
| M250 | LOW AFFINITY | − | − | ++ | − | − | − | − | − | − |
| | IMMUNOGLOBULIN | | | | | | | | | |
| | GAMMA FC REGION | | | | | | | | | |
| | RECEPTOR II-A | | | | | | | | | |
| | PRECURSOR (FC-GAMMA | | | | | | | | | |
| | RII-A) (FCRII-A) (IGG FC | | | | | | | | | |
| | RECEPTOR II-A) (FC- | | | | | | | | | |
| | GAMMA-RIIA) (CD32) | | | | | | | | | |
| | (CDW32) | | | | | | | | | |
| M251 | immunoglobulin lambda | + | − | − | − | − | − | − | − | − |
| | chain variable region | | | | | | | | | |
| M252 | CYTOCHROME C | − | + | − | − | − | − | − | − | − |
| M253 | Ig kappa chain V-IV region | − | ++ | − | − | − | − | − | ++ | − |
| | (Dep) | | | | | | | | | |
| M254 | immunoglobulin kappa | − | ++ | − | ++ | − | − | − | − | − |
| | orphon (AA 95) | | | | | | | | | |
| M255 | immunoglobulin kappa chain | − | − | − | − | ++ | − | − | − | − |
| | variable region | | | | | | | | | |
| M256 | immunoglobulin kappa light | − | − | − | ++ | ++ | − | − | − | − |
| | chain variable region | | | | | | | | | |
| M257 | immunoglobulin lambda | − | ++ | − | ++ | − | − | − | − | − |
| | light chain variable region | | | | | | | | | |
| M258 | gamma-glutamyl hydrolase | − | − | + | − | − | − | − | − | − |
| | (conjugase, | | | | | | | | | |
| | folylpolygammaglutamyl | | | | | | | | | |
| | hydrolase) precursor | | | | | | | | | |
| M259 | breakpoint cluster region | − | ++ | − | − | − | − | − | − | − |
| | protein 1 | | | | | | | | | |
| M260 | immunoglobulin kappa chain | − | − | − | − | ++ | − | − | − | − |
| | variable region | | | | | | | | | |
| M261 | BCL3 | − | − | − | − | − | − | − | − | − |
| M262 | immunoglobulin lambda | + | − | − | − | − | − | − | − | − |
| | light chain variable region | | | | | | | | | |
| M263 | immunoglobulin kappa chain | − | ++ | − | − | ++ | + | − | − | − |
| | variable region | | | | | | | | | |
| M264 | macrophage migration | − | ++ | − | − | − | − | − | − | − |
| | inhibitory factor | | | | | | | | | |
| M265 | immunoglobulin kappa chain | − | − | − | − | ++ | − | − | − | − |
| | variable region | | | | | | | | | |
| M266 | unnamed protein product | − | − | − | + | − | − | − | − | − |
| M267 | ribosomal protein S28, | − | + | ++ | − | + | − | − | − | − |
| | cytosolic | | | | | | | | | |
| M268 | MICROFIBRIL- | − | − | − | − | − | + | − | − | − |
| | ASSOCIATED | | | | | | | | | |
| | GLYCOPROTEIN 2 | | | | | | | | | |
| | PRECURSOR (MAGP-2) | | | | | | | | | |
| | (MP25) | | | | | | | | | |
| M269 | cathepsin S | − | ++ | − | − | − | − | − | − | − |
| M270 | lambda-chain C-region C- | − | − | − | − | − | − | − | − | − |
| | lambda-2 | | | | | | | | | |
| M271 | anti-porcine VCAM mAb | − | − | − | ++ | − | − | − | − | − |
| | 3F4 light chain variable | | | | | | | | | |
| | region | | | | | | | | | |
| M272 | immunoglobulin lambda-3 | − | − | − | − | − | ++ | − | − | − |
| | variable region | | | | | | | | | |
| M273 | immunoglobulin kappa chain | − | ++ | ++ | − | − | − | − | − | − |
| | variable region | | | | | | | | | |
| M274 | IgM light chain variable | − | − | − | ++ | − | − | − | − | − |
| | region | | | | | | | | | |
| M275 | CATHEPSIN D | + | + | − | − | − | − | − | − | − |
| M276 | immunoglobulin kappa light | − | − | − | − | − | − | − | − | − |
| | chain variable region | | | | | | | | | |
| M277 | putative | − | − | − | − | − | ++ | − | − | − |
| M278 | transaldolase | − | + | − | − | − | − | − | − | − |
| M279 | immunoglobulin V lambda/J | − | − | − | − | − | − | − | − | − |
| | lambda light chain | | | | | | | | | |
| M280 | myeloid inhibitory siglec | − | − | − | − | − | − | − | − | − |
| M281 | granulocyte inhibitory | − | − | − | − | ++ | − | − | − | − |
| | protein | | | | | | | | | |
| M282 | GROWTH FACTOR | + | − | − | − | − | − | − | − | − |
| | RECEPTOR-BOUND | | | | | | | | | |
| | PROTEIN 2 (GRB2 | | | | | | | | | |
| | ADAPTER PROTEIN) | | | | | | | | | |
| | (SH2/SH3 ADAPTER | | | | | | | | | |
| | GRB2) (ASH PROTEIN) | | | | | | | | | |
| M283 | immunoglobulin kappa light | − | − | − | − | − | − | − | − | − |
| | chain variable region | | | | | | | | | |
| M284 | UBIQUITIN-LIKE | − | ++ | − | − | − | − | − | − | − |
| | PROTEIN SMT3B | | | | | | | | | |
| | (SENTRIN 2) | | | | | | | | | |
| M285 | immunoglobulin kappa chain | − | − | − | − | − | − | − | − | ++ |
| M286 | HLA CLASS I | − | + | − | − | − | − | − | − | − |
| | HISTOCOMPATIBILITY | | | | | | | | | |
| | ANTIGEN, CW-1 CW*0102 | | | | | | | | | |
| | ALPHA CHAIN | | | | | | | | | |
| | PRECURSOR (CW1.2) | | | | | | | | | |
| M287 | 40S RIBOSOMAL | − | − | ++ | − | − | − | − | − | − |
| | PROTEIN S17 | | | | | | | | | |
| M288 | immunoglobulin kappa light | − | ++ | − | ++ | − | − | − | ++ | − |
| | chain variable region | | | | | | | | | |
| M289 | dJ747L4.1 (Brachyury (T | − | − | − | ++ | − | − | − | − | − |
| | box protein)-LIKE protein | | | | | | | | | |
| M290 | anti-oxidized LDL | ++ | − | − | − | ++ | − | − | − | − |
| | autoantibody variable kappa | | | | | | | | | |
| | chain | | | | | | | | | |
| M291 | immunoglobulin light chain | − | − | + | − | − | − | − | − | − |
| | variable region | | | | | | | | | |
| M292 | immunoglobulin kappa light | − | − | − | ++ | ++ | − | − | − | − |
| | chain variable region | | | | | | | | | |
| M293 | Ig kappa chain | − | − | − | ++ | − | − | − | − | − |
| M294 | immunoglobulin kappa chain | − | − | − | − | − | − | − | − | ++ |
| | variable region | | | | | | | | | |
| M295 | Ig kappa chain V-III region | − | − | − | − | ++ | − | − | − | − |
| | (Lew) | | | | | | | | | |
| M296 | similar to TROPOMYOSIN, | − | − | − | − | ++ | − | − | − | − |
| | CYTOSKELETAL TYPE | | | | | | | | | |
| | (TM30-NM) (H. sapiens) | | | | | | | | | |
| M297 | immunoglobulin kappa light | − | − | − | − | − | − | − | − | − |
| | chain, variable region | | | | | | | | | |
| M298 | ZYXIN (ZYXIN 2) | − | − | + | − | − | − | − | − | − |
| M299 | immunoglobulin lambda | − | − | − | − | − | − | ++ | − | − |
| | chain variable region | | | | | | | | | |
| M300 | ENHANCER OF | − | ++ | − | − | − | − | − | − | − |
| | RUDIMENTARY | | | | | | | | | |
| | HOMOLOG | | | | | | | | | |
| M301 | regulator of G-protein | − | − | − | − | − | − | − | − | ++ |
| | signaling 4 | | | | | | | | | |
| M302 | immunoglobulin lambda | − | − | − | − | − | + | − | − | − |
| | light chain variable region | | | | | | | | | |
| M303 | immunoglobulin kappa chain | − | − | − | − | ++ | − | − | − | − |
| M304 | immunoglobulin kappa light | − | − | − | ++ | − | − | − | − | − |
| | chain variable region | | | | | | | | | |
| M305 | anti-DNA immunoglobulin | − | − | − | − | − | − | − | − | − |
| | light chain IgG | | | | | | | | | |
| M306 | Ig kappa chain (Vk) V | − | − | − | − | − | − | − | − | + |
| | region (VJ) | | | | | | | | | |
| M307 | UBIQUITIN-LIKE | − | + | − | − | − | − | − | − | − |
| | PROTEIN NEDD8 | | | | | | | | | |
| M308 | immunoglobulin light chain | − | − | − | − | ++ | − | − | − | − |
| | variable region | | | | | | | | | |
| M309 | immunoglobulin lambda | − | − | ++ | − | − | ++ | − | − | − |
| | light chain variable region | | | | | | | | | |
| M310 | immunoglobulin lambda | ++ | ++ | − | − | − | − | − | − | − |
| | light chain | | | | | | | | | |
| M311 | SCRAPIE-RESPONSIVE | − | − | − | − | − | − | − | ++ | − |
| | PROTEIN 1 PRECURSOR | | | | | | | | | |
| | (SCRG-1) | | | | | | | | | |
| M312 | gonadotropin releasing | − | − | − | + | − | − | ++ | ++ | − |
| | peptide | | | | | | | | | |
| M313 | immunoglobulin light chain | − | − | ++ | − | − | − | − | − | − |
| | variable region | | | | | | | | | |
| M314 | IG HEAVY CHAIN V-III | − | − | − | ++ | ++ | + | − | − | − |
| | REGION HIL | | | | | | | | | |
| M315 | IRT-1 | − | ++ | − | − | − | − | − | − | − |
| M316 | recombinant antibody light | − | − | − | ++ | − | − | − | − | − |
| | chain VL domain | | | | | | | | | |
| M317 | putative | − | − | − | − | − | − | − | − | ++ |
| M318 | CYSTATIN M | − | − | − | − | − | − | − | ++ | − |
| | PRECURSOR (CYSTATIN | | | | | | | | | |
| | E) | | | | | | | | | |
| M319 | immunoglobulin rearranged | − | − | − | ++ | − | − | − | − | − |
| | light chain | | | | | | | | | |
| M320 | proteasome activator PA28 | − | − | − | − | ++ | − | − | − | − |
| | beta chain | | | | | | | | | |
| M321 | AGGRECAN CORE | − | − | − | − | − | ++ | − | − | − |
| | PROTEIN PRECURSOR | | | | | | | | | |
| | (CARTILAGE-SPECIFIC | | | | | | | | | |
| | PROTEOGLYCAN CORE | | | | | | | | | |
| | PROTEIN) (CSPCP) | | | | | | | | | |
| | (CHONDROITIN SULFATE | | | | | | | | | |
| | PROTEOGLYCAN CORE | | | | | | | | | |
| | PROTEIN 1) | | | | | | | | | |
| M322 | KIAA0185 protein | − | − | ++ | − | − | + | − | − | − |
| M323 | hypothetical protein | − | − | − | ++ | − | − | − | − | − |
| | FLJ13465 | | | | | | | | | |
| M324 | CARBOXYPEPTIDASE N | − | − | − | − | − | − | − | − | + |
| | 83 KDA CHAIN | | | | | | | | | |
| | (CARBOXYPEPTIDASE N | | | | | | | | | |
| | REGULATORY SUBUNIT) | | | | | | | | | |
| M325 | Ig kappa chain | − | − | − | − | − | − | − | − | − |
| M326 | R33729_1 | − | − | + | − | − | − | − | − | − |
| M327 | lymphocyte surface antigen | − | ++ | − | − | − | − | − | − | − |
| | precursor CD44 | | | | | | | | | |
| M328 | PYRIDOXINE KINASE | + | − | − | − | − | − | − | − | − |
| | (PYRIDOXAL KINASE) | | | | | | | | | |
| M329 | unnamed protein product | − | − | − | − | − | + | − | − | − |
| M330 | CALPACTIN I LIGHT | − | − | ++ | − | − | − | − | − | − |
| | CHAIN (P10 PROTEIN) | | | | | | | | | |
| | (P11) (CELLULAR | | | | | | | | | |
| | LIGAND OF ANNEXIN II) | | | | | | | | | |
| | (NERVE GROWTH | | | | | | | | | |
| | FACTOR INDUCED | | | | | | | | | |
| | PROTEIN 42C) | | | | | | | | | |
| M331 | c360B4.1 (PUTATIVE | + | − | − | − | − | − | ++ | − | ++ |
| | novel protein similar to | | | | | | | | | |
| | predicted bacterial and worm | | | | | | | | | |
| | proteins) | | | | | | | | | |
| M332 | latent transforming growth | − | − | − | − | − | − | − | ++ | − |
| | factor-beta binding protein | | | | | | | | | |
| | 4S | | | | | | | | | |
| M333 | hypothetical protein | − | − | − | − | − | − | − | − | − |
| | XP_047083 | | | | | | | | | |
| M334 | similar to | ++ | ++ | ++ | − | − | − | + | − | − |
| | GASTRIN/CHOLECYSTOKININ | | | | | | | | | |
| | TYPE B RECEPTOR | | | | | | | | | |
| | (CCK-B RECEPTOR) | | | | | | | | | |
| | (CCK-BR) (H. sapiens) | | | | | | | | | |
| M335 | immunoglobulin kappa chain | − | − | − | − | − | − | ++ | − | − |
| | variable region | | | | | | | | | |
| M336 | similar to LINE-1 | − | − | − | − | − | − | − | − | − |
| | REVERSE | | | | | | | | | |
| | TRANSCRIPTASE | | | | | | | | | |
| | HOMOLOG (H. sapiens) | | | | | | | | | |
| M337 | TYROSINE-PROTEIN | − | − | − | − | − | − | ++ | − | ++ |
| | KINASE CSK (C-SRC | | | | | | | | | |
| | KINASE) | | | | | | | | | |
| M338 | Similar to dystroglycan 1 | − | − | − | − | − | − | − | ++ | − |
| | (dystrophin-associated | | | | | | | | | |
| | glycoprotein 1) | | | | | | | | | |
| M339 | complement component 1, s | − | − | − | − | − | ++ | − | − | − |
| | subcomponent | | | | | | | | | |
| M340 | lysophospholipase II | − | − | − | − | − | − | − | − | − |
| M341 | Ig kappa chain V region (V- | − | − | − | ++ | − | − | + | − | − |
| | kappa 3) | | | | | | | | | |
| M342 | immunoglobulin V lambda/J | ++ | − | − | − | − | − | − | − | − |
| | lambda light chain | | | | | | | | | |
| M343 | immunoglobulin kappa light | − | − | − | − | − | − | − | − | − |
| | chain variable region | | | | | | | | | |
| M344 | TELOMERASE-BINDING | + | − | − | − | − | − | − | − | − |
| | PROTEIN P23 (HSP90 CO- | | | | | | | | | |
| | CHAPERONE) | | | | | | | | | |
| | (PROGESTERONE | | | | | | | | | |
| | RECEPTOR COMPLEX | | | | | | | | | |
| | P23) | | | | | | | | | |
| M345 | immunoglobulin lambda | − | − | − | − | − | ++ | − | − | − |
| | light chain variable region | | | | | | | | | |
| M346 | inwardly rectifying | − | − | − | − | − | − | ++ | − | − |
| | potassium channel protein | | | | | | | | | |
| | Kir6.2 | | | | | | | | | |
| M347 | Ig kappa chain | − | − | − | − | − | − | − | − | − |
| M348 | immunoglobulin light chain | − | − | − | ++ | − | − | − | − | − |
| M349 | dnaJ protein homolog | − | − | − | ++ | − | − | − | − | − |
| M350 | 40S RIBOSOMAL | − | ++ | − | − | − | − | − | − | − |
| | PROTEIN S29 | | | | | | | | | |
| M351 | IgA1 kappa light chain | − | − | ++ | − | ++ | − | − | − | − |
| M352 | MYOGLOBIN | − | − | − | − | − | − | − | − | − |
| M353 | cathepsin X precursor | − | ++ | − | − | − | − | − | − | − |
| M354 | unknown | − | ++ | − | − | − | − | − | − | − |
| M355 | immunoglobulin lambda | + | − | − | − | − | − | − | − | − |
| | chain variable region | | | | | | | | | |
| M356 | HYPOTHETICAL | − | − | − | − | − | − | + | − | − |
| | PROTEIN CGI-109 | | | | | | | | | |
| | PRECURSOR | | | | | | | | | |
| M357 | LITHOSTATHINE 1 BETA | + | − | − | − | − | + | − | − | − |
| | PRECURSOR | | | | | | | | | |
| | (REGENERATING | | | | | | | | | |
| | PROTEIN I BETA) | | | | | | | | | |
| M358 | human leucocyte antigen B | − | − | − | − | − | − | − | − | − |
| M359 | immunoglobulin light chain | − | − | − | − | − | − | + | − | − |
| | variable region | | | | | | | | | |
| M360 | This CDS feature is included | − | − | − | − | ++ | − | − | − | − |
| | to show the translation of the | | | | | | | | | |
| | corresponding V_region. | | | | | | | | | |
| | Presently translation | | | | | | | | | |
| | qualifiers on V_region | | | | | | | | | |
| | features are illegal. | | | | | | | | | |
| M361 | Ig kappa chain V-I region | − | − | − | ++ | − | − | − | − | − |
| | (ISE) | | | | | | | | | |
| M362 | KIAA0336 | ++ | − | − | ++ | − | − | − | − | − |
| M363 | Similar to expressed | − | − | − | − | − | − | − | − | − |
| | sequence 2 embryonic lethal | | | | | | | | | |
| M364 | immunoglobulin kappa light | − | − | − | − | ++ | − | − | − | − |
| | chain variable region | | | | | | | | | |
| M365 | Ig kappa chain V region (V- | ++ | − | − | − | − | − | − | − | − |
| | kappa 3) | | | | | | | | | |
| M366 | NEUROBLASTOMA | − | − | − | + | − | − | − | − | − |
| | SUPPRESSOR OF | | | | | | | | | |
| | TUMORIGENICITY 1 | | | | | | | | | |
| | (ZINC FINGER PROTEIN | | | | | | | | | |
| | DAN) (N03) | | | | | | | | | |
| M367 | Ig kappa chain | − | − | − | − | ++ | − | − | − | − |
| M368 | immunoglobulin lambda | − | − | ++ | − | − | − | − | − | − |
| | chain variable region | | | | | | | | | |
| M369 | immunoglobulin light chain | − | − | − | ++ | − | − | − | − | − |
| | variable region | | | | | | | | | |
| M370 | putative | − | − | − | − | − | ++ | − | − | − |
| M371 | immunoglobulin light chain | − | − | − | − | − | + | − | − | − |
| | VL region | | | | | | | | | |
| M372 | alpha IF calcium channel | − | − | − | − | − | − | − | − | + |
| | subunit | | | | | | | | | |
| M373 | sa1 (Drosophila)-like 2 | − | − | − | − | − | + | − | − | − |
| M374 | AF15ql4 protein | − | − | − | ++ | − | − | − | − | − |
| M375 | putative | − | − | − | − | − | − | − | − | − |
| M376 | fibroblast growth factor 13 | − | − | − | +++ | ++ | ++ | − | − | ++ |
| | isoform 1y1v | | | | | | | | | |
| M377 | immunoglobulin heavy chain | − | − | − | − | − | − | − | − | − |
| M378 | immunoglobulin heavy chain | − | − | ++ | − | − | − | − | − | − |
| M379 | KIAA0433 | + | + | − | − | − | − | − | − | − |
| M380 | anti-c-erbB-2 | − | − | − | − | − | ++ | − | − | − |
| | immunoglobulin light chain | | | | | | | | | |
| | V | | | | | | | | | |
| M381 | ATP-BINDING | − | − | − | ++ | − | − | − | − | − |
| | CASSETTE, SUB-FAMILY | | | | | | | | | |
| | A, MEMBER 2 (ATP- | | | | | | | | | |
| | BINDING CASSETTE | | | | | | | | | |
| | TRANSPORTER 2) (ATP- | | | | | | | | | |
| | BINDING CASSETTE 2) | | | | | | | | | |
| M382 | G protein-coupled receptor | − | − | − | ++ | − | − | − | − | − |
| | kinase-associated ADP | | | | | | | | | |
| | ribosylation factor GTPase- | | | | | | | | | |
| | activating protein | | | | | | | | | |
| M383 | immunoglobulin kappa chain | − | − | − | − | − | ++ | − | − | − |
| | V-J region | | | | | | | | | |
| M384 | Ig kappa chain V region | − | − | − | − | − | − | − | − | − |
| | (patient 17) | | | | | | | | | |
| M385 | immunoglobulin lambda | − | − | ++ | − | − | − | − | − | − |
| | light chain variable region | | | | | | | | | |
| M386 | immunoglobulin lambda | − | ++ | − | − | − | − | − | − | − |
| | chain | | | | | | | | | |
| M387 | farnesyl-protein transferase | − | − | ++ | − | + | ++ | ++ | ++ | ++ |
| | beta chain | | | | | | | | | |
| M388 | KIAA1813 protein | − | − | − | − | ++ | − | − | − | − |
| M389 | hypothetical protein | − | − | − | − | − | − | − | − | ++ |
| | FLJ11937 | | | | | | | | | |
| M390 | immunoglobulin light chain | − | − | − | − | ++ | − | − | − | − |
| | variable region | | | | | | | | | |
| M391 | ribosomal protein L29 | − | ++ | − | − | + | − | − | − | − |
| M392 | Ig kappa light chain (VJC) | − | − | − | − | + | − | − | − | − |
| M393 | inhibitor PI, alpha I | − | − | + | ++ | − | − | − | − | − |
| | proteinase | | | | | | | | | |
| M394 | dJ467L1.3 (period | − | − | − | − | − | − | − | ++ | − |
| | (Drosophila) homolog 3) | | | | | | | | | |
| M395 | cylindromatosis (turban | − | − | − | ++ | − | − | − | − | − |
| | tumor syndrome) | | | | | | | | | |
| M396 | T cell receptor alpha chain V | − | − | − | − | − | − | − | − | ++ |
| | region (clone 2V alpha 23-2) | | | | | | | | | |
| M397 | immunoglobulin lambda | − | − | − | − | − | ++ | − | − | − |
| | light chain variable region | | | | | | | | | |
| M398 | Ig kappa chain V-region (V- | − | − | − | − | − | + | − | − | − |
| | J2-C) | | | | | | | | | |
| M399 | anti-pneumococcal Ig L- | − | − | − | − | − | − | ++ | − | − |
| | chain Fab fragment | | | | | | | | | |
| M400 | IG HEAVY CHAIN V | − | − | − | ++ | − | − | − | − | − |
| | REGION MOO | | | | | | | | | |
| M401 | Unknown (protein for | − | − | − | − | − | + | − | − | − |
| | MGC: 16498) | | | | | | | | | |
| M402 | immunoglobulin lambda | − | − | ++ | − | − | − | − | − | − |
| | light chain VJ region | | | | | | | | | |
| M403 | plasma protease (C1) | − | − | − | − | − | − | − | − | + |
| | inhibitor precursor | | | | | | | | | |
| M404 | similar to INITIATION | − | − | − | − | − | − | − | − | − |
| | FACTOR 5A (EIF-5A) (EIF- | | | | | | | | | |
| | 4D) (REV-BINDING | | | | | | | | | |
| | FACTOR) (H. sapiens) | | | | | | | | | |
| M405 | Ig light chain VL1 region | − | − | ++ | − | − | − | − | − | − |
| M406 | CGI-202 | + | − | − | − | − | − | − | − | − |
| M407 | Ig kappa chain V-III region | − | − | − | − | ++ | − | − | − | − |
| | (She) | | | | | | | | | |
| M408 | Ig light chain variable | − | ++ | − | − | − | − | − | − | − |
| | domain | | | | | | | | | |
| M409 | alternatively spliced | − | − | − | − | − | ++ | − | − | − |
| M410 | neuronal leucine-rich repeat | − | − | − | ++ | − | − | − | − | − |
| | protein-3 | | | | | | | | | |
| M411 | Unknown (protein for | − | − | − | − | − | − | − | − | + |
| | IMAGE: 3587716) | | | | | | | | | |
| M412 | PROTEASOME SUBUNIT | − | ++ | − | − | − | − | − | − | − |
| | ALPHA TYPE 1 | | | | | | | | | |
| | (PROTEASOME | | | | | | | | | |
| | COMPONENT C2) | | | | | | | | | |
| | (MACROPAIN SUBUNIT | | | | | | | | | |
| | C2) (MULTICATALYTIC | | | | | | | | | |
| | ENDOPEPTIDASE | | | | | | | | | |
| | COMPLEX SUBUNIT C2) | | | | | | | | | |
| | (PROTEASOME NU | | | | | | | | | |
| | CHAIN) | | | | | | | | | |
| M413 | NIF-like protein | ++ | − | − | − | − | − | − | − | − |
| M414 | IgM rearranged heavy chain | − | − | − | − | − | ++ | − | − | − |
| | mRNA V-D-J | | | | | | | | | |
| M415 | unnamed protein product | − | ++ | + | − | − | − | − | − | + |
| M416 | immunoglobulin kappa chain | − | − | − | − | ++ | − | − | − | − |
| | variable region | | | | | | | | | |
| M417 | hairy/enhancer of split 6 | + | − | − | − | − | + | − | − | + |
| M418 | immunoglobulin kappa light | − | − | − | − | − | − | − | − | − |
| | chain | | | | | | | | | |
| M419 | sperm tail associated protein | − | − | − | − | − | − | − | − | − |
| M420 | NEURABIN-II (NEURAL | − | ++ | ++ | ++ | − | − | − | − | − |
| | TISSUE-SPECIFIC F- | | | | | | | | | |
| | ACTIN BINDING PROTEIN | | | | | | | | | |
| | II) (SPINOPHILIN) (P130) | | | | | | | | | |
| | (PP1BP134) | | | | | | | | | |
| M421 | TFNR | − | + | − | − | − | − | − | − | − |
| M422 | TROPOMYOSIN 4, | − | ++ | − | − | − | − | − | − | − |
| | EMBRYONIC | | | | | | | | | |
| | FIBROBLAST ISOFORM | | | | | | | | | |
| | (TM-4) | | | | | | | | | |
| M423 | ZINC FINGER Y- | − | − | − | − | − | − | + | − | − |
| | CHROMOSOMAL | | | | | | | | | |
| | PROTEIN | | | | | | | | | |
| M424 | CD27L RECEPTOR | − | − | − | − | − | − | − | − | − |
| | PRECURSOR (T-CELL | | | | | | | | | |
| | ACTIVATION ANTIGEN | | | | | | | | | |
| | CD27) (T14) | | | | | | | | | |
| M425 | FIBROLEUKIN | − | + | − | − | − | − | − | − | − |
| | PRECURSOR | | | | | | | | | |
| | (FIBRINOGEN-LIKE | | | | | | | | | |
| | PROTEIN 2) (PT49) | | | | | | | | | |
| M426 | haptoglobin | − | − | ++ | ++ | − | − | − | − | − |
| M427 | IgA heavy chain variable | − | − | − | − | − | − | + | − | − |
| | region | | | | | | | | | |
| M428 | putative | − | − | − | ++ | ++ | + | − | − | − |
| M429 | unnamed protein product | − | − | − | − | − | ++ | − | − | − |
| M430 | immunoglobulin kappa- | − | − | − | − | − | − | − | − | ++ |
| | chain VK-1 | | | | | | | | | |
| M431 | hypothetical protein | − | − | − | ++ | − | − | − | − | − |
| M432 | immunoglobulin kappa light | − | − | ++ | ++ | − | ++ | − | − | − |
| | chain variable region | | | | | | | | | |
| M433 | myosin-VIIb | − | − | − | − | − | − | − | − | − |
| M434 | immunoglobulin light chain | − | − | − | − | + | − | − | − | − |
| | variable region | | | | | | | | | |
| M435 | hypothetical protein | ++ | ++ | + | − | − | − | ++ | − | − |
| | FLJ20516 | | | | | | | | | |
| M436 | similar to ribosomal protein | − | − | − | − | − | − | + | − | − |
| | S26 (H. sapiens) | | | | | | | | | |
| M437 | immunoglobulin variable | − | − | − | ++ | − | − | − | − | − |
| | region, kappa light chain | | | | | | | | | |
| M438 | immunoglobulin lambda | − | − | − | ++ | − | − | − | − | − |
| | light chain variable region | | | | | | | | | |
| M439 | C-terminus matches | − | − | − | − | − | ++ | − | − | − |
| | KIAA0559, N-terminus | | | | | | | | | |
| | similar to Bassoon protein | | | | | | | | | |
| M440 | immunoglobulin lambda | − | − | ++ | − | − | − | − | − | − |
| | light chain variable region | | | | | | | | | |
| M441 | arfaptin 1 | ++ | − | − | − | − | ++ | + | − | − |
| M442 | apolipoprotein CI | − | − | − | − | − | ++ | − | − | − |
| M443 | dJ591C20.1 (novel protein | − | − | − | − | − | − | − | − | + |
| | similar to mouse NG26) | | | | | | | | | |
| M444 | VESICLE TRANSPORT V- | − | − | − | ++ | − | − | − | − | − |
| | SNARE PROTEIN VTI1- | | | | | | | | | |
| | LIKE 1 (VTI1-RP1) | | | | | | | | | |
| M445 | retinoblastoma-associated | + | − | − | − | − | − | − | − | − |
| | protein HEC | | | | | | | | | |
| M446 | ZINC FINGER PROTEIN | ++ | − | − | − | − | − | − | − | − |
| | GFI-1 (GROWTH FACTOR | | | | | | | | | |
| | INDEPENDENCE-1) | | | | | | | | | |
| M447 | 5- | − | − | − | − | − | − | − | − | − |
| | HYDROXYTRYPTAMINE | | | | | | | | | |
| | 5A RECEPTOR (5-HT-5A) | | | | | | | | | |
| | (SEROTONIN RECEPTOR) | | | | | | | | | |
| | (REC17) | | | | | | | | | |
| M448 | This CDS feature is included | − | − | − | ++ | − | − | − | − | − |
| | to show the translation of the | | | | | | | | | |
| | corresponding V_region. | | | | | | | | | |
| | Presently translation | | | | | | | | | |
| | qualifiers on V_region | | | | | | | | | |
| | features are illegal | | | | | | | | | |
| M449 | immunoglobulin lambda | − | − | ++ | − | − | − | − | − | − |
| | light chain variable region | | | | | | | | | |
| M450 | Ig kappa chain V-II region | − | − | − | ++ | − | − | − | − | − |
| | (Inc) | | | | | | | | | |
| M451 | KIAA1664 protein | ++ | − | − | − | − | − | − | − | − |
| M452 | METALLOTHIONEIN-II | − | − | + | − | − | − | − | − | − |
| | (MT-II) | | | | | | | | | |
| M453 | microseminoprotein beta | − | − | − | − | − | − | − | ++ | − |
| M454 | SACSIN | ++ | − | − | − | − | − | − | − | − |
| M455 | tenascin-R | − | − | − | − | ++ | − | − | − | − |
| M456 | immunoglobulin kappa chain | − | − | − | − | ++ | − | − | − | − |
| | variable region | | | | | | | | | |
| M457 | SERINE PROTEASE | − | − | − | ++ | − | − | − | − | − |
| | INHIBITOR KAZAL-TYPE | | | | | | | | | |
| | 5 PRECURSOR (LYMPHO- | | | | | | | | | |
| | EPITHELIAL KAZAL- | | | | | | | | | |
| | TYPE RELATED | | | | | | | | | |
| | INHIBITOR) (LEKTI) | | | | | | | | | |
| | [CONTAINS: | | | | | | | | | |
| | HEMOFILTRATE PEPTIDE | | | | | | | | | |
| | HF6478 | | | | | | | | | |
| M458 | CALGRANULIN C | − | + | − | − | − | − | − | − | − |
| | (CAGC) | | | | | | | | | |
| M459 | galactose-specific lectin | + | − | − | − | − | − | − | − | − |
| M460 | non-muscle myosin heavy | − | − | − | ++ | − | − | − | − | − |
| | chain | | | | | | | | | |
| M461 | DOCK180 protein | − | − | ++ | − | − | − | − | − | − |
| M462 | serine protease | − | − | − | − | − | − | − | ++ | − |
| M463 | nuclear zinc finger protein | − | − | + | − | − | − | − | − | − |
| | Np95 | | | | | | | | | |
| M464 | LIM protein (similar to rat | − | − | − | ++ | − | − | − | − | − |
| | protein kinase C-binding | | | | | | | | | |
| | enigma) | | | | | | | | | |
| M465 | SECIS binding protein 2 | − | − | ++ | − | − | − | − | − | − |
| M466 | HSPC055 protein | − | − | + | − | − | − | + | − | − |
| M467 | immunoglobulin V lambda/J | − | − | − | ++ | − | − | − | − | − |
| | lambda light chain | | | | | | | | | |
| M468 | ribosomal protein L14 | − | − | − | − | − | − | + | − | − |
| M469 | immunoglobulin kappa chain | − | − | − | − | − | ++ | − | − | − |
| M470 | chloride channel protein 3, | − | − | − | − | − | − | − | − | ++ |
| | long form | | | | | | | | | |
| M471 | MITOGEN-ACTIVATED | − | − | − | ++ | − | − | − | − | − |
| | PROTEIN KINASE | | | | | | | | | |
| | KINASE KINASE 3 | | | | | | | | | |
| | (MAPK/ERK KINASE | | | | | | | | | |
| | KINASE 3) (MEK KINASE | | | | | | | | | |
| | 3) (MEKK 3) | | | | | | | | | |
| M472 | unnamed protein product | − | − | − | − | − | − | − | − | + |
| M473 | anti-HIV gp 120 antibody | − | − | − | − | − | − | − | − | ++ |
| | light chain variable region | | | | | | | | | |
| M474 | CARBONIC ANHYDRASE | − | − | − | − | − | − | − | − | + |
| | IV PRECURSOR | | | | | | | | | |
| | (CARBONATE | | | | | | | | | |
| | DEHYDRATASE IV) (CA- | | | | | | | | | |
| | IV) | | | | | | | | | |
| M475 | hypothetical protein | − | − | + | − | − | − | − | − | − |
| | FLJ11016 | | | | | | | | | |
| M476 | putative gene with | − | − | + | − | − | − | − | − | − |
| | similarities to KIAA1074 and | | | | | | | | | |
| | KIAA0565 | | | | | | | | | |
| M477 | S-MYC PROTO- | + | − | − | − | − | − | − | − | − |
| | ONCOGENE PROTEIN | | | | | | | | | |
| M478 | immunoglobulin kappa light | − | − | − | ++ | − | − | − | − | − |
| | chain variable region | | | | | | | | | |
| M479 | immunoglobulin heavy- | − | − | − | − | − | + | − | − | − |
| | chain subgroup VIII V-D-J | | | | | | | | | |
| | region | | | | | | | | | |
| M480 | unnamed protein product | − | − | − | ++ | − | − | − | − | − |
| M481 | immunoglobulin lambda | − | − | − | − | − | + | − | − | − |
| | light chain | | | | | | | | | |
| M482 | immunoglobulin kappa light | − | − | − | − | − | − | − | − | − |
| | chain variable region | | | | | | | | | |
| M483 | proapolipoprotein Varient | − | − | + | − | + | − | − | − | − |
| M484 | Unknown | − | − | + | + | − | − | − | − | − |
| M485 | neutrophil lactoferrin | − | − | + | − | − | − | − | − | − |
| M486 | alpha-2-HS glycoprotein | − | − | − | − | − | − | − | − | + |
| | Varient | | | | | | | | | |
| M487 | MT-11 protein Varient | − | + | − | − | − | − | − | − | − |
| M488 | immunoglobulin lambda light | − | − | + | − | − | − | − | − | − |
| | chain homolog | | | | | | | | | |
| M489 | Unknown | − | − | − | − | − | − | − | − | + |
| M490 | Unknown | − | − | − | − | − | − | + | − | − |

| | | | | | SEQ | SEQ |
|---|---|---|---|---|---|---|
| | | | | | ID | ID |
| | | N_66 | MW | GI | NO | NO |
| Marker | Gene Name | Br39 | (Da) | number | (nts) | (AA) |
| M1 | HAPTOGLOBIN-2 | +++ | 45205.6 | 4826762 | | |
| | PRECURSOR | | | | | |
| M2 | SEROTRANSFERRIN | +++ | 77050.4 | 4557871 | | |
| | PRECURSOR | | | | | |
| | (SIDEROPHILIN) (BETA-1- | | | | | |
| | METAL BINDING | | | | | |
| | GLOBULIN) | | | | | |
| M3 | SERUM ALBUMIN | +++ | 69367.1 | 4502027 | | |
| | PRECURSOR | | | | | |
| M4 | COMPLEMENT C3 | +++ | 187165 | 4557385 | | |
| | PRECURSOR [CONTAINS: | | | | | |
| | C3A ANAPHYLATOXIN] | | | | | |
| M5 | proapolipoprotein | +++ | 28961.7 | 178775 | | |
| M6 | ALPHA-1-ANTITRYPSIN | +++ | 46736.8 | 1703025 | | |
| | PRECURSOR (ALPHA-1 | | | | | |
| | PROTEASE INHIBITOR) | | | | | |
| | (ALPHA-1- | | | | | |
| | ANTIPROTEINASE) | | | | | |
| M7 | FIBRINOGEN | +++ | 94973.5 | 4503689 | | |
| | ALPHA/ALPHA-E CHAIN | | | | | |
| | PRECURSOR | | | | | |
| M8 | FIBRINOGEN BETA | ++ | 55928.5 | 399492 | | |
| | CHAIN PRECURSOR | | | | | |
| M9 | vitamin D-binding protein | +++ | 52950 | 2119656 | | |
| M10 | TRANSTHYRETIN | +++ | 15887.1 | 4507725 | | |
| | PRECURSOR | | | | | |
| | (PREALBUMIN) (TBPA) | | | | | |
| | (TTR) (ATTR) | | | | | |
| M11 | CERULOPLASMIN | +++ | 122206 | 4557485 | | |
| | PRECURSOR | | | | | |
| | (FERROXIDASE) | | | | | |
| M12 | apolipoprotein A-IV | ++ | 43384.7 | 178779 | | |
| | precursor | | | | | |
| M13 | alpha 1-acid glycoprotein | +++ | 23366.1 | 1197209 | | |
| M14 | hemopexin | ++ | 49295.7 | 1335098 | | |
| M15 | macroglobulin alpha2 | ++ | 160807 | 224053 | | |
| M16 | IgG rearranged gamma chain | +++ | 37720.3 | 184848 | | |
| M17 | alpha-2-glycoprotein 1, zinc | +++ | 38194.4 | 14749011 | | |
| M18 | Ig lambda chain | +++ | 24489.4 | 106653 | | |
| M19 | immunoglobulin alpha-1 | +++ | 37583.8 | 184749 | | |
| | heavy chain constant region | | | | | |
| M20 | HEMOGLOBIN BETA | +++ | 15998.5 | 4504349 | | |
| | CHAIN | | | | | |
| M21 | ANTITHROMBIN-III | ++ | 52602.7 | 4502261 | | |
| | PRECURSOR (ATIII) | | | | | |
| M22 | AMBP PROTEIN | +++ | 38999.7 | 4502067 | | |
| | PRECURSOR [CONTAINS: | | | | | |
| | ALPHA-1- | | | | | |
| | MICROGLOBUUN | | | | | |
| | (PROTEIN HC) | | | | | |
| | (COMPLEX-FORMING | | | | | |
| | GLYCOPROTEIN | | | | | |
| | HETEROGENEOUS IN | | | | | |
| | CHARGE) | | | | | |
| M23 | PROTHROMBIN | ++ | 70037.3 | 4503635 | | |
| | PRECURSOR | | | | | |
| | (COAGULATION FACTOR | | | | | |
| | II) | | | | | |
| M24 | ALPHA-2-HS- | +++ | 39324.9 | 4502005 | | |
| | GLYCOPROTEIN | | | | | |
| | PRECURSOR (FETUIN-A) | | | | | |
| | (ALPHA-2-Z-GLOBULIN) | | | | | |
| | (BA-ALPHA-2- | | | | | |
| | GLYCOPROTEIN) | | | | | |
| M25 | actin beta | ++ | 41737 | 4501885 | | |
| M26 | CLUSTERIN PRECURSOR | ++ | 52494.9 | 4502905 | | |
| | (COMPLEMENT- | | | | | |
| | ASSOCIATED PROTEIN | | | | | |
| | SP-40, 40) (COMPLEMENT | | | | | |
| | CYTOLYSIS INHIBITOR) | | | | | |
| | (CLI) (NA1 AND NA2) | | | | | |
| | (APOLIPOPROTEIN J) | | | | | |
| | (APO-J) (TRPM-2) | | | | | |
| M27 | bA120D12.1 (CD5 antigen- | ++ | 38088.1 | 5174411 | | |
| | like (scavenger receptor | | | | | |
| | cysteine rich family)) | | | | | |
| M28 | fibrinogen gamma-B chain | ++ | 51511.9 | 71828 | | |
| | precursor | | | | | |
| M29 | polyubiquitin 4 | − | 30303.9 | 2118964 | | |
| M30 | KININOGEN PRECURSOR | − | 71945.7 | 125507 | | |
| | (ALPHA-2-THIOL | | | | | |
| | PROTEINASE INHIBITOR) | | | | | |
| | [CONTAINS: | | | | | |
| | BRADYKININ] | | | | | |
| M31 | CALGRANULIN B | ++ | 13242.1 | 4506773 | | |
| | (MIGRATION | | | | | |
| | INHIBITORY FACTOR- | | | | | |
| | RELATED PROTEIN 14) | | | | | |
| | (MRP-14) (P14) | | | | | |
| | (LEUKOCYTE LI | | | | | |
| | COMPLEX HEAVY | | | | | |
| | CHAIN) (S100 CALCIUM- | | | | | |
| | BINDING PROTEIN A9) | | | | | |
| M32 | PLASMA RETINOL- | +++ | 22868 | 5803139 | | |
| | BINDING PROTEIN | | | | | |
| | PRECURSOR (PRBP) (RBP) | | | | | |
| M33 | Ig mu chain precursor, | ++ | 68510.5 | 87919 | | |
| | membrane-bound (clone 201) | | | | | |
| M34 | APOLIPOPROTEIN A-II | +++ | 11175.1 | 4502149 | | |
| | PRECURSOR (APO-AII) | | | | | |
| M35 | LEUCINE-RICH ALPHA-2- | ++ | 34346.6 | 112908 | | |
| | GLYCOPROTEIN (LRG) | | | | | |
| M36 | dJ34F7.4 (complement | ++ | 192753 | 7671645 | | |
| | component 4A) | | | | | |
| M37 | similar to phosphoglycerate | ++ | 28850.2 | 15301114 | | |
| | mutase 1 (brain) | | | | | |
| M38 | alpha-1-antichymotrypsin | ++ | 45482.4 | 177933 | | |
| | precursor | | | | | |
| M39 | ALPHA-1B- | ++ | 51941 | 112892 | | |
| | GLYCOPROTEIN | | | | | |
| M40 | HEMOGLOBIN ALPHA | +++ | 15257.6 | 4504345 | | |
| | CHAIN | | | | | |
| M41 | lipoprotein CIII | ++ | 8764.7 | 224917 | | |
| M42 | TETRANECTIN | +++ | 22567 | 4507557 | | |
| | PRECURSOR (TN) | | | | | |
| | (PLASMINOGEN- | | | | | |
| | KRINGLE 4 BINDING | | | | | |
| | PROTEIN) | | | | | |
| M43 | amyloid related serum | + | 11682.8 | 4506777 | | |
| | protein SAA | | | | | |
| M44 | CALGRANULIN A | + | 10834.6 | 14729628 | | |
| | (MIGRATION | | | | | |
| | INHIBITORY FACTOR- | | | | | |
| | RELATED PROTEIN 8) | | | | | |
| | (MRP-8) (CYSTIC | | | | | |
| | FIBROSIS ANTIGEN) | | | | | |
| | (CFAG) (P8) (LEUKOCYTE | | | | | |
| | LI COMPLEX LIGHT | | | | | |
| | CHAIN) (S100 CALCIUM- | | | | | |
| | BINDING PROTEIN A8) | | | | | |
| M43 | C-REACTIVE PROTEIN | ++ | 25038.7 | 14728083 | | |
| | PRECURSOR | | | | | |
| M46 | RHO GDP-DISSOCIATION | ++ | 22988.1 | 10835002 | | |
| | INHIBITOR 2 (RHO GDI 2) | | | | | |
| | (RHO-GDI BETA) (LY- | | | | | |
| | GDI) | | | | | |
| M47 | PROFILIN I | ++ | 15054.3 | 4826898 | | |
| M48 | IMMUNOGLOBULIN J | +++ | 15594.6 | 400044 | | |
| | CHAIN | | | | | |
| M49 | APOLIPOPROTEIN D | ++ | 21275.7 | 4502163 | | |
| | PRECURSOR | | | | | |
| M50 | inter-alpha-trypsin inhibitor | − | 103373 | 4096840 | | |
| | family heavy chain-related | | | | | |
| | protein | | | | | |
| M51 | 14-3-3 PROTEIN | ++ | 28082.5 | 4507949 | | |
| | BETA/ALPHA (PROTEIN | | | | | |
| | KINASE C INHIBITOR | | | | | |
| | PROTEIN- 1) (KCIP-1) | | | | | |
| | (PROTEIN 1054) | | | | | |
| M52 | Ig G1 H Nie | − | 49207.8 | 229601 | | |
| M53 | OSTEOPONTIN | − | 35422.9 | 14724978 | | |
| | PRECURSOR (BONE | | | | | |
| | SIALOPROTEIN 1) | | | | | |
| | (URINARY STONE | | | | | |
| | PROTEIN) (SECRETED | | | | | |
| | PHOSPHOPROTEIN 1) | | | | | |
| | (SPP-1) (NEPHROPONTIN) | | | | | |
| | (UROPONTIN) | | | | | |
| M54 | AFAMIN PRECURSOR | ++ | 69069.6 | 4501987 | | |
| | (ALPHA-ALBUMIN) | | | | | |
| | (ALPHA-ALB) | | | | | |
| M55 | TRIOSEPHOSPHATE | ++ | 26669.6 | 4507645 | | |
| | ISOMERASE (TIM) | | | | | |
| M56 | pre-serum amyloid P | − | 25397.3 | 337758 | | |
| | component | | | | | |
| M57 | COMP_HUMAN | − | 89149 | 2623750 | | |
| M58 | prosaposin (variant Gaucher | − | 50307.8 | 15298143 | | |
| | disease and variant | | | | | |
| | metachromatic | | | | | |
| | leukodystrophy) | | | | | |
| M59 | VITRONECTIN | ++ | 54305.9 | 14774022 | | |
| | PRECURSOR (SERUM | | | | | |
| | SPREADING FACTOR) (S- | | | | | |
| | PROTEIN) [CONTAINS: | | | | | |
| | SOMATOMEDIN B] | | | | | |
| M60 | CALGRANULIN C (S100 | − | 10443.9 | 2146972 | | |
| | A12 protein) | | | | | |
| M61 | cofilin 1 (non-muscle) | +++ | 16811.7 | 14784011 | | |
| M62 | cathepsin B | − | 37821.8 | 4503139 | | |
| M63 | plasmin (EC 3.4.21.7) | + | 90568.6 | 625234 | | |
| | precursor [validated[ | | | | | |
| M64 | PLASMA GLUTATHIONE | ++ | 25505.6 | 121672 | | |
| | PEROXIDASE | | | | | |
| | PRECURSOR (GSHPX-P) | | | | | |
| M65 | lumican | ++ | 38429.2 | 4505047 | | |
| M66 | apolipoprotein C-II | − | 10183.5 | 2134777 | | |
| M67 | calmodulin 2 (phosphorylase | ++ | 16836.7 | 14250065 | | |
| | kinase, delta) | | | | | |
| M68 | THYMOSIN BETA-4 | ++ | 5062.7 | 14730886 | | |
| M69 | uracil DNA glycosylase | − | 35492.9 | 35053 | | |
| M70 | defensin alpha-3 precursor, | ++ | 10245 | 4885179 | | |
| | neutrophil-specific | | | | | |
| | [validated] | | | | | |
| M71 | CD14 antigen | − | 40076.4 | 4557417 | | |
| M72 | peptidylprolyl isomerase | ++ | 19008.7 | 12804335 | | |
| | (EC 5.2.1.8) A | | | | | |
| M73 | similar to transgelin 2 | − | 24454 | 14728128 | | |
| M74 | BETA-2-GLYCOPROTEIN | − | 38298.4 | 14771355 | | |
| | I PRECURSOR | | | | | |
| | (APOLIPOPROTEIN H) | | | | | |
| | (APO-H) (B2GPI) | | | | | |
| | (BETA(2)GPI) | | | | | |
| | (ACTIVATED PROTEIN C- | | | | | |
| | BINDING PROTEIN) (APC | | | | | |
| | INHIBITOR) | | | | | |
| M75 | complement 9 | − | 60398.5 | 2258128 | | |
| M76 | alpha2 plasmin inhibitor | − | 54596.1 | 11386143 | | |
| M77 | FICOLIN 3 PRECURSOR | − | 32889.1 | 4504331 | | |
| | (COLLAGEN/FIBRINOGEN | | | | | |
| | DOMAIN-CONTAINING | | | | | |
| | PROTEIN 3) | | | | | |
| | (COLLAGEN/FIBRINOGEN | | | | | |
| | DOMAIN-CONTAINING | | | | | |
| | LECTIN 3 P35) (HAKATA | | | | | |
| | ANTIGEN) | | | | | |
| M78 | glutathione transferase | − | 23463.2 | 14766346 | | |
| M79 | Ig kappa chain | ++ | 10963.4 | 106717 | | |
| M80 | TRYPSIN PRECURSOR | ++ | 24409.6 | 136429 | | |
| M81 | complement factor B | − | 85505.3 | 4502397 | | |
| M82 | CALGIZZARIN (S100C | − | 11740.5 | 5032057 | | |
| | PROTEIN) (MLN 70) | | | | | |
| M83 | BETA-2- | ++ | 13714.6 | 4757826 | | |
| | MICROGLOBULIN | | | | | |
| | PRECURSOR | | | | | |
| M84 | CYSTEINE-RICH | − | 27630.5 | 5174675 | | |
| | SECRETORY PROTEIN-3 | | | | | |
| | PRECURSOR (CRISP-3) | | | | | |
| | (SGP28 PROTEIN) | | | | | |
| M85 | rearranged Ig kappa-chain | ++ | 12301 | 973416 | | |
| M86 | megakaryocyte stimulating | ++ | 151092 | 5031925 | | |
| | factor | | | | | |
| M87 | inter-alpha-trypsin inhibitor | ++ | 101388 | 478685 | | |
| | heavy chain HI precursor | | | | | |
| M88 | keratin 9, cytoskeletal | + | 62129.7 | 1082558 | | |
| M89 | FERRITIN HEAVY CHAIN | + | 21225.8 | 14784648 | | |
| | (FERRITIN H SUBUNIT) | | | | | |
| M90 | HISTONE H1.1 | + | 21734.2 | 356168 | | |
| M91 | keratin 1 | + | 66067 | 11935049 | | |
| M92 | ferritin light subunit | − | 16394.7 | 182516 | | |
| M93 | PROTEASOME SUBUNIT | − | 25153.9 | 12804095 | | |
| | ALPHA TYPE 2 | | | | | |
| | (PROTEASOME | | | | | |
| | COMPONENT C3) | | | | | |
| | (MACROPAIN SUBUNIT | | | | | |
| | C3) (MULTICATALYTIC | | | | | |
| | ENDOPEPTIDASE | | | | | |
| | COMPLEX SUBUNIT C3) | | | | | |
| M94 | CARBONIC ANHYDRASE | ++ | 28870.3 | 4502517 | | |
| | I (CARBONATE | | | | | |
| | DEHYDRATASE I) (CA-I) | | | | | |
| M95 | LYMPHOCYTE-SPECIFIC | − | 37191.8 | 10880979 | | |
| | PROTEIN LSP1 (PP52 | | | | | |
| | PROTEIN) (52 KDA | | | | | |
| | PHOSPHOPROTEIN) | | | | | |
| | (LYMPHOCYTE-SPECIFIC | | | | | |
| | ANTIGEN WP34) | | | | | |
| M96 | apolipoprotein F | − | 35399.7 | 4502165 | | |
| M97 | immunoglobulin lambda | − | 13394.7 | 587410 | | |
| | chain variable region | | | | | |
| M98 | OSTEOINDUCTIVE | ++ | 33922.4 | 7661704 | | |
| | FACTOR PRECURSOR | | | | | |
| | (OIF) (OSTEOGLYCIN) | | | | | |
| | (MIMECAN) | | | | | |
| M99 | APOLIPOPROTEIN E | − | 36154.3 | 4557325 | | |
| | PRECURSOR (APO-E) | | | | | |
| M100 | PHOSPHATIDYLETHANO- | + | 21056.9 | 4505621 | | |
| | LAMINE-BINDING | | | | | |
| | PROTEIN (PEBP) | | | | | |
| | (NEUROPOLYPEPTIDE | | | | | |
| | H3) (HIPPOCAMPAL | | | | | |
| | CHOLINERGIC | | | | | |
| | NEUROSTIMULATING | | | | | |
| | PEPTIDE) (HCNP) (RAF | | | | | |
| | KINASE INHIBITOR | | | | | |
| | PROTEIN) (RKIP) | | | | | |
| M101 | SUPEROXIDE | ++ | 15935.8 | 4507149 | | |
| | DISMUTASE [CU-ZN] | | | | | |
| M102 | HISTONE H1A (H1.1) | − | 22178.7 | 121916 | | |
| M103 | neutrophil lipocalin | − | 20547.6 | 4261868 | | |
| M104 | serum | − | 39731.5 | 14752059 | | |
| | paraoxonasearylesterase 1 | | | | | |
| M105 | CYTIDINE DEAMINASE | − | 16184.8 | 11386157 | | |
| | (CYTIDINE | | | | | |
| | AMINOHYDROLASE) | | | | | |
| M106 | CYSTATIN B (LIVER | + | 11139.6 | 4503117 | | |
| | THIOL PROTEINASE | | | | | |
| | INHIBITOR) (CPI-B) | | | | | |
| | (STEFIN B) | | | | | |
| M107 | immunoglobulin kappa light | − | 11799.3 | 6492203 | | |
| | chain variable region | | | | | |
| M108 | angiotensinogen (serine (or | − | 53114.4 | 15079348 | | |
| | cysteine) proteinase inhibitor, | | | | | |
| | clade A (alpha-1 | | | | | |
| | antiproteinase, antitrypsin), | | | | | |
| | member 8) | | | | | |
| M109 | COLLAGEN ALPHA 3(VI) | ++ | 343554 | 4758028 | | |
| | CHAIN PRECURSOR | | | | | |
| M110 | beta galactoside binding | − | 14584.6 | 227920 | | |
| | lectin | | | | | |
| M111 | SH3BGRL3-like protein | − | 10437.8 | 13775198 | | |
| M112 | proteoglycan link protein 2 | − | 40165.8 | 4503053 | | |
| M113 | LYSOZYME C | − | 16537.1 | 4557894 | | |
| | PRECURSOR (1,4-BETA-N- | | | | | |
| | ACETYLMURAMIDASE | | | | | |
| | C) | | | | | |
| M114 | cysteine-rich secreted A12- | − | 11419.5 | 9966777 | | |
| | alpha-like protein 2 | | | | | |
| M115 | LEUKOCYTE ELASTASE | − | 42742 | 13489087 | | |
| | INHIBITOR (LEI) | | | | | |
| | (MONOCYTE/NEUTROPHIL | | | | | |
| | ELASTASE INHIBITOR) | | | | | |
| | (M/NEI) (EI) | | | | | |
| M116 | INSULIN-LIKE GROWTH | + | 27934.2 | 13653947 | | |
| | FACTOR BINDING | | | | | |
| | PROTEIN 4 PRECURSOR | | | | | |
| | (IGFBP-4) (IBP-4) (IGF- | | | | | |
| | BINDING PROTEIN 4) | | | | | |
| M117 | LOW AFFINITY | ++ | 29089.3 | 12056967 | | |
| | IMMUNOGLOBULIN | | | | | |
| | GAMMA FC REGION | | | | | |
| | RECEPTOR III-A | | | | | |
| | PRECURSOR (IGG FC | | | | | |
| | RECEPTOR III-2) (FC- | | | | | |
| | GAMMA RIII-ALPHA) (FC- | | | | | |
| | GAMMA RIIIA) (FCRIIIA) | | | | | |
| | (FC-GAMMA RIII) (FCRIII) | | | | | |
| | (CD16-A) (FCR-10) | | | | | |
| M118 | sex hormone-binding | + | 37488 | 14770624 | | |
| | globulin | | | | | |
| M119 | Annexin I (Lipocortin I) | − | 38714.5 | 4502101 | | |
| | (Calpactin II) (Chromobindin | | | | | |
| | 9) (P35) (Phospholipase A2 | | | | | |
| | Inhibitory Protein) | | | | | |
| M120 | MnSOD precursor | + | 24721.2 | 34711 | | |
| M121 | EPIDIDYMAL | − | 16570.3 | 5453678 | | |
| | SECRETORY PROTEIN El | | | | | |
| | PRECURSOR (NIEMANN- | | | | | |
| | PICK DISEASE TYPE C2 | | | | | |
| | PROTEIN) (EPI-1) (HE1) | | | | | |
| | (EPIDIDYMAL | | | | | |
| | SECRETORY PROTEIN | | | | | |
| | 14.6) (ESP14.6) | | | | | |
| M122 | monoclonal antibody HW1 | ++ | 11556 | 2995691 | | |
| | immunoglobulin light chain | | | | | |
| | variable region | | | | | |
| M123 | METASTATIN | − | 11846.7 | 4506765 | | |
| M124 | inter-alpha (globulin) | − | 106464 | 14742977 | | |
| | inhibitor, H2 polypeptide | | | | | |
| M125 | alpha-1 type 1 collagen | ++ | 55060.9 | 179594 | | |
| M126 | GELSOLIN PRECURSOR, | ++ | 85697.9 | 4504165 | | |
| | PLASMA (ACTIN- | | | | | |
| | DEPOLYMERIZING | | | | | |
| | FACTOR) (ADF) (BREVIN) | | | | | |
| | (AGEL) | | | | | |
| M127 | PROTEASOME SUBUNIT | − | 29484 | 4506185 | | |
| | ALPHA TYPE 4 | | | | | |
| | (PROTEASOME | | | | | |
| | COMPONENT C9) | | | | | |
| | (MACROPAIN SUBUNIT | | | | | |
| | C9) (MULTICATALYTIC | | | | | |
| | ENDOPEPTIDASE | | | | | |
| | COMPLEX SUBUNIT C9) | | | | | |
| | (PROTEASOME SUBUNIT | | | | | |
| | L) | | | | | |
| M128 | SIMIALR TO COACTOSIN- | − | 15945.1 | 1196417 | | |
| | LIKE PROTEIN | | | | | |
| M129 | THIOREDOXIN (ATL- | − | 11737.6 | 14740403 | | |
| | DERIVED FACTOR) (ADF) | | | | | |
| | (SURFACE ASSOCIATED | | | | | |
| | SULPHYDRYL PROTEIN) | | | | | |
| | (SASP) | | | | | |
| M130 | K12 PROTEIN | − | 27039.2 | 4506869 | | |
| | PRECURSOR | | | | | |
| M131 | VITAMIN-K DEPENDENT | − | 52071.6 | 4506115 | | |
| | PROTEIN C PRECURSOR | | | | | |
| | (AUTOPROTHROMBIN | | | | | |
| | IIA) (ANTICOAGULANT | | | | | |
| | PROTEIN C) (BLOOD | | | | | |
| | COAGULATION FACTOR | | | | | |
| | XIV) | | | | | |
| M132 | titin, cardiac muscle | − | 2993535 | 2136280 | | |
| | [validated] | | | | | |
| M133 | MBL-associated protein | − | 20629.3 | 14726286 | | |
| | MAp19 | | | | | |
| M134 | EOSINOPHIL CATIONIC | + | 18440.5 | 4506551 | | |
| | PROTEIN PRECURSOR | | | | | |
| | (ECP) (RIBONUCLEASE 3) | | | | | |
| | (RNASE 3) | | | | | |
| M135 | This CDS feature is included | ++ | 11577.6 | 886295 | | |
| | to show the translation of the | | | | | |
| | corresponding V_region. | | | | | |
| | Presently translation | | | | | |
| | qualifiers on V_region | | | | | |
| | features are illegal | | | | | |
| M136 | enhancer protein | − | 22127.5 | 2135068 | | |
| M137 | cysteine-rich protein 1 | + | 8532.9 | 4503047 | | |
| M138 | anti-Gd cold agglutinin | ++ | 10365.6 | 545723 | | |
| | monoclonal IgMK light chain | | | | | |
| | variable region | | | | | |
| M139 | CYSTATIN A (STEFIN A) | − | 11006.5 | 4885165 | | |
| | (CYSTATIN AS) | | | | | |
| M140 | ANNEXIN II | − | 38604.2 | 4757756 | | |
| | (LIPOCORTIN II) | | | | | |
| | (CALPACTIN I HEAVY | | | | | |
| | CHAIN) (CHROMOBINDIN | | | | | |
| | 8) (P36) (PROTEIN I) | | | | | |
| | (PLACENTAL | | | | | |
| | ANTICOAGULANT | | | | | |
| | PROTEIN IV) (PAP-IV) | | | | | |
| M141 | diazepam binding inhibitor | − | 11793.4 | 10140853 | | |
| M142 | bA139H14.1 (lymphocyte | − | 70288.8 | 8217500 | | |
| | cytosolic protein 1 (L- | | | | | |
| | plastin)) | | | | | |
| M143 | heparan sulfate proteoglycan | ++ | 122673 | 14733263 | | |
| | 2 (perlecan) | | | | | |
| M144 | cathepsin C | − | 51854.1 | 13631727 | | |
| M145 | MYELOBLASTIN | − | 27807.2 | 14765501 | | |
| | PRECURSOR | | | | | |
| | (LEUKOCYTE | | | | | |
| | PROTEINASE 3) (PR-3) | | | | | |
| | (PR3) (AGP7) | | | | | |
| | (WEGENER'S | | | | | |
| | AUTO ANTIGEN) (P29) (C- | | | | | |
| | ANCA ANTIGEN) | | | | | |
| M146 | glutaredoxin | − | 11761.8 | 643695 | | |
| M147 | TRANSLATION ALLY | + | 19595.5 | 4507669 | | |
| | CONTROLLED TUMOR | | | | | |
| | PROTEIN (TCTP) | | | | | |
| M148 | PROTEASOME | − | 30354.5 | 1172602 | | |
| | COMPONENT C13 | | | | | |
| | PRECURSOR | | | | | |
| | (MACROPAIN SUBUNIT | | | | | |
| | C13) (MULTICATALYTIC | | | | | |
| | ENDOPEPTIDASE | | | | | |
| | COMPLEX SUBUNIT C 13) | | | | | |
| M149 | ENDOTHELIAL PROTEIN | + | 26671.6 | 11420547 | | |
| | C RECEPTOR | | | | | |
| | PRECURSOR | | | | | |
| | (ENDOTHELIAL CELL | | | | | |
| | PROTEIN C RECEPTOR) | | | | | |
| | (ACTIVATED PROTEIN C | | | | | |
| | RECEPTOR) (APC | | | | | |
| | RECEPTOR) | | | | | |
| M150 | Ig superfamily protein | − | 43987.3 | 6005958 | | |
| M151 | D-DOPACHROME | − | 12711.8 | 4503291 | | |
| | TAUTOMERASE | | | | | |
| | (PHENYLPYRUVATE | | | | | |
| | TAUTOMERASE II) | | | | | |
| M152 | p80 protein | ++ | 75356.7 | 1483131 | | |
| M153 | COLLAGEN ALPHA 1(V) | ++ | 183619 | 4502957 | | |
| | CHAIN PRECURSOR | | | | | |
| M154 | immunoglobulin light chain | − | 11837.5 | 882304 | | |
| | variable region | | | | | |
| M155 | rho GDP dissociation | ++ | 23193.2 | 36038 | | |
| | inhibitor (GDI) | | | | | |
| M156 | FIBRONECTIN | + | 262608 | 2506872 | | |
| | PRECURSOR (FN) (COLD- | | | | | |
| | INSOLUBLE GLOBULIN) | | | | | |
| | (CIG) | | | | | |
| M157 | FK506-BIND[NG | − | 11950.8 | 4503725 | | |
| | PROTEIN (FKBP-12) | | | | | |
| | (PEPTIDYL-PROLYL CIS- | | | | | |
| | TRANS ISOMERASE) | | | | | |
| | (PPIASE) (ROTAMASE) | | | | | |
| | (IMMUNOPHILIN FKBP12) | | | | | |
| M158 | IG KAPPA CHAIN V | − | 12199.7 | 10636525 | | |
| | REGION GOM | | | | | |
| M159 | similar to osteoclast | − | 23786.9 | 14738380 | | |
| | stimulating factor 1 (H. | | | | | |
| | sapiens) | | | | | |
| M160 | Ig heavy chain V-III region | − | 10853.2 | 87860 | | |
| | (TD-Vr) | | | | | |
| M161 | PEROXIREDOXIN 2 | ++ | 21892 | 13631440 | | |
| | (THIOREDOXIN | | | | | |
| | PEROXIDASE 1) | | | | | |
| | (THIOREDOXIN- | | | | | |
| | DEPENDENT PEROXIDE | | | | | |
| | REDUCTASE 1) (THIOL- | | | | | |
| | SPECIFIC ANTIOXIDANT | | | | | |
| | PROTEIN) (TSA) (PRP) | | | | | |
| | (NATURAL KILLER CELL | | | | | |
| | ENHANCING FACTOR B) | | | | | |
| | (NKEF-B) | | | | | |
| M162 | myosin catalytic light chain | − | 16931.1 | 10440556 | | |
| | LC17b | | | | | |
| M163 | protein SPY75 | − | 53998.3 | 4885405 | | |
| M164 | 2-phosphopyruvate- | − | 47109.1 | 693933 | | |
| | hydratase alpha-enolase | | | | | |
| M165 | IG LAMBDA CHAIN V-V | ++ | 11342.6 | 126571 | | |
| | REGION DEL | | | | | |
| M166 | pancreatic secretory trypsin | + | 6247.1 | 671743 | | |
| | inhibitor | | | | | |
| M167 | GLIA MATURATION | − | 16801.4 | 4758440 | | |
| | FACTOR GAMMA (GMF- | | | | | |
| | GAMMA) | | | | | |
| M168 | CALCYCLIN (LUNG 10 | − | 10153.8 | 1173337 | | |
| | KDA PROTEIN) | | | | | |
| M169 | SH3 DOMAIN-BINDING | − | 12774.3 | 4506925 | | |
| | GLUTAMIC ACID-RICH- | | | | | |
| | LIKE PROTEIN | | | | | |
| M170 | CHYMOTRYPSINOGEN A | − | 25666.3 | 117615 | | |
| M171 | DJ-1 protein | − | 19847.1 | 6005749 | | |
| M172 | phosphoprotein enriched in | − | 15040.2 | 4505705 | | |
| | astrocytes 15 | | | | | |
| M173 | EGF-CONTAINING | + | 54641 | 9665262 | | |
| | FIBULIN-LIKE | | | | | |
| | EXTRACELLULAR | | | | | |
| | MATRIX PROTEIN I | | | | | |
| | PRECURSOR (FIBULIN-3) | | | | | |
| | (FIBL-3) (T16 PROTEIN) | | | | | |
| M174 | superoxide dismutase 3, | − | 25851.1 | 14733169 | | |
| | extracellular | | | | | |
| M175 | CLARA CELL | − | 9993.8 | 4507809 | | |
| | PHOSPHOLIPID-BINDING | | | | | |
| | PROTEIN PRECURSOR | | | | | |
| | (CCPBP) (CLARA CELLS | | | | | |
| | 10 KDA SECRETORY | | | | | |
| | PROTEIN) (CC10) | | | | | |
| | (UTEROGLOBIN) (URINE | | | | | |
| | PROTEIN 1) (UP1) | | | | | |
| M176 | Similar to LIM and SH3 | − | 29658.3 | 15214662 | | |
| | protein 1 | | | | | |
| M177 | PROSTAGLANDIN-H2 D- | ++ | 21028.9 | 4506251 | | |
| | ISOMERASE PRECURSOR | | | | | |
| | (PROSTAGLANDIN-D | | | | | |
| | SYNTHASE) | | | | | |
| | (GLUTATHIONE- | | | | | |
| | INDEPENDENT PGD | | | | | |
| | SYNTHETASE) | | | | | |
| | (PROSTAGLANDIN D2 | | | | | |
| | SYNTHASE) (PGD2 | | | | | |
| | SYNTHASE) (PGDS2) | | | | | |
| | (PGDS) (BETA-TRACE | | | | | |
| | PROTEIN) | | | | | |
| M178 | mannose 6- | − | 273400 | 6981078 | | |
| | phosphate/insulin-like growth | | | | | |
| | factor II receptor | | | | | |
| M179 | OXYGEN-REGULATED | − | 240663 | 5454016 | | |
| | PROTEIN 1 (RETINITIS | | | | | |
| | PIGMENTOSA RP1 | | | | | |
| | PROTEIN) (RETINITIS | | | | | |
| | PIGMENTOSA 1 PROTEIN) | | | | | |
| M180 | SERUM AMYLOID A | − | 12289.5 | 7531274 | | |
| | PROTEIN (SAA) | | | | | |
| | [CONTAINS: AMYLOID | | | | | |
| | PROTEIN A (AMYLOID | | | | | |
| | FIBRIL PROTEIN AA)[ | | | | | |
| M181 | CARGO SELECTION | − | 47033.1 | 5032183 | | |
| | PROTEIN TIP47 (47 KDA | | | | | |
| | MANNOSE 6-PHOSPHATE | | | | | |
| | RECEPTOR-BINDING | | | | | |
| | PROTEIN) (47 KDA MPR- | | | | | |
| | BINDING PROTEIN) | | | | | |
| | (PLACENTAL PROTEIN | | | | | |
| | 17) | | | | | |
| M182 | adenylyl cyclase-associated | − | 51749.4 | 15296533 | | |
| | protein | | | | | |
| M183 | aldolase C | − | 39456.1 | 4885063 | | |
| M184 | similar to PROTEASOME | − | 27330.7 | 14719929 | | |
| | SUBUNIT ALPHA TYPE 6 | | | | | |
| | (PROTEASOME IOTA | | | | | |
| | CHAIN) (MACROPAIN | | | | | |
| | IOTA CHAIN) | | | | | |
| | (MULTICATALYTIC | | | | | |
| | ENDOPEPTIDASE | | | | | |
| | COMPLEX IOTA CHAIN) | | | | | |
| | (27 KDA PROSOMAL | | | | | |
| | PROTEIN) (PROS-27) | | | | | |
| | (P27K) (H. sapiens) | | | | | |
| M185 | immunoglobulin light chain | ++ | 12160.7 | 5419711 | | |
| | variable region | | | | | |
| M186 | FOLLISTATIN-RELATED | − | 34985.7 | 5901956 | | |
| | PROTEIN 1 PRECURSOR | | | | | |
| M187 | GAMMA-INTERFERON | − | 29149.1 | 12643406 | | |
| | INDUCIBLE LYSOSOMAL | | | | | |
| | THIOL REDUCTASE | | | | | |
| | PRECURSOR (GAMMA- | | | | | |
| | INTERFERON-INDUCIBLE | | | | | |
| | PROTEIN IP-30) | | | | | |
| M188 | F-ACTIN CAPPING | − | 31350.7 | 13124696 | | |
| | PROTEIN BETA SUBUNIT | | | | | |
| | (CAPZ BETA) | | | | | |
| M189 | coagulation factor X | − | 52535.2 | 180336 | | |
| M190 | histone H3 | − | 15328 | 4504279 | | |
| M191 | adiponectin | ++ | 26413.8 | 4757760 | | |
| M192 | 16G2 | − | 42055.2 | 14738192 | | |
| M193 | Ig kappa chain precursor V | − | 12430.1 | 87866 | | |
| | region (A10) | | | | | |
| M194 | immunoglobulin lambda | − | 9483.3 | 10945949 | | |
| | light chain variable region | | | | | |
| M195 | S-100P PROTEIN | − | 10400 | 5174663 | | |
| M196 | INTERCELLULAR | + | 30653.5 | 4504557 | | |
| | ADHESION MOLECULE-2 | | | | | |
| | PRECURSOR (ICAM-2) | | | | | |
| | (CD102) | | | | | |
| M197 | MANNOSYL- | − | 70821.3 | 5174521 | | |
| | OLIGOSACCHARIDE | | | | | |
| | ALPHA-1,2- | | | | | |
| | MANNOSIDASE (MAN(9)- | | | | | |
| | ALPHA-MANNOSIDASE) | | | | | |
| M198 | NAD+-isocitrate | ++ | 39592 | 5031777 | | |
| | dehydrogenase, alpha subunit | | | | | |
| M199 | CONNECTIVE TISSUE | − | 38069.8 | 4503123 | | |
| | GROWTH FACTOR | | | | | |
| | PRECURSOR | | | | | |
| | (HYPERTROPHIC | | | | | |
| | CHONDROCYTE- | | | | | |
| | SPECIFIC PROTEIN 24) | | | | | |
| M200 | complement factor H-related | − | 27868.8 | 2134940 | | |
| | protein FHR-2 | | | | | |
| M201 | RIBONUCLEASE, | − | 16377.3 | 133237 | | |
| | SEMINAL PRECURSOR | | | | | |
| | (SEMINAL RNASE) (S- | | | | | |
| | RNASE) (RIBONUCLEASE | | | | | |
| | BS-1) | | | | | |
| M202 | immunoglobulin kappa chain | − | 10661.9 | 722612 | | |
| M203 | putative | − | 12859.9 | 12832737 | | |
| M204 | SERUM AMYLOID A-4 | − | 14806.8 | 10835095 | | |
| | PROTEIN PRECURSOR | | | | | |
| | (CONSTITUTIVELY | | | | | |
| | EXPRESSED SERUM | | | | | |
| | AMYLOID A PROTEIN) | | | | | |
| | (C-SAA) | | | | | |
| M205 | HP1-BP74 | + | 61207.3 | 11424882 | | |
| M206 | myristoylated alanine-rich | − | 3276.6 | 187385 | | |
| | C-kinase substrate | | | | | |
| M207 | CORTICOSTEROID- | + | 45141.1 | 4502595 | | |
| | BINDING GLOBULIN | | | | | |
| | PRECURSOR (CBG) | | | | | |
| | (TRANSCORTIN) | | | | | |
| M208 | mannose-binding lectin | − | 26090.6 | 5911809 | | |
| M209 | APOLIPOPROTEIN L | − | 42383.5 | 14916953 | | |
| | PRECURSOR (APO-L) | | | | | |
| M210 | GLUTATHIONE | − | 27566 | 4758484 | | |
| | TRANSFERASE OMEGA 1 | | | | | |
| | (GSTO 1-1) | | | | | |
| M211 | Somatomedin A | − | 7578.8 | 224061 | | |
| M212 | ganglioside M2 activator | − | 17623.5 | 106058 | | |
| | protein | | | | | |
| M213 | Ig heavy chain V region | ++ | 13876.6 | 110106 | | |
| | (174.2E10) | | | | | |
| M214 | FATTY ACID-BINDING | − | 15164.5 | 4557581 | | |
| | PROTEIN, EPIDERMAL (E- | | | | | |
| | FABP) (PSORIASIS- | | | | | |
| | ASSOCIATED FATTY | | | | | |
| | ACID-BINDING PROTEIN | | | | | |
| | HOMOLOG) (PA-FABP) | | | | | |
| M215 | MYELOID CELL | − | 45836.3 | 4505227 | | |
| | NUCLEAR | | | | | |
| | DIFFERENTIATION | | | | | |
| | ANTIGEN | | | | | |
| M216 | protein-L-isoaspartate(D- | − | 24679.5 | 14781911 | | |
| | aspartate) O- | | | | | |
| | methyltransferase (EC | | | | | |
| | 2.1.1.77) splice form II | | | | | |
| M217 | pancreatic ribonuclease (EC | − | 17239.9 | 2135882 | | |
| | 3.1.27.5) precursor | | | | | |
| M218 | IG KAPPA CHAIN V-I | − | 11870.2 | 125776 | | |
| | REGION MEV | | | | | |
| M219 | anti-MSPI MAD20 block2 | − | 11668.2 | 12836991 | | |
| | ScFv Ig light chain variable | | | | | |
| | region | | | | | |
| M220 | vimentin | + | 53714 | 340219 | | |
| M221 | HLA CLASS II | − | 33460.7 | 399888 | | |
| | HISTOCOMPATIBILITY | | | | | |
| | ANTIGEN, GAMMA | | | | | |
| | CHAIN (HLA-DR | | | | | |
| | ANTIGENS ASSOCIATED | | | | | |
| | INVARIANT CHAIN) (P33) | | | | | |
| | (CD74 ANTIGEN) | | | | | |
| M222 | IMP dehydrogenase | + | 55920.4 | 4504689 | | |
| M223 | granulin | − | 61441.5 | 14772463 | | |
| M224 | p97 | − | 80170.1 | 3786308 | | |
| M225 | antigen HLA SB beta, MHC | − | 19069.9 | 224042 | | |
| | II | | | | | |
| M226 | immunoglobulin rearranged | ++ | 11825.1 | 2072274 | | |
| | light chain | | | | | |
| M227 | kinesin like protein 9 | + | 89942.6 | 6754442 | | |
| M228 | PROTEASOME | − | 28936.5 | 4506191 | | |
| | COMPONENT MECL-1 | | | | | |
| | PRECURSOR | | | | | |
| | (MACROPAIN SUBUNIT | | | | | |
| | MECL-1) | | | | | |
| | (MULTICATALYTIC | | | | | |
| | ENDOPEPTIDASE | | | | | |
| | COMPLEX SUBUNIT | | | | | |
| | MECL-1) | | | | | |
| M229 | immunoglobulin kappa light | + | 11005.3 | 13448025 | | |
| | chain variable region | | | | | |
| M230 | immunogloblin light chain | − | 11719.1 | 1905799 | | |
| M231 | IG Lambda Chain V-1V | − | 11272.3 | 126570 | | |
| | Region Mol | | | | | |
| M232 | immunoglobulin variable | ++ | 12618.2 | 791177 | | |
| | region used by the ITC48 | | | | | |
| | kappa light chain (subgroup | | | | | |
| | V kappa FV) (anti- | | | | | |
| | cytomeglovirus glycoprotein | | | | | |
| | B(A | | | | | |
| M233 | hypothetical protein | ++ | 17934.8 | 14773888 | | |
| | DKFZp761B15121.1 | | | | | |
| M234 | immunoglobulin kappa light | − | 12315.9 | 5731229 | | |
| | chain variable region B3 | | | | | |
| M235 | VON EBNER'S GLAND | − | 19250.1 | 4504963 | | |
| | PROTEIN PRECURSOR | | | | | |
| | (VEG PROTEIN) (TEAR | | | | | |
| | PREALBUMIN) (TP) | | | | | |
| | (TEAR LIPOCALIN) | | | | | |
| | (LIPOCALIN-1) | | | | | |
| M236 | Ribonuclease Pancreatic | − | 16461 | 133198 | | |
| | Precursor (RNASE 1) | | | | | |
| | (RNASE A) | | | | | |
| M237 | immunoglobulin kappa chain | − | 11159.5 | 12655532 | | |
| | variable region | | | | | |
| M238 | immunoglobulin kappa light | − | 11696.1 | 4324014 | | |
| | chain variable region | | | | | |
| M239 | immunoglobulin kappa chain | − | 11941.5 | 416338 | | |
| | V region | | | | | |
| M240 | Carbonic Anhydrase II | − | 29246.2 | 4557395 | | |
| | (Carbonate Dehydratase II) | | | | | |
| | (CA-II) | | | | | |
| M241 | HRV Fab 027-VL | − | 12383 | 2385498 | | |
| M242 | immunoglobulin variable | − | 12748.6 | 2597940 | | |
| | region, kappa light chain | | | | | |
| M243 | IG Kappa Chain V-II Region | − | 12060.5 | 125786 | | |
| | MIL | | | | | |
| M244 | 364K Golgi complex- | − | 364299 | 7441640 | | |
| | associated protein | | | | | |
| M245 | histone H2A.5 | − | 14059.5 | 70686 | | |
| M246 | antibody, light chain variable | − | 12356.8 | 732744 | | |
| | regin to HIV1 p25 | | | | | |
| M247 | immunoglobulin heavy- | − | 12854.4 | 348180 | | |
| | chain subgroup VIII V-D-J | | | | | |
| | region | | | | | |
| M248 | TRYPSINOGEN, | − | 25424.9 | 2507249 | | |
| | CATIONIC PRECURSOR | | | | | |
| | (BETA-TRYPSIN) | | | | | |
| M249 | putative | − | 26422.2 | 12963645 | | |
| M250 | LOW AFFINITY | − | 34989.9 | 399476 | | |
| | IMMUNOGLOBULIN | | | | | |
| | GAMMA FC REGION | | | | | |
| | RECEPTOR II-A | | | | | |
| | PRECURSOR (FC-GAMMA | | | | | |
| | RII-A) (FCRII-A) (IGG FC | | | | | |
| | RECEPTOR II-A) (FC- | | | | | |
| | GAMMA-RIIA) (CD32) | | | | | |
| | (CDW32) | | | | | |
| M251 | immunoglobulin lambda | − | 11519.6 | 9968388 | | |
| | chain variable region | | | | | |
| M252 | CYTOCHROME C | − | 11888 | 14782885 | | |
| M253 | Ig kappa chain V-1V region | − | 11476.8 | 106620 | | |
| | (Dep) | | | | | |
| M254 | immunoglobulin kappa | − | 10013.1 | 1335366 | | |
| | orphon (AA 95) | | | | | |
| M255 | immunoglobulin kappa chain | − | 11192.6 | 12655663 | | |
| | variable region | | | | | |
| M256 | immunoglobulin kappa light | − | 10627.9 | 14625921 | | |
| | chain variable region | | | | | |
| M257 | immunoglobulin lambda | − | 12685.3 | 3142565 | | |
| | light chain variable region | | | | | |
| M258 | gamma-glutamyl hydrolase | − | 26023.5 | 13646249 | | |
| | (conjugase, | | | | | |
| | folylpolygammaglutamyl | | | | | |
| | hydrolase) precursor | | | | | |
| M259 | breakpoint cluster region | − | 15521.8 | 3002951 | | |
| | protein 1 | | | | | |
| M260 | immunoglobulin kappa chain | − | 10457.6 | 12655486 | | |
| | variable region | | | | | |
| M261 | BCL3 | + | 46432.3 | 3928845 | | |
| M262 | immunoglobulin lambda | + | 11482.7 | 6643255 | | |
| | light chain variable region | | | | | |
| M263 | immunoglobulin kappa chain | ++ | 12131.5 | 5019539 | | |
| | variable region | | | | | |
| M264 | macrophage migration | − | 11428.1 | 187181 | | |
| | inhibitory factor | | | | | |
| M265 | immunoglobulin kappa chain | − | 12277.8 | 5578792 | | |
| | variable region | | | | | |
| M266 | unnamed protein product | − | 47994.5 | 10436374 | | |
| M267 | ribosomal protein S28, | − | 2336.6 | 7440562 | | |
| | cytosolic | | | | | |
| M268 | MICROFIBRIL- | + | 19611.7 | 4505089 | | |
| | ASSOCIATED | | | | | |
| | GLYCOPROTEIN 2 | | | | | |
| | PRECURSOR (MAGP-2) | | | | | |
| | (MP25) | | | | | |
| M269 | cathepsin S | − | 37495.9 | 11427057 | | |
| M270 | lambda-chain C-region C- | ++ | 11515.3 | 165427 | | |
| | lambda-2 | | | | | |
| M271 | anti-porcine VCAM mAb | − | 12178.8 | 4098515 | | |
| | 3F4 light chain variable | | | | | |
| | region | | | | | |
| M272 | immunoglobulin lambda-3 | − | 10959.2 | 13016692 | | |
| | variable region | | | | | |
| M273 | immunoglobulin kappa chain | ++ | 15757.9 | 598166 | | |
| | variable region | | | | | |
| M274 | IgM light chain variable | − | 11707.3 | 1673593 | | |
| | region | | | | | |
| M275 | CATHEPSIN D | − | 44552.5 | 4503143 | | |
| M276 | immunoglobulin kappa light | ++ | 11451.8 | 9246439 | | |
| | chain variable region | | | | | |
| M277 | putative | − | 19274.4 | 12843372 | | |
| M278 | transaldolase | − | 37540.3 | 5803187 | | |
| M279 | immunoglobulin V lambda/J | ++ | 10793.9 | 6643727 | | |
| | lambda light chain | | | | | |
| M280 | myeloid inhibitory siglec | ++ | 51760.6 | 13936734 | | |
| M281 | granulocyte inhibitory | − | 2046.3 | 106167 | | |
| | protein | | | | | |
| M282 | GROWTH FACTOR | − | 25206.5 | 4504111 | | |
| | RECEPTOR-BOUND | | | | | |
| | PROTEIN 2 (GRB2 | | | | | |
| | ADAPTER PROTEIN) | | | | | |
| | (SH2/SH3 ADAPTER | | | | | |
| | GRB2) (ASH PROTEIN) | | | | | |
| M283 | immunoglobulin kappa light | ++ | 10835 | 4323812 | | |
| | chain variable region | | | | | |
| M284 | UBIQUITIN-LIKE | − | 10871.3 | 5902098 | | |
| | PROTEIN SMT3B | | | | | |
| | (SENTRIN 2) | | | | | |
| M285 | immunoglobulin kappa chain | − | 11003.4 | 722434 | | |
| M286 | HLA CLASS I | − | 40950.1 | 231427 | | |
| | HISTOCOMPATIBILITY | | | | | |
| | ANTIGEN, CW-1 CW*0102 | | | | | |
| | ALPHA CHAIN | | | | | |
| | PRECURSOR (CW1.2) | | | | | |
| M287 | 40S RIBOSOMAL | − | 15550.2 | 4506693 | | |
| | PROTEINS 17 | | | | | |
| M288 | immunoglobulin kappa light | − | 12460 | 4378294 | | |
| | chain variable region | | | | | |
| M289 | dJ747L4.1 (Brachyury (T | − | 47004.1 | 3900891 | | |
| | box protein)-LIKE protein | | | | | |
| M290 | anti-oxidized LDL | − | 11825.3 | 15277620 | | |
| | autoantibody variable kappa | | | | | |
| | chain | | | | | |
| M291 | immunoglobulin light chain | − | 10721.2 | 5532843 | | |
| | variable region | | | | | |
| M292 | immunoglobulin kappa light | − | 11444.7 | 9246545 | | |
| | chain variable region | | | | | |
| M293 | Ig kappa chain | − | 12040.5 | 346170 | | |
| M294 | immunoglobulin kappa chain | − | 12174.7 | 5578780 | | |
| | variable region | | | | | |
| M295 | Ig kappa chain V-III region | − | 4685.3 | 106605 | | |
| | (Lew) | | | | | |
| M296 | similar to TROPOMYOSIN, | − | 16974.3 | 14786818 | | |
| | CYTOSKELETAL TYPE | | | | | |
| | (TM30-NM) (H. sapiens) | | | | | |
| M297 | immunoglobulin kappa light | ++ | 9388.3 | 619686 | | |
| | chain, variable region | | | | | |
| M298 | ZYXIN (ZYXIN 2) | − | 61277.7 | 4508047 | | |
| M299 | immunoglobulin lambda | − | 11057.3 | 5019492 | | |
| | chain variable region | | | | | |
| M300 | ENHANCER OF | − | 12259 | 4758302 | | |
| | RUDIMENTARY | | | | | |
| | HOMOLOG | | | | | |
| M301 | regulator of G-protein | − | 23255.8 | 5032039 | | |
| | signaling 4 | | | | | |
| M302 | immunoglobulin lambda | − | 11543.7 | 6643601 | | |
| | light chain variable region | | | | | |
| M303 | immunoglobulin kappa chain | − | 9539.7 | 11137019 | | |
| M304 | immunoglobulin kappa light | − | 11640.1 | 4378186 | | |
| | chain variable region | | | | | |
| M305 | anti-DNA immunoglobulin | + | 11047.2 | 1870506 | | |
| | light chain IgG | | | | | |
| M306 | Ig kappa chain (Vk) V | − | 9157.2 | 861002 | | |
| | region (VJ) | | | | | |
| M307 | UBIQUITIN-LIKE | − | 9071.6 | 5453760 | | |
| | PROTEIN NEDD8 | | | | | |
| M308 | immunoglobulin light chain | − | 11544 | 13171344 | | |
| | variable region | | | | | |
| M309 | immunoglobulin lambda | − | 11326.5 | 4324210 | | |
| | light chain variable region | | | | | |
| M310 | immunoglobulin lambda | − | 9521.5 | 4566035 | | |
| | light chain | | | | | |
| M311 | SCRAPE-RESPONSIVE | − | 11081.2 | 6005870 | | |
| | PROTEIN 1 PRECURSOR | | | | | |
| | (SCRG-1) | | | | | |
| M312 | gonadotropin releasing | − | 1651.6 | 225500 | | |
| | peptide | | | | | |
| M313 | immunoglobulin light chain | − | 11709.1 | 5419695 | | |
| | variable region | | | | | |
| M314 | IG HEAVY CHAIN V-III | − | 13566.4 | 123850 | | |
| | REGION HIL | | | | | |
| M315 | IRT-1 | − | 14617.3 | 4758612 | | |
| M316 | recombinant antibody light | − | 11556.9 | 12957386 | | |
| | chain VL domain | | | | | |
| M317 | putative | − | 56695 | 12836433 | | |
| M318 | CYSTATIN M | − | 16511.2 | 4503113 | | |
| | PRECURSOR (CYSTATIN | | | | | |
| | E) | | | | | |
| M319 | immunoglobulin rearranged | − | 11815.1 | 2218124 | | |
| | light chain | | | | | |
| M320 | proteasome activator PA28 | − | 27348.7 | 4506237 | | |
| | beta chain | | | | | |
| M321 | AGGRECAN CORE | − | 250194 | 129886 | | |
| | PROTEIN PRECURSOR | | | | | |
| | (CARTILAGE-SPECIFIC | | | | | |
| | PROTEOGLYCAN CORE | | | | | |
| | PROTEIN) (CSPCP) | | | | | |
| | (CHONDROITIN SULFATE | | | | | |
| | PROTEOGLYCAN CORE | | | | | |
| | PROTEIN 1) | | | | | |
| M322 | KIAA0185 protein | − | 57547.8 | 15298593 | | |
| M323 | hypothetical protein | − | 97949.7 | 14745846 | | |
| | FLJ13465 | | | | | |
| M324 | CARBOXYPEPTIDASE N | − | 58649.4 | 115877 | | |
| | 83 KDA CHAIN | | | | | |
| | (CARBOXYPEPTIDASE N | | | | | |
| | REGULATORY SUBUNIT) | | | | | |
| M325 | Ig kappa chain | + | 11664 | 631236 | | |
| M326 | R33729_1 | − | 11332.9 | 3355455 | | |
| M327 | lymphocyte surface antigen | − | 32074.7 | 106924 | | |
| | precursor CD44 | | | | | |
| M328 | PYRIDOXINE KINASE | − | 35102.5 | 4505701 | | |
| | (PYRIDOXAL KINASE) | | | | | |
| M329 | unnamed protein product | − | 42442.7 | 10436670 | | |
| M330 | CALPACTIN I LIGHT | − | 11203.2 | 4506761 | | |
| | CHAIN (P10 PROTEIN) | | | | | |
| | (PI 1) (CELLULAR | | | | | |
| | LIGAND OF ANNEXIN II) | | | | | |
| | (NERVE GROWTH | | | | | |
| | FACTOR INDUCED | | | | | |
| | PROTEIN 42C) | | | | | |
| M331 | c360B4.1 (PUTATIVE | − | 23267.9 | 5912545 | | |
| | novel protein similar to | | | | | |
| | predicted bacterial and worm | | | | | |
| | proteins) | | | | | |
| M332 | latent transforming growth | − | 161160 | 3327808 | | |
| | factor-beta binding protein | | | | | |
| | 4S | | | | | |
| M323 | hypothetical protein | + | 49735 | 14744245 | | |
| | XP_047083 | | | | | |
| M334 | similar to | + | 45421.1 | 13633718 | | |
| | GASTRIN/CHOLECYSTOKININ | | | | | |
| | TYPE B RECEPTOR | | | | | |
| | (CCK-B RECEPTOR) | | | | | |
| | (CCK-BR) (H. sapiens) | | | | | |
| M335 | immunoglobulin kappa chain | − | 10304.4 | 12655482 | | |
| | variable region | | | | | |
| M336 | similar to LINE-1 | + | 16237.5 | 14766164 | | |
| | REVERSE | | | | | |
| | TRANSCRIPTASE | | | | | |
| | HOMOLOG (H. sapiens) | | | | | |
| M337 | TYROSINE-PROTEIN | − | 50704.6 | 4758078 | | |
| | KINASE CSK (C-SRC | | | | | |
| | KINASE) | | | | | |
| M338 | Similar to dystroglycan 1 | − | 97541.1 | 15215308 | | |
| | (dystrophin-associated | | | | | |
| | glycoprotein 1) | | | | | |
| M339 | complement component 1, s | − | 37337.1 | 14766592 | | |
| | subcomponent | | | | | |
| M340 | lysophospholipase 11 | + | 24737.1 | 9966764 | | |
| M341 | Ig kappa chain V region (V- | + | 10009.2 | 480919 | | |
| | kappa 3) | | | | | |
| M342 | immunoglobulin V lambda/J | − | 10942.9 | 6643633 | | |
| | lambda light chain | | | | | |
| M343 | immunoglobulin kappa light | ++ | 11826.3 | 4378192 | | |
| | chain variable region | | | | | |
| M344 | TELOMERASE-BINDING | − | 18721.5 | 9790017 | | |
| | PROTEIN P23 (HSP90 CO- | | | | | |
| | CHAPERONE) | | | | | |
| | (PROGESTERONE | | | | | |
| | RECEPTOR COMPLEX | | | | | |
| | P23) | | | | | |
| M345 | immunoglobulin lambda | − | 11275.5 | 4324094 | | |
| | light chain variable region | | | | | |
| M346 | inwardly rectifying | − | 43580.1 | 2143813 | | |
| | potassium channel protein | | | | | |
| | Kir6.2 | | | | | |
| M347 | Ig kappa chain | ++ | 26153.4 | 1220492 | | |
| M348 | immunoglobulin light chain | − | 13979.9 | 1813654 | | |
| M349 | dnaJ protein homolog | − | 30611.7 | 478645 | | |
| M350 | 40S RIBOSOMAL | − | 6676.8 | 4506717 | | |
| | PROTEIN S29 | | | | | |
| M351 | IgA1 kappa light chain | − | 13708.5 | 6110570 | | |
| M352 | MYOGLOBIN | + | 17042.7 | 127656 | | |
| M353 | cathepsin X precursor | − | 33943.2 | 3650498 | | |
| M354 | unknown | − | 17297.2 | 3860020 | | |
| M355 | immunoglobulin lambda | − | 10925 | 12655626 | | |
| | chain variable region | | | | | |
| M356 | HYPOTHETICAL | − | 24353.8 | 12585535 | | |
| | PROTEIN CGI- 109 | | | | | |
| | PRECURSOR | | | | | |
| M357 | LITHOSTATHINE 1 BETA | + | 18664.9 | 10835248 | | |
| | PRECURSOR | | | | | |
| | (REGENERATING | | | | | |
| | PROTEIN I BETA) | | | | | |
| M358 | human leucocyte antigen B | + | 31344.8 | 9027550 | | |
| M359 | immunoglobulin light chain | − | 8948.8 | 5457339 | | |
| | variable region | | | | | |
| M360 | This CDS feature is included | − | 11607 | 681900 | | |
| | to show the translation of the | | | | | |
| | corresponding V_region. | | | | | |
| | Presently translation | | | | | |
| | qualifiers on V_region | | | | | |
| | features are illegal. | | | | | |
| M361 | Ig kappa chain V-I region | − | 15481.9 | 477501 | | |
| | (ISE) | | | | | |
| M362 | KIAA0336 | − | 184659 | 7662062 | | |
| M363 | Similar to expressed | ++ | 52604.1 | 15489206 | | |
| | sequence 2 embryonic lethal | | | | | |
| M364 | immunoglobulin kappa light | − | 11884.3 | 4378310 | | |
| | chain variable region | | | | | |
| M365 | Ig kappa chain V region (V- | − | 10302.6 | 480915 | | |
| | kappa 3) | | | | | |
| M366 | NEUROBLASTOMA | − | 19276.9 | 4885509 | | |
| | SUPPRESSOR OF | | | | | |
| | TUMORIGENICITY 1 | | | | | |
| | (ZINC FINGER PROTEIN | | | | | |
| | DAN) (N03) | | | | | |
| M367 | Ig kappa chain | − | 14845.1 | 482015 | | |
| M368 | immunoglobulin lambda | − | 9719.8 | 12044111 | | |
| | chain variable region | | | | | |
| M369 | immunoglobulin light chain | − | 11768.1 | 14573267 | | |
| | variable region | | | | | |
| M370 | putative | − | 25193.3 | 12847919 | | |
| M371 | immunoglobulin light chain | − | 13194.5 | 7716048 | | |
| | VL region | | | | | |
| M372 | alpha IF calcium channel | − | 221137 | 14669577 | | |
| | subunit | | | | | |
| M373 | sa1 (Drosophila)-like 2 | − | 105310 | 14751528 | | |
| M374 | AF15ql4 protein | − | 205685 | 9966807 | | |
| M375 | putative | +++ | 30469.3 | 12836789 | | |
| M376 | fibroblast growth factor 13 | − | 8239.5 | 4512024 | | |
| | isoform 1y1v | | | | | |
| M377 | immunoglobulin heavy chain | ++ | 10787.2 | 7161009 | | |
| M378 | immunoglobulin heavy chain | − | 11041.3 | 7161005 | | |
| M379 | KIAA0433 | − | 140408 | 7662118 | | |
| M380 | anti-c-erbB-2 | − | 11608.9 | 1145350 | | |
| | immunoglobulin light chain | | | | | |
| | V | | | | | |
| M381 | ATP-BINDING | − | 269976 | 14916523 | | |
| | CASSETTE, SUB-FAMILY | | | | | |
| | A, MEMBER 2 (ATP- | | | | | |
| | BINDING CASSETTE | | | | | |
| | TRANSPORTER 2) (ATP- | | | | | |
| | BINDING CASSETTE 2) | | | | | |
| M382 | G protein-coupled receptor | − | 85231.4 | 13929158 | | |
| | kinase-associated ADP | | | | | |
| | ribosylation factor GTPase- | | | | | |
| | activating protein | | | | | |
| M383 | immunoglobulin kappa chain | − | 12042.6 | 1235765 | | |
| | V-J region | | | | | |
| M384 | Ig kappa chain V region | ++ | 9166.2 | 7438723 | | |
| | (patient 17) | | | | | |
| M385 | immunoglobulin lambda | − | 9469.4 | 9714348 | | |
| | light chain variable region | | | | | |
| M386 | immunoglobulin lambda | − | 10019.1 | 11137154 | | |
| | chain | | | | | |
| M387 | farnesyl-protein transferase | ++ | 43087.6 | 2135098 | | |
| | beta chain | | | | | |
| M388 | KIAA1813 protein | − | 73090.1 | 14017843 | | |
| M389 | hypothetical protein | − | 117875 | 13644578 | | |
| | FLJ11937 | | | | | |
| M390 | immunoglobulin light chain | − | 11592 | 13171334 | | |
| | variable region | | | | | |
| M391 | ribosomal protein L29 | − | 17667.1 | 1082766 | | |
| M392 | Ig kappa light chain (VJC) | − | 14464.6 | 441357 | | |
| M393 | inhibitor PI, alpha I | − | 2431 | 223058 | | |
| | proteinase | | | | | |
| M394 | dJ467L1.3 (period | − | 126247 | 6580412 | | |
| | (Drosophila) homolog 3) | | | | | |
| M395 | cylindromatosis (turban | − | 82125.4 | 14779751 | | |
| | tumor syndrome) | | | | | |
| M396 | T cell receptor alpha chain V | − | 1710 | 478461 | | |
| | region (clone 2V alpha 23-2) | | | | | |
| M397 | immunoglobulin lambda | − | 11503.7 | 6643529 | | |
| | light chain variable region | | | | | |
| M398 | Ig kappa chain V-region (V- | − | 8746.8 | 185903 | | |
| | J2-C) | | | | | |
| M399 | anti-pneumococcal Ig L- | − | 11358.6 | 3603383 | | |
| | chain Fab fragment | | | | | |
| M400 | IG HEAVY CHAIN V | − | 12703.2 | 123774 | | |
| | REGION MOO | | | | | |
| M401 | Unknown (protein for | − | 45446.9 | 15530249 | | |
| | MGC: 16498) | | | | | |
| M402 | immunoglobulin lambda | ++ | 11645 | 3093884 | | |
| | light chain VJ region | | | | | |
| M403 | plasma protease (C1) | − | 55182.5 | 179619 | | |
| | inhibitor precursor | | | | | |
| M404 | similar to INITIATION | ++ | 16789.2 | 13642507 | | |
| | FACTOR 5A (EIF-5A) (EIF- | | | | | |
| | 4D) (REV-BINDING | | | | | |
| | FACTOR) (H. sapiens) | | | | | |
| M405 | Ig light chain VL1 region | − | 12477.9 | 520416 | | |
| M406 | CGI-202 | − | 13832.6 | 8895093 | | |
| M407 | Ig kappa chain V-III region | − | 2519.9 | 106612 | | |
| | (She) | | | | | |
| M408 | Ig light chain variable | − | 12142.7 | 1864119 | | |
| | domain | | | | | |
| M409 | alternatively spliced | − | 14203.2 | 2125864 | | |
| M410 | neuronal leucine-rich repeat | − | 79424.7 | 14751034 | | |
| | protein-3 | | | | | |
| M411 | Unknown (protein for | − | 71702.7 | 15277493 | | |
| | IMAGE: 3587716) | | | | | |
| M412 | PROTEASOME SUBUNIT | − | 29546.7 | 9910833 | | |
| | ALPHA TYPE 1 | | | | | |
| | (PROTEASOME | | | | | |
| | COMPONENT C2) | | | | | |
| | (MACROPAIN SUBUNIT | | | | | |
| | C2) (MULTICATALYTIC | | | | | |
| | ENDOPEPTIDASE | | | | | |
| | COMPLEX SUBUNIT C2) | | | | | |
| | (PROTEASOME NU | | | | | |
| | CHAIN) | | | | | |
| M413 | NIF-like protein | − | 38532.3 | 15487262 | | |
| M414 | IgM rearranged heavy chain | − | 12868.5 | 688441 | | |
| | mRNA V-D-J | | | | | |
| M415 | unnamed protein product | − | 27742.6 | 10436722 | | |
| M416 | immunoglobulin kappa chain | − | 11211.6 | 12655666 | | |
| | variable region | | | | | |
| M417 | hairy/enhancer of split 6 | − | 23902 | 14009498 | | |
| M418 | immunoglobulin kappa light | ++ | 11605.1 | 1561612 | | |
| | chain | | | | | |
| M419 | sperm tail associated protein | ++ | 119401 | 9910570 | | |
| M420 | NEURABIN-II (NEURAL | − | 89646.8 | 13431725 | | |
| | TISSUE-SPECIFIC F- | | | | | |
| | ACTIN BINDING PROTEIN | | | | | |
| | II) (SPINOPHILIN) (P130) | | | | | |
| | (PP1BP134) | | | | | |
| M421 | TFNR | − | 245729 | 12232589 | | |
| M422 | TROPOMYOSIN 4, | − | 28509.8 | 6981672 | | |
| | EMBRYONIC | | | | | |
| | FIBROBLAST ISOFORM | | | | | |
| | (TM-4) | | | | | |
| M423 | ZINC FINGER Y- | − | 90531.5 | 4507967 | | |
| | CHROMOSOMAL | | | | | |
| | PROTEIN | | | | | |
| M424 | CD27L RECEPTOR | + | 29156.6 | 4507587 | | |
| | PRECURSOR (T-CELL | | | | | |
| | ACTIVATION ANTIGEN | | | | | |
| | CD27) (T14) | | | | | |
| M425 | FIBROLEUKIN | − | 50228.9 | 5730075 | | |
| | PRECURSOR | | | | | |
| | (FIBRINOGEN-LIKE | | | | | |
| | PROTEIN 2) (PT49) | | | | | |
| M426 | haptoglobin | ++ | 45205.6 | 4826762 | | |
| M427 | IgA heavy chain variable | − | 6205.1 | 13123502 | | |
| | region | | | | | |
| M428 | putative | − | 2213.6 | 553734 | | |
| M429 | unnamed protein product | − | 94254.2 | 10435664 | | |
| M430 | immunoglobulin kappa- | − | 13454.4 | 197425 | | |
| | chain VK-1 | | | | | |
| M431 | hypothetical protein | − | 101555 | 14720628 | | |
| M432 | immunoglobulin kappa light | − | 11061.5 | 4323922 | | |
| | chain variable region | | | | | |
| M433 | myosin-VIIb | + | 240788 | 14161694 | | |
| M434 | immunoglobulin light chain | − | 11526.8 | 6735446 | | |
| | variable region | | | | | |
| M435 | hypothetical protein | − | 34555.4 | 15300488 | | |
| | FLJ20516 | | | | | |
| M436 | similar to ribosomal protein | − | 13043.6 | 13642964 | | |
| | S26 (H. sapiens) | | | | | |
| M437 | immunoglobulin variable | − | 11926.5 | 2597936 | | |
| | region, kappa light chain | | | | | |
| M438 | immunoglobulin lambda -- | − | 11511.7 | 3142584 | | |
| | light chain variable region | | | | | |
| M439 | C-terminus matches | − | 375564 | 4522026 | | |
| | KIAA0559, N-terminus | | | | | |
| | similar to Bassoon protein | | | | | |
| M440 | immunoglobulin lambda | − | 11380.6 | 3142574 | | |
| | light chain variable region | | | | | |
| M441 | arfaptin 1 | + | 40779.3 | 11120714 | | |
| M442 | apolipoprotein CI | − | 7750.9 | 178834 | | |
| M443 | dJ591C20.1 (novel protein | − | 52554.9 | 11125672 | | |
| | similar to mouse NG26) | | | | | |
| M444 | VESICLE TRANSPORT V- | − | 26687.6 | 13124617 | | |
| | SNARE PROTEIN VTI1- | | | | | |
| | LIKE 1 (VTI1-RP1) | | | | | |
| M445 | retinoblastoma-associated | − | 73913.1 | 5174457 | | |
| | protein HEC | | | | | |
| M446 | ZINC FINGER PROTEIN | − | 45586 | 4885267 | | |
| | GFI-1 (GROWTH FACTOR | | | | | |
| | INDEPENDENCE- 1) | | | | | |
| M447 | 5- | ++ | 40672.8 | 6981062 | | |
| | HYDROXYTRYPTAMINE | | | | | |
| | 5A RECEPTOR (5-HT-5A) | | | | | |
| | (SEROTONIN RECEPTOR) | | | | | |
| | (REC17) | | | | | |
| M448 | This CDS feature is included | − | 11581.9 | 845530 | | |
| | to show the translation of the | | | | | |
| | corresponding V_region. | | | | | |
| | Presently translation | | | | | |
| | qualifiers on V_region | | | | | |
| | features are illegal | | | | | |
| M449 | immunoglobulin lambda | − | 11511.7 | 3091160 | | |
| | light chain variable region | | | | | |
| M450 | Ig kappa chain V-II region | − | 13106.9 | 87880 | | |
| | (Inc) | | | | | |
| M451 | KIAA1664 protein | − | 99851.7 | 13359201 | | |
| M452 | METALLOTHIONEIN-II | − | 6042.3 | 5174764 | | |
| | (MT-II) | | | | | |
| M453 | microseminoprotein beta | − | 10651.1 | 225159 | | |
| M454 | SACSIN | − | 436757 | 7657536 | | |
| M455 | tenascin-R | − | 149575 | 1617316 | | |
| M456 | immunoglobulin kappa chain | − | 11352.8 | 12655503 | | |
| | variable region | | | | | |
| M457 | SERINE PROTEASE | − | 120760 | 13959398 | | |
| | INHIBITOR KAZAL-TYPE | | | | | |
| | 5 PRECURSOR (LYMPHO- | | | | | |
| | EPITHELIAL KAZAL- | | | | | |
| | TYPE RELATED | | | | | |
| | INHIBITOR) (LEKTI) | | | | | |
| | [CONTAINS: | | | | | |
| | HEMOFILTRATE PEPTIDE | | | | | |
| | HF6478 | | | | | |
| M458 | CALGRANULIN C | − | 10614.1 | 461678 | | |
| | (CAGC) | | | | | |
| M459 | galactose-specific lectin | − | 26148.5 | 1196442 | | |
| M460 | non-muscle myosin heavy | − | 72371.4 | 3205211 | | |
| | chain | | | | | |
| M461 | DOCK 180 protein | − | 215377 | 4503355 | | |
| M462 | serine protease | − | 40239.3 | 3777621 | | |
| M463 | nuclear zinc finger protein | − | 89814.4 | 14764016 | | |
| | Np95 | | | | | |
| M464 | LIM protein (similar to rat | − | 63972.5 | 14250573 | | |
| | protein kinase C-binding | | | | | |
| | enigma) | | | | | |
| M465 | SECIS binding protein 2 | − | 95478.4 | 14211829 | | |
| M466 | HSPC055 protein | − | 83654.5 | 14775320 | | |
| M467 | immunoglobulin V lambda/J | − | 11099.1 | 6643881 | | |
| | lambda light chain | | | | | |
| M468 | ribosomal protein LI 4 | − | 23803.4 | 4506601 | | |
| M469 | immunoglobulin kappa chain | − | 10755.2 | 722526 | | |
| M470 | chloride channel protein 3, | − | 91243.1 | 4502869 | | |
| | long form | | | | | |
| M471 | MITOGEN-ACTIVATED | − | 70970.1 | 4505153 | | |
| | PROTEIN KINASE | | | | | |
| | KINASE KINASE 3 | | | | | |
| | (MAPK/ERK KINASE | | | | | |
| | KINASE 3) (MEK KINASE | | | | | |
| | 3) (MEKK3) | | | | | |
| M472 | unnamed protein product | − | 62332 | 12383086 | | |
| M473 | anti-HIV gp 120 antibody | − | 11498.5 | 460857 | | |
| | light chain variable region | | | | | |
| M474 | CARBONIC ANHYDRASE | − | 34394.2 | 1345657 | | |
| | IV PRECURSOR | | | | | |
| | (CARBONATE | | | | | |
| | DEHYDRATASE IV) (CA- | | | | | |
| | IV) | | | | | |
| M475 | hypothetical protein | − | 47126.1 | 13937775 | | |
| | FLJ11016 | | | | | |
| M476 | putative gene with | − | 50038.1 | 7717246 | | |
| | similarities to KIAA1074 and | | | | | |
| | KIAA0565 | | | | | |
| M477 | S-MYC PROTO- | − | 47002.5 | 11177868 | | |
| | ONCOGENE PROTEIN | | | | | |
| M478 | immunoglobulin kappa light | − | 11322.7 | 4378208 | | |
| | chain variable region | | | | | |
| M479 | immunoglobulin heavy- | − | 13556.2 | 348178 | | |
| | chain subgroup VIII V-D-J | | | | | |
| | region | | | | | |
| M480 | unnamed protein product | − | 42680.2 | 10432636 | | |
| M481 | immunoglobulin lambda | − | 9964.2 | 4566006 | | |
| | light chain | | | | | |
| M482 | immunoglobulin kappa light | + | 11412.8 | 1699304 | | |
| | chain variable region | | | | | |
| M483 | proapolipoprotein Varient | − | 25119.7 | | 1 | 2 |
| M484 | Unknown | + | 33413.9 | | 3 | 4 |
| M485 | neutrophil lactoferrin | − | 59529.6 | 186818 | | |
| | | | | (GenPept) | | |
| M486 | alpha-2-HS glycoprotein | − | 12290.2 | 10289662 | | |
| | Varient | | | (dbEST) | | |
| M487 | MT-11 protein Varient | − | 10172.9 | 7378207 | | |
| | | | | (dbEST) | | |
| M488 | immunoglobulin lambda light | − | 16740.9 | | 5 | 6 |
| | chain homolog | | | | | |
| M489 | Unknown | − | 21461.8 | | 7 | 8 |
| M490 | Unknown | − | 20895 | | 9 | 10 |

10¹⁰ and above +++
10⁸ to 10¹⁰ ++
10⁶ to 10⁸ +
0

TABLE 2

| | | E_51 | E_59 | E_63 | E_67 | E_70 | N_52 | N_54 | N_58 | N_65 |
|---|---|---|---|---|---|---|---|---|---|---|
| Marker | Gene Name | Br38 | Br26 | Br35 | Br42 | Br43 | Br29 | Br28 | Br34 | Br36 |
| M4 | COMPLEMENT C3 | ++ | +++ | +++ | +++ | +++ | +++ | +++ | +++ | +++ |
| | PRECURSOR | | | | | | | | | |
| | [CONTAINS: C3A | | | | | | | | | |
| | ANAPHYLATOXIN] | | | | | | | | | |
| M5 | proapolipoprotein | +++ | +++ | +++ | +++ | +++ | +++ | +++ | +++ | +++ |
| M7 | FIBRINOGEN | +++ | +++ | +++ | +++ | +++ | +++ | +++ | +++ | ++ |
| | ALPHA/ALPHA-E CHAIN | | | | | | | | | |
| | PRECURSOR | | | | | | | | | |
| M8 | FIBRINOGEN BETA | +++ | ++ | +++ | +++ | +++ | +++ | +++ | +++ | ++ |
| | CHAIN PRECURSOR | | | | | | | | | |
| M10 | TRANSTHYRETIN | +++ | +++ | +++ | +++ | +++ | +++ | +++ | +++ | +++ |
| | PRECURSOR | | | | | | | | | |
| | (PREALBUMIN) (TBPA) | | | | | | | | | |
| | (TTR) (ATTR) | | | | | | | | | |
| M12 | apolipoprotein A-IV | ++ | ++ | +++ | +++ | +++ | +++ | +++ | +++ | ++ |
| | precursor | | | | | | | | | |
| M13 | alpha 1-acid glycoprotein | +++ | +++ | +++ | +++ | +++ | +++ | +++ | +++ | +++ |
| M14 | hemopexin | +++ | +++ | +++ | +++ | +++ | +++ | ++ | +++ | ++ |
| M15 | macroglobulin alpha2 | ++ | +++ | ++ | +++ | ++ | ++ | ++ | +++ | ++ |
| M17 | alpha-2-glycoprotein 1, | +++ | +++ | +++ | +++ | +++ | +++ | +++ | +++ | +++ |
| | zinc | | | | | | | | | |
| M21 | ANTITHROMBIN-III | ++ | ++ | +++ | +++ | ++ | ++ | + | +++ | ++ |
| | PRECURSOR (ATIII) | | | | | | | | | |
| M22 | AMBP PROTEIN | ++ | +++ | +++ | +++ | +++ | +++ | ++ | +++ | +++ |
| | PRECURSOR | | | | | | | | | |
| | [CONTAINS: ALPHA-1- | | | | | | | | | |
| | MICROGLOBULIN | | | | | | | | | |
| | (PROTEIN HC) | | | | | | | | | |
| | (COMPLEX-FORMING | | | | | | | | | |
| | GLYCOPROTEIN | | | | | | | | | |
| | HETEROGENEOUS IN | | | | | | | | | |
| | CHARGE) | | | | | | | | | |
| M23 | PROTHROMBIN | ++ | +++ | +++ | +++ | +++ | ++ | ++ | +++ | ++ |
| | PRECURSOR | | | | | | | | | |
| | (COAGULATION | | | | | | | | | |
| | FACTOR II) | | | | | | | | | |
| M24 | ALPHA-2-HS- | +++ | +++ | +++ | +++ | +++ | +++ | +++ | +++ | +++ |
| | GLYCOPROTEIN | | | | | | | | | |
| | PRECURSOR (FETUIN-A) | | | | | | | | | |
| | (ALPHA-2-Z-GLOBULIN) | | | | | | | | | |
| | (BA-ALPHA-2- | | | | | | | | | |
| | GLYCOPROTEIN) | | | | | | | | | |
| M25 | actin beta | ++ | +++ | +++ | ++ | +++ | ++ | ++ | − | ++ |
| M26 | CLUSTERIN | +++ | ++ | ++ | ++ | ++ | +++ | +++ | +++ | +++ |
| | PRECURSOR | | | | | | | | | |
| | (COMPLEMENT- | | | | | | | | | |
| | ASSOCIATED PROTEIN | | | | | | | | | |
| | SP-40, 40) | | | | | | | | | |
| | (COMPLEMENT | | | | | | | | | |
| | CYTOLYSIS INHIBITOR) | | | | | | | | | |
| | (CLI) (NA1 AND NA2) | | | | | | | | | |
| | (APOLIPOPROTEIN J) | | | | | | | | | |
| | (APO-J) (TRPM-2) | | | | | | | | | |
| M27 | bA120D12.1 (CD5 antigen- | ++ | ++ | ++ | +++ | ++ | +++ | ++ | +++ | ++ |
| | like (scavenger receptor | | | | | | | | | |
| | cysteine rich family)) | | | | | | | | | |
| M28 | fibrinogen gamma-B chain | +++ | ++ | +++ | +++ | ++ | +++ | +++ | +++ | ++ |
| | precursor | | | | | | | | | |
| M29 | polyubiquitin 4 | ++ | +++ | ++ | ++ | + | ++ | ++ | − | − |
| M30 | KININOGEN | ++ | − | − | − | − | ++ | ++ | + | ++ |
| | PRECURSOR (ALPHA-2- | | | | | | | | | |
| | THIOL PROTEINASE | | | | | | | | | |
| | INHIBITOR) [CONTAINS: | | | | | | | | | |
| | BRADYKININ] | | | | | | | | | |
| M31 | CALGRANULIN B | +++ | +++ | +++ | +++ | +++ | +++ | +++ | ++ | ++ |
| | (MIGRATION | | | | | | | | | |
| | INHIBITORY FACTOR- | | | | | | | | | |
| | RELATED PROTEIN 14) | | | | | | | | | |
| | (MRP-14) (P14) | | | | | | | | | |
| | (LEUKOCYTE L1 | | | | | | | | | |
| | COMPLEX HEAVY | | | | | | | | | |
| | CHAIN) (S100 CALCIUM- | | | | | | | | | |
| | BINDING PROTEIN A9) | | | | | | | | | |
| M32 | PLASMA RETINOL- | +++ | ++ | ++ | +++ | ++ | +++ | +++ | +++ | +++ |
| | BINDING PROTEIN | | | | | | | | | |
| | PRECURSOR (PRBP) | | | | | | | | | |
| | (RBP) | | | | | | | | | |
| M33 | Ig mu chain precursor, | ++ | +++ | ++ | +++ | ++ | +++ | − | ++ | ++ |
| | membrane-bound (clone | | | | | | | | | |
| | 201) | | | | | | | | | |
| M34 | APOLIPOPROTEIN A-II | ++ | +++ | +++ | +++ | +++ | +++ | +++ | +++ | +++ |
| | PRECURSOR (APO-AII) | | | | | | | | | |
| M35 | LEUCINE-RICH ALPHA- | ++ | +++ | ++ | +++ | +++ | +++ | ++ | +++ | ++ |
| | 2-GLYCOPROTEIN (LRG) | | | | | | | | | |
| M36 | dJ34F7.4 (complement | ++ | − | ++ | +++ | ++ | + | ++ | − | ++ |
| | component 4A) | | | | | | | | | |
| M37 | similar to phosphoglycerate | ++ | ++ | ++ | ++ | ++ | ++ | ++ | − | − |
| | mutase 1 (brain) | | | | | | | | | |
| M38 | alpha-1-antichymotrypsin | − | ++ | ++ | ++ | ++ | ++ | − | ++ | + |
| | precursor | | | | | | | | | |
| M39 | ALPHA-1B- | ++ | ++ | +++ | +++ | ++ | +++ | ++ | +++ | ++ |
| | GLYCOPROTEIN | | | | | | | | | |
| M41 | lipoprotein CIII | ++ | ++ | ++ | ++ | +++ | +++ | +++ | +++ | +++ |
| M42 | TETRANECTIN | ++ | ++ | +++ | +++ | ++ | +++ | ++ | +++ | +++ |
| | PRECURSOR (TN) | | | | | | | | | |
| | (PLASMINOGEN- | | | | | | | | | |
| | KRINGLE 4 BINDING | | | | | | | | | |
| | PROTEIN) | | | | | | | | | |
| M43 | amyloid related serum | ++ | ++ | +++ | +++ | +++ | +++ | ++ | − | ++ |
| | protein SAA | | | | | | | | | |
| M44 | CALGRANULIN A | +++ | ++ | +++ | +++ | +++ | ++ | +++ | ++ | + |
| | (MIGRATION | | | | | | | | | |
| | INHIBITORY FACTOR- | | | | | | | | | |
| | RELATED PROTEIN 8) | | | | | | | | | |
| | (MRP-8) (CYSTIC | | | | | | | | | |
| | FIBROSIS ANTIGEN) | | | | | | | | | |
| | (CFAG) (P8) | | | | | | | | | |
| | (LEUKOCYTE L1 | | | | | | | | | |
| | COMPLEX LIGHT | | | | | | | | | |
| | CHAIN) (S100 CALCIUM- | | | | | | | | | |
| | BINDING PROTEIN A8) | | | | | | | | | |
| M45 | C-REACTIVE PROTEIN | − | +++ | ++ | +++ | +++ | ++ | + | − | ++ |
| | PRECURSOR | | | | | | | | | |
| M46 | RHO GDP- | ++ | ++ | +++ | ++ | ++ | ++ | ++ | − | + |
| | DISSOCIATION | | | | | | | | | |
| | INHIBITOR 2 (RHO GDI | | | | | | | | | |
| | 2) (RHO-GDI BETA) (LY- | | | | | | | | | |
| | GDI) | | | | | | | | | |
| M47 | PROFILIN I | +++ | +++ | +++ | +++ | ++ | ++ | ++ | ++ | ++ |
| M49 | APOLIPOPROTEIN D | ++ | − | ++ | − | ++ | +++ | ++ | +++ | +++ |
| | PRECURSOR | | | | | | | | | |
| M50 | inter-alpha-trypsin inhibitor | ++ | ++ | ++ | ++ | ++ | +++ | + | ++ | ++ |
| | family heavy chain-related | | | | | | | | | |
| | protein | | | | | | | | | |
| M51 | 14-3-3 PROTEIN | ++ | ++ | ++ | ++ | ++ | − | + | − | + |
| | BETA/ALPHA (PROTEIN | | | | | | | | | |
| | KINASE C INHIBITOR | | | | | | | | | |
| | PROTEIN-1) (KCIP-1) | | | | | | | | | |
| | (PROTEIN 1054) | | | | | | | | | |
| M52 | Ig G1 H Nie | − | ++ | ++ | ++ | ++ | − | − | − | − |
| M53 | OSTEOPONTIN | + | ++ | ++ | − | − | − | − | − | − |
| | PRECURSOR (BONE | | | | | | | | | |
| | SIALOPROTEIN 1) | | | | | | | | | |
| | (URINARY STONE | | | | | | | | | |
| | PROTEIN) (SECRETED | | | | | | | | | |
| | PHOSPHOPROTEIN 1) | | | | | | | | | |
| | (SPP-1) | | | | | | | | | |
| | (NEPHROPONTIN) | | | | | | | | | |
| | (UROPONTIN) | | | | | | | | | |
| M54 | AFAMIN PRECURSOR | ++ | − | ++ | − | − | ++ | − | ++ | + |
| | (ALPHA-ALBUMIN) | | | | | | | | | |
| | (ALPHA-ALB) | | | | | | | | | |
| M55 | TRIOSEPHOSPHATE | ++ | ++ | ++ | − | ++ | − | − | − | − |
| | ISOMERASE (TIM) | | | | | | | | | |
| M56 | pre-serum amyloid P | ++ | − | − | − | ++ | +++ | − | − | − |
| | component | | | | | | | | | |
| M57 | COMP_HUMAN | ++ | ++ | ++ | − | + | ++ | ++ | ++ | − |
| M58 | prosaposin (variant | − | ++ | ++ | ++ | − | ++ | − | − | − |
| | Gaucher disease and variant | | | | | | | | | |
| | metachromatic | | | | | | | | | |
| | leukodystrophy) | | | | | | | | | |
| M59 | VITRONECTIN | ++ | ++ | ++ | ++ | ++ | ++ | ++ | +++ | ++ |
| | PRECURSOR (SERUM | | | | | | | | | |
| | SPREADING FACTOR) | | | | | | | | | |
| | (S-PROTEIN) | | | | | | | | | |
| | [CONTAINS: | | | | | | | | | |
| | SOMATOMEDIN B] | | | | | | | | | |
| M60 | CALGRANULIN C (S100 | ++ | +++ | ++ | ++ | ++ | ++ | ++ | − | − |
| | A12 protein) | | | | | | | | | |
| M61 | cofilin 1 (non-muscle) | ++ | ++ | ++ | ++ | ++ | ++ | − | ++ | ++ |
| M62 | cathepsin B | ++ | ++ | ++ | ++ | ++ | − | − | − | − |
| M63 | plasmin (EC 3.4.21.7) | + | + | − | ++ | + | ++ | − | ++ | + |
| | precursor [validated] | | | | | | | | | |
| M64 | PLASMA | ++ | ++ | ++ | +++ | ++ | +++ | ++ | +++ | ++ |
| | GLUTATHIONE | | | | | | | | | |
| | PEROXIDASE | | | | | | | | | |
| | PRECURSOR (GSHPX-P) | | | | | | | | | |
| M65 | lumican | − | ++ | ++ | ++ | ++ | − | ++ | +++ | ++ |
| M66 | apolipoprotein C-II | ++ | + | − | − | − | ++ | +++ | ++ | ++ |
| M67 | calmodulin 2 | ++ | ++ | ++ | − | ++ | ++ | ++ | − | + |
| | (phosphorylase kinase, | | | | | | | | | |
| | delta) | | | | | | | | | |
| M68 | THYMOSIN BETA-4 | ++ | + | ++ | ++ | ++ | − | − | − | − |
| M69 | uracil DNA glycosylase | ++ | ++ | ++ | ++ | − | − | − | − | − |
| M70 | defensin alpha-3 precursor, | +++ | +++ | +++ | +++ | ++ | − | ++ | − | ++ |
| | neutrophil-specific | | | | | | | | | |
| | [validated] | | | | | | | | | |
| M71 | CD14 antigen | − | − | ++ | ++ | ++ | − | ++ | − | − |
| M72 | peptidylprolyl isomerase | ++ | ++ | ++ | ++ | ++ | − | − | − | ++ |
| | (EC 5.2.1.8) A | | | | | | | | | |
| M73 | similar to transgelin 2 (H. | ++ | ++ | ++ | − | ++ | + | − | − | − |
| | sapiens) | | | | | | | | | |
| M74 | BETA-2- | ++ | − | − | ++ | +++ | − | ++ | ++ | − |
| | GLYCOPROTEIN I | | | | | | | | | |
| | PRECURSOR | | | | | | | | | |
| | (APOLIPOPROTEIN H) | | | | | | | | | |
| | (APO-H) (B2GPI) | | | | | | | | | |
| | (BETA(2)GPI) | | | | | | | | | |
| | (ACTIVATED PROTEIN | | | | | | | | | |
| | C-BINDING PROTEIN) | | | | | | | | | |
| | (APC INHIBITOR) | | | | | | | | | |
| M75 | complement 9 | − | − | − | − | − | ++ | + | − | − |
| M76 | alpha2 plasmin inhibitor | ++ | ++ | ++ | ++ | ++ | + | ++ | − | − |
| M77 | FICOLIN 3 PRECURSOR | − | − | ++ | − | − | ++ | ++ | − | ++ |
| | (COLLAGEN/FIBRINOGEN | | | | | | | | | |
| | DOMAIN- | | | | | | | | | |
| | CONTAINING PROTEIN | | | | | | | | | |
| | 3) | | | | | | | | | |
| | (COLLAGEN/FIBRINOGEN | | | | | | | | | |
| | DOMAIN- | | | | | | | | | |
| | CONTAINING LECTIN 3 | | | | | | | | | |
| | P35) (Hakata Antigen) | | | | | | | | | |
| M78 | glutathione transferase | ++ | ++ | ++ | − | − | ++ | − | − | − |
| M81 | complement factor B | ++ | ++ | ++ | ++ | ++ | +++ | ++ | − | − |
| M82 | CALGIZZARIN (S100C | ++ | ++ | ++ | ++ | ++ | − | − | − | + |
| | PROTEIN) (MLN 70) | | | | | | | | | |
| M83 | BETA-2- | ++ | +++ | +++ | +++ | +++ | ++ | ++ | +++ | ++ |
| | MICROGLOBULIN | | | | | | | | | |
| | PRECURSOR | | | | | | | | | |
| M84 | CYSTEINE-RICH | + | − | ++ | ++ | − | ++ | − | ++ | ++ |
| | SECRETORY PROTEIN-3 | | | | | | | | | |
| | PRECURSOR (CRISP-3) | | | | | | | | | |
| | (SGP28 PROTEIN) | | | | | | | | | |
| M86 | megakaryocyte stimulating | ++ | ++ | ++ | ++ | − | ++ | − | ++ | ++ |
| | factor | | | | | | | | | |
| M87 | inter-alpha-trypsin inhibitor | + | − | − | ++ | ++ | + | + | ++ | − |
| | heavy chain H1 precursor | | | | | | | | | |
| M88 | keratin 9, cytoskeletal | − | − | + | − | − | + | − | − | + |
| M89 | FERRITIN HEAVY | − | ++ | ++ | − | − | + | + | − | − |
| | CHAIN (FERRITIN H | | | | | | | | | |
| | SUBUNIT) | | | | | | | | | |
| M90 | HISTONE H1.1 | + | ++ | ++ | − | + | − | ++ | − | − |
| M91 | keratin 1 | − | − | − | − | ++ | ++ | − | − | + |
| M92 | ferritin light subunit | − | ++ | ++ | ++ | − | + | + | − | − |
| M93 | PROTEASOME | + | ++ | ++ | − | − | − | − | − | − |
| | SUBUNIT ALPHA TYPE 2 | | | | | | | | | |
| | (PROTEASOME | | | | | | | | | |
| | COMPONENT C3) | | | | | | | | | |
| | (MACROPAIN SUBUNIT | | | | | | | | | |
| | C3) (MULTICATALYTIC | | | | | | | | | |
| | ENDOPEPTIDASE | | | | | | | | | |
| | COMPLEX SUBUNIT C3) | | | | | | | | | |
| M94 | CARBONIC | − | ++ | ++ | − | − | − | − | − | − |
| | ANHYDRASE I | | | | | | | | | |
| | (CARBONATE | | | | | | | | | |
| | DEHYDRATASE I) (CA-I) | | | | | | | | | |
| M95 | LYMPHOCYTE- | − | − | ++ | − | − | − | − | − | − |
| | SPECIFIC PROTEIN LSP1 | | | | | | | | | |
| | (PP52 PROTEIN) (52 KDA | | | | | | | | | |
| | PHOSPHOPROTEIN) | | | | | | | | | |
| | (LYMPHOCYTE- | | | | | | | | | |
| | SPECIFIC ANTIGEN | | | | | | | | | |
| | WP34) | | | | | | | | | |
| M96 | apolipoprotein F | ++ | − | ++ | − | − | ++ | ++ | − | ++ |
| M98 | OSTEOINDUCTIVE | − | − | ++ | − | − | − | − | ++ | − |
| | FACTOR PRECURSOR | | | | | | | | | |
| | (OIF) (OSTEOGLYCIN) | | | | | | | | | |
| | (MIMECAN) | | | | | | | | | |
| M99 | APOLIPOPROTEIN E | − | ++ | − | + | − | ++ | ++ | ++ | − |
| | PRECURSOR (APO-E) | | | | | | | | | |
| M100 | PHOSPHATIDYLETHANOL- | ++ | + | ++ | − | − | − | − | − | − |
| | AMINE-BINDING | | | | | | | | | |
| | PROTEIN (PEBP) | | | | | | | | | |
| | (NEUROPOLYPEPTIDE | | | | | | | | | |
| | H3) (HIPPOCAMPAL | | | | | | | | | |
| | CHOLINERGIC | | | | | | | | | |
| | NEUROSTIMULATING | | | | | | | | | |
| | PEPTIDE) (HCNP) (RAF | | | | | | | | | |
| | KINASE INHIBITOR | | | | | | | | | |
| | PROTEIN) (RKIP) | | | | | | | | | |
| M101 | SUPEROXIDE | ++ | − | + | − | + | ++ | − | − | − |
| | DISMUTASE [CU—ZN] | | | | | | | | | |
| M102 | HISTONE H1A (H1.1) | + | ++ | ++ | − | + | − | + | − | − |
| M103 | neutrophil lipocalin | ++ | − | ++ | − | − | − | − | − | − |
| M104 | serum | − | − | − | ++ | ++ | − | − | − | ++ |
| | paraoxonasearylesterase 1 | | | | | | | | | |
| M105 | CYTIDINE DEAMINASE | ++ | − | ++ | − | − | ++ | − | − | − |
| | (CYTIDINE | | | | | | | | | |
| | AMINOHYDROLASE) | | | | | | | | | |
| M106 | CYSTATIN B (LIVER | ++ | ++ | ++ | ++ | ++ | − | − | + | − |
| | THIOL PROTEINASE | | | | | | | | | |
| | INHIBITOR) (CPI-B) | | | | | | | | | |
| | (STEFIN B) | | | | | | | | | |
| M108 | angiotensinogen (serine (or | − | − | − | ++ | − | − | − | ++ | − |
| | cysteine) proteinase | | | | | | | | | |
| | inhibitor, clade A (alpha-1 | | | | | | | | | |
| | antiproteinase, antitrypsin), | | | | | | | | | |
| | member 8) | | | | | | | | | |
| M109 | COLLAGEN ALPHA | − | − | ++ | ++ | − | − | − | ++ | − |
| | 3 (VI) CHAIN | | | | | | | | | |
| | PRECURSOR | | | | | | | | | |
| M110 | beta galactoside binding | − | ++ | − | ++ | − | − | − | ++ | − |
| | lectin | | | | | | | | | |
| M111 | SH3BGRL3-like protein | ++ | ++ | ++ | ++ | − | − | − | ++ | − |
| M112 | proteoglycan link protein 2 | − | − | − | ++ | − | − | − | ++ | − |
| M113 | LYSOZYME C | − | ++ | − | ++ | − | − | − | − | − |
| | PRECURSOR (1,4-BETA- | | | | | | | | | |
| | N- | | | | | | | | | |
| | ACETYLMURAMIDASE | | | | | | | | | |
| | C) | | | | | | | | | |
| M114 | cysteine-rich secreted A12- | − | + | ++ | ++ | − | − | − | − | − |
| | alpha-like protein 2 | | | | | | | | | |
| M115 | LEUKOCYTE | − | ++ | + | − | − | − | − | − | − |
| | ELASTASE INHIBITOR | | | | | | | | | |
| | (LEI) | | | | | | | | | |
| | (MONOCYTE/NEUTROPHIL | | | | | | | | | |
| | ELASTASE | | | | | | | | | |
| | INHIBITOR) (M/NEI) (EI) | | | | | | | | | |
| M116 | INSULIN-LIKE | + | + | − | ++ | ++ | − | − | ++ | − |
| | GROWTH FACTOR | | | | | | | | | |
| | BINDING PROTEIN 4 | | | | | | | | | |
| | PRECURSOR (IGFBP-4) | | | | | | | | | |
| | (IBP-4) (IGF-BINDING | | | | | | | | | |
| | PROTEIN 4) | | | | | | | | | |
| M117 | LOW AFFINITY | ++ | ++ | ++ | ++ | ++ | − | ++ | − | − |
| | IMMUNOGLOBULIN | | | | | | | | | |
| | GAMMA FC REGION | | | | | | | | | |
| | RECEPTOR III-A | | | | | | | | | |
| | PRECURSOR (IGG FC | | | | | | | | | |
| | RECEPTOR III-2) (FC- | | | | | | | | | |
| | GAMMA RIII-ALPHA) | | | | | | | | | |
| | (FC-GAMMA RIIIA) | | | | | | | | | |
| | (FCRIIIA) (FC-GAMMA | | | | | | | | | |
| | RIII) (FCRIII) (CD16-A) | | | | | | | | | |
| | (FCR-10) | | | | | | | | | |
| M118 | sex hormone-binding | − | − | + | ++ | − | − | + | ++ | − |
| | globulin | | | | | | | | | |
| M119 | ANNEXIN I | − | + | ++ | ++ | − | − | ++ | − | − |
| | (LIPOCORTIN I) | | | | | | | | | |
| | (CALPACTIN II) | | | | | | | | | |
| | (CHROMOBINDIN 9) | | | | | | | | | |
| | (P35) (PHOSPHOLIPASE | | | | | | | | | |
| | A2 INHIBITORY | | | | | | | | | |
| | PROTEIN) | | | | | | | | | |
| M120 | MnSOD precursor | − | − | ++ | − | + | + | − | − | − |
| M121 | EPIDIDYMAL | ++ | ++ | ++ | ++ | − | − | − | ++ | − |
| | SECRETORY PROTEIN | | | | | | | | | |
| | E1 PRECURSOR | | | | | | | | | |
| | (NIEMANN-PICK | | | | | | | | | |
| | DISEASE TYPE C2 | | | | | | | | | |
| | PROTEIN) (EPI-1) (HE1) | | | | | | | | | |
| | (EPIDIDYMAL | | | | | | | | | |
| | SECRETORY PROTEIN | | | | | | | | | |
| | 14.6) (ESP14.6) | | | | | | | | | |
| M123 | metastasin | ++ | ++ | ++ | − | ++ | − | − | ++ | − |
| M124 | inter-alpha (globulin) | − | − | − | − | − | ++ | + | − | − |
| | inhibitor, H2 polypeptide | | | | | | | | | |
| M125 | alpha-1 type I collagen | − | ++ | + | ++ | − | ++ | − | ++ | ++ |
| M126 | GELSOLIN | − | ++ | − | − | − | − | − | ++ | − |
| | PRECURSOR, PLASMA | | | | | | | | | |
| | (ACTIN- | | | | | | | | | |
| | DEPOLYMERIZING | | | | | | | | | |
| | FACTOR) (ADF) | | | | | | | | | |
| | (BREVIN) (AGEL) | | | | | | | | | |
| M127 | PROTEASOME | − | ++ | ++ | − | − | − | − | − | − |
| | SUBUNIT ALPHA TYPE 4 | | | | | | | | | |
| | (PROTEASOME | | | | | | | | | |
| | COMPONENT C9) | | | | | | | | | |
| | (MACROPAIN SUBUNIT | | | | | | | | | |
| | C9) (MULTICATALYTIC | | | | | | | | | |
| | ENDOPEPTIDASE | | | | | | | | | |
| | COMPLEX SUBUNIT C9) | | | | | | | | | |
| | (PROTEASOME | | | | | | | | | |
| | SUBUNIT L) | | | | | | | | | |
| M128 | Similar to coactosin-like | − | ++ | ++ | ++ | − | + | − | − | − |
| | protein | | | | | | | | | |
| M129 | THIOREDOXIN (ATL- | ++ | ++ | ++ | − | − | − | + | − | − |
| | DERIVED FACTOR) | | | | | | | | | |
| | (ADF) (SURFACE | | | | | | | | | |
| | ASSOCIATED | | | | | | | | | |
| | SULPHYDRYL PROTEIN) | | | | | | | | | |
| | (SASP) | | | | | | | | | |
| M130 | K12 protein precursor | − | ++ | − | ++ | − | − | − | ++ | − |
| M131 | VITAMIN-K | − | − | − | − | − | ++ | + | − | − |
| | DEPENDENT PROTEIN C | | | | | | | | | |
| | PRECURSOR | | | | | | | | | |
| | (AUTOPROTHROMBIN | | | | | | | | | |
| | IIA) (ANTICOAGULANT | | | | | | | | | |
| | PROTEIN C) (BLOOD | | | | | | | | | |
| | COAGULATION | | | | | | | | | |
| | FACTOR XIV) | | | | | | | | | |
| M132 | titin, cardiac muscle | − | − | − | − | − | − | − | − | ++ |
| | [validated] | | | | | | | | | |
| M133 | MBL-associated protein | − | − | − | ++ | − | ++ | − | ++ | ++ |
| | MAp19 | | | | | | | | | |
| M134 | EOSINOPHIL CATIONIC | − | ++ | ++ | ++ | + | − | ++ | − | − |
| | PROTEIN PRECURSOR | | | | | | | | | |
| | (ECP) (RIBONUCLEASE | | | | | | | | | |
| | 3) (RNASE 3) | | | | | | | | | |
| M135 | This CDS feature is | − | − | ++ | − | ++ | − | − | − | − |
| | included to show the | | | | | | | | | |
| | translation of the | | | | | | | | | |
| | corresponding V_region. | | | | | | | | | |
| | Presently translation | | | | | | | | | |
| | qualifiers on V_region | | | | | | | | | |
| | features are illegal | | | | | | | | | |
| M136 | enhancer protein | − | ++ | ++ | − | − | − | − | − | − |
| M137 | cysteine-rich protein 1 | − | ++ | − | ++ | − | + | − | ++ | − |
| M138 | anti-Gd cold agglutinin | − | ++ | − | ++ | ++ | ++ | ++ | ++ | − |
| | monoclonal IgMK light | | | | | | | | | |
| | chain variable region | | | | | | | | | |
| M139 | CYSTATIN A (STEFIN | + | − | − | ++ | ++ | − | − | − | − |
| | A) (CYSTATIN AS) | | | | | | | | | |
| M140 | ANNEXIN II | − | ++ | ++ | − | − | − | − | − | − |
| | (LIPOCORTIN II) | | | | | | | | | |
| | (CALPACTIN 1 HEAVY | | | | | | | | | |
| | CHAIN) | | | | | | | | | |
| | (CHROMOBINDIN 8) | | | | | | | | | |
| | (P36) (PROTEIN I) | | | | | | | | | |
| | (PLACENTAL | | | | | | | | | |
| | ANTICOAGULANT | | | | | | | | | |
| | PROTEIN IV) (PAP-IV) | | | | | | | | | |
| M141 | diazepam binding inhibitor | − | ++ | ++ | + | − | − | − | − | − |
| M142 | bA139H14.1 (lymphocyte | − | − | ++ | − | − | − | − | − | + |
| | cytosolic protein 1 (L- | | | | | | | | | |
| | plastin)) | | | | | | | | | |
| M143 | heparan sulfate | − | − | − | ++ | − | − | − | ++ | − |
| | proteoglycan 2 (perlecan) | | | | | | | | | |
| M144 | cathepsin C | − | − | + | + | − | − | − | − | − |
| M145 | MYELOBLASTIN | + | − | − | − | ++ | + | − | − | − |
| | PRECURSOR | | | | | | | | | |
| | (LEUKOCYTE | | | | | | | | | |
| | PROTEINASE 3) (PR-3) | | | | | | | | | |
| | (PR3) (AGP7) | | | | | | | | | |
| | (WEGENER'S | | | | | | | | | |
| | AUTOANTIGEN) (P29) | | | | | | | | | |
| | (C-ANCA ANTIGEN) | | | | | | | | | |
| M146 | glutaredoxin | − | + | ++ | − | ++ | − | − | − | − |
| M147 | TRANSLATIONALLY | − | ++ | ++ | − | − | + | − | − | − |
| | CONTROLLED TUMOR | | | | | | | | | |
| | PROTEIN (TCTP) | | | | | | | | | |
| M148 | PROTEASOME | − | − | − | − | − | ++ | − | − | − |
| | COMPONENT C13 | | | | | | | | | |
| | PRECURSOR | | | | | | | | | |
| | (MACROPAIN SUBUNIT | | | | | | | | | |
| | C13) (MULTICATALYTIC | | | | | | | | | |
| | ENDOPEPTIDASE | | | | | | | | | |
| | COMPLEX SUBUNIT | | | | | | | | | |
| | C13) | | | | | | | | | |
| M149 | ENDOTHELIAL | − | ++ | ++ | − | − | − | − | − | − |
| | PROTEIN C RECEPTOR | | | | | | | | | |
| | PRECURSOR | | | | | | | | | |
| | (ENDOTHELIAL CELL | | | | | | | | | |
| | PROTEIN C RECEPTOR) | | | | | | | | | |
| | (ACTIVATED PROTEIN C | | | | | | | | | |
| | RECEPTOR) (APC | | | | | | | | | |
| | RECEPTOR) | | | | | | | | | |
| M151 | D-DOPACHROME | ++ | + | ++ | − | + | − | − | − | − |
| | TAUTOMERASE | | | | | | | | | |
| | (PHENYLPYRUVATE | | | | | | | | | |
| | TAUTOMERASE II) | | | | | | | | | |
| M152 | p80 protein | − | ++ | − | − | − | − | − | − | − |
| M153 | COLLAGEN ALPHA 1(V) | − | − | ++ | − | − | − | − | ++ | − |
| | CHAIN PRECURSOR | | | | | | | | | |
| M155 | rho GDP dissociation | − | − | ++ | − | − | − | − | − | − |
| | inhibitor (GDI) | | | | | | | | | |
| M156 | FIBRONECTIN | − | − | ++ | − | − | + | − | − | − |
| | PRECURSOR (FN) | | | | | | | | | |
| | (COLD-INSOLUBLE | | | | | | | | | |
| | GLOBULIN) (CIG) | | | | | | | | | |
| M157 | FK506-BINDING | ++ | ++ | − | − | − | − | − | − | − |
| | PROTEIN (FKBP-12) | | | | | | | | | |
| | (PEPTIDYL-PROLYL CIS- | | | | | | | | | |
| | TRANS ISOMERASE) | | | | | | | | | |
| | (PPIASE) (ROTAMASE) | | | | | | | | | |
| | (IMMUNOPHILIN | | | | | | | | | |
| | FKBP12) | | | | | | | | | |
| M159 | similar to osteoclast | ++ | − | − | − | − | − | − | − | − |
| | stimulating factor 1 (H. | | | | | | | | | |
| | sapiens) | | | | | | | | | |
| M161 | PEROXIREDOXIN 2 | ++ | − | + | − | − | − | − | − | − |
| | (THIOREDOXIN | | | | | | | | | |
| | PEROXIDASE 1) | | | | | | | | | |
| | (THIOREDOXIN- | | | | | | | | | |
| | DEPENDENT PEROXIDE | | | | | | | | | |
| | REDUCTASE 1) (THIOL- | | | | | | | | | |
| | SPECIFIC | | | | | | | | | |
| | ANTIOXIDANT | | | | | | | | | |
| | PROTEIN) (TSA) (PRP) | | | | | | | | | |
| | (NATURAL KILLER | | | | | | | | | |
| | CELL ENHANCING | | | | | | | | | |
| | FACTOR B) (NKEF-B) | | | | | | | | | |
| M162 | myosin catalytic light chain | ++ | ++ | − | − | − | − | − | − | − |
| | LC17b | | | | | | | | | |
| M163 | protein SPY75 | + | + | − | − | − | − | − | − | − |
| M164 | 2-phosphopyruvate- | − | ++ | − | − | ++ | − | − | − | − |
| | hydratase alpha-enolase | | | | | | | | | |
| M166 | pancreatic secretory trypsin | − | + | − | ++ | + | − | − | − | − |
| | inhibitor | | | | | | | | | |
| M167 | GLIA MATURATION | ++ | − | + | ++ | − | − | − | − | − |
| | FACTOR GAMMA (GMF- | | | | | | | | | |
| | GAMMA) | | | | | | | | | |
| M168 | CALCYCLIN (LUNG 10 | − | + | ++ | − | − | + | − | − | − |
| | KDA PROTEIN) | | | | | | | | | |
| M169 | SH3 DOMAIN-BINDING | − | ++ | + | − | − | − | − | − | − |
| | GLUTAMIC ACID-RICH- | | | | | | | | | |
| | LIKE PROTEIN | | | | | | | | | |
| M170 | CHYMOTRYPSINOGENA | − | − | − | − | − | ++ | − | − | − |
| M171 | DJ-1 protein | ++ | − | ++ | − | ++ | + | − | − | − |
| M172 | phosphoprotein enriched in | − | − | + | − | − | − | − | − | − |
| | astrocytes 15 | | | | | | | | | |
| M173 | EGF-CONTAINING | − | − | − | ++ | − | + | − | − | − |
| | FIBULIN-LIKE | | | | | | | | | |
| | EXTRACELLULAR | | | | | | | | | |
| | MATRIX PROTEIN 1 | | | | | | | | | |
| | PRECURSOR (FIBULIN- | | | | | | | | | |
| | 3) (FIBL-3) (T16 | | | | | | | | | |
| | PROTEIN) | | | | | | | | | |
| M174 | superoxide dismutase 3, | + | − | − | − | − | ++ | − | − | − |
| | extracellular | | | | | | | | | |
| M175 | CLARA CELL | − | + | + | ++ | − | − | − | ++ | − |
| | PHOSPHOLIPID- | | | | | | | | | |
| | BINDING PROTEIN | | | | | | | | | |
| | PRECURSOR (CCPBP) | | | | | | | | | |
| | (CLARA CELLS 10 KDA | | | | | | | | | |
| | SECRETORY PROTEIN) | | | | | | | | | |
| | (CC10) (UTEROGLOBIN) | | | | | | | | | |
| | (URINE PROTEIN 1) | | | | | | | | | |
| | (UP1) | | | | | | | | | |
| M176 | Similar to LIM and SH3 | + | − | ++ | − | − | − | − | − | − |
| | protein 1 | | | | | | | | | |
| M177 | PROSTAGLANDIN-H2 D- | + | − | ++ | ++ | − | − | ++ | ++ | − |
| | ISOMERASE | | | | | | | | | |
| | PRECURSOR | | | | | | | | | |
| | (PROSTAGLANDIN-D | | | | | | | | | |
| | SYNTHASE) | | | | | | | | | |
| | (GLUTATHIONE- | | | | | | | | | |
| | INDEPENDENT PGD | | | | | | | | | |
| | SYNTHETASE) | | | | | | | | | |
| | (PROSTAGLANDIN D2 | | | | | | | | | |
| | SYNTHASE) (PGD2 | | | | | | | | | |
| | SYNTHASE) (PGDS2) | | | | | | | | | |
| | (PGDS) (BETA-TRACE | | | | | | | | | |
| | PROTEIN) | | | | | | | | | |
| M178 | mannose 6- | − | ++ | − | − | − | − | − | − | − |
| | phosphate/insulin-like | | | | | | | | | |
| | growth factor II receptor | | | | | | | | | |
| M179 | OXYGEN-REGULATED | − | ++ | − | − | − | − | − | − | − |
| | PROTEIN 1 (RETINITIS | | | | | | | | | |
| | PIGMENTOSA RP1 | | | | | | | | | |
| | PROTEIN) (RETINITIS | | | | | | | | | |
| | PIGMENTOSA 1 | | | | | | | | | |
| | PROTEIN) | | | | | | | | | |
| M180 | SERUM AMYLOID A | − | − | ++ | + | + | − | − | − | − |
| | PROTEIN (SAA) | | | | | | | | | |
| | [CONTAINS: AMYLOID | | | | | | | | | |
| | PROTEIN A (AMYLOID | | | | | | | | | |
| | FIBRIL PROTEIN AA)] | | | | | | | | | |
| M181 | CARGO SELECTION | − | − | ++ | − | − | − | − | − | − |
| | PROTEIN TIP47 (47 KDA | | | | | | | | | |
| | MANNOSE 6- | | | | | | | | | |
| | PHOSPHATE | | | | | | | | | |
| | RECEPTOR-BINDING | | | | | | | | | |
| | PROTEIN) (47 KDA MPR- | | | | | | | | | |
| | BINDING PROTEIN) | | | | | | | | | |
| | (PLACENTAL PROTEIN | | | | | | | | | |
| | 17) | | | | | | | | | |
| M182 | adenylyl cyclase-associated | − | − | ++ | − | − | ++ | − | − | − |
| | protein | | | | | | | | | |
| M183 | aldolase C | − | ++ | − | − | − | − | − | − | − |
| M184 | similar to PROTEASOME | − | − | ++ | − | − | − | − | − | − |
| | SUBUNIT ALPHA TYPE 6 | | | | | | | | | |
| | (PROTEASOME IOTA | | | | | | | | | |
| | CHAIN) (MACROPAIN | | | | | | | | | |
| | IOTA CHAIN) | | | | | | | | | |
| | (MULTICATALYTIC | | | | | | | | | |
| | ENDOPEPTIDASE | | | | | | | | | |
| | COMPLEX IOTA CHAIN) | | | | | | | | | |
| | (27 KDA PROSOMAL | | | | | | | | | |
| | PROTEIN) (PROS-27) | | | | | | | | | |
| | (P27K) (H. sapiens) | | | | | | | | | |
| M186 | FOLLISTATIN- | − | − | + | ++ | ++ | ++ | − | − | − |
| | RELATED PROTEIN 1 | | | | | | | | | |
| | PRECURSOR | | | | | | | | | |
| M187 | GAMMA-INTERFERON | − | ++ | ++ | − | − | ++ | − | − | − |
| | INDUCIBLE | | | | | | | | | |
| | LYSOSOMAL THIOL | | | | | | | | | |
| | REDUCTASE | | | | | | | | | |
| | PRECURSOR (GAMMA- | | | | | | | | | |
| | INTERFERON- | | | | | | | | | |
| | INDUCIBLE PROTEIN IP- | | | | | | | | | |
| | 30) | | | | | | | | | |
| M188 | F-ACTIN CAPPING | ++ | ++ | − | − | − | − | ++ | − | − |
| | PROTEIN BETA | | | | | | | | | |
| | SUBUNIT (CAPZ BETA) | | | | | | | | | |
| M189 | coagulation factor X | ++ | − | − | − | − | − | − | − | + |
| M190 | histone H3 | − | − | − | − | − | − | ++ | − | − |
| M191 | adiponectin | − | − | − | − | ++ | − | − | − | − |
| M192 | 16G2 | − | + | + | − | − | − | − | ++ | − |
| M195 | S-100P PROTEIN | ++ | + | − | − | + | − | − | − | − |
| M196 | INTERCELLULAR | − | + | − | ++ | − | − | − | ++ | − |
| | ADHESION MOLECULE- | | | | | | | | | |
| | 2 PRECURSOR (ICAM-2) | | | | | | | | | |
| | (CD102) | | | | | | | | | |
| M197 | MANNOSYL- | − | − | − | ++ | − | − | − | − | − |
| | OLIGOSACCHARIDE | | | | | | | | | |
| | ALPHA-1,2- | | | | | | | | | |
| | MANNOSIDASE | | | | | | | | | |
| | (MAN(9)-ALPHA- | | | | | | | | | |
| | MANNOSIDASE) | | | | | | | | | |
| M198 | NAD+-isocitrate | − | − | ++ | ++ | − | ++ | − | ++ | ++ |
| | dehydrogenase, alpha | | | | | | | | | |
| | subunit | | | | | | | | | |
| M199 | CONNECTIVE TISSUE | − | − | − | − | − | − | − | ++ | − |
| | GROWTH FACTOR | | | | | | | | | |
| | PRECURSOR | | | | | | | | | |
| | (HYPERTROPHIC | | | | | | | | | |
| | CHONDROCYTE- | | | | | | | | | |
| | SPECIFIC PROTEIN 24) | | | | | | | | | |
| M200 | complement factor H- | ++ | − | − | − | − | − | − | − | − |
| | related protein FHR-2 | | | | | | | | | |
| M201 | RIBONUCLEASE, | − | − | − | − | − | ++ | − | − | − |
| | SEMINAL PRECURSOR | | | | | | | | | |
| | (SEMINAL RNASE) (S- | | | | | | | | | |
| | RNASE) | | | | | | | | | |
| | (RIBONUCLEASE BS-1) | | | | | | | | | |
| M203 | putative | + | − | − | − | − | − | − | − | − |
| M204 | SERUM AMYLOID A-4 | − | − | − | − | − | ++ | − | − | − |
| | PROTEIN PRECURSOR | | | | | | | | | |
| | (CONSTITUTIVELY | | | | | | | | | |
| | EXPRESSED SERUM | | | | | | | | | |
| | AMYLOID A PROTEIN) | | | | | | | | | |
| | (C-SAA) | | | | | | | | | |
| M205 | HP1-BP74 | − | − | − | − | − | − | + | − | − |
| M206 | myristoylated alanine-rich | − | − | − | − | − | + | − | − | − |
| | C-kinase substrate | | | | | | | | | |
| M207 | CORTICOSTEROID- | − | ++ | − | − | − | + | − | ++ | − |
| | BINDING GLOBULIN | | | | | | | | | |
| | PRECURSOR (CBG) | | | | | | | | | |
| | (TRANSCORTIN) | | | | | | | | | |
| M208 | mannose-binding lectin | ++ | − | + | − | − | − | − | − | − |
| M209 | APOLIPOPROTEIN L | − | − | − | − | − | − | − | − | ++ |
| | PRECURSOR (APO-L) | | | | | | | | | |
| M210 | GLUTATHIONE | ++ | ++ | − | − | − | − | − | − | − |
| | TRANSFERASE OMEGA | | | | | | | | | |
| | 1 (GSTO 1-1) | | | | | | | | | |
| M211 | Somatomedin A | − | − | + | − | − | − | − | − | − |
| M212 | ganglioside M2 activator | − | ++ | − | − | − | − | − | − | + |
| | protein | | | | | | | | | |
| M214 | FATTY ACID-BINDING | − | + | ++ | − | − | − | − | − | − |
| | PROTEIN, EPIDERMAL | | | | | | | | | |
| | (E-FABP) (PSORIASIS- | | | | | | | | | |
| | ASSOCIATED FATTY | | | | | | | | | |
| | ACID-BINDING | | | | | | | | | |
| | PROTEIN HOMOLOG) | | | | | | | | | |
| | (PA-FABP) | | | | | | | | | |
| M215 | MYELOID CELL | − | − | + | − | − | − | − | − | − |
| | NUCLEAR | | | | | | | | | |
| | DIFFERENTIATION | | | | | | | | | |
| | ANTIGEN | | | | | | | | | |
| M216 | protein-L-isoaspartate (D- | ++ | − | + | − | − | − | − | − | − |
| | aspartate) O- | | | | | | | | | |
| | methyltransferase (EC | | | | | | | | | |
| | 2.1.1.77) splice form II | | | | | | | | | |
| M217 | pancreatic ribonuclease | − | − | + | − | − | − | − | − | − |
| | (EC 3.1.27.5) precursor | | | | | | | | | |
| M219 | anti-MSP1 MAD20 block2 | − | − | − | ++ | − | − | − | − | − |
| | ScFv Ig light chain variable | | | | | | | | | |
| | region | | | | | | | | | |
| M220 | vimentin | + | − | ++ | − | + | − | − | − | + |
| M221 | HLA CLASS II | − | − | + | − | − | − | − | − | − |
| | HISTOCOMPATIBILITY | | | | | | | | | |
| | ANTIGEN, GAMMA | | | | | | | | | |
| | CHAIN (HLA-DR | | | | | | | | | |
| | ANTIGENS | | | | | | | | | |
| | ASSOCIATED | | | | | | | | | |
| | INVARIANT CHAIN) | | | | | | | | | |
| | (P33) (CD74 ANTIGEN) | | | | | | | | | |
| M222 | IMP dehydrogenase | + | ++ | + | − | − | − | − | − | − |
| M223 | granulin | − | + | − | − | − | ++ | − | − | − |
| M224 | p97 | − | − | − | − | − | + | − | − | − |
| M225 | antigen HLA SB beta, MHC | − | − | + | − | − | − | − | − | − |
| | II | | | | | | | | | |
| M227 | kinesin like protein 9 | − | ++ | − | − | − | − | − | − | − |
| M228 | PROTEASOME | − | − | ++ | − | − | − | − | − | − |
| | COMPONENT MECL-1 | | | | | | | | | |
| | PRECURSOR | | | | | | | | | |
| | (MACROPAIN SUBUNIT | | | | | | | | | |
| | MECL-1) | | | | | | | | | |
| | (MULTICATALYTIC | | | | | | | | | |
| | ENDOPEPTIDASE | | | | | | | | | |
| | COMPLEX SUBUNIT | | | | | | | | | |
| | MECL-1) | | | | | | | | | |
| M233 | hypothetical protein | − | − | − | ++ | − | − | − | − | − |
| | DKFZp761B15121.1 | | | | | | | | | |
| M235 | VON EBNER'S GLAND | − | − | − | − | + | − | − | − | − |
| | PROTEIN PRECURSOR | | | | | | | | | |
| | (VEG PROTEIN) (TEAR | | | | | | | | | |
| | PREALBUMIN) (TP) | | | | | | | | | |
| | (TEAR LIPOCALIN) | | | | | | | | | |
| | (LIPOCALIN-1) | | | | | | | | | |
| M236 | RIBONUCLEASE | − | − | − | − | − | + | − | − | − |
| | PANCREATIC | | | | | | | | | |
| | PRECURSOR (RNASE 1) | | | | | | | | | |
| | (RNASE A) | | | | | | | | | |
| M240 | Carbonic Anhydrase II | − | − | + | − | − | − | − | − | − |
| | (Carbonate Dehydratase 11) | | | | | | | | | |
| | (CAII) | | | | | | | | | |
| M241 | HRV Fab 027-VL | − | − | − | ++ | ++ | − | − | − | − |
| M244 | 364K Golgi complex- | − | − | + | − | − | − | − | − | − |
| | associated protein | | | | | | | | | |
| M245 | histone H2A.5 | + | − | − | − | − | − | + | − | − |
| M246 | antibody, light chain | − | − | − | ++ | − | − | − | − | − |
| | variable regin to HIV1 p25 | | | | | | | | | |
| M248 | TRYPSINOGEN, | − | − | − | − | − | − | − | − | + |
| | CATIONIC PRECURSOR | | | | | | | | | |
| | (BETA-TRYPSIN) | | | | | | | | | |
| M249 | putative | − | − | ++ | − | − | − | − | ++ | − |
| M250 | LOW AFFINITY | − | − | ++ | − | − | − | − | − | − |
| | IMMUNOGLOBULIN | | | | | | | | | |
| | GAMMA FC REGION | | | | | | | | | |
| | RECEPTOR II-A | | | | | | | | | |
| | PRECURSOR (FC- | | | | | | | | | |
| | GAMMA RII-A) (FCRII-A) | | | | | | | | | |
| | (IGG FC RECEPTOR II-A) | | | | | | | | | |
| | (FC-GAMMA-RIIA) | | | | | | | | | |
| | (CD32) (CDW32) | | | | | | | | | |
| M252 | CYTOCHROME C | − | + | − | − | − | − | − | − | − |
| M258 | gamma-glutamyl hydrolase | − | − | + | − | − | − | − | − | − |
| | (conjugase, | | | | | | | | | |
| | folylpolygammaglutamyl | | | | | | | | | |
| | hydrolase) precursor | | | | | | | | | |
| M259 | breakpoint cluster region | − | ++ | − | − | − | − | − | − | − |
| | protein 1 | | | | | | | | | |
| M261 | BCL3 | − | − | − | − | − | − | − | − | − |
| M264 | macrophage migration | − | ++ | − | − | − | − | − | − | − |
| | inhibitory factor | | | | | | | | | |
| M266 | unnamed protein product | − | − | − | + | − | − | − | − | − |
| M267 | ribosomal protein S28, | − | + | ++ | − | + | − | − | − | − |
| | cytosolic | | | | | | | | | |
| M268 | MICROFIBRIL- | − | − | − | − | − | + | − | − | − |
| | ASSOCIATED | | | | | | | | | |
| | GLYCOPROTEIN 2 | | | | | | | | | |
| | PRECURSOR (MAGP-2) | | | | | | | | | |
| | (MP25) | | | | | | | | | |
| M269 | cathepsin S | − | ++ | − | − | − | − | − | − | − |
| M270 | lambda-chain C-region C- | − | − | − | − | − | − | − | − | − |
| | lambda-2 | | | | | | | | | |
| M271 | anti-porcine VCAM mAb | − | − | − | ++ | − | − | − | − | − |
| | 3F4 light chain variable | | | | | | | | | |
| | region | | | | | | | | | |
| M275 | CATHEPSIN D | + | + | − | − | − | − | − | − | − |
| M277 | putative | − | − | − | − | − | ++ | − | − | − |
| M278 | transaldolase | − | + | − | − | − | − | − | − | − |
| M280 | myeloid inhibitory siglec | − | − | − | − | − | − | − | − | − |
| M281 | granulocyte inhibitory | − | − | − | − | ++ | − | − | − | − |
| | protein | | | | | | | | | |
| M282 | GROWTH FACTOR | + | − | − | − | − | − | − | − | − |
| | RECEPTOR-BOUND | | | | | | | | | |
| | PROTEIN 2 (GRB2 | | | | | | | | | |
| | ADAPTER PROTEIN) | | | | | | | | | |
| | (SH2/SH3 ADAPTER | | | | | | | | | |
| | GRB2) (ASH PROTEIN) | | | | | | | | | |
| M284 | UBIQUITIN-LIKE | − | ++ | − | − | − | − | − | − | − |
| | PROTEIN SMT3B | | | | | | | | | |
| | (SENTRIN 2) | | | | | | | | | |
| M286 | HLA CLASS I | − | + | − | − | − | − | − | − | − |
| | HISTOCOMPATIBILITY | | | | | | | | | |
| | ANTIGEN, CW-1 | | | | | | | | | |
| | CW*0102 ALPHA CHAIN | | | | | | | | | |
| | PRECURSOR (CW1.2) | | | | | | | | | |
| M287 | 40S RIBOSOMAL | − | − | ++ | − | − | − | − | − | − |
| | PROTEIN S17 | | | | | | | | | |
| M289 | dJ747L4.1 (Brachyury (T | − | − | − | ++ | − | − | − | − | − |
| | box protein)-LIKE protein | | | | | | | | | |
| M290 | anti-oxidized LDL | ++ | − | − | − | ++ | − | − | − | − |
| | autoantibody variable kappa | | | | | | | | | |
| | chain | | | | | | | | | |
| M296 | similar to | − | − | − | − | ++ | − | − | − | − |
| | TROPOMYOSIN, | | | | | | | | | |
| | CYTOSKELETAL TYPE | | | | | | | | | |
| | (TM30-NM) (H. sapiens) | | | | | | | | | |
| M298 | ZYXIN (ZYXIN 2) | − | − | + | − | − | − | − | − | − |
| M300 | ENHANCER OF | − | ++ | − | − | − | − | − | − | − |
| | RUDIMENTARY | | | | | | | | | |
| | HOMOLOG | | | | | | | | | |
| M301 | regulator of G-protein | − | − | − | − | − | − | − | − | ++ |
| | signaling 4 | | | | | | | | | |
| M305 | anti-DNA immunoglobulin | − | − | − | − | − | − | − | − | − |
| | light chain IgG | | | | | | | | | |
| M307 | UBIQUITIN-LIKE | − | + | − | − | − | − | − | − | − |
| | PROTEIN NEDD8 | | | | | | | | | |
| M311 | SCRAPIE-RESPONSIVE | − | − | − | − | − | − | − | ++ | − |
| | PROTEIN 1 PRECURSOR | | | | | | | | | |
| | (SCRG-1) | | | | | | | | | |
| M312 | gonadotropin releasing | − | − | − | + | − | − | ++ | ++ | − |
| | peptide | | | | | | | | | |
| M315 | IRT-1 | − | ++ | − | − | − | − | − | − | − |
| M316 | recombinant antibody light | − | − | − | ++ | − | − | − | − | − |
| | chain VL domain | | | | | | | | | |
| M317 | putative | − | − | − | − | − | − | − | − | ++ |
| M318 | CYSTATIN M | − | − | − | − | − | − | − | ++ | − |
| | PRECURSOR (CYSTATIN | | | | | | | | | |
| | E) | | | | | | | | | |
| M320 | proteasome activator PA28 | − | − | − | − | ++ | − | − | − | − |
| | beta chain | | | | | | | | | |
| M321 | AGGRECAN CORE | − | − | − | − | − | ++ | − | − | − |
| | PROTEIN PRECURSOR | | | | | | | | | |
| | (CARTILAGE-SPECIFIC | | | | | | | | | |
| | PROTEOGLYCAN CORE | | | | | | | | | |
| | PROTEIN) (CSPCP) | | | | | | | | | |
| | (CHONDROITIN | | | | | | | | | |
| | SULFATE | | | | | | | | | |
| | PROTEOGLYCAN CORE | | | | | | | | | |
| | PROTEIN 1) | | | | | | | | | |
| M322 | KIAA0185 protein | − | − | ++ | − | − | + | − | − | − |
| M323 | hypothetical protein | − | − | − | ++ | − | − | − | − | − |
| | FLJ13465 | | | | | | | | | |
| M324 | CARBOXYPEPTIDASE N | − | − | − | − | − | − | − | − | + |
| | 83 KDA CHAIN | | | | | | | | | |
| | (CARBOXYPEPTIDASE | | | | | | | | | |
| | N REGULATORY | | | | | | | | | |
| | SUBUNIT) | | | | | | | | | |
| M326 | R33729_1 | − | − | + | − | − | − | − | − | − |
| M327 | lymphocyte surface antigen | − | ++ | − | − | − | − | − | − | − |
| | precursor CD44 | | | | | | | | | |
| M328 | PYRIDOXINE KINASE | + | − | − | − | − | − | − | − | − |
| | (PYRIDOXAL KINASE) | | | | | | | | | |
| M329 | unnamed protein product | − | − | − | − | − | + | − | − | − |
| M330 | CALPACTIN I LIGHT | − | − | ++ | − | − | − | − | − | − |
| | CHAIN (P10 PROTEIN) | | | | | | | | | |
| | (P11) (CELLULAR | | | | | | | | | |
| | LIGAND OF ANNEXIN II) | | | | | | | | | |
| | (NERVE GROWTH | | | | | | | | | |
| | FACTOR INDUCED | | | | | | | | | |
| | PROTEIN 42C) | | | | | | | | | |
| M331 | C360B4.1 (PUTATIVE | + | − | − | − | − | − | ++ | − | ++ |
| | novel protein similar to | | | | | | | | | |
| | predicted bacterial and | | | | | | | | | |
| | worm proteins) | | | | | | | | | |
| M332 | latent transforming growth | − | − | − | − | − | − | − | ++ | − |
| | factor-beta binding protein | | | | | | | | | |
| | 4S | | | | | | | | | |
| M333 | hypothetical protein | − | − | − | − | − | − | − | − | − |
| | XP_047083 | | | | | | | | | |
| M334 | similar to | ++ | ++ | ++ | − | − | − | + | − | − |
| | GASTRIN/CHOLECYSTOKININ | | | | | | | | | |
| | TYPE B | | | | | | | | | |
| | RECEPTOR (CCK-B | | | | | | | | | |
| | RECEPTOR) (CCK-BR) | | | | | | | | | |
| | (H. sapiens) | | | | | | | | | |
| M336 | similar to LINE-1 | − | − | − | − | − | − | − | − | − |
| | REVERSE | | | | | | | | | |
| | TRANSCRIPTASE | | | | | | | | | |
| | HOMOLOG (H. sapiens) | | | | | | | | | |
| M337 | TYROSINE-PROTEIN | − | − | − | − | − | − | ++ | − | ++ |
| | KINASE CSK (C-SRC | | | | | | | | | |
| | KINASE) | | | | | | | | | |
| M338 | Similar to dystroglycan 1 | − | − | − | − | − | − | − | ++ | − |
| | (dystrophin-associated | | | | | | | | | |
| | glycoprotein 1) | | | | | | | | | |
| M339 | complement component 1, | − | − | − | − | − | ++ | − | − | − |
| | s subcomponent | | | | | | | | | |
| M340 | lysophospholipase II | − | − | − | − | − | − | − | − | − |
| M344 | TELOMERASE-BINDING | + | − | − | − | − | − | − | − | − |
| | PROTEIN P23 (HSP90 CO- | | | | | | | | | |
| | CHAPERONE) | | | | | | | | | |
| | (PROGESTERONE | | | | | | | | | |
| | RECEPTOR COMPLEX | | | | | | | | | |
| | P23) | | | | | | | | | |
| M346 | inwardly rectifying | − | − | − | − | − | − | ++ | − | − |
| | potassium channel protein | | | | | | | | | |
| | Kir6.2 | | | | | | | | | |
| M349 | dnaJ protein homolog | − | − | − | ++ | − | − | − | − | − |
| M350 | 40S RIBOSOMAL | − | ++ | − | − | − | − | − | − | − |
| | PROTEIN S29 | | | | | | | | | |
| M352 | MYOGLOBIN | − | − | − | − | − | − | − | − | − |
| M353 | cathepsin X precursor | − | ++ | − | − | − | − | − | − | − |
| M354 | unknown | − | ++ | − | − | − | − | − | − | − |
| M356 | HYPOTHETICAL | − | − | − | − | − | − | + | − | − |
| | PROTEIN CGI-109 | | | | | | | | | |
| | PRECURSOR | | | | | | | | | |
| M357 | LITHOSTATHINE 1 | + | − | − | − | − | + | − | − | − |
| | BETA PRECURSOR | | | | | | | | | |
| | (REGENERATING | | | | | | | | | |
| | PROTEIN I BETA) | | | | | | | | | |
| M358 | human leucocyte antigen B | − | − | − | − | − | − | − | − | − |
| M360 | This CDS feature is | − | − | − | − | ++ | − | − | − | − |
| | included to show the | | | | | | | | | |
| | translation of the | | | | | | | | | |
| | corresponding V_region. | | | | | | | | | |
| | Presently translation | | | | | | | | | |
| | qualifiers on V_region | | | | | | | | | |
| | features are illegal. | | | | | | | | | |
| M362 | KIAA0336 | ++ | − | − | ++ | − | − | − | − | − |
| M363 | Similar to expressed | − | − | − | − | − | − | − | − | − |
| | sequence 2 embryonic lethal | | | | | | | | | |
| M366 | NEUROBLASTOMA | − | − | − | + | − | − | − | − | − |
| | SUPPRESSOR OF | | | | | | | | | |
| | TUMORIGENICITY 1 | | | | | | | | | |
| | (ZINC FINGER PROTEIN | | | | | | | | | |
| | DAN) (N03) | | | | | | | | | |
| M370 | putative | − | − | − | − | − | ++ | − | − | − |
| M372 | alpha 1F calcium channel | − | − | − | − | − | − | − | − | + |
| | subunit | | | | | | | | | |
| M373 | sa1 (Drosophila)-like 2 | − | − | − | − | − | + | − | − | − |
| M374 | AF15q14 protein | − | − | − | ++ | − | − | − | − | − |
| M375 | putative | − | − | − | − | − | − | − | − | − |
| M376 | fibroblast growth factor 13 | − | − | − | +++ | ++ | ++ | − | − | ++ |
| | isoform lylv | | | | | | | | | |
| M379 | KIAA0433 | + | + | − | − | − | − | − | − | − |
| M380 | anti-c-erbB-2 | − | − | − | − | − | ++ | − | − | − |
| | immunoglobulin light chain | | | | | | | | | |
| | V | | | | | | | | | |
| M381 | ATP-BINDING | − | − | − | ++ | − | − | − | − | − |
| | CASSETTE, SUB- | | | | | | | | | |
| | FAMILY A, MEMBER 2 | | | | | | | | | |
| | (ATP-BINDING | | | | | | | | | |
| | CASSETTE | | | | | | | | | |
| | TRANSPORTER 2) (ATP- | | | | | | | | | |
| | BINDING CASSETTE 2) | | | | | | | | | |
| M382 | G protein-coupled receptor | − | − | − | ++ | − | − | − | − | − |
| | kinase-associated ADP | | | | | | | | | |
| | ribosylation factor GTPase- | | | | | | | | | |
| | activating protein | | | | | | | | | |
| M387 | farnesyl-protein transferase | − | − | ++ | − | + | ++ | ++ | ++ | ++ |
| | beta chain | | | | | | | | | |
| M388 | KIAA1813 protein | − | − | − | − | ++ | − | − | − | − |
| M389 | hypothetical protein | − | − | − | − | − | − | − | − | ++ |
| | FLJ11937 | | | | | | | | | |
| M391 | ribosomal protein L29 | − | ++ | − | − | + | − | − | − | − |
| M393 | inhibitor PI, alpha1 | − | − | + | ++ | − | − | − | − | − |
| | proteinase | | | | | | | | | |
| M394 | dJ467L1.3 (period | − | − | − | − | − | − | − | ++ | − |
| | (Drosophila) homolog 3) | | | | | | | | | |
| M395 | cylindromatosis (turban | − | − | − | ++ | − | − | − | − | − |
| | tumor syndrome) | | | | | | | | | |
| M396 | T cell receptor alpha chain | − | − | − | − | − | − | − | − | ++ |
| | V region (clone 2V alpha | | | | | | | | | |
| | 23-2) | | | | | | | | | |
| M399 | anti-pneumococcal Ig L- | − | − | − | − | − | − | ++ | − | − |
| | chain Fab fragment | | | | | | | | | |
| M401 | Unknown (protein for | − | − | − | − | − | + | − | − | − |
| | MGC: 16498) | | | | | | | | | |
| M403 | plasma protease (C1) | − | − | − | − | − | − | − | − | + |
| | inhibitor precursor | | | | | | | | | |
| M404 | similar to INITIATION | − | − | − | − | − | − | − | − | − |
| | FACTOR 5A (EIF-5A) | | | | | | | | | |
| | (EIF-4D) (REV-BINDING | | | | | | | | | |
| | FACTOR) (H. sapiens) | | | | | | | | | |
| M406 | CGI-202 | + | − | − | − | − | − | − | − | − |
| M409 | alternatively spliced | − | − | − | − | − | ++ | − | − | − |
| M410 | neuronal leucine-rich | − | − | − | ++ | − | − | − | − | − |
| | repeat protein-3 | | | | | | | | | |
| M411 | Unknown (protein for | − | − | − | − | − | − | − | − | + |
| | IMAGE: 3587716) | | | | | | | | | |
| M412 | PROTEASOME | − | ++ | − | − | − | − | − | − | − |
| | SUBUNIT ALPHA TYPE 1 | | | | | | | | | |
| | (PROTEASOME | | | | | | | | | |
| | COMPONENT C2) | | | | | | | | | |
| | (MACROPAIN SUBUNIT | | | | | | | | | |
| | C2) (MULTICATALYTIC | | | | | | | | | |
| | ENDOPEPTIDASE | | | | | | | | | |
| | COMPLEX SUBUNIT C2) | | | | | | | | | |
| | (PROTEASOME NU | | | | | | | | | |
| | CHAIN) | | | | | | | | | |
| M413 | NIF-like protein | ++ | − | − | − | − | − | − | − | − |
| M415 | unnamed protein product | − | ++ | + | − | − | − | − | − | + |
| M417 | hairy/enhancer of split 6 | + | − | − | − | − | + | − | − | + |
| M419 | sperm tail associated | − | − | − | − | − | − | − | − | − |
| | protein | | | | | | | | | |
| M420 | NEURABIN-II (NEURAL | − | ++ | ++ | ++ | − | − | − | − | − |
| | TISSUE-SPECIFIC F- | | | | | | | | | |
| | ACTIN BINDING | | | | | | | | | |
| | PROTEIN II) | | | | | | | | | |
| | (SPINOPHILIN) (P130) | | | | | | | | | |
| | (PP1BP134) | | | | | | | | | |
| M421 | TFNR | − | + | − | − | − | − | − | − | − |
| M422 | TROPOMYOSIN 4, | − | ++ | − | − | − | − | − | − | − |
| | EMBRYONIC | | | | | | | | | |
| | FIBROBLAST ISOFORM | | | | | | | | | |
| | (TM-4) | | | | | | | | | |
| M423 | ZINC FINGER Y- | − | − | − | − | − | − | + | − | − |
| | CHROMOSOMAL | | | | | | | | | |
| | PROTEIN | | | | | | | | | |
| M424 | CD27L RECEPTOR | − | − | − | − | − | − | − | − | − |
| | PRECURSOR (T-CELL | | | | | | | | | |
| | ACTIVATION ANTIGEN | | | | | | | | | |
| | CD27) (T14) | | | | | | | | | |
| M425 | FIBROLEUKIN | − | + | − | − | − | − | − | − | − |
| | PRECURSOR | | | | | | | | | |
| | (FIBRINOGEN-LIKE | | | | | | | | | |
| | PROTEIN 2) (PT49) | | | | | | | | | |
| M428 | putative | − | − | − | ++ | ++ | + | − | − | − |
| M429 | unnamed protein product | − | − | − | − | − | ++ | − | − | − |
| M431 | hypothetical protein | − | − | − | ++ | − | − | − | − | − |
| M433 | myosin-VIIb | − | − | − | − | − | − | − | − | − |
| M435 | hypothetical protein | ++ | ++ | + | − | − | − | ++ | − | − |
| | FLJ20516 | | | | | | | | | |
| M436 | similar to ribosomal protein | − | − | − | − | − | − | + | − | − |
| | S26 (H. sapiens) | | | | | | | | | |
| M439 | C-terminus matches | − | − | − | − | − | ++ | − | − | − |
| | KIAA0559, N-terminus | | | | | | | | | |
| | similar to Bassoon protein | | | | | | | | | |
| M441 | arfaptin 1 | ++ | − | − | − | − | ++ | + | − | − |
| M442 | apolipoprotein CI | − | − | − | − | − | ++ | − | − | − |
| M443 | dJ591C20.1 (novel protein | − | − | − | − | − | − | − | − | + |
| | similar to mouse NG26) | | | | | | | | | |
| M444 | VESICLE TRANSPORT | − | − | − | ++ | − | − | − | − | − |
| | V-SNARE PROTEIN | | | | | | | | | |
| | VTI1-LIKE 1 (VTI1-RP1) | | | | | | | | | |
| M445 | retinoblastoma-associated | + | − | − | − | − | − | − | − | − |
| | protein HEC | | | | | | | | | |
| M446 | ZINC FINGER PROTEIN | ++ | − | − | − | − | − | − | − | − |
| | GFI-1 (GROWTH | | | | | | | | | |
| | FACTOR | | | | | | | | | |
| | INDEPENDENCE-1) | | | | | | | | | |
| M447 | 5- | − | − | − | − | − | − | − | − | − |
| | HYDROXYTRYPTAMINE | | | | | | | | | |
| | 5A RECEPTOR (5-HT- | | | | | | | | | |
| | 5A) (SEROTONIN | | | | | | | | | |
| | RECEPTOR) (REC17) | | | | | | | | | |
| M448 | This CDS feature is | − | − | − | ++ | − | − | − | − | − |
| | included to show the | | | | | | | | | |
| | translation of the | | | | | | | | | |
| | corresponding V_region. | | | | | | | | | |
| | Presently translation | | | | | | | | | |
| | qualifiers on V_region | | | | | | | | | |
| | features are illegal | | | | | | | | | |
| M451 | KIAA1664 protein | ++ | − | − | − | − | − | − | − | − |
| M452 | METALLOTHIONEIN-II | − | − | + | − | − | − | − | − | − |
| | (MT-II) | | | | | | | | | |
| M453 | microseminoprotein beta | − | − | − | − | − | − | − | ++ | − |
| M454 | SACSIN | ++ | − | − | − | − | − | − | − | − |
| M455 | tenascin-R | − | − | − | − | ++ | − | − | − | − |
| M457 | SERINE PROTEASE | − | − | − | ++ | − | − | − | − | − |
| | INHIBITOR KAZAL- | | | | | | | | | |
| | TYPE 5 PRECURSOR | | | | | | | | | |
| | (LYMPHO-EPITHELIAL | | | | | | | | | |
| | KAZAL-TYPE RELATED | | | | | | | | | |
| | INHIBITOR) (LEKTI) | | | | | | | | | |
| | [CONTAINS: | | | | | | | | | |
| | HEMOFILTRATE | | | | | | | | | |
| | PEPTIDE HF6478 | | | | | | | | | |
| M458 | CALGRANULIN C | − | + | − | − | − | − | − | − | − |
| | (CAGC) | | | | | | | | | |
| M459 | galactose-specific lectin | + | − | − | − | − | − | − | − | − |
| M460 | non-muscle myosin heavy | − | − | − | ++ | − | − | − | − | − |
| | chain | | | | | | | | | |
| M461 | DOCK180 protein | − | − | ++ | − | − | − | − | − | − |
| M462 | serine protease | − | − | − | − | − | − | − | ++ | − |
| M463 | nuclear zinc finger protein | − | − | + | − | − | − | − | − | − |
| | Np95 | | | | | | | | | |
| M464 | LIM protein (similar to rat | − | − | − | ++ | − | − | − | − | − |
| | protein kinase C-binding | | | | | | | | | |
| | enigma) | | | | | | | | | |
| M465 | SECIS binding protein 2 | − | − | ++ | − | − | − | − | − | − |
| M466 | HSPC055 protein | − | − | + | − | − | − | + | − | − |
| M468 | ribosomal protein L14 | − | − | − | − | − | − | + | − | − |
| M470 | chloride channel protein 3, | − | − | − | − | − | − | − | − | ++ |
| | long form | | | | | | | | | |
| M471 | MITOGEN-ACTIVATED | − | − | − | ++ | − | − | − | − | − |
| | PROTEIN KINASE | | | | | | | | | |
| | KINASE KINASE 3 | | | | | | | | | |
| | (MAPK/ERK KINASE | | | | | | | | | |
| | KINASE 3) (MEK | | | | | | | | | |
| | KINASE 3) (MEKK 3) | | | | | | | | | |
| M472 | unnamed protein product | − | − | − | − | − | − | − | − | + |
| M473 | anti-HIV gp120 antibody | − | − | − | − | − | − | − | − | ++ |
| | light chain variable region | | | | | | | | | |
| M474 | CARBONIC | − | − | − | − | − | − | − | − | + |
| | ANHYDRASE IV | | | | | | | | | |
| | PRECURSOR | | | | | | | | | |
| | (CARBONATE | | | | | | | | | |
| | DEHYDRATASE IV) (CA- | | | | | | | | | |
| | IV) | | | | | | | | | |
| M475 | hypothetical protein | − | − | + | − | − | − | − | − | − |
| | FLJ11016 | | | | | | | | | |
| M476 | putative gene with | − | − | + | − | − | − | − | − | − |
| | similarities to KIAA1074 | | | | | | | | | |
| | and KIAA0565 | | | | | | | | | |
| M477 | S-MYC PROTO- | + | − | − | − | − | − | − | − | − |
| | ONCOGENE PROTEIN | | | | | | | | | |
| M480 | unnamed protein product | − | − | − | ++ | − | − | − | − | − |
| M483 | proapolipoprotein Varient | − | − | + | − | + | − | − | − | − |
| M484 | Unknown | − | − | + | + | − | − | − | − | − |
| M485 | neutrophil lactoferrin | − | − | + | − | − | − | − | − | − |
| M486 | alpha-2-HS glycoprotein | − | − | − | − | − | − | − | − | + |
| | Varient | | | | | | | | | |
| M487 | MT-11 protein Varient | − | + | − | − | − | − | − | − | − |
| M488 | immunoglobulin lambda | − | − | + | − | − | − | − | − | − |
| | light chain homolog | | | | | | | | | |
| M489 | Unknown | − | − | − | − | − | − | − | − | + |
| M490 | Unknown | − | − | − | − | − | − | + | − | − |

| | | | | | SEQ | SEQ |
|---|---|---|---|---|---|---|
| | | | | | ID | ID |
| | | N_66 | MW | GI | NO | NO |
| Marker | Gene Name | Br39 | (Da) | number | (nts) | (AA) |
| M4 | COMPLEMENT C3 | +++ | 187165 | 4557385 | | |
| | PRECURSOR | | | | | |
| | [CONTAINS: C3A | | | | | |
| | ANAPHYLATOXIN] | | | | | |
| M5 | proapolipoprotein | +++ | 28961.7 | 178775 | | |
| M7 | FIBRINOGEN | +++ | 94973.5 | 4503689 | | |
| | ALPHA/ALPHA-E CHAIN | | | | | |
| | PRECURSOR | | | | | |
| M8 | FIBRINOGEN BETA | ++ | 55928.5 | 399492 | | |
| | CHAIN PRECURSOR | | | | | |
| M10 | TRANSTHYRETIN | +++ | 15887.1 | 4507725 | | |
| | PRECURSOR | | | | | |
| | (PREALBUMIN) (TBPA) | | | | | |
| | (TTR) (ATTR) | | | | | |
| M12 | apolipoprotein A-IV | ++ | 43384.7 | 178779 | | |
| | precursor | | | | | |
| M13 | alpha 1-acid glycoprotein | +++ | 23366.1 | 1197209 | | |
| M14 | hemopexin | ++ | 49295.7 | 1335098 | | |
| M15 | macroglobulin alpha2 | ++ | 160807 | 224053 | | |
| M17 | alpha-2-glycoprotein 1, | +++ | 38194.4 | 14749011 | | |
| | zinc | | | | | |
| M21 | ANTITHROMBIN-III | ++ | 52602.7 | 4502261 | | |
| | PRECURSOR (ATIII) | | | | | |
| M22 | AMBP PROTEIN | +++ | 38999.7 | 4502067 | | |
| | PRECURSOR | | | | | |
| | [CONTAINS: ALPHA-1- | | | | | |
| | MICROGLOBULIN | | | | | |
| | (PROTEIN HC) | | | | | |
| | (COMPLEX-FORMING | | | | | |
| | GLYCOPROTEIN | | | | | |
| | HETEROGENEOUS IN | | | | | |
| | CHARGE) | | | | | |
| M23 | PROTHROMBIN | ++ | 70037.3 | 4503635 | | |
| | PRECURSOR | | | | | |
| | (COAGULATION | | | | | |
| | FACTOR II) | | | | | |
| M24 | ALPHA-2-HS- | +++ | 39324.9 | 4502005 | | |
| | GLYCOPROTEIN | | | | | |
| | PRECURSOR (FETUIN-A) | | | | | |
| | (ALPHA-2-Z-GLOBULIN) | | | | | |
| | (BA-ALPHA-2- | | | | | |
| | GLYCOPROTEIN) | | | | | |
| M25 | actin beta | ++ | 41737 | 4501885 | | |
| M26 | CLUSTERIN | ++ | 52494.9 | 4502905 | | |
| | PRECURSOR | | | | | |
| | (COMPLEMENT- | | | | | |
| | ASSOCIATED PROTEIN | | | | | |
| | SP-40, 40) | | | | | |
| | (COMPLEMENT | | | | | |
| | CYTOLYSIS INHIBITOR) | | | | | |
| | (CLI) (NA1 AND NA2) | | | | | |
| | (APOLIPOPROTEIN J) | | | | | |
| | (APO-J) (TRPM-2) | | | | | |
| M27 | bA120D12.1 (CD5 antigen- | ++ | 38088.1 | 5174411 | | |
| | like (scavenger receptor | | | | | |
| | cysteine rich family)) | | | | | |
| M28 | fibrinogen gamma-B chain | ++ | 51511.9 | 71828 | | |
| | precursor | | | | | |
| M29 | polyubiquitin 4 | − | 30303.9 | 2118964 | | |
| M30 | KININOGEN | − | 71945.7 | 125507 | | |
| | PRECURSOR (ALPHA-2- | | | | | |
| | THIOL PROTEINASE | | | | | |
| | INHIBITOR) [CONTAINS: | | | | | |
| | BRADYKININ] | | | | | |
| M31 | CALGRANULIN B | ++ | 13242.1 | 4506773 | | |
| | (MIGRATION | | | | | |
| | INHIBITORY FACTOR- | | | | | |
| | RELATED PROTEIN 14) | | | | | |
| | (MRP-14) (P14) | | | | | |
| | (LEUKOCYTE L1 | | | | | |
| | COMPLEX HEAVY | | | | | |
| | CHAIN) (S100 CALCIUM- | | | | | |
| | BINDING PROTEIN A9) | | | | | |
| M32 | PLASMA RETINOL- | +++ | 22868 | 5803139 | | |
| | BINDING PROTEIN | | | | | |
| | PRECURSOR (PRBP) | | | | | |
| | (RBP) | | | | | |
| M33 | Ig mu chain precursor, | ++ | 68510.5 | 87919 | | |
| | membrane-bound (clone | | | | | |
| | 201) | | | | | |
| M34 | APOLIPOPROTEIN A-II | +++ | 11175.1 | 4502149 | | |
| | PRECURSOR (APO-AII) | | | | | |
| M35 | LEUCINE-RICH ALPHA- | ++ | 34346.6 | 112908 | | |
| | 2-GLYCOPROTEIN (LRG) | | | | | |
| M36 | dJ34F7.4 (complement | ++ | 192753 | 7671645 | | |
| | component 4A) | | | | | |
| M37 | similar to phosphoglycerate | ++ | 28850.2 | 15301114 | | |
| | mutase 1 (brain) | | | | | |
| M38 | alpha-1-antichymotrypsin | ++ | 45482.4 | 177933 | | |
| | precursor | | | | | |
| M39 | ALPHA-1B- | ++ | 51941 | 112892 | | |
| | GLYCOPROTEIN | | | | | |
| M41 | lipoprotein CIII | ++ | 8764.7 | 224917 | | |
| M42 | TETRANECTIN | +++ | 22567 | 4507557 | | |
| | PRECURSOR (TN) | | | | | |
| | (PLASMINOGEN- | | | | | |
| | KRINGLE 4 BINDING | | | | | |
| | PROTEIN) | | | | | |
| M43 | amyloid related serum | + | 11682.8 | 4506777 | | |
| | protein SAA | | | | | |
| M44 | CALGRANULIN A | + | 10834.6 | 14729628 | | |
| | (MIGRATION | | | | | |
| | INHIBITORY FACTOR- | | | | | |
| | RELATED PROTEIN 8) | | | | | |
| | (MRP-8) (CYSTIC | | | | | |
| | FIBROSIS ANTIGEN) | | | | | |
| | (CFAG) (P8) | | | | | |
| | (LEUKOCYTE L1 | | | | | |
| | COMPLEX LIGHT | | | | | |
| | CHAIN) (S100 CALCIUM- | | | | | |
| | BINDING PROTEIN A8) | | | | | |
| M45 | C-REACTIVE PROTEIN | ++ | 25038.7 | 14728083 | | |
| | PRECURSOR | | | | | |
| M46 | RHO GDP- | ++ | 22988.1 | 10835002 | | |
| | DISSOCIATION | | | | | |
| | INHIBITOR 2 (RHO GDI | | | | | |
| | 2) (RHO-GDI BETA) (LY- | | | | | |
| | GDI) | | | | | |
| M47 | PROFILIN I | ++ | 15054.3 | 4826898 | | |
| M49 | APOLIPOPROTEIN D | ++ | 21275.7 | 4502163 | | |
| | PRECURSOR | | | | | |
| M50 | inter-alpha-trypsin inhibitor | − | 103373 | 4096840 | | |
| | family heavy chain-related | | | | | |
| | protein | | | | | |
| M51 | 14-3-3 PROTEIN | ++ | 28082.5 | 4507949 | | |
| | BETA/ALPHA (PROTEIN | | | | | |
| | KINASE C INHIBITOR | | | | | |
| | PROTEIN-1) (KCIP-1) | | | | | |
| | (PROTEIN 1054) | | | | | |
| M52 | Ig G1 H Nie | − | 49207.8 | 229601 | | |
| M53 | OSTEOPONTIN | − | 35422.9 | 14724978 | | |
| | PRECURSOR (BONE | | | | | |
| | SIALOPROTEIN 1) | | | | | |
| | (URINARY STONE | | | | | |
| | PROTEIN) (SECRETED | | | | | |
| | PHOSPHOPROTEIN 1) | | | | | |
| | (SPP-1) | | | | | |
| | (NEPHROPONTIN) | | | | | |
| | (UROPONTIN) | | | | | |
| M54 | AFAMIN PRECURSOR | ++ | 69069.6 | 4501987 | | |
| | (ALPHA-ALBUMIN) | | | | | |
| | (ALPHA-ALB) | | | | | |
| M55 | TRIOSEPHOSPHATE | ++ | 26669.6 | 4507645 | | |
| | ISOMERASE (TIM) | | | | | |
| M56 | pre-serum amyloid P | − | 25397.3 | 337758 | | |
| | component | | | | | |
| M57 | COMP_HUMAN | − | 89149 | 2623750 | | |
| M58 | prosaposin (variant | − | 50307.8 | 15298143 | | |
| | Gaucher disease and variant | | | | | |
| | metachromatic | | | | | |
| | leukodystrophy) | | | | | |
| M59 | VITRONECTIN | ++ | 54305.9 | 14774022 | | |
| | PRECURSOR (SERUM | | | | | |
| | SPREADING FACTOR) | | | | | |
| | (S-PROTEIN) | | | | | |
| | [CONTAINS: | | | | | |
| | SOMATOMEDIN B] | | | | | |
| M60 | CALGRANULIN C (S100 | − | 10443.9 | 2146972 | | |
| | A12 protein) | | | | | |
| M61 | cofilin 1 (non-muscle) | +++ | 16811.7 | 14784011 | | |
| M62 | cathepsin B | − | 37821.8 | 4503139 | | |
| M63 | plasmin (EC 3.4.21.7) | + | 90568.6 | 625234 | | |
| | precursor [validated] | | | | | |
| M64 | PLASMA | ++ | 25505.6 | 121672 | | |
| | GLUTATHIONE | | | | | |
| | PEROXIDASE | | | | | |
| | PRECURSOR (GSHPX-P) | | | | | |
| M65 | lumican | ++ | 38429.2 | 4505047 | | |
| M66 | apolipoprotein C-II | − | 10183.5 | 2134777 | | |
| M67 | calmodulin 2 | ++ | 16836.7 | 14250065 | | |
| | (phosphorylase kinase, | | | | | |
| | delta) | | | | | |
| M68 | THYMOSIN BETA-4 | ++ | 5062.7 | 14730886 | | |
| M69 | uracil DNA glycosylase | − | 35492.9 | 35053 | | |
| M70 | defensin alpha-3 precursor, | ++ | 10245 | 4885179 | | |
| | neutrophil-specific | | | | | |
| | [validated] | | | | | |
| M71 | CD14 antigen | − | 40076.4 | 4557417 | | |
| M72 | peptidylprolyl isomerase | ++ | 19008.7 | 12804335 | | |
| | (EC 5.2.1.8) A | | | | | |
| M73 | similar to transgelin 2 (H. | − | 24454 | 14728128 | | |
| | sapiens) | | | | | |
| M74 | BETA-2- | − | 38298.4 | 14771355 | | |
| | GLYCOPROTEIN I | | | | | |
| | PRECURSOR | | | | | |
| | (APOLIPOPROTEIN H) | | | | | |
| | (APO-H) (B2GPI) | | | | | |
| | (BETA(2)GPI) | | | | | |
| | (ACTIVATED PROTEIN | | | | | |
| | C-BINDING PROTEIN) | | | | | |
| | (APC INHIBITOR) | | | | | |
| M75 | complement 9 | − | 60398.5 | 2258128 | | |
| M76 | alpha2 plasmin inhibitor | − | 54596.1 | 11386143 | | |
| M77 | FICOLIN 3 PRECURSOR | − | 32889.1 | 4504331 | | |
| | (COLLAGEN/FIBRINOGEN | | | | | |
| | DOMAIN- | | | | | |
| | CONTAINING PROTEIN | | | | | |
| | 3) | | | | | |
| | (COLLAGEN/FIBRINOGEN | | | | | |
| | DOMAIN- | | | | | |
| | CONTAINING LECTIN 3 | | | | | |
| | P35) (Hakata Antigen) | | | | | |
| M78 | glutathione transferase | − | 23463.2 | 14766346 | | |
| M81 | complement factor B | − | 85505.3 | 4502397 | | |
| M82 | CALGIZZARIN (S100C | − | 11740.5 | 5032057 | | |
| | PROTEIN) (MLN 70) | | | | | |
| M83 | BETA-2- | ++ | 13714.6 | 4757826 | | |
| | MICROGLOBULIN | | | | | |
| | PRECURSOR | | | | | |
| M84 | CYSTEINE-RICH | − | 27630.5 | 5174675 | | |
| | SECRETORY PROTEIN-3 | | | | | |
| | PRECURSOR (CRISP-3) | | | | | |
| | (SGP28 PROTEIN) | | | | | |
| M86 | megakaryocyte stimulating | ++ | 151092 | 5031925 | | |
| | factor | | | | | |
| M87 | inter-alpha-trypsin inhibitor | ++ | 101388 | 478685 | | |
| | heavy chain H1 precursor | | | | | |
| M88 | keratin 9, cytoskeletal | + | 62129.7 | 1082558 | | |
| M89 | FERRITIN HEAVY | + | 21225.8 | 14784648 | | |
| | CHAIN (FERRITIN H | | | | | |
| | SUBUNIT) | | | | | |
| M90 | HISTONE H1.1 | + | 21734.2 | 356168 | | |
| M91 | keratin 1 | + | 66067 | 11935049 | | |
| M92 | ferritin light subunit | − | 16394.7 | 182516 | | |
| M93 | PROTEASOME | − | 25153.9 | 12804095 | | |
| | SUBUNIT ALPHA TYPE 2 | | | | | |
| | (PROTEASOME | | | | | |
| | COMPONENT C3) | | | | | |
| | (MACROPAIN SUBUNIT | | | | | |
| | C3) (MULTICATALYTIC | | | | | |
| | ENDOPEPTIDASE | | | | | |
| | COMPLEX SUBUNIT C3) | | | | | |
| M94 | CARBONIC | ++ | 28870.3 | 4502517 | | |
| | ANHYDRASE I | | | | | |
| | (CARBONATE | | | | | |
| | DEHYDRATASE I) (CA-I) | | | | | |
| M95 | LYMPHOCYTE- | − | 37191.8 | 10880979 | | |
| | SPECIFIC PROTEIN LSP1 | | | | | |
| | (PP52 PROTEIN) (52 KDA | | | | | |
| | PHOSPHOPROTEIN) | | | | | |
| | (LYMPHOCYTE- | | | | | |
| | SPECIFIC ANTIGEN | | | | | |
| | WP34) | | | | | |
| M96 | apolipoprotein F | − | 35399.7 | 4502165 | | |
| M98 | OSTEOINDUCTIVE | ++ | 33922.4 | 7661704 | | |
| | FACTOR PRECURSOR | | | | | |
| | (OIF) (OSTEOGLYCIN) | | | | | |
| | (MIMECAN) | | | | | |
| M99 | APOLIPOPROTEIN E | − | 36154.3 | 4557325 | | |
| | PRECURSOR (APO-E) | | | | | |
| M100 | PHOSPHATIDYLETHANOL- | + | 21056.9 | 4505621 | | |
| | AMINE-BINDING | | | | | |
| | PROTEIN (PEBP) | | | | | |
| | (NEUROPOLYPEPTIDE | | | | | |
| | H3) (HIPPOCAMPAL | | | | | |
| | CHOLINERGIC | | | | | |
| | NEUROSTIMULATING | | | | | |
| | PEPTIDE) (HCNP) (RAF | | | | | |
| | KINASE INHIBITOR | | | | | |
| | PROTEIN) (RKIP) | | | | | |
| M101 | SUPEROXIDE | ++ | 15935.8 | 4507149 | | |
| | DISMUTASE [CU—ZN] | | | | | |
| M102 | HISTONE H1A (H1.1) | − | 22178.7 | 121916 | | |
| M103 | neutrophil lipocalin | − | 20547.6 | 4261868 | | |
| M104 | serum | − | 39731.5 | 14752059 | | |
| | paraoxonasearylesterase 1 | | | | | |
| M105 | CYTIDINE DEAMINASE | − | 16184.8 | 11386157 | | |
| | (CYTIDINE | | | | | |
| | AMINOHYDROLASE) | | | | | |
| M106 | CYSTATIN B (LIVER | + | 11139.6 | 4503117 | | |
| | THIOL PROTEINASE | | | | | |
| | INHIBITOR) (CPI-B) | | | | | |
| | (STEFIN B) | | | | | |
| M108 | angiotensinogen (serine (or | − | 53114.4 | 15079348 | | |
| | cysteine) proteinase | | | | | |
| | inhibitor, clade A (alpha-1 | | | | | |
| | antiproteinase, antitrypsin), | | | | | |
| | member 8) | | | | | |
| M109 | COLLAGEN ALPHA | ++ | 343554 | 4758028 | | |
| | 3 (VI) CHAIN | | | | | |
| | PRECURSOR | | | | | |
| M110 | beta galactoside binding | − | 14584.6 | 227920 | | |
| | lectin | | | | | |
| M111 | SH3BGRL3-like protein | − | 10437.8 | 13775198 | | |
| M112 | proteoglycan link protein 2 | − | 40165.8 | 4503053 | | |
| M113 | LYSOZYME C | − | 16537.1 | 4557894 | | |
| | PRECURSOR (1,4-BETA- | | | | | |
| | N- | | | | | |
| | ACETYLMURAMIDASE | | | | | |
| | C) | | | | | |
| M114 | cysteine-rich secreted A12- | − | 11419.5 | 9966777 | | |
| | alpha-like protein 2 | | | | | |
| M115 | LEUKOCYTE | − | 42742 | 13489087 | | |
| | ELASTASE INHIBITOR | | | | | |
| | (LEI) | | | | | |
| | (MONOCYTE/NEUTROPHIL | | | | | |
| | ELASTASE | | | | | |
| | INHIBITOR) (M/NEI) (EI) | | | | | |
| M116 | INSULIN-LIKE | + | 27934.2 | 13653947 | | |
| | GROWTH FACTOR | | | | | |
| | BINDING PROTEIN 4 | | | | | |
| | PRECURSOR (IGFBP-4) | | | | | |
| | (IBP-4) (IGF-BINDING | | | | | |
| | PROTEIN 4) | | | | | |
| M117 | LOW AFFINITY | ++ | 29089.3 | 12056967 | | |
| | IMMUNOGLOBULIN | | | | | |
| | GAMMA FC REGION | | | | | |
| | RECEPTOR III-A | | | | | |
| | PRECURSOR (IGG FC | | | | | |
| | RECEPTOR III-2) (FC- | | | | | |
| | GAMMA RIII-ALPHA) | | | | | |
| | (FC-GAMMA RIIIA) | | | | | |
| | (FCRIIIA) (FC-GAMMA | | | | | |
| | RIII) (FCRIII) (CD16-A) | | | | | |
| | (FCR-10) | | | | | |
| M118 | sex hormone-binding | + | 37488 | 14770624 | | |
| | globulin | | | | | |
| M119 | ANNEXIN I | − | 38714.5 | 4502101 | | |
| | (LIPOCORTIN I) | | | | | |
| | (CALPACTIN II) | | | | | |
| | (CHROMOBINDIN 9) | | | | | |
| | (P35) (PHOSPHOLIPASE | | | | | |
| | A2 INHIBITORY | | | | | |
| | PROTEIN) | | | | | |
| M120 | MnSOD precursor | + | 24721.2 | 34711 | | |
| M121 | EPIDIDYMAL | − | 16570.3 | 5453678 | | |
| | SECRETORY PROTEIN | | | | | |
| | E1 PRECURSOR | | | | | |
| | (NIEMANN-PICK | | | | | |
| | DISEASE TYPE C2 | | | | | |
| | PROTEIN) (EPI-1) (HE1) | | | | | |
| | (EPIDIDYMAL | | | | | |
| | SECRETORY PROTEIN | | | | | |
| | 14.6) (ESP14.6) | | | | | |
| M123 | metastasin | − | 11846.7 | 4506765 | | |
| M124 | inter-alpha (globulin) | − | 106464 | 14742977 | | |
| | inhibitor, H2 polypeptide | | | | | |
| M125 | alpha-1 type I collagen | ++ | 55060.9 | 179594 | | |
| M126 | GELSOLIN | ++ | 85697.9 | 4504165 | | |
| | PRECURSOR, PLASMA | | | | | |
| | (ACTIN- | | | | | |
| | DEPOLYMERIZING | | | | | |
| | FACTOR) (ADF) | | | | | |
| | (BREVIN) (AGEL) | | | | | |
| M127 | PROTEASOME | − | 29484 | 4506185 | | |
| | SUBUNIT ALPHA TYPE 4 | | | | | |
| | (PROTEASOME | | | | | |
| | COMPONENT C9) | | | | | |
| | (MACROPAIN SUBUNIT | | | | | |
| | C9) (MULTICATALYTIC | | | | | |
| | ENDOPEPTIDASE | | | | | |
| | COMPLEX SUBUNIT C9) | | | | | |
| | (PROTEASOME | | | | | |
| | SUBUNIT L) | | | | | |
| M128 | Similar to coactosin-like | − | 15945.1 | 1196417 | | |
| | protein | | | | | |
| M129 | THIOREDOXIN (ATL- | − | 11737.6 | 14740403 | | |
| | DERIVED FACTOR) | | | | | |
| | (ADF) (SURFACE | | | | | |
| | ASSOCIATED | | | | | |
| | SULPHYDRYL PROTEIN) | | | | | |
| | (SASP) | | | | | |
| M130 | K12 protein precursor | − | 27039.2 | 4506869 | | |
| M131 | VITAMIN-K | − | 52071.6 | 4506115 | | |
| | DEPENDENT PROTEIN C | | | | | |
| | PRECURSOR | | | | | |
| | (AUTOPROTHROMBIN | | | | | |
| | IIA) (ANTICOAGULANT | | | | | |
| | PROTEIN C) (BLOOD | | | | | |
| | COAGULATION | | | | | |
| | FACTOR XIV) | | | | | |
| M132 | titin, cardiac muscle | − | 2993535 | 2136280 | | |
| | [validated] | | | | | |
| M133 | MBL-associated protein | − | 20629.3 | 14726286 | | |
| | MAp19 | | | | | |
| M134 | EOSINOPHIL CATIONIC | + | 18440.5 | 4506551 | | |
| | PROTEIN PRECURSOR | | | | | |
| | (ECP) (RIBONUCLEASE | | | | | |
| | 3) (RNASE 3) | | | | | |
| M135 | This CDS feature is | ++ | 11577.6 | 886295 | | |
| | included to show the | | | | | |
| | translation of the | | | | | |
| | corresponding V_region. | | | | | |
| | Presently translation | | | | | |
| | qualifiers on V_region | | | | | |
| | features are illegal | | | | | |
| M136 | enhancer protein | − | 22127.5 | 2135068 | | |
| M137 | cysteine-rich protein 1 | + | 8532.9 | 4503047 | | |
| M138 | anti-Gd cold agglutinin | ++ | 10365.6 | 545723 | | |
| | monoclonal IgMK light | | | | | |
| | chain variable region | | | | | |
| M139 | CYSTATIN A (STEFIN | − | 11006.5 | 4885165 | | |
| | A) (CYSTATIN AS) | | | | | |
| M140 | ANNEXIN II | − | 38604.2 | 4757756 | | |
| | (LIPOCORTIN II) | | | | | |
| | (CALPACTIN 1 HEAVY | | | | | |
| | CHAIN) | | | | | |
| | (CHROMOBINDIN 8) | | | | | |
| | (P36) (PROTEIN I) | | | | | |
| | (PLACENTAL | | | | | |
| | ANTICOAGULANT | | | | | |
| | PROTEIN IV) (PAP-IV) | | | | | |
| M141 | diazepam binding inhibitor | − | 11793.4 | 10140853 | | |
| M142 | bA139H14.1 (lymphocyte | − | 70288.8 | 8217500 | | |
| | cytosolic protein 1 (L- | | | | | |
| | plastin)) | | | | | |
| M143 | heparan sulfate | ++ | 122673 | 14733263 | | |
| | proteoglycan 2 (perlecan) | | | | | |
| M144 | cathepsin C | − | 51854.1 | 13631727 | | |
| M145 | MYELOBLASTIN | − | 27807.2 | 14765501 | | |
| | PRECURSOR | | | | | |
| | (LEUKOCYTE | | | | | |
| | PROTEINASE 3) (PR-3) | | | | | |
| | (PR3) (AGP7) | | | | | |
| | (WEGENER'S | | | | | |
| | AUTOANTIGEN) (P29) | | | | | |
| | (C-ANCA ANTIGEN) | | | | | |
| M146 | glutaredoxin | − | 11761.8 | 643695 | | |
| M147 | TRANSLATIONALLY | + | 19595.5 | 4507669 | | |
| | CONTROLLED TUMOR | | | | | |
| | PROTEIN (TCTP) | | | | | |
| M148 | PROTEASOME | − | 30354.5 | 1172602 | | |
| | COMPONENT C13 | | | | | |
| | PRECURSOR | | | | | |
| | (MACROPAIN SUBUNIT | | | | | |
| | C13) (MULTICATALYTIC | | | | | |
| | ENDOPEPTIDASE | | | | | |
| | COMPLEX SUBUNIT | | | | | |
| | C13) | | | | | |
| M149 | ENDOTHELIAL | + | 26671.6 | 11420547 | | |
| | PROTEIN C RECEPTOR | | | | | |
| | PRECURSOR | | | | | |
| | (ENDOTHELIAL CELL | | | | | |
| | PROTEIN C RECEPTOR) | | | | | |
| | (ACTIVATED PROTEIN C | | | | | |
| | RECEPTOR) (APC | | | | | |
| | RECEPTOR) | | | | | |
| M151 | D-DOPACHROME | − | 12711.8 | 4503291 | | |
| | TAUTOMERASE | | | | | |
| | (PHENYLPYRUVATE | | | | | |
| | TAUTOMERASE II) | | | | | |
| M152 | p80 protein | ++ | 75356.7 | 1483131 | | |
| M153 | COLLAGEN ALPHA 1(V) | ++ | 183619 | 4502957 | | |
| | CHAIN PRECURSOR | | | | | |
| M155 | rho GDP dissociation | ++ | 23193.2 | 36038 | | |
| | inhibitor (GDI) | | | | | |
| M156 | FIBRONECTIN | + | 262608 | 2506872 | | |
| | PRECURSOR (FN) | | | | | |
| | (COLD-INSOLUBLE | | | | | |
| | GLOBULIN) (CIG) | | | | | |
| M157 | FK506-BINDING | − | 11950.8 | 4503725 | | |
| | PROTEIN (FKBP-12) | | | | | |
| | (PEPTIDYL-PROLYL CIS- | | | | | |
| | TRANS ISOMERASE) | | | | | |
| | (PPIASE) (ROTAMASE) | | | | | |
| | (IMMUNOPHILIN | | | | | |
| | FKBP12) | | | | | |
| M159 | similar to osteoclast | − | 23786.9 | 14738380 | | |
| | stimulating factor 1 (H. | | | | | |
| | sapiens) | | | | | |
| M161 | PEROXIREDOXIN 2 | ++ | 21892 | 13631440 | | |
| | (THIOREDOXIN | | | | | |
| | PEROXIDASE 1) | | | | | |
| | (THIOREDOXIN- | | | | | |
| | DEPENDENT PEROXIDE | | | | | |
| | REDUCTASE 1) (THIOL- | | | | | |
| | SPECIFIC | | | | | |
| | ANTIOXIDANT | | | | | |
| | PROTEIN) (TSA) (PRP) | | | | | |
| | (NATURAL KILLER | | | | | |
| | CELL ENHANCING | | | | | |
| | FACTOR B) (NKEF-B) | | | | | |
| M162 | myosin catalytic light chain | − | 16931.1 | 10440556 | | |
| | LC17b | | | | | |
| M163 | protein SPY75 | − | 53998.3 | 4885405 | | |
| M164 | 2-phosphopyruvate- | − | 47109.1 | 693933 | | |
| | hydratase alpha-enolase | | | | | |
| M166 | pancreatic secretory trypsin | + | 6247.1 | 671743 | | |
| | inhibitor | | | | | |
| M167 | GLIA MATURATION | − | 16801.4 | 4758440 | | |
| | FACTOR GAMMA (GMF- | | | | | |
| | GAMMA) | | | | | |
| M168 | CALCYCLIN (LUNG 10 | − | 10153.8 | 1173337 | | |
| | KDA PROTEIN) | | | | | |
| M169 | SH3 DOMAIN-BINDING | − | 12774.3 | 4506925 | | |
| | GLUTAMIC ACID-RICH- | | | | | |
| | LIKE PROTEIN | | | | | |
| M170 | CHYMOTRYPSINOGENA | − | 25666.3 | 117615 | | |
| M171 | DJ-1 protein | − | 19847.1 | 6005749 | | |
| M172 | phosphoprotein enriched in | − | 15040.2 | 4505705 | | |
| | astrocytes 15 | | | | | |
| M173 | EGF-CONTAINING | + | 54641 | 9665262 | | |
| | FIBULIN-LIKE | | | | | |
| | EXTRACELLULAR | | | | | |
| | MATRIX PROTEIN 1 | | | | | |
| | PRECURSOR (FIBULIN- | | | | | |
| | 3) (FIBL-3) (T16 | | | | | |
| | PROTEIN) | | | | | |
| M174 | superoxide dismutase 3, | − | 25851.1 | 14733169 | | |
| | extracellular | | | | | |
| M175 | CLARA CELL | − | 9993.8 | 4507809 | | |
| | PHOSPHOLIPID- | | | | | |
| | BINDING PROTEIN | | | | | |
| | PRECURSOR (CCPBP) | | | | | |
| | (CLARA CELLS 10 KDA | | | | | |
| | SECRETORY PROTEIN) | | | | | |
| | (CC10) (UTEROGLOBIN) | | | | | |
| | (URINE PROTEIN 1) | | | | | |
| | (UP1) | | | | | |
| M176 | Similar to LIM and SH3 | − | 29658.3 | 15214662 | | |
| | protein 1 | | | | | |
| M177 | PROSTAGLANDIN-H2 D- | ++ | 21028.9 | 4506251 | | |
| | ISOMERASE | | | | | |
| | PRECURSOR | | | | | |
| | (PROSTAGLANDIN-D | | | | | |
| | SYNTHASE) | | | | | |
| | (GLUTATHIONE- | | | | | |
| | INDEPENDENT PGD | | | | | |
| | SYNTHETASE) | | | | | |
| | (PROSTAGLANDIN D2 | | | | | |
| | SYNTHASE) (PGD2 | | | | | |
| | SYNTHASE) (PGDS2) | | | | | |
| | (PGDS) (BETA-TRACE | | | | | |
| | PROTEIN) | | | | | |
| M178 | mannose 6- | − | 273400 | 6981078 | | |
| | phosphate/insulin-like | | | | | |
| | growth factor II receptor | | | | | |
| M179 | OXYGEN-REGULATED | − | 240663 | 5454016 | | |
| | PROTEIN 1 (RETINITIS | | | | | |
| | PIGMENTOSA RP1 | | | | | |
| | PROTEIN) (RETINITIS | | | | | |
| | PIGMENTOSA 1 | | | | | |
| | PROTEIN) | | | | | |
| M180 | SERUM AMYLOID A | − | 12289.5 | 7531274 | | |
| | PROTEIN (SAA) | | | | | |
| | [CONTAINS: AMYLOID | | | | | |
| | PROTEIN A (AMYLOID | | | | | |
| | FIBRIL PROTEIN AA)] | | | | | |
| M181 | CARGO SELECTION | − | 47033.1 | 5032183 | | |
| | PROTEIN TIP47 (47 KDA | | | | | |
| | MANNOSE 6- | | | | | |
| | PHOSPHATE | | | | | |
| | RECEPTOR-BINDING | | | | | |
| | PROTEIN) (47 KDA MPR- | | | | | |
| | BINDING PROTEIN) | | | | | |
| | (PLACENTAL PROTEIN | | | | | |
| | 17) | | | | | |
| M182 | adenylyl cyclase-associated | − | 51749.4 | 15296533 | | |
| | protein | | | | | |
| M183 | aldolase C | − | 39456.1 | 4885063 | | |
| M184 | similar to PROTEASOME | − | 27330.7 | 14719929 | | |
| | SUBUNIT ALPHA TYPE 6 | | | | | |
| | (PROTEASOME IOTA | | | | | |
| | CHAIN) (MACROPAIN | | | | | |
| | IOTA CHAIN) | | | | | |
| | (MULTICATALYTIC | | | | | |
| | ENDOPEPTIDASE | | | | | |
| | COMPLEX IOTA CHAIN) | | | | | |
| | (27 KDA PROSOMAL | | | | | |
| | PROTEIN) (PROS-27) | | | | | |
| | (P27K) (H. sapiens) | | | | | |
| M186 | FOLLISTATIN- | − | 34985.7 | 5901956 | | |
| | RELATED PROTEIN 1 | | | | | |
| | PRECURSOR | | | | | |
| M187 | GAMMA-INTERFERON | − | 29149.1 | 12643406 | | |
| | INDUCIBLE | | | | | |
| | LYSOSOMAL THIOL | | | | | |
| | REDUCTASE | | | | | |
| | PRECURSOR (GAMMA- | | | | | |
| | INTERFERON- | | | | | |
| | INDUCIBLE PROTEIN IP- | | | | | |
| | 30) | | | | | |
| M188 | F-ACTIN CAPPING | − | 31350.7 | 13124696 | | |
| | PROTEIN BETA | | | | | |
| | SUBUNIT (CAPZ BETA) | | | | | |
| M189 | coagulation factor X | − | 52535.2 | 180336 | | |
| M190 | histone H3 | − | 15328 | 4504279 | | |
| M191 | adiponectin | ++ | 26413.8 | 4757760 | | |
| M192 | 16G2 | − | 42055.2 | 14738192 | | |
| M195 | S-100P PROTEIN | − | 10400 | 5174663 | | |
| M196 | INTERCELLULAR | + | 30653.5 | 4504557 | | |
| | ADHESION MOLECULE- | | | | | |
| | 2 PRECURSOR (ICAM-2) | | | | | |
| | (CD102) | | | | | |
| M197 | MANNOSYL- | − | 70821.3 | 5174521 | | |
| | OLIGOSACCHARIDE | | | | | |
| | ALPHA-1,2- | | | | | |
| | MANNOSIDASE | | | | | |
| | (MAN(9)-ALPHA- | | | | | |
| | MANNOSIDASE) | | | | | |
| M198 | NAD+-isocitrate | ++ | 39592 | 5031777 | | |
| | dehydrogenase, alpha | | | | | |
| | subunit | | | | | |
| M199 | CONNECTIVE TISSUE | − | 38069.8 | 4503123 | | |
| | GROWTH FACTOR | | | | | |
| | PRECURSOR | | | | | |
| | (HYPERTROPHIC | | | | | |
| | CHONDROCYTE- | | | | | |
| | SPECIFIC PROTEIN 24) | | | | | |
| M200 | complement factor H- | − | 27868.8 | 2134940 | | |
| | related protein FHR-2 | | | | | |
| M201 | RIBONUCLEASE, | − | 16377.3 | 133237 | | |
| | SEMINAL PRECURSOR | | | | | |
| | (SEMINAL RNASE) (S- | | | | | |
| | RNASE) | | | | | |
| | (RIBONUCLEASE BS-1) | | | | | |
| M203 | putative | − | 12859.9 | 12832737 | | |
| M204 | SERUM AMYLOID A-4 | − | 14806.8 | 10835095 | | |
| | PROTEIN PRECURSOR | | | | | |
| | (CONSTITUTIVELY | | | | | |
| | EXPRESSED SERUM | | | | | |
| | AMYLOID A PROTEIN) | | | | | |
| | (C-SAA) | | | | | |
| M205 | HP1-BP74 | + | 61207.3 | 11424882 | | |
| M206 | myristoylated alanine-rich | − | 3276.6 | 187385 | | |
| | C-kinase substrate | | | | | |
| M207 | CORTICOSTEROID- | + | 45141.1 | 4502595 | | |
| | BINDING GLOBULIN | | | | | |
| | PRECURSOR (CBG) | | | | | |
| | (TRANSCORTIN) | | | | | |
| M208 | mannose-binding lectin | − | 26090.6 | 5911809 | | |
| M209 | APOLIPOPROTEIN L | − | 42383.5 | 14916953 | | |
| | PRECURSOR (APO-L) | | | | | |
| M210 | GLUTATHIONE | − | 27566 | 4758484 | | |
| | TRANSFERASE OMEGA | | | | | |
| | 1 (GSTO 1-1) | | | | | |
| M211 | Somatomedin A | − | 7578.8 | 224061 | | |
| M212 | ganglioside M2 activator | − | 17623.5 | 106058 | | |
| | protein | | | | | |
| M214 | FATTY ACID-BINDING | − | 15164.5 | 4557581 | | |
| | PROTEIN, EPIDERMAL | | | | | |
| | (E-FABP) (PSORIASIS- | | | | | |
| | ASSOCIATED FATTY | | | | | |
| | ACID-BINDING | | | | | |
| | PROTEIN HOMOLOG) | | | | | |
| | (PA-FABP) | | | | | |
| M215 | MYELOID CELL | − | 45836.3 | 4505227 | | |
| | NUCLEAR | | | | | |
| | DIFFERENTIATION | | | | | |
| | ANTIGEN | | | | | |
| M216 | protein-L-isoaspartate (D- | − | 24679.5 | 14781911 | | |
| | aspartate) O- | | | | | |
| | methyltransferase (EC | | | | | |
| | 2.1.1.77) splice form II | | | | | |
| M217 | pancreatic ribonuclease | − | 17239.9 | 2135882 | | |
| | (EC 3.1.27.5) precursor | | | | | |
| M219 | anti-MSP1 MAD20 block2 | − | 11668.2 | 12836991 | | |
| | ScFv Ig light chain variable | | | | | |
| | region | | | | | |
| M220 | vimentin | + | 53714 | 340219 | | |
| M221 | HLA CLASS II | − | 33460.7 | 399888 | | |
| | HISTOCOMPATIBILITY | | | | | |
| | ANTIGEN, GAMMA | | | | | |
| | CHAIN (HLA-DR | | | | | |
| | ANTIGENS | | | | | |
| | ASSOCIATED | | | | | |
| | INVARIANT CHAIN) | | | | | |
| | (P33) (CD74 ANTIGEN) | | | | | |
| M222 | IMP dehydrogenase | + | 55920.4 | 4504689 | | |
| M223 | granulin | − | 61441.5 | 14772463 | | |
| M224 | p97 | − | 80170.1 | 3786308 | | |
| M225 | antigen HLA SB beta, MHC | − | 19069.9 | 224042 | | |
| | II | | | | | |
| M227 | kinesin like protein 9 | + | 89942.6 | 6754442 | | |
| M228 | PROTEASOME | − | 28936.5 | 4506191 | | |
| | COMPONENT MECL-1 | | | | | |
| | PRECURSOR | | | | | |
| | (MACROPAIN SUBUNIT | | | | | |
| | MECL-1) | | | | | |
| | (MULTICATALYTIC | | | | | |
| | ENDOPEPTIDASE | | | | | |
| | COMPLEX SUBUNIT | | | | | |
| | MECL-1) | | | | | |
| M233 | hypothetical protein | ++ | 17934.8 | 14773888 | | |
| | DKFZp761B15121.1 | | | | | |
| M235 | VON EBNER'S GLAND | − | 19250.1 | 4504963 | | |
| | PROTEIN PRECURSOR | | | | | |
| | (VEG PROTEIN) (TEAR | | | | | |
| | PREALBUMIN) (TP) | | | | | |
| | (TEAR LIPOCALIN) | | | | | |
| | (LIPOCALIN-1) | | | | | |
| M236 | RIBONUCLEASE | − | 16461 | 133198 | | |
| | PANCREATIC | | | | | |
| | PRECURSOR (RNASE 1) | | | | | |
| | (RNASE A) | | | | | |
| M240 | Carbonic Anhydrase II | − | 29246.2 | 4557395 | | |
| | (Carbonate Dehydratase 11) | | | | | |
| | (CAII) | | | | | |
| M241 | HRV Fab 027-VL | − | 12383 | 2385498 | | |
| M244 | 364K Golgi complex- | − | 364299 | 7441640 | | |
| | associated protein | | | | | |
| M245 | histone H2A.5 | − | 14059.5 | 70686 | | |
| M246 | antibody, light chain | − | 12356.8 | 732744 | | |
| | variable regin to HIV1 p25 | | | | | |
| M248 | TRYPSINOGEN, | − | 25424.9 | 2507249 | | |
| | CATIONIC PRECURSOR | | | | | |
| | (BETA-TRYPSIN) | | | | | |
| M249 | putative | − | 26422.2 | 12963645 | | |
| M250 | LOW AFFINITY | − | 34989.9 | 399476 | | |
| | IMMUNOGLOBULIN | | | | | |
| | GAMMA FC REGION | | | | | |
| | RECEPTOR II-A | | | | | |
| | PRECURSOR (FC- | | | | | |
| | GAMMA RII-A) (FCRII-A) | | | | | |
| | (IGG FC RECEPTOR II-A) | | | | | |
| | (FC-GAMMA-RIIA) | | | | | |
| | (CD32) (CDW32) | | | | | |
| M252 | CYTOCHROME C | − | 11888 | 14782885 | | |
| M258 | gamma-glutamyl hydrolase | − | 26023.5 | 13646249 | | |
| | (conjugase, | | | | | |
| | folylpolygammaglutamyl | | | | | |
| | hydrolase) precursor | | | | | |
| M259 | breakpoint cluster region | − | 15521.8 | 3002951 | | |
| | protein 1 | | | | | |
| M261 | BCL3 | + | 46432.3 | 3928845 | | |
| M264 | macrophage migration | − | 11428.1 | 187181 | | |
| | inhibitory factor | | | | | |
| M266 | unnamed protein product | − | 47994.5 | 10436374 | | |
| M267 | ribosomal protein S28, | − | 2336.6 | 7440562 | | |
| | cytosolic | | | | | |
| M268 | MICROFIBRIL- | + | 19611.7 | 4505089 | | |
| | ASSOCIATED | | | | | |
| | GLYCOPROTEIN 2 | | | | | |
| | PRECURSOR (MAGP-2) | | | | | |
| | (MP25) | | | | | |
| M269 | cathepsin S | − | 37495.9 | 11427057 | | |
| M270 | lambda-chain C-region C- | ++ | 11515.3 | 165427 | | |
| | lambda-2 | | | | | |
| M271 | anti-porcine VCAM mAb | − | 12178.8 | 4098515 | | |
| | 3F4 light chain variable | | | | | |
| | region | | | | | |
| M275 | CATHEPSIN D | − | 44552.5 | 4503143 | | |
| M277 | putative | − | 19274.4 | 12843372 | | |
| M278 | transaldolase | − | 37540.3 | 5803187 | | |
| M280 | myeloid inhibitory siglec | ++ | 51760.6 | 13936734 | | |
| M281 | granulocyte inhibitory | − | 2046.3 | 106167 | | |
| | protein | | | | | |
| M282 | GROWTH FACTOR | − | 25206.5 | 4504111 | | |
| | RECEPTOR-BOUND | | | | | |
| | PROTEIN 2 (GRB2 | | | | | |
| | ADAPTER PROTEIN) | | | | | |
| | (SH2/SH3 ADAPTER | | | | | |
| | GRB2) (ASH PROTEIN) | | | | | |
| M284 | UBIQUITIN-LIKE | − | 10871.3 | 5902098 | | |
| | PROTEIN SMT3B | | | | | |
| | (SENTRIN 2) | | | | | |
| M286 | HLA CLASS I | − | 40950.1 | 231427 | | |
| | HISTOCOMPATIBILITY | | | | | |
| | ANTIGEN, CW-1 | | | | | |
| | CW*0102 ALPHA CHAIN | | | | | |
| | PRECURSOR (CW1.2) | | | | | |
| M287 | 40S RIBOSOMAL | − | 15550.2 | 4506693 | | |
| | PROTEIN S17 | | | | | |
| M289 | dJ747L4.1 (Brachyury (T | − | 47004.1 | 3900891 | | |
| | box protein)-LIKE protein | | | | | |
| M290 | anti-oxidized LDL | − | 11825.3 | 15277620 | | |
| | autoantibody variable kappa | | | | | |
| | chain | | | | | |
| M296 | similar to | − | 16974.3 | 14786818 | | |
| | TROPOMYOSIN, | | | | | |
| | CYTOSKELETAL TYPE | | | | | |
| | (TM30-NM) (H. sapiens) | | | | | |
| M298 | ZYXIN (ZYXIN 2) | − | 61277.7 | 4508047 | | |
| M300 | ENHANCER OF | − | 12259 | 4758302 | | |
| | RUDIMENTARY | | | | | |
| | HOMOLOG | | | | | |
| M301 | regulator of G-protein | − | 23255.8 | 5032039 | | |
| | signaling 4 | | | | | |
| M305 | anti-DNA immunoglobulin | + | 11047.2 | 1870506 | | |
| | light chain IgG | | | | | |
| M307 | UBIQUITIN-LIKE | − | 9071.6 | 5453760 | | |
| | PROTEIN NEDD8 | | | | | |
| M311 | SCRAPIE-RESPONSIVE | − | 11081.2 | 6005870 | | |
| | PROTEIN 1 PRECURSOR | | | | | |
| | (SCRG-1) | | | | | |
| M312 | gonadotropin releasing | − | 1651.6 | 225500 | | |
| | peptide | | | | | |
| M315 | IRT-1 | − | 14617.3 | 4758612 | | |
| M316 | recombinant antibody light | − | 11556.9 | 12957386 | | |
| | chain VL domain | | | | | |
| M317 | putative | − | 56695 | 12836433 | | |
| M318 | CYSTATIN M | − | 16511.2 | 4503113 | | |
| | PRECURSOR (CYSTATIN | | | | | |
| | E) | | | | | |
| M320 | proteasome activator PA28 | − | 27348.7 | 4506237 | | |
| | beta chain | | | | | |
| M321 | AGGRECAN CORE | − | 250194 | 129886 | | |
| | PROTEIN PRECURSOR | | | | | |
| | (CARTILAGE-SPECIFIC | | | | | |
| | PROTEOGLYCAN CORE | | | | | |
| | PROTEIN) (CSPCP) | | | | | |
| | (CHONDROITIN | | | | | |
| | SULFATE | | | | | |
| | PROTEOGLYCAN CORE | | | | | |
| | PROTEIN 1) | | | | | |
| M322 | KIAA0185 protein | − | 57547.8 | 15298593 | | |
| M323 | hypothetical protein | − | 97949.7 | 14745846 | | |
| | FLJ13465 | | | | | |
| M324 | CARBOXYPEPTIDASE N | − | 58649.4 | 115877 | | |
| | 83 KDA CHAIN | | | | | |
| | (CARBOXYPEPTIDASE | | | | | |
| | N REGULATORY | | | | | |
| | SUBUNIT) | | | | | |
| M326 | R33729_1 | − | 11332.9 | 3355455 | | |
| M327 | lymphocyte surface antigen | − | 32074.7 | 106924 | | |
| | precursor CD44 | | | | | |
| M328 | PYRIDOXINE KINASE | − | 35102.5 | 4505701 | | |
| | (PYRIDOXAL KINASE) | | | | | |
| M329 | unnamed protein product | − | 42442.7 | 10436670 | | |
| M330 | CALPACTIN I LIGHT | − | 11203.2 | 4506761 | | |
| | CHAIN (P10 PROTEIN) | | | | | |
| | (P11) (CELLULAR | | | | | |
| | LIGAND OF ANNEXIN II) | | | | | |
| | (NERVE GROWTH | | | | | |
| | FACTOR INDUCED | | | | | |
| | PROTEIN 42C) | | | | | |
| M331 | C360B4.1 (PUTATIVE | − | 23267.9 | 5912545 | | |
| | novel protein similar to | | | | | |
| | predicted bacterial and | | | | | |
| | worm proteins) | | | | | |
| M332 | latent transforming growth | − | 161160 | 3327808 | | |
| | factor-beta binding protein | | | | | |
| | 4S | | | | | |
| M333 | hypothetical protein | + | 49735 | 14744245 | | |
| | XP_047083 | | | | | |
| M334 | similar to | + | 45421.1 | 13633718 | | |
| | GASTRIN/CHOLECYSTOKININ | | | | | |
| | TYPE B | | | | | |
| | RECEPTOR (CCK-B | | | | | |
| | RECEPTOR) (CCK-BR) | | | | | |
| | (H. sapiens) | | | | | |
| M336 | similar to LINE-1 | + | 16237.5 | 14766164 | | |
| | REVERSE | | | | | |
| | TRANSCRIPTASE | | | | | |
| | HOMOLOG (H. sapiens) | | | | | |
| M337 | TYROSINE-PROTEIN | − | 50704.6 | 4758078 | | |
| | KINASE CSK (C-SRC | | | | | |
| | KINASE) | | | | | |
| M338 | Similar to dystroglycan 1 | − | 97541.1 | 15215308 | | |
| | (dystrophin-associated | | | | | |
| | glycoprotein 1) | | | | | |
| M339 | complement component 1, | − | 37337.1 | 14766592 | | |
| | s subcomponent | | | | | |
| M340 | lysophospholipase II | + | 24737.1 | 9966764 | | |
| M344 | TELOMERASE-BINDING | − | 18721.5 | 9790017 | | |
| | PROTEIN P23 (HSP90 CO- | | | | | |
| | CHAPERONE) | | | | | |
| | (PROGESTERONE | | | | | |
| | RECEPTOR COMPLEX | | | | | |
| | P23) | | | | | |
| M346 | inwardly rectifying | − | 43580.1 | 2143813 | | |
| | potassium channel protein | | | | | |
| | Kir6.2 | | | | | |
| M349 | dnaJ protein homolog | − | 30611.7 | 478645 | | |
| M350 | 40S RIBOSOMAL | − | 6676.8 | 4506717 | | |
| | PROTEIN S29 | | | | | |
| M352 | MYOGLOBIN | + | 17042.7 | 127656 | | |
| M353 | cathepsin X precursor | − | 33943.2 | 3650498 | | |
| M354 | unknown | − | 17297.2 | 3860020 | | |
| M356 | HYPOTHETICAL | − | 24353.8 | 12585535 | | |
| | PROTEIN CGI-109 | | | | | |
| | PRECURSOR | | | | | |
| M357 | LITHOSTATHINE 1 | + | 18664.9 | 10835248 | | |
| | BETA PRECURSOR | | | | | |
| | (REGENERATING | | | | | |
| | PROTEIN I BETA) | | | | | |
| M358 | human leucocyte antigen B | + | 31344.8 | 9027550 | | |
| M360 | This CDS feature is | − | 11607 | 681900 | | |
| | included to show the | | | | | |
| | translation of the | | | | | |
| | corresponding V_region. | | | | | |
| | Presently translation | | | | | |
| | qualifiers on V_region | | | | | |
| | features are illegal. | | | | | |
| M362 | KIAA0336 | − | 184659 | 7662062 | | |
| M363 | Similar to expressed | ++ | 52604.1 | 15489206 | | |
| | sequence 2 embryonic lethal | | | | | |
| M366 | NEUROBLASTOMA | − | 19276.9 | 4885509 | | |
| | SUPPRESSOR OF | | | | | |
| | TUMORIGENICITY 1 | | | | | |
| | (ZINC FINGER PROTEIN | | | | | |
| | DAN) (N03) | | | | | |
| M370 | putative | − | 25193.3 | 12847919 | | |
| M372 | alpha 1F calcium channel | − | 221137 | 14669577 | | |
| | subunit | | | | | |
| M373 | sa1 (Drosophila)-like 2 | − | 105310 | 14751528 | | |
| M374 | AF15q14 protein | − | 205685 | 9966807 | | |
| M375 | putative | +++ | 30469.3 | 12836789 | | |
| M376 | fibroblast growth factor 13 | − | 8239.5 | 4512024 | | |
| | isoform lylv | | | | | |
| M379 | KIAA0433 | − | 140408 | 7662118 | | |
| M380 | anti-c-erbB-2 | − | 11608.9 | 1145350 | | |
| | immunoglobulin light chain | | | | | |
| | V | | | | | |
| M381 | ATP-BINDING | − | 269976 | 14916523 | | |
| | CASSETTE, SUB- | | | | | |
| | FAMILY A, MEMBER 2 | | | | | |
| | (ATP-BINDING | | | | | |
| | CASSETTE | | | | | |
| | TRANSPORTER 2) (ATP- | | | | | |
| | BINDING CASSETTE 2) | | | | | |
| M382 | G protein-coupled receptor | − | 85231.4 | 13929158 | | |
| | kinase-associated ADP | | | | | |
| | ribosylation factor GTPase- | | | | | |
| | activating protein | | | | | |
| M387 | farnesyl-protein transferase | ++ | 43087.6 | 2135098 | | |
| | beta chain | | | | | |
| M388 | KIAA1813 protein | − | 73090.1 | 14017843 | | |
| M389 | hypothetical protein | − | 117875 | 13644578 | | |
| | FLJ11937 | | | | | |
| M391 | ribosomal protein L29 | − | 17667.1 | 1082766 | | |
| M393 | inhibitor PI, alpha1 | − | 2431 | 223058 | | |
| | proteinase | | | | | |
| M394 | dJ467L1.3 (period | − | 126247 | 6580412 | | |
| | (Drosophila) homolog 3) | | | | | |
| M395 | cylindromatosis (turban | − | 82125.4 | 14779751 | | |
| | tumor syndrome) | | | | | |
| M396 | T cell receptor alpha chain | − | 1710 | 478461 | | |
| | V region (clone 2V alpha | | | | | |
| | 23-2) | | | | | |
| M399 | anti-pneumococcal Ig L- | − | 11358.6 | 3603383 | | |
| | chain Fab fragment | | | | | |
| M401 | Unknown (protein for | − | 45446.9 | 15530249 | | |
| | MGC: 16498) | | | | | |
| M403 | plasma protease (C1) | − | 55182.5 | 179619 | | |
| | inhibitor precursor | | | | | |
| M404 | similar to INITIATION | ++ | 16789.2 | 13642507 | | |
| | FACTOR 5A (EIF-5A) | | | | | |
| | (EIF-4D) (REV-BINDING | | | | | |
| | FACTOR) (H. sapiens) | | | | | |
| M406 | CGI-202 | − | 13832.6 | 8895093 | | |
| M409 | alternatively spliced | − | 14203.2 | 2125864 | | |
| M410 | neuronal leucine-rich | − | 79424.7 | 14751034 | | |
| | repeat protein-3 | | | | | |
| M411 | Unknown (protein for | − | 71702.7 | 15277493 | | |
| | IMAGE: 3587716) | | | | | |
| M412 | PROTEASOME | − | 29546.7 | 9910833 | | |
| | SUBUNIT ALPHA TYPE 1 | | | | | |
| | (PROTEASOME | | | | | |
| | COMPONENT C2) | | | | | |
| | (MACROPAIN SUBUNIT | | | | | |
| | C2) (MULTICATALYTIC | | | | | |
| | ENDOPEPTIDASE | | | | | |
| | COMPLEX SUBUNIT C2) | | | | | |
| | (PROTEASOME NU | | | | | |
| | CHAIN) | | | | | |
| M413 | NIF-like protein | − | 38532.3 | 15487262 | | |
| M415 | unnamed protein product | − | 27742.6 | 10436722 | | |
| M417 | hairy/enhancer of split 6 | − | 23902 | 14009498 | | |
| M419 | sperm tail associated | ++ | 119401 | 9910570 | | |
| | protein | | | | | |
| M420 | NEURABIN-II (NEURAL | − | 89646.8 | 13431725 | | |
| | TISSUE-SPECIFIC F- | | | | | |
| | ACTIN BINDING | | | | | |
| | PROTEIN II) | | | | | |
| | (SPINOPHILIN) (P130) | | | | | |
| | (PP1BP134) | | | | | |
| M421 | TFNR | − | 245729 | 12232589 | | |
| M422 | TROPOMYOSIN 4, | − | 28509.8 | 6981672 | | |
| | EMBRYONIC | | | | | |
| | FIBROBLAST ISOFORM | | | | | |
| | (TM-4) | | | | | |
| M423 | ZINC FINGER Y- | − | 90531.5 | 4507967 | | |
| | CHROMOSOMAL | | | | | |
| | PROTEIN | | | | | |
| M424 | CD27L RECEPTOR | + | 29156.6 | 4507587 | | |
| | PRECURSOR (T-CELL | | | | | |
| | ACTIVATION ANTIGEN | | | | | |
| | CD27) (T14) | | | | | |
| M425 | FIBROLEUKIN | − | 50228.9 | 5730075 | | |
| | PRECURSOR | | | | | |
| | (FIBRINOGEN-LIKE | | | | | |
| | PROTEIN 2) (PT49) | | | | | |
| M428 | putative | − | 2213.6 | 553734 | | |
| M429 | unnamed protein product | − | 94254.2 | 10435664 | | |
| M431 | hypothetical protein | − | 101555 | 14720628 | | |
| M433 | myosin-VIIb | + | 240788 | 14161694 | | |
| M435 | hypothetical protein | − | 34555.4 | 15300488 | | |
| | FLJ20516 | | | | | |
| M436 | similar to ribosomal protein | − | 13043.6 | 13642964 | | |
| | S26 (H. sapiens) | | | | | |
| M439 | C-terminus matches | − | 375564 | 4522026 | | |
| | KIAA0559, N-terminus | | | | | |
| | similar to Bassoon protein | | | | | |
| M441 | arfaptin 1 | + | 40779.3 | 11120714 | | |
| M442 | apolipoprotein CI | − | 7750.9 | 178834 | | |
| M443 | dJ591C20.1 (novel protein | − | 52554.9 | 11125672 | | |
| | similar to mouse NG26) | | | | | |
| M444 | VESICLE TRANSPORT | − | 26687.6 | 13124617 | | |
| | V-SNARE PROTEIN | | | | | |
| | VTI1-LIKE 1 (VTI1-RP1) | | | | | |
| M445 | retinoblastoma-associated | − | 73913.1 | 5174457 | | |
| | protein HEC | | | | | |
| M446 | ZINC FINGER PROTEIN | − | 45586 | 4885267 | | |
| | GFI-1 (GROWTH | | | | | |
| | FACTOR | | | | | |
| | INDEPENDENCE-1) | | | | | |
| M447 | 5- | ++ | 40672.8 | 6981062 | | |
| | HYDROXYTRYPTAMINE | | | | | |
| | 5A RECEPTOR (5-HT- | | | | | |
| | 5A) (SEROTONIN | | | | | |
| | RECEPTOR) (REC17) | | | | | |
| M448 | This CDS feature is | − | 11581.9 | 845530 | | |
| | included to show the | | | | | |
| | translation of the | | | | | |
| | corresponding V_region. | | | | | |
| | Presently translation | | | | | |
| | qualifiers on V_region | | | | | |
| | features are illegal | | | | | |
| M451 | KIAA1664 protein | − | 99851.7 | 13359201 | | |
| M452 | METALLOTHIONEIN-II | − | 6042.3 | 5174764 | | |
| | (MT-II) | | | | | |
| M453 | microseminoprotein beta | − | 10651.1 | 225159 | | |
| M454 | SACSIN | − | 436757 | 7657536 | | |
| M455 | tenascin-R | − | 149575 | 1617316 | | |
| M457 | SERINE PROTEASE | − | 120760 | 13959398 | | |
| | INHIBITOR KAZAL- | | | | | |
| | TYPE 5 PRECURSOR | | | | | |
| | (LYMPHO-EPITHELIAL | | | | | |
| | KAZAL-TYPE RELATED | | | | | |
| | INHIBITOR) (LEKTI) | | | | | |
| | [CONTAINS: | | | | | |
| | HEMOFILTRATE | | | | | |
| | PEPTIDE HF6478 | | | | | |
| M458 | CALGRANULIN C | − | 10614.1 | 461678 | | |
| | (CAGC) | | | | | |
| M459 | galactose-specific lectin | − | 26148.5 | 1196442 | | |
| M460 | non-muscle myosin heavy | − | 72371.4 | 3205211 | | |
| | chain | | | | | |
| M461 | DOCK180 protein | − | 215377 | 4503355 | | |
| M462 | serine protease | − | 40239.3 | 3777621 | | |
| M463 | nuclear zinc finger protein | − | 89814.4 | 14764016 | | |
| | Np95 | | | | | |
| M464 | LIM protein (similar to rat | − | 63972.5 | 14250573 | | |
| | protein kinase C-binding | | | | | |
| | enigma) | | | | | |
| M465 | SECIS binding protein 2 | − | 95478.4 | 14211829 | | |
| M466 | HSPC055 protein | − | 83654.5 | 14775320 | | |
| M468 | ribosomal protein L14 | − | 23803.4 | 4506601 | | |
| M470 | chloride channel protein 3, | − | 91243.1 | 4502869 | | |
| | long form | | | | | |
| M471 | MITOGEN-ACTIVATED | − | 70970.1 | 4505153 | | |
| | PROTEIN KINASE | | | | | |
| | KINASE KINASE 3 | | | | | |
| | (MAPK/ERK KINASE | | | | | |
| | KINASE 3) (MEK | | | | | |
| | KINASE 3) (MEKK 3) | | | | | |
| M472 | unnamed protein product | − | 62332 | 12383086 | | |
| M473 | anti-HIV gp120 antibody | − | 11498.5 | 460857 | | |
| | light chain variable region | | | | | |
| M474 | CARBONIC | − | 34394.2 | 1345657 | | |
| | ANHYDRASE IV | | | | | |
| | PRECURSOR | | | | | |
| | (CARBONATE | | | | | |
| | DEHYDRATASE IV) (CA- | | | | | |
| | IV) | | | | | |
| M475 | hypothetical protein | − | 47126.1 | 13937775 | | |
| | FLJ11016 | | | | | |
| M476 | putative gene with | − | 50038.1 | 7717246 | | |
| | similarities to KIAA1074 | | | | | |
| | and KIAA0565 | | | | | |
| M477 | S-MYC PROTO- | − | 47002.5 | 11177868 | | |
| | ONCOGENE PROTEIN | | | | | |
| M480 | unnamed protein product | − | 42680.2 | 10432636 | | |
| M483 | proapolipoprotein Varient | − | 25119.7 | | 1 | 2 |
| M484 | Unknown | + | 33413.9 | | 3 | 4 |
| M485 | neutrophil lactoferrin | − | 59529.6 | 186818 | | |
| | | | | (GenPept) | | |
| M486 | alpha-2-HS glycoprotein | − | 12290.2 | 10289662 | | |
| | Varient | | | (dbEST) | | |
| M487 | MT-11 protein Varient | − | 10172.9 | 7378207 | | |
| | | | | (dbEST) | | |
| M488 | immunoglobulin lambda | − | 16740.9 | | 5 | 6 |
| | light chain homolog | | | | | |
| M489 | Unknown | − | 21461.8 | | 7 | 8 |
| M490 | Unknown | − | 20895 | | 9 | 10 |

| | |
|---|---|
| 10¹⁰ and above | +++ |
| 10⁸ to 10¹⁰ | ++ |
| 10⁶ to 10⁸ | + |
| 0 | − |

TABLE 3

| | | E_51 | E_59 | E_63 | E_67 | E_70 | N_52 | N_54 | N_58 | N_65 |
|---|---|---|---|---|---|---|---|---|---|---|
| Marker | Gene Name | Br38 | Br26 | Br35 | Br42 | Br43 | Br29 | Br28 | Br34 | Br36 |
| M30 | KININOGEN | ++ | − | − | − | − | ++ | ++ | + | ++ |
| | PRECURSOR (ALPHA-2- | | | | | | | | | |
| | THIOL PROTEINASE | | | | | | | | | |
| | INHIBITOR) [CONTAINS: | | | | | | | | | |
| | BRADYKININ] | | | | | | | | | |
| M31 | CALGRANULIN B | +++ | +++ | +++ | +++ | +++ | +++ | +++ | ++ | ++ |
| | (MIGRATION | | | | | | | | | |
| | INHIBITORY FACTOR- | | | | | | | | | |
| | RELATED PROTEIN 14) | | | | | | | | | |
| | (MRP-14) (P14) | | | | | | | | | |
| | (LEUKOCYTE L1 | | | | | | | | | |
| | COMPLEX HEAVY | | | | | | | | | |
| | CHAIN) (S100 CALCIUM- | | | | | | | | | |
| | BINDING PROTEIN A9) | | | | | | | | | |
| M37 | similar to phosphoglycerate | ++ | ++ | ++ | ++ | ++ | ++ | ++ | − | − |
| | mutase 1 (brain) | | | | | | | | | |
| M43 | amyloid related serum | ++ | ++ | +++ | +++ | +++ | +++ | ++ | − | ++ |
| | protein SAA | | | | | | | | | |
| M44 | CALGRANULIN A | +++ | ++ | +++ | +++ | +++ | ++ | +++ | ++ | + |
| | (MIGRATION | | | | | | | | | |
| | INHIBITORY FACTOR- | | | | | | | | | |
| | RELATED PROTEIN 8) | | | | | | | | | |
| | (MRP-8) (CYSTIC | | | | | | | | | |
| | FIBROSIS ANTIGEN) | | | | | | | | | |
| | (CFAG) (P8) | | | | | | | | | |
| | (LEUKOCYTE L1 | | | | | | | | | |
| | COMPLEX LIGHT | | | | | | | | | |
| | CHAIN) (S100 CALCIUM- | | | | | | | | | |
| | BINDING PROTEIN A8) | | | | | | | | | |
| M46 | RHO GDP- | ++ | ++ | +++ | ++ | ++ | ++ | ++ | − | + |
| | DISSOCIATION | | | | | | | | | |
| | INHIBITOR 2 (RHO GDI | | | | | | | | | |
| | 2) (RHO-GDI BETA) (LY- | | | | | | | | | |
| | GDI) | | | | | | | | | |
| M51 | 14-3-3 PROTEIN | ++ | ++ | ++ | ++ | ++ | − | + | − | + |
| | BETA/ALPHA (PROTEIN | | | | | | | | | |
| | KINASE C INHIBITOR | | | | | | | | | |
| | PROTEIN-1) (KCIP-1) | | | | | | | | | |
| | (PROTEIN 1054) | | | | | | | | | |
| M52 | Ig G1 H Nie | − | ++ | ++ | ++ | ++ | − | − | − | − |
| M53 | OSTEOPONTIN | + | ++ | ++ | − | − | − | − | − | − |
| | PRECURSOR (BONE | | | | | | | | | |
| | SIALOPROTEIN 1) | | | | | | | | | |
| | (URINARY STONE | | | | | | | | | |
| | PROTEIN) (SECRETED | | | | | | | | | |
| | PHOSPHOPROTEIN 1) | | | | | | | | | |
| | (SPP-1) | | | | | | | | | |
| | (NEPHROPONTIN) | | | | | | | | | |
| | (UROPONTIN) | | | | | | | | | |
| M55 | TRIOSEPHOSPHATE | ++ | ++ | ++ | − | ++ | − | − | − | − |
| | ISOMERASE (TIM) | | | | | | | | | |
| M58 | prosaposin (variant | − | ++ | ++ | ++ | − | ++ | − | − | − |
| | Gaucher disease and variant | | | | | | | | | |
| | metachromatic | | | | | | | | | |
| | leukodystrophy) | | | | | | | | | |
| M60 | CALGRANULIN C (S100 | ++ | +++ | ++ | ++ | ++ | ++ | ++ | − | − |
| | A12 protein) | | | | | | | | | |
| M62 | cathepsin B | ++ | ++ | ++ | ++ | ++ | − | − | − | − |
| M66 | apolipoprotein C-II | ++ | + | − | − | − | ++ | +++ | ++ | ++ |
| M68 | THYMOSIN BETA-4 | ++ | + | ++ | ++ | ++ | − | − | − | − |
| M69 | uracil DNA glycosylase | ++ | ++ | ++ | ++ | − | − | − | − | − |
| M70 | defensin alpha-3 precursor, | +++ | +++ | +++ | +++ | ++ | − | ++ | − | ++ |
| | neutrophil-specific | | | | | | | | | |
| | [validated] | | | | | | | | | |
| M71 | CD14 antigen | − | − | ++ | ++ | ++ | − | ++ | − | − |
| M72 | peptidylprolyl isomerase | ++ | ++ | ++ | ++ | ++ | − | − | − | ++ |
| | (EC 5.2.1.8) A | | | | | | | | | |
| M73 | similar to transgelin 2 (H. | ++ | ++ | ++ | − | ++ | + | − | − | − |
| | sapiens) | | | | | | | | | |
| M76 | alpha2 plasmin inhibitor | ++ | ++ | ++ | ++ | ++ | + | ++ | − | − |
| M78 | glutathione transferase | ++ | ++ | ++ | − | − | ++ | − | − | − |
| M82 | CALGIZZARIN (S100C | ++ | ++ | ++ | ++ | ++ | − | − | − | + |
| | PROTEIN) (MLN 70) | | | | | | | | | |
| M103 | neutrophil lipocalin | ++ | − | ++ | − | − | − | − | − | − |
| M106 | CYSTATIN B (LIVER | ++ | ++ | ++ | ++ | ++ | − | − | + | − |
| | THIOL PROTEINASE | | | | | | | | | |
| | INHIBITOR) (CPI-B) | | | | | | | | | |
| | (STEFIN B) | | | | | | | | | |
| M111 | SH3BGRL3-like protein | ++ | ++ | ++ | ++ | − | − | − | ++ | − |
| M117 | LOW AFFINITY | ++ | ++ | ++ | ++ | ++ | − | ++ | − | − |
| | IMMUNOGLOBULIN | | | | | | | | | |
| | GAMMA FC REGION | | | | | | | | | |
| | RECEPTOR III-A | | | | | | | | | |
| | PRECURSOR (IGG FC | | | | | | | | | |
| | RECEPTOR III-2) (FC- | | | | | | | | | |
| | GAMMA RIII-ALPHA) | | | | | | | | | |
| | (FC-GAMMA RIIIA) | | | | | | | | | |
| | (FCRIIIA) (FC-GAMMA | | | | | | | | | |
| | RIII) (FCRIII) (CD16-A) | | | | | | | | | |
| | (FCR-10) | | | | | | | | | |
| M121 | EPIDIDYMAL | ++ | ++ | ++ | ++ | − | − | − | ++ | − |
| | SECRETORY PROTEIN | | | | | | | | | |
| | E1 PRECURSOR | | | | | | | | | |
| | (NIEMANN-PICK | | | | | | | | | |
| | DISEASE TYPE C2 | | | | | | | | | |
| | PROTEIN) (EPI-1) (HE1) | | | | | | | | | |
| | (EPIDIDYMAL | | | | | | | | | |
| | SECRETORY PROTEIN | | | | | | | | | |
| | 14.6) (ESP14.6) | | | | | | | | | |
| M123 | metastasin | ++ | ++ | ++ | − | ++ | − | − | ++ | − |
| M128 | Similar to coactosin-like | − | ++ | ++ | ++ | − | + | − | − | − |
| | protein | | | | | | | | | |
| M131 | VITAMIN-K | − | − | − | − | − | ++ | + | − | − |
| | DEPENDENT PROTEIN C | | | | | | | | | |
| | PRECURSOR | | | | | | | | | |
| | (AUTOPROTHROMBIN | | | | | | | | | |
| | IIA) (ANTICOAGULANT | | | | | | | | | |
| | PROTEIN C) (BLOOD | | | | | | | | | |
| | COAGULATION | | | | | | | | | |
| | FACTOR XIV) | | | | | | | | | |
| M151 | D-DOPACHROME | ++ | + | ++ | − | + | − | − | − | − |
| | TAUTOMERASE | | | | | | | | | |
| | (PHENYLPYRUVATE | | | | | | | | | |
| | TAUTOMERASE II) | | | | | | | | | |
| M180 | SERUM AMYLOID A | − | − | ++ | + | + | − | − | − | − |
| | PROTEIN (SAA) | | | | | | | | | |
| | [CONTAINS: AMYLOID | | | | | | | | | |
| | PROTEIN A (AMYLOID | | | | | | | | | |
| | FIBRIL PROTEIN AA)] | | | | | | | | | |
| M195 | S-100P PROTEIN | ++ | + | − | − | + | − | − | − | − |
| M204 | SERUM AMYLOID A-4 | − | − | − | − | − | ++ | − | − | − |
| | PROTEIN PRECURSOR | | | | | | | | | |
| | (CONSTITUTIVELY | | | | | | | | | |
| | EXPRESSED SERUM | | | | | | | | | |
| | AMYLOID A PROTEIN) | | | | | | | | | |
| | (C-SAA) | | | | | | | | | |
| M484 | Unknown | − | − | + | + | − | − | − | − | − |

| | | | | | SEQ | SEQ |
|---|---|---|---|---|---|---|
| | | | | | ID | ID |
| | | N_66 | MW | GI | NO | NO |
| Marker | Gene Name | Br39 | (Da) | number | (nts) | (AA) |
| M30 | KININOGEN | − | 71945.7 | 125507 | | |
| | PRECURSOR (ALPHA-2- | | | | | |
| | THIOL PROTEINASE | | | | | |
| | INHIBITOR) [CONTAINS: | | | | | |
| | BRADYKININ] | | | | | |
| M31 | CALGRANULIN B | ++ | 13242.1 | 4506773 | | |
| | (MIGRATION | | | | | |
| | INHIBITORY FACTOR- | | | | | |
| | RELATED PROTEIN 14) | | | | | |
| | (MRP-14) (P14) | | | | | |
| | (LEUKOCYTE L1 | | | | | |
| | COMPLEX HEAVY | | | | | |
| | CHAIN) (S100 CALCIUM- | | | | | |
| | BINDING PROTEIN A9) | | | | | |
| M37 | similar to phosphoglycerate | ++ | 28850.2 | 15301114 | | |
| | mutase 1 (brain) | | | | | |
| M43 | amyloid related serum | + | 11682.8 | 4506777 | | |
| | protein SAA | | | | | |
| M44 | CALGRANULIN A | + | 10834.6 | 14729628 | | |
| | (MIGRATION | | | | | |
| | INHIBITORY FACTOR- | | | | | |
| | RELATED PROTEIN 8) | | | | | |
| | (MRP-8) (CYSTIC | | | | | |
| | FIBROSIS ANTIGEN) | | | | | |
| | (CFAG) (P8) | | | | | |
| | (LEUKOCYTE L1 | | | | | |
| | COMPLEX LIGHT | | | | | |
| | CHAIN) (S100 CALCIUM- | | | | | |
| | BINDING PROTEIN A8) | | | | | |
| M46 | RHO GDP- | ++ | 22988.1 | 10835002 | | |
| | DISSOCIATION | | | | | |
| | INHIBITOR 2 (RHO GDI | | | | | |
| | 2) (RHO-GDI BETA) (LY- | | | | | |
| | GDI) | | | | | |
| M51 | 14-3-3 PROTEIN | ++ | 28082.5 | 4507949 | | |
| | BETA/ALPHA (PROTEIN | | | | | |
| | KINASE C INHIBITOR | | | | | |
| | PROTEIN-1) (KCIP-1) | | | | | |
| | (PROTEIN 1054) | | | | | |
| M52 | Ig G1 H Nie | − | 49207.8 | 229601 | | |
| M53 | OSTEOPONTIN | − | 35422.9 | 14724978 | | |
| | PRECURSOR (BONE | | | | | |
| | SIALOPROTEIN 1) | | | | | |
| | (URINARY STONE | | | | | |
| | PROTEIN) (SECRETED | | | | | |
| | PHOSPHOPROTEIN 1) | | | | | |
| | (SPP-1) | | | | | |
| | (NEPHROPONTIN) | | | | | |
| | (UROPONTIN) | | | | | |
| M55 | TRIOSEPHOSPHATE | ++ | 26669.6 | 4507645 | | |
| | ISOMERASE (TIM) | | | | | |
| M58 | prosaposin (variant | − | 50307.8 | 15298143 | | |
| | Gaucher disease and variant | | | | | |
| | metachromatic | | | | | |
| | leukodystrophy) | | | | | |
| M60 | CALGRANULIN C (S100 | − | 10443.9 | 2146972 | | |
| | A12 protein) | | | | | |
| M62 | cathepsin B | − | 37821.8 | 4503139 | | |
| M66 | apolipoprotein C-II | − | 10183.5 | 2134777 | | |
| M68 | THYMOSIN BETA-4 | ++ | 5062.7 | 14730886 | | |
| M69 | uracil DNA glycosylase | − | 35492.9 | 35053 | | |
| M70 | defensin alpha-3 precursor, | ++ | 10245 | 4885179 | | |
| | neutrophil-specific | | | | | |
| | [validated] | | | | | |
| M71 | CD14 antigen | − | 40076.4 | 4557417 | | |
| M72 | peptidylprolyl isomerase | ++ | 19008.7 | 12804335 | | |
| | (EC 5.2.1.8) A | | | | | |
| M73 | similar to transgelin 2 (H. | − | 24454 | 14728128 | | |
| | sapiens) | | | | | |
| M76 | alpha2 plasmin inhibitor | − | 54596.1 | 11386143 | | |
| M78 | glutathione transferase | − | 23463.2 | 14766346 | | |
| M82 | CALGIZZARIN (S100C | − | 11740.5 | 5032057 | | |
| | PROTEIN) (MLN 70) | | | | | |
| M103 | neutrophil lipocalin | − | 20547.6 | 4261868 | | |
| M106 | CYSTATIN B (LIVER | + | 11139.6 | 4503117 | | |
| | THIOL PROTEINASE | | | | | |
| | INHIBITOR) (CPI-B) | | | | | |
| | (STEFIN B) | | | | | |
| M111 | SH3BGRL3-like protein | − | 10437.8 | 13775198 | | |
| M117 | LOW AFFINITY | ++ | 29089.3 | 12056967 | | |
| | IMMUNOGLOBULIN | | | | | |
| | GAMMA FC REGION | | | | | |
| | RECEPTOR III-A | | | | | |
| | PRECURSOR (IGG FC | | | | | |
| | RECEPTOR III-2) (FC- | | | | | |
| | GAMMA RIII-ALPHA) | | | | | |
| | (FC-GAMMA RIIIA) | | | | | |
| | (FCRIIIA) (FC-GAMMA | | | | | |
| | RIII) (FCRIII) (CD16-A) | | | | | |
| | (FCR-10) | | | | | |
| M121 | EPIDIDYMAL | − | 16570.3 | 5453678 | | |
| | SECRETORY PROTEIN | | | | | |
| | E1 PRECURSOR | | | | | |
| | (NIEMANN-PICK | | | | | |
| | DISEASE TYPE C2 | | | | | |
| | PROTEIN) (EPI-1) (HE1) | | | | | |
| | (EPIDIDYMAL | | | | | |
| | SECRETORY PROTEIN | | | | | |
| | 14.6) (ESP14.6) | | | | | |
| M123 | metastasin | − | 11846.7 | 4506765 | | |
| M128 | Similar to coactosin-like | − | 15945.1 | 1196417 | | |
| | protein | | | | | |
| M131 | VITAMIN-K | − | 52071.6 | 4506115 | | |
| | DEPENDENT PROTEIN C | | | | | |
| | PRECURSOR | | | | | |
| | (AUTOPROTHROMBIN | | | | | |
| | IIA) (ANTICOAGULANT | | | | | |
| | PROTEIN C) (BLOOD | | | | | |
| | COAGULATION | | | | | |
| | FACTOR XIV) | | | | | |
| M151 | D-DOPACHROME | − | 12711.8 | 4503291 | | |
| | TAUTOMERASE | | | | | |
| | (PHENYLPYRUVATE | | | | | |
| | TAUTOMERASE II) | | | | | |
| M180 | SERUM AMYLOID A | − | 12289.5 | 7531274 | | |
| | PROTEIN (SAA) | | | | | |
| | [CONTAINS: AMYLOID | | | | | |
| | PROTEIN A (AMYLOID | | | | | |
| | FIBRIL PROTEIN AA)] | | | | | |
| M195 | S-100P PROTEIN | − | 10400 | 5174663 | | |
| M204 | SERUM AMYLOID A-4 | − | 14806.8 | 10835095 | | |
| | PROTEIN PRECURSOR | | | | | |
| | (CONSTITUTIVELY | | | | | |
| | EXPRESSED SERUM | | | | | |
| | AMYLOID A PROTEIN) | | | | | |
| | (C-SAA) | | | | | |
| M484 | Unknown | + | 33413.9 | | 3 | 4 |

| | |
|---|---|
| 10¹⁰ and above | +++ |
| 10⁸ to 10¹⁰ | ++ |
| 10⁶ to 10⁸ | + |
| 0 | − |

TABLE 3A

| | | E_51 | E_59 | E_63 | E_67 | E_0 | N_52 | N_54 | N_58 | N_65 |
|---|---|---|---|---|---|---|---|---|---|---|
| Marker | Gene Name | Br38 | Br26 | Br35 | Br42 | Br43 | Br29 | Br28 | Br34 | Br36 |
| M31 | CALGRANULIN B | +++ | +++ | +++ | +++ | +++ | +++ | +++ | ++ | ++ |
| | (MIGRATION | | | | | | | | | |
| | INHIBITORY FACTOR- | | | | | | | | | |
| | RELATED PROTEIN 14) | | | | | | | | | |
| | (MRP-14) (P14) | | | | | | | | | |
| | (LEUKOCYTE L1 | | | | | | | | | |
| | COMPLEX HEAVY | | | | | | | | | |
| | CHAIN) (S100 CALCIUM- | | | | | | | | | |
| | BINDING PROTEIN A9) | | | | | | | | | |
| M43 | amyloid related serum | ++ | ++ | +++ | +++ | +++ | +++ | ++ | − | ++ |
| | protein SAA | | | | | | | | | |
| M44 | CALGRANULIN A | +++ | ++ | +++ | +++ | +++ | ++ | +++ | ++ | + |
| | (MIGRATION | | | | | | | | | |
| | INHIBITORY FACTOR- | | | | | | | | | |
| | RELATED PROTEIN 8) | | | | | | | | | |
| | (MRP-8) (CYSTIC | | | | | | | | | |
| | FIBROSIS ANTIGEN) | | | | | | | | | |
| | (CFAG) (P8) | | | | | | | | | |
| | (LEUKOCYTE L1 | | | | | | | | | |
| | COMPLEX LIGHT | | | | | | | | | |
| | CHAIN) (S100 CALCIUM- | | | | | | | | | |
| | BINDING PROTEIN A8) | | | | | | | | | |
| M60 | CALGRANULIN C (S100 | ++ | +++ | ++ | ++ | ++ | ++ | ++ | − | − |
| | A12 protein) | | | | | | | | | |
| M180 | SERUM AMYLOID A | − | − | ++ | + | + | − | − | − | − |
| | PROTEIN (SAA) | | | | | | | | | |
| | [CONTAINS: AMYLOID | | | | | | | | | |
| | PROTEIN A (AMYLOID | | | | | | | | | |
| | FIBRIL PROTEIN AA)] | | | | | | | | | |
| M204 | SERUM AMYLOID A-4 | − | − | − | − | − | ++ | − | − | − |
| | PROTEIN PRECURSOR | | | | | | | | | |
| | (CONSTITUTIVELY | | | | | | | | | |
| | EXPRESSED SERUM | | | | | | | | | |
| | AMYLOID A PROTEIN) | | | | | | | | | |
| | (C-SAA) | | | | | | | | | |

| | | | | | SEQ | SEQ |
|---|---|---|---|---|---|---|
| | | | | | ID | ID |
| | | N_66 | MW | GI | NO | NO |
| Marker | Gene Name | Br39 | (Da) | number | (nts) | (AA) |
| M31 | CALGRANULIN B | ++ | 13242.1 | 4506773 | | |
| | (MIGRATION | | | | | |
| | INHIBITORY FACTOR- | | | | | |
| | RELATED PROTEIN 14) | | | | | |
| | (MRP-14) (P14) | | | | | |
| | (LEUKOCYTE L1 | | | | | |
| | COMPLEX HEAVY | | | | | |
| | CHAIN) (S100 CALCIUM- | | | | | |
| | BINDING PROTEIN A9) | | | | | |
| M43 | amyloid related serum | + | 11682.8 | 4506777 | | |
| | protein SAA | | | | | |
| M44 | CALGRANULIN A | + | 10834.6 | 14729628 | | |
| | (MIGRATION | | | | | |
| | INHIBITORY FACTOR- | | | | | |
| | RELATED PROTEIN 8) | | | | | |
| | (MRP-8) (CYSTIC | | | | | |
| | FIBROSIS ANTIGEN) | | | | | |
| | (CFAG) (P8) | | | | | |
| | (LEUKOCYTE L1 | | | | | |
| | COMPLEX LIGHT | | | | | |
| | CHAIN) (S100 CALCIUM- | | | | | |
| | BINDING PROTEIN A8) | | | | | |
| M60 | CALGRANULIN C (S100 | − | 10443.9 | 2146972 | | |
| | A12 protein) | | | | | |
| M180 | SERUM AMYLOID A | − | 12289.5 | 7531274 | | |
| | PROTEIN (SAA) | | | | | |
| | [CONTAINS: AMYLOID | | | | | |
| | PROTEIN A (AMYLOID | | | | | |
| | FIBRIL PROTEIN AA)] | | | | | |
| M204 | SERUM AMYLOID A-4 | − | 14806.8 | 10835095 | | |
| | PROTEIN PRECURSOR | | | | | |
| | (CONSTITUTIVELY | | | | | |
| | EXPRESSED SERUM | | | | | |
| | AMYLOID A PROTEIN) | | | | | |
| | (C-SAA) | | | | | |

| | |
|---|---|
| 10¹⁰ and above | +++ |
| 10⁸ to 10¹⁰ | ++ |
| 10⁶ to 10⁸ | + |
| 0 | − |

TABLE 4

| | | E_51 | E_59 | E_63 | E_67 | E_70 | N_52 | N_54 | N_58 | N_65 |
|---|---|---|---|---|---|---|---|---|---|---|
| Marker | Gene Name | Br38 | Br26 | Br35 | Br42 | Br43 | Br29 | Br28 | Br34 | Br36 |
| M31 | CALGRANULIN B | +++ | +++ | +++ | +++ | +++ | +++ | +++ | ++ | ++ |
| | (MIGRATION | | | | | | | | | |
| | INHIBITORY FACTOR- | | | | | | | | | |
| | RELATED PROTEIN 14) | | | | | | | | | |
| | (MRP-14) (P14) | | | | | | | | | |
| | (LEUKOCYTE L1 | | | | | | | | | |
| | COMPLEX HEAVY | | | | | | | | | |
| | CHAIN) (S100 CALCIUM- | | | | | | | | | |
| | BINDING PROTEIN A9) | | | | | | | | | |
| M43 | amyloid related serum | ++ | ++ | +++ | +++ | +++ | +++ | ++ | − | ++ |
| | protein SAA | | | | | | | | | |
| M44 | CALGRANULIN A | +++ | ++ | +++ | +++ | +++ | ++ | +++ | ++ | + |
| | (MIGRATION | | | | | | | | | |
| | INHIBITORY FACTOR- | | | | | | | | | |
| | RELATED PROTEIN 8) | | | | | | | | | |
| | (MRP-8) (CYSTIC | | | | | | | | | |
| | FIBROSIS ANTIGEN) | | | | | | | | | |
| | (CFAG) (P8) | | | | | | | | | |
| | (LEUKOCYTE L1 | | | | | | | | | |
| | COMPLEX LIGHT | | | | | | | | | |
| | CHAIN) (S100 CALCIUM- | | | | | | | | | |
| | BINDING PROTEIN A8) | | | | | | | | | |
| M60 | CALGRANULIN C (S100 | ++ | +++ | ++ | ++ | ++ | ++ | ++ | − | − |
| | A12 protein) | | | | | | | | | |
| M65 | lumican | − | ++ | ++ | ++ | ++ | − | ++ | +++ | ++ |
| M86 | megakaryocyte stimulating | ++ | ++ | ++ | ++ | − | ++ | − | ++ | ++ |
| | factor | | | | | | | | | |

| | | | | | SEQ | SEQ |
|---|---|---|---|---|---|---|
| | | | | | ID | ID |
| | | N_66 | MW | GI | NO | NO |
| Marker | Gene Name | Br39 | (Da) | number | (nts) | (AA) |
| M31 | CALGRANULIN B | ++ | 13242.1 | 4506773 | | |
| | (MIGRATION | | | | | |
| | INHIBITORY FACTOR- | | | | | |
| | RELATED PROTEIN 14) | | | | | |
| | (MRP-14) (P14) | | | | | |
| | (LEUKOCYTE L1 | | | | | |
| | COMPLEX HEAVY | | | | | |
| | CHAIN) (S100 CALCIUM- | | | | | |
| | BINDING PROTEIN A9) | | | | | |
| M43 | amyloid related serum | + | 11682.8 | 4506777 | | |
| | protein SAA | | | | | |
| M44 | CALGRANULIN A | + | 10834.6 | 14729628 | | |
| | (MIGRATION | | | | | |
| | INHIBITORY FACTOR- | | | | | |
| | RELATED PROTEIN 8) | | | | | |
| | (MRP-8) (CYSTIC | | | | | |
| | FIBROSIS ANTIGEN) | | | | | |
| | (CFAG) (P8) | | | | | |
| | (LEUKOCYTE L1 | | | | | |
| | COMPLEX LIGHT | | | | | |
| | CHAIN) (S100 CALCIUM- | | | | | |
| | BINDING PROTEIN A8) | | | | | |
| M60 | CALGRANULIN C (S100 | − | 10443.9 | 2146972 | | |
| | A12 protein) | | | | | |
| M65 | lumican | ++ | 38429.2 | 4505047 | | |
| M86 | megakaryocyte stimulating | ++ | 151092 | 5031925 | | |
| | factor | | | | | |

| | |
|---|---|
| 10¹⁰ and above | +++ |
| 10⁸ to 10¹⁰ | ++ |
| 10⁶ to 10⁸ | + |
| 0 | − |

TABLE 5

| | | E_51 | E_59 | E_63 | E_67 | E_70 | N_52 | N_54 | N_58 | N_65 |
|---|---|---|---|---|---|---|---|---|---|---|
| Marker | Gene Name | Br38 | Br26 | Br35 | Br42 | Br43 | Br29 | Br28 | Br34 | Br36 |
| M483 | proapolipoprotein Varient | − | − | + | − | + | − | − | − | − |
| M484 | Unknown | − | − | + | + | − | − | − | − | − |
| M485 | neutrophil lactoferrin | − | − | + | − | − | − | − | − | − |
| M486 | alpha-2-HS glycoprotein | − | − | − | − | − | − | − | − | + |
| | Varient | | | | | | | | | |
| M487 | MT-11 protein Varient | − | + | − | − | − | − | − | − | − |
| M488 | immunoglobulin lambda | − | − | + | − | − | − | − | − | − |
| | light chain homolog | | | | | | | | | |
| M489 | Unknown | − | − | − | − | − | − | − | − | + |
| M490 | Unknown | − | − | − | − | − | − | + | − | − |

| | | | | | SEQ | SEQ |
|---|---|---|---|---|---|---|
| | | | | | ID | ID |
| | | N_66 | MW | GI | NO | NO |
| Marker | Gene Name | Br39 | (Da) | number | (nts) | (AA) |
| M483 | proapolipoprotein Varient | − | 25119.7 | | 1 | 2 |
| M484 | Unknown | + | 33413.9 | | 3 | 4 |
| M485 | neutrophil lactoferrin | − | 59529.6 | 186818 | | |
| | | | | (GenPept) | | |
| M486 | alpha-2-HS glycoprotein | − | 12290.2 | 10289662 | | |
| | Varient | | | (dbEST) | | |
| M487 | MT-11 protein Varient | − | 10172.9 | 7378207 | | |
| | | | | (dbEST) | | |
| M488 | immunoglobulin lambda | − | 16740.9 | | 5 | 6 |
| | light chain homolog | | | | | |
| M489 | Unknown | − | 21461.8 | | 7 | 8 |
| M490 | Unknown | − | 20895 | | 9 | 10 |

| | |
|---|---|
| 10¹⁰ and above | +++ |
| 10⁸ to 10¹⁰ | ++ |
| 10⁶ to 10⁸ | + |
| 0 | − |

TABLE 6
Calgranulin A protein concentration in pool
of SEC fractions 7, 8 and 9 of human serum

| | [Protein] | [Protein] | [Protein] | | |
|---|---|---|---|---|---|
| | (ng prot/ml | (ng prot/ml | (ng prot/ml | | |
| | serum) | serum) | serum) | | % |
| Sample Type | 1^{st} Injection | 2^{nd} Injection | 3^{rd} Injection | Average | RSD |
| Healthy | 7.6 | 6.0 | 11.6 | 8.4 | 28.0 |
| Non-Erosive | 7.0 | 5.0 | 5.7 | 5.9 | 14.1 |
| Erosive | 27.1 | 28.2 | 30.0 | 28.4 | 4.3 |

TABLE 7
Calgranulin B protein concentration in pool
of SEC fractions 7, 8 and 9 of human serum

| | [Protein] | [Protein] | [Protein] | | |
|---|---|---|---|---|---|
| | (ng prot/ml | (ng prot/ml | (ng prot/ml | | |
| | serum) | serum) | serum) | | % |
| Sample Type | 1^{st} Injection | 2^{nd} Injection | 3^{rd} Injection | Average | RSD |
| Healthy | 9.3 | 10.2 | 6.8 | 8.7 | 16.3 |
| Non-Erosive | 9.7 | 5.6 | 3.7 | 6.3 | 39.4 |
| Erosive | 129.1 | 112.4 | 127.6 | 123.0 | 6.2 |

TABLE 8A
Calgranulin C protein concentration in pool of SEC
fractions 7, 8 and 9 of human serum (Peptide 1)

| | [Protein] | [Protein] | [Protein] | | |
|---|---|---|---|---|---|
| | (ng prot/ml | (ng prot/ml | (ng prot/ml | | |
| | serum) | serum) | serum) | | % |
| Sample Type | 1^{st} Injection | 2^{nd} Injection | 3^{rd} Injection | Average | RSD |
| Healthy | 5.3 | 5.5 | 5.1 | 5.3 | 2.9 |
| Non-Erosive | 9.1 | 10.4 | 9.0 | 9.5 | 6.7 |
| Erosive | 81.7 | 78.3 | 80.2 | 80.1 | 1.7 |

TABLE 8B
Calgranulin C protein concentration in pool of SEC
fractions 7, 8 and 9 of human serum (Peptide 2)

| | [Protein] | [Protein] | [Protein] | | |
|---|---|---|---|---|---|
| | (ng prot/ml | (ng prot/ml | (ng prot/ml | | |
| | serum) | serum) | serum) | | % |
| Sample Type | 1^{st} Injection | 2^{nd} Injection | 3^{rd} Injection | Average | RSD |
| Healthy | 0 | 0 | 0 | 0 | 0 |
| Non-Erosive | 11.7 | 8.1 | 8.1 | 9.3 | 18.5 |
| Erosive | 92.3 | 69.6 | 93.2 | 85.1 | 12.8 |

TABLE 9A
Serum Amyloid A Concentration in Human Serum

| | Average SAA | Patient Number |
|---|---|---|
| Serum Sample | concentration (mg/L) | (n=) |
| Healthy | 4.8 | 35 |
| Disease Control | 5.5 | 9 |
| Non-erosive | 34.3 | 16 |
| Erosive | 115.8 | 17 |

TABLE 9B
One tail t-test Comparison of the SAA Concentrations of Erosive
and Non-Erosive to Healthy and Disease Controls

| Sample Comparison | 1 tail t-test p value |
|---|---|
| Erosive vs. Healthy | 0.000151 |
| Erosive vs. Disease Control | 0.033543 |
| Non-erosive vs. Healthy | 0.000001 |
| Non-erosive vs. Disease Control | 0.007497 |

TABLE 9C
One tail t-test Comparison of SAA concentrations
in Erosive and Non-erosive Serum Samples

| Sample Comparison | 1 tail t-test p value |
|---|---|
| Erosive vs. Non-erosive | 0.034980 |

What is claimed is:

1. A method of assessing whether a patient is afflicted with RA, the method comprising:
(a) determining the level of expression of one or more markers in a patient sample, wherein the one or more markers are selected from the group consisting of markers listed in Table 2;
(b) determining the normal level of expression of the one or more markers in a control sample; and
(c) comparing the level of expression of the one or more markers in the patient sample to the level of expression of the one or more markers in the control sample,
wherein a significant difference in the level of expression of the one or more markers in the patient sample compared to the normal level is an indication that the patient is afflicted with RA.

2. The method of claim 1, wherein the level of expression is determined by detecting the amount of marker protein present in the sample.

3. The method of claim 1, wherein the level of expression is determined by detecting the amount of mRNA that encodes a marker protein present in the sample.

4. A method of assessing whether a patient is afflicted with RA, the method comprising:
(a) determining the level of expression of a plurality of markers in a patient sample, wherein at least one of the markers is selected from Table 3A and the one or more additional markers are selected from the group consisting of markers listed in Table 2;
(b) determining the normal level of expression of the plurality of markers in a control sample; and
(c) comparing the level of expression of the plurality of markers in the patient sample to the level of expression of the plurality of markers in the control sample,
wherein a significant difference in the level of expression of the plurality of markers in the patient sample compared to the normal level is an indication that the patient is afflicted with RA.

5. A method of assessing whether a patient is afflicted with erosive RA, the method comprising:
(a) determining the level of expression of one or more markers in a patient sample, wherein the one or more markers are selected from the group consisting of markers listed in Table 2;
(b) determining the level of expression of the one or more markers in a control sample; and
(c) comparing the level of expression of the one or more markers in the patient sample to the level of expression of the one or more markers in the control sample,
wherein a significant difference between the level of expression of the one or more markers in the patient sample and the control is an indication that the patient is afflicted with erosive RA.

6. The method of claim 5, wherein the control is the level of expression of the one or more markers in a non-erosive RA patient sample.

7. The method of claim 5, wherein the level of expression is determined by detecting the amount of marker protein present in the sample.

8. The method of claim 5, wherein the level of expression is determined by detecting the amount of mRNA that encodes a marker protein present in the sample.

9. A method of assessing whether a patient is afflicted with RA, the method comprising:
(a) determining the level of expression of one or more markers in a patient sample, wherein the one or more markers are selected from the group consisting of markers listed in Table 3A;
(b) determining the level of expression of the one or more markers in a control sample; and
(c) comparing the level of expression of the one or more markers in the patient sample to the level of expression of the one or more markers in the control sample,
wherein a significant difference between the level of expression of the one or more markers in the patient sample and the control is an indication that the patient is afflicted with RA.

10. The method of claim 9, wherein the level of expression is determined by detecting the amount of marker protein present in the sample.

11. The method of claim 9, wherein the level of expression is determined by detecting the amount of mRNA that encodes a marker protein present in the sample.

12. An isolated nucleic acid molecule comprising a nucleotide sequence selected from the group consisting of SEQ ID NOs: 1, 3, 5, 7 and 9.

13. A vector which contains the nucleic acid molecule of claim 12.

14. A host cell which contains the nucleic acid molecule of claim 12.

15. An isolated polypeptide which is encoded by a nucleic acid molecule comprising a nucleotide sequence selected from the group consisting of SEQ ID NOs: 1, 3, 5, 7 and 9.

16. An antibody which selectively binds to the polypeptide of claim 15.

17. An isolated polypeptide comprising an amino acid sequence selected from the group consisting of SEQ ID NOs: 2, 4, 6, 8 and 10.

18. An antibody which selectively binds to the polypeptide of claim 17.
